(19) United States
(12) Patent Application Publication
(10) Pub. No.: US 2019/0040417 A1
(43) Pub. Date: Feb. 7, 2019

(54) BIOLOGICAL FERMENTATION USING DIHYDROXYACETONE AS A SOURCE OF CARBON

(71) Applicant: KEMBIOTIX LLC, Westford, MA (US)

(72) Inventors: Jay L. Milan, Westford, MA (US); Ramasamy Mannar Mannan, Weston, MA (US)

(73) Assignee: KEMBIOTIX LLC, Westford, MA (US)

(21) Appl. No.: 16/076,681

(22) PCT Filed: Feb. 8, 2017
(86) PCT No.: PCT/US17/17069

§ 371 Date: Aug. 8, 2018

Related U.S. Application Data
(60) Provisional application No. 62/292,924, filed on Feb. 9, 2016.

(51) Int. Cl. C12P 7/06; C12P 7/16; C12P 7/56; C12R 1/19; C12N 9/12

(52) U.S. Cl. CPC C12P 7/06; C12P 7/16; C12Y 207/0103; C12R 1/19; C12N 9/12; C12P 7/56

(57) ABSTRACT

The present invention relates to the use of hydrocarbons derived from natural gas in the fermentative production of biochemicals including biofuels. More specifically, the present invention provides the method for manufacturing dihydroxyacetone (“DHA”) from natural gas, biogas, biomass and CO₂ released from industrial plants including electricity-generating plants, steel mills and cement factories and the use of DHA as a source of organic carbon in the fermentative production of biochemicals including biofuels. The present invention comprises three stages. In the first stage of the present invention, syngas and formaldehyde are produced from natural gas, biogas, biomass and CO₂ released from industrial plants. In the second stage of the present invention, formaldehyde and syngas are condensed to produce DHA. In the third stage of the present invention, biochemicals including biofuels are produced from DHA using fermentation process involving wild type or genetically modified microbial biocatalysts.

### CROSS REFERENCE TO RELATED APPLICATION

This application claims the priority to the U.S. Provisional Application Ser. No. 62/292,924, filed on Feb. 9, 2016.

### FIELD OF THE INVENTION

This invention is in the field of producing a family of biochemicals including biofuels from natural gas, biogas, biomass and CO₂ using microbial biocatalysts.

### BACKGROUND OF THE INVENTION

There has been an impressive growth in manufacturing chemicals using microbial biocatalysts. Besides reducing toxic by-products, bio-based routes to chemical synthesis involving microbial biocatalysts may allow the use of new class of feedstocks. There is a growing expectation that lowered costs, increase in production speed, flexibility of manufacturing plants, and increased production capacity can be achieved using bio-based routes for chemical synthesis. The bio-based routes for chemical biosynthesis involve biological fermentation process. A number industrial fermentation processes for manufacturing a broad range of biochemicals including biofuels have been commercialized. The industrial biomanufacturing is considered to hold a great promise in meeting the evolving demands of chemical production in the current century and beyond (Clomburg et al. Industrial biomanufacturing: The future of chemical production. Science, 2017, 355: 6320 aag0804).

During the year 2015, the demand for the chemicals manufactured through fermentation process was 56.98 million tons and this demand is expected to reach 85.66 million tons by 2024 representing a compound annual growth rate of 4.6% during the period 2016-2024.

Representative examples of biochemicals that are suitable industrial scale production using biological fermentation include but not limited to ethanol, acetic acid, propionic acid, lactic acid, 3-hydroxy propionic acid, 1-3, propanediol, butanol, succinic acid, and muconic acid.

In the industrial fermentation process for the production of chemicals through bio-based routes, organic carbon in the form of fermentable carbohydrates such as glucose, sucrose and glycerol have been the primary raw material and often account for the largest single input cost. At present, dextrose derived from starch in grains and sucrose from sugarcane are primarily used in the industrial production of chemicals through biological fermentation. There has been some effort to use glycerol, obtained as a by-product from the biodiesel industry and fermentable sugars derived from the hydrolysis of cellulosic materials as a source of carbon in the industrial scale fermentation. Hexose and pentose sugars derived from cellulose are considered to yield cost-effective fermentable sugars. However, the technology to produce fermentable sugars from cellulose is not yet matured enough to support industrial scale fermentation process.

The cost of the feedstock used in the fermentation processes generally accounts for over 50%-70% of the product cost. It can be higher than 70% where the fermentation yields are low. When the biochemical products are made using fermentation processes, the cost of feedstocks is an important factor in the overall economy of the process. In most cases, petroleum feedstocks are abundant and cheap, making the products derived from petrochemical processes economically more competitive than the similar products derived from biological fermentation using microbial biocatalysts. For a fermentation process to be competitive against petrochemical processes in manufacturing a desired biochemical, the feedstock used in the fermentation process needs to be cost competitive. The cost of the feedstocks used in the biological fermentation process can be significantly reduced if they can be derived from fossil hydrocarbons. Methane is a fossil hydrocarbon and is abundantly available around the world. Methane can be converted into dihydroxyacetone (“DHA”) which can be used as a feedstock in the biological fermentation as described in this invention. The method to convert methane to DHA is scalable and relatively inexpensive process. One of the biggest advantages of using methane and fossil hydrocarbons to produce DHA according to the present invention is that methane is a gas and DHA is a solid which can be easily stored and transported from one place to another. On the other hand, the conventional dextrose feedstock used in the industrial fermentation process is available as 70% solution in water and needs to be kept above ambient temperature during transportation and storage to prevent crystallization.

Natural gas primarily composed of methane is reported to be present in abundant quantities in several regions of the world and is fast becoming a cheap feedstock to replace petrochemical feedstock. For example, natural gas is replacing the feedstocks derived from naptha-crackers for the manufacturing many chemical products.

There has been growing interest in manufacturing value added commodity chemicals from methane present in natural gas as feedstock. For examples, oxidative coupling of methane (OCM) has been developed to produce methanol, ethylene, propylene and butadiene using natural gas as a feedstock. Methanol to olefins (MTO) process is used to convert methanol into dimethyl ether (DME) and water. DME derived from MTO process is converted to olefins by a pyrolysis reaction. In another industrial application, methane present in the natural gas is converted to Fisher-Tropsch (FT) liquid and subsequently the FT liquid is converted to olefins by means of steam cracking. The olefins derived from natural gas are used as a primary building block to produce various polymers and consumer-focused functional materials. However, most of the current processes suffer from poor selectivity, high-energy cost and large CO2 emission.

There is a growing interest in using methane and its derivatives such as methanol, syngas, formate or formaldehyde as a source of organic carbon in the biological fermentation. A number of methanogenic microorganisms are considered as potential biocatalysts for the fermentative production of industrial chemicals using methane as a feedstock in the biological fermentation. However, we need to overcome several challenges before we develop a biocatalyst useful in commercial scale fermentative production of industrial chemicals using methane as a feedstock since methane is only sparingly soluble in aqueous media. On the other hand, it is highly desirable to convert methane present in the natural gas into simple carbohydrates which can be used as a feedstock in the fermentative production of various products such as organic acids, alcohols, fatty acid, isoprenoids, flavonoid, vitamins, olefins, antibiotics and amino acids using microbial biocatalysts.

The present invention provides fermentation processes to produce biochemicals including, but not limited to ethanol, lactic acid and butanol using DHA as a feedstock. Conventional biocatalysts using dextrose, glycerol, cellulosic hydrolysate and sucrose in the fermentative production of biochemicals are not able to use DHA as a feedstock. DHA is generally considered toxic to microbial growth beyond certain minimal concentrations. Therefore, it is necessary to subject the conventional biocatalysts to certain genetic modifications to confer the ability to use DHA as a sole or major source of carbon in the fermentative production of biochemicals. The present invention provides microbial biocatalysts and the processes in which DHA is used as a primary feedstock in the fermentative production of variety of biochemicals. According to the present invention, DHA can be used a sole source of organic carbon in the fermentation process. However, for the purpose of achieving an appropriate redox balance within the microbial biocatalyst, the microbial growth medium comprising DHA as source of carbon may be supplemented with an additional source of organic carbon such as glucose or glycerol.

The present invention provides novel approach to use methane as a feedstock in the commercial scale fermentative production of industrial biochemicals. Methane derived from natural gas as well as methane derived from biogas is suitable for the method for producing fermentable carbohydrates according to the present invention. Similarly, syngas derived from a number of industrial processes, biomass and carbon dioxide (“CO₂”) released from industrial plants are also suitable for producing fermentable carbohydrates useful in the present invention.

In the first stage of the present invention, formaldehyde is derived from methane, methanol, syngas or CO₂ released from industrial plants followed by the conversion of formaldehyde to DHA. DHA is a 3-carbon glycolytic intermediate in the metabolism of carbohydrate by almost all biological organisms. DHA does not accumulate in significant quantities within the living organisms and therefore beyond certain threshold level, DHA is considered as toxic to microbial biocatalysts generally used in the industrial scale fermentation process. In the second stage, the present invention provides a method to overcome the toxic effect of DHA on the growth of microbial organisms used in the industrial scale production of biochemicals including biofuels. According to the present invention, the toxic effect of DHA can be overcome by means of phosphorylating DHA as soon as it enters the microbial cells. to yield DHA phosphate (DHAP), a biological intermediate in the carbohydrate metabolism. The phosphorylation of DHA can be achieved using the endogenous enzymes already present within the microbial biocatalysts or introducing exogenous genes encoding for an enzyme that phosphorylates DHA.

### SUMMARY OF THE INVENTION

The present invention provides a biological fermentation process for producing biochemicals, including biofuels using wild type or genetically modified microorganism. The biological fermentation according to the present invention uses DHA as a source of carbon and energy. DHA suitable for the biological fermentation is derived from the condensation of formaldehyde which in turn is derived from natural gas, biogas, syngas or carbon dioxide (CO₂) released from industrial plants.

The wild type and genetically modified organisms useful for the present invention has the ability to grow in a medium comprising DHA in substantial quantities and are able to produce one or other commercially important biochemicals in commercially significant quantities. The genetically modified organism useful in the present invention is selected from a group consisting of gram-negative bacterium, gram-positive bacterium, cyanobacterium, archaea, algae, yeast and filamentous fungi. The list of the biochemicals that can be produced according to the present invention includes, but not limited to, ethanol, lactic acid, 3-hydroxypropionic acid, 1,3-propanediol, butanediol, butanol, succinic acid, amino acids and adipic acid.

The microbial biocatalysts suitable for the present invention has the ability to grow in the medium comprising DHA as a major source of carbon and energy. The ability of the microbial biocatalysts to grow in the medium containing substantial amount of DHA results either from an inherent ability to utilize DHA as a major source of carbon and energy or from genetic manipulations that confer the ability to utilize DHA as a major source of carbon and energy. Genetic manipulations of the microbial biocatalysts that are useful for the present invention are aimed at increasing the activity of one or other enzymes involved in the phosphorylation of DHA. In one aspect of the present invention, the genetic manipulations of the microbial biocatalysts according to the present invention involves the introduction of an exogenous gene coding for the protein involved in the phosphorylation of DHA. In another aspect of the present invention, the genetic manipulation of the microbial biocatalysts according to the present invention involves the manipulation of the endogenous genes coding for the proteins involved in the phosphorylation of DHA. The phosphorylation of DHA besides helping the microbial biocatalysts in overcoming the toxic effect of DHA, facilitates the entry of DHA into glycolytic cycle. In one aspect of the present invention, the increased phosphorylation of DHA is achieved by an increase in the activity of glycerol kinase which has the ability to phosphorylate DHA besides its natural substrate glycerol. In another aspect of the present invention, the increased phosphorylation of DHA results from an increase in the activity of the DHA kinase.

The increased activity of glycerol kinase and DHA kinase enzymes results either from an increased transcriptional activity of endogenous genes or from the introduction of one or more copies of an exogenous genes. In one aspect of the present invention, the exogenous DHA kinase uses ATP as the source of phosphate in phosphorylating DHA. In another aspect of the present invention, the exogenous DHA kinase depends on phopsphoenol pyruvate (“PEP”) for phosphorylating DHA.

In one embodiment of the present invention, the genetically modified microorganisms have the ability to grow and produce one or other biochemicals using DHA as a sole source of carbon and energy. In another embodiment of the present invention, the growth medium containing substantial amount of DHA is further supplemented with one or other carbohydrates hexose sugars such as glucose and fructose, pentose sugars, tetrose sugars, triose sugars, sucrose, glycerol or cellulosic hydrolysate.

In yet another embodiment of the present invention, the DHA derived from formaldehyde is subjected to an isomerization reaction to yield glyceraldehyde. DHA and glyceraldehyde can be condensed together using an aldolase enzyme to yield a hexose sugar which can be used as a source of organic carbon and energy in the fermentative production of one or other commercially useful biochemicals including biofuel. In another aspect of this embodiment, DHA can combine with one or two formaldehyde molecules to yield a tetrose or pentose sugars which in turn can be used as a source of organic carbon and energy in the fermentative production of commercially useful biochemicals including biofuels.

### BRIEF DESCRIPTION OF THE DRAWINGS

The following figures are included to illustrate certain aspects of the present invention, and should not be viewed as exclusive embodiments. The subject matter disclosed is capable of considerable modifications, alterations, combinations, and equivalents in form and function, as will occur to those skilled in the art and having the benefit of this disclosure.

FIG. 1. DHA production from methane through the intermediates including syngas, methanol and formaldehyde. Methane useful for the present invention is derived either from natural gas or bio-gas obtained from biogenic waste through anaerobic digestion. Syngas is produced from methane via oxidation reaction using chemical catalyst. Syngas could also be produced from biomass and other fossil hydrocarbon sources. Syngas comprising carbon monoxide and hydrogen reacts over chemical catalyst to produce methanol. Methanol could also be produced from methane through partial oxidation process. Formaldehyde useful for the present invention is produced from methanol through a catalytic oxidation method or catalytic dehydrogenation methods. Formaldehyde could also be directly produced through a partial oxidation of methane. DHA is derived from formaldehyde and syngas through chemical condensation reaction. DHA is capable of undergoing isomerization reaction to yield glyceraldehyde. Both DHA and glyceraldehyde can undergo hydrogenation reaction to yield glycerol which can be used as a source of organic carbon in the biological fermentation process for the production of commercially useful biochemicals including biofuels. In a preferred embodiment of the present invention, DHA is used as a source of organic carbon in the biological fermentation process.

FIG. 2. Multiple routes for the production of DHA. Syngas useful for the present invention is produced from natural gas by oxidation reactions using various chemical means. Syngas could also be produced from biomass and other fossil hydrocarbons. Methanol useful for the present invention is produced by reacting syngas over chemical catalyst. Methanol could also be produced from methane through partial oxidation process. Formaldehyde useful for the present invention is produced from methanol through a catalytic oxidation or catalytic dehydrogenation methods. Formaldehyde could also be directly produced through a partial oxidation of methane. Condensation of three molecules of formaldehyde over a chemical catalyst yields DHA. Condensation of two molecules of formaldehyde yields glycoldehyde. Condensation of a molecule of glycoldehyde with a molecule of formaldehyde yields DHA. DHA could also be produced by reacting formaldehyde and syngas over a catalyst. In another method, syngas comprising carbon monoxide and hydrogen is passed over glycoldehyde in the presence of a catalyst to produce DHA.

FIG. 3. Production of ethanol through biological fermentation using DHA as a source of organic carbon and energy. DHA useful in the ethanol fermentation is derived from CO₂ from industrial plants. CO₂ from industrial plants is reacted over chemical catalysts to produce methanol which in turn is subjected to chemical catalytic reaction to yield formaldehyde. Formaldehyde is subjected to self-condensation reaction to produce DHA useful in the fermentative production of ethanol.

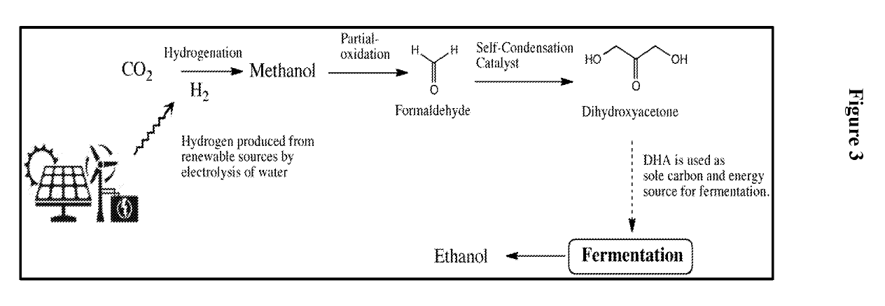

FIG. 4. Production of butanol through biological fermentation using DHA as a source of organic carbon and energy. DHA useful in the butanol production process is derived from biogas produced from biogenic waste through anaerobic digestion. Biogas comprising methane and CO₂ is subjected to CO₂ reforming process to yield syngas comprising carbon monoxide and hydrogen. Subsequently syngas is subjected to hydrogenation reaction to yield methanol which in turn is subjected to partial oxidation reaction to produce formaldehyde. In the presence of suitable chemical catalysts, formaldehyde undergoes self-condensation reaction to yield DHA which can be used as a sole carbon and energy source in the biological fermentation for producing butanol using a suitable biocatalyst.

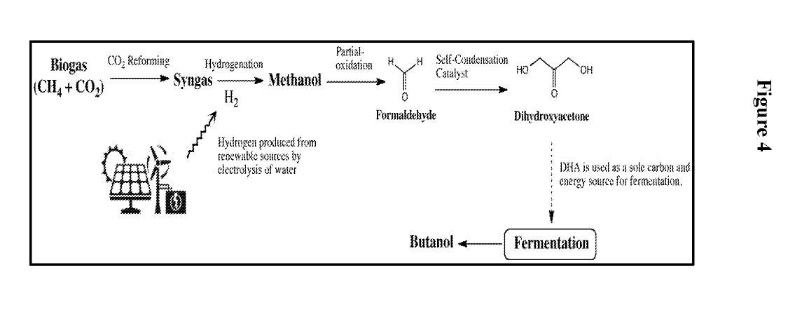

FIG. 5. Production of lactic acid through biological fermentation using DHA as a source of organic carbon and energy. DHA used in the fermentative production of lactic acid is derived from natural gas. In the first step of the process, natural gas is subjected to steam reforming or partial oxidation to yield syngas comprising carbon monoxide and hydrogen. In the next step syngas is hydrogenated to yield methanol which in turn is subjected to partial oxidation process to produce formaldehyde. In the presence of suitable chemical catalysts, formaldehyde undergoes self-condensation reaction to yield DHA which can be used as a sole carbon and energy source in the biological fermentation for producing D(−) lactic acid using a suitable biocatalyst.

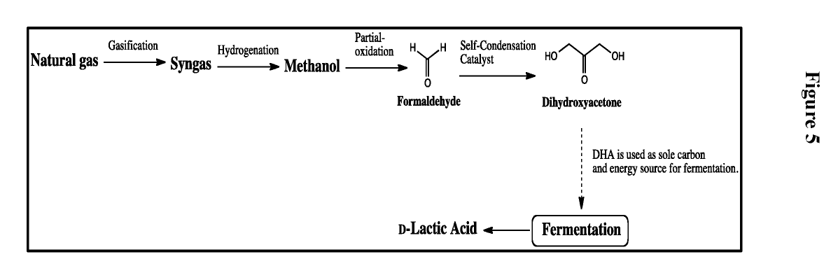

FIG. 6. The phosphotransferase system (“PTS”) is an energy-transducing system involved in carbohydrate uptake and control of carbon metabolism, which is ubiquitous in eubacteria. The PTS-dependent kinase of Escherichia coli consists of three subunits: DhaK contains the DHA binding site; DhaL contains ADP as cofactor for the double displacement of phosphate from DhaM to DHA; and DhaM provides a phosphor-histidine relay between the PTS and DhaL::ADP.

FIG. 7. A process flow diagram for a plant manufacturing methanol from methane. The methane or biogas is passed through desulfurization reactor (2) packed with zinc oxide beads. The gas is then heated in the central furnace (1) to approx. 420° C. From the furnace, the gas flows into the scrubber (4) to be saturated with the steam generated by water evaporator (3). Methane is heated in the furnace to 800° C. after being saturated with more steam to form desired mixture, and then directed into the reforming unit (5) for steam reformation reaction. Syngas at nearly 900° C. is then separated from excess steam by steam separator (6) and compressed to 60 atm by compressor (7) and is then combined with the unreacted reactants being recirculated from the methanol condenser and sent to two serially connected methanol synthesis reactors (8, 9). After the second synthesis reactor (9) the post reaction mixture is passed through water cooled methanol condenser (10). From the bottom part of the methanol separator, a crude methanol flows into serially connected methanol distillation columns. After the last distillation column, the purity of the methanol product is a minimum of 99.99%.

FIG. 8. A process flow diagram for a formaldehyde production from methanol. Compressed air is feed to the bottom of the methanol vaporizer (1). The ratio of methanol and air is maintained about 35-45%. This methanol:air mixture is heated to the reaction temperature of 550-600° C. by series of preheaters before entering the reactor (2). The reactor is a fixed bed type filled reactor with silver catalyst for converting methanol to formaldehyde. The product stream from the reactor is sent to the absorber (3) where the gaseous formaldehyde is absorbed into dioxane to form 30% formaldehyde solution. The product stream is sent to purification and then to recovery section (4). Unreacted methanol is fed back to the methanol vaporizer. Formaldehyde obtained as heavy end of the alcohol stripper column in the form of 30% solution in dioxane. Overall process yield is 95% on weight basis.

### DETAILED DESCRIPTION OF THE PREFERRED EMBODIMENTS

The present invention provides methods for producing DHA from fossil hydrocarbons, biogas, CO2 released from industrial plants and biomass. The DHA produced according to the present invention is useful as a source of organic carbon and energy in biological fermentation process involving microbial biocatalysts for producing biochemicals including biofuels.

The term “fossil hydrocarbons” as used in the present invention refers to hydrocarbons that have accumulated in the geological formations below the surface of the earth. Fossil hydrocarbons contain high percentages of carbon and include coal, petroleum and natural gas, shale gas, town gas, gaseous fuel obtained during crude oil drilling process, liquid refinery gas (LRG), crystallized natural gas such as methane clathrates. The term fossil hydrocarbons as used in the present invention also includes coal related products such as peat, lignite, sub-bituminous coal, bituminous coal, stream coal, anthracite, graphite, coke, tar sand, coal gas, coal to liquid products, refined coal and other coal derived products that can be converted to syngas. The term fossil hydrocarbons as used in the present invention also refers to gaseous fuels such as methane, ethane, propane and butane and liquid fuels including liquefied petroleum gas (LPG), fuel oil, gasoline, jet fuel, diesel fuel, heavy fuel oil, kerosene, liquefied refinery gas, still gas, coke, heating oil, lubricants, wax, asphalt, petroleum coke and petrochemicals. In preferred embodiment of the present invention, DHA is derived from methane present in the natural gas or biogas derived from anaerobic digestion of biogenic waste. In the most preferred embodiment of the present invention, DHA is derived from CO₂ released from industrial plants using fossil hydrocarbons. The carbon derived from fossil hydrocarbon can be differentiated from the carbon derived from biomass through C14/C13 carbon dating.

The term “methane” as used in the present invention refers to hydrocarbon with chemical structure “CH₄”. Methane used in the present invention is derived from natural gas, biogas, shale gas, town gas, liquefied natural gas (LNG), liquefied petroleum gas (LPG), liquefied refinery gas (LRG), gaseous hydrocarbon products released during crude oil drilling process, gaseous hydrocarbon products released during coal mining process, gaseous hydrocarbons trapped inside coal, gaseous hydrocarbons trapped inside rock, gaseous hydrocarbons trapped under ocean, crystallized natural gas such as methane clathrates, and methane hydrates. The term “C1 carbon source” as used in this invention refers to one carbon chemical building blocks such as methane present in the natural gas and in the biogas, methanol, formaldehyde and syngas.

The term “natural gas” as used in the present invention refers to the fossil hydrocarbons that occur in the gaseous form in the geological deposits under the surface of the earth.

The term “syngas” as used in the present invention refers to a “blend” of gaseous products carbon monoxide and hydrogen with chemical structure “(CO+H₂)”. The ratio of carbon monoxide and hydrogen in the “blend” varies depending on the feedstock used to produce syngas. The term “Syngas” used in the present invention refers to a blend of carbon monoxide and hydrogen with ratio from 1:1 to 1:4. Syngas used in the present invention is derived from methane, ethane, propane, butane, and other fossil hydrocarbon sources such as natural gas, shale gas, town gas, liquefied natural gas (LNG), liquefied petroleum gas (LPG), liquefied refinery gas (LRG), liquid fuels including fuel oil, gasoline, jet fuel, diesel fuel, heavy fuel oil, kerosene, coke, heating oil, lubricants, wax, asphalt, petroleum coke, petrochemical tar sand, hydrocarbon products released during crude oil drilling process, gaseous hydrocarbon products released during coal mining process, gaseous hydrocarbons trapped inside coal, gaseous hydrocarbons trapped inside rock, gaseous hydrocarbons trapped under ocean, crystallized natural gas, coal and coal related products such as peat, lignite, sub-bituminous, bituminous, stream coal, anthracite, graphite, coke, tar sand, coal gas, coal to liquid products, refined coal and other coal derived products. Syngas could also be derived from renewable carbon sources such as biomass, vegetable oil, municipal waste, and bagasse and distillers grain.

The term “biochemicals” as used in the present invention includes the biochemicals that can be manufactured in commercial scale using biological fermentation. The list of biochemicals that can be manufactured in commercial scale according to the present invention includes, but not limited to, organic acid, C2-C3 alcohols, C4-C10 alcohols, diols, isoprenoids and terpenoids, fatty acids, amino acids and their derivatives including aromatic amino acids and branched chain amino acids, vitamins, sterols, antibiotics, olefins and flavonoids. When a particular biochemical is present in isomeric forms, such as D(−) and L(+) lactic acid, it is possible to produce isomerically pure D(−) lactic acid or L(+) lactic acid using DHA as a source of carbon and the biological fermentation process according to the present invention.

The term “biomass” used in the present invention refers to renewable plant resources, which can be used to produce syngas used in this invention for the production of biochemicals including biofuels. Biomass is a product of photosynthetic carbon assimilation by green plants. The categories of biomass include. (1) Virgin wood from forestry and arboricultural activities; (2) Wood waste from wood processing; (3) High yield energy crops grown specifically for energy applications; (4) Agricultural residues from agricultural harvesting or processing; (5) Food waste from food and drink manufacturing and post-consumer waste; and (6) Industrial waste and co-products from manufacturing and industrial processes.

The term “carbohydrates” as used in this invention refers to the chemical compounds comprising carbon hydrogen and oxygen in the ratio of 1:2:1 and any derivatives thereof which can be used as source of carbon by the microbial biocatalysts useful for the biological fermentation for producing biochemical including biofuels according to the present invention. For example, aldotriose and ketotriose described below are carbohydrates useful for the present invention. The chemical derivatives of aldotriose and ketotriose such as glycerol will be considered as a carbohydrate according to the present invention.

The term “fermentable carbohydrates” as used in the present invention includes any of the aldose and ketose carbohydrates that can be used in the biological fermentation as a source of carbon and energy.

The term “biological feedstocks” or “feedstocks” as used in the present invention refers to any hydroxylcarbon substrate starting materials containing hydrogen, carbon and oxygen that are consumed and metabolized by organisms for their growth, used as a source of energy (fuel) and carbon used for the production of biochemicals. Biological feedstocks are typically derived from renewable resources such as agricultural products. Biological feedstocks are also derived from non-renewable resources such as fossil hydrocarbons through syngas by various chemical means. A typical example of a biological feedstock included in this invention is sugars such as glucose, sucrose and fructose, hydroxyl hydrocarbons such as glycerol, DHA and glyceraldehyde, cellulosic materials such as biomass. In some cases, organisms are engineered to directly metabolize unconventional feedstocks such as methane, syngas and liquid hydrocarbons. However, in the present invention, chemical methods are used to convert fossil based feedstocks to conventional sugar based biological feedstocks that are more easily consumed and metabolized by organisms.

The term “fermentation” as used in the present invention refers to the microbial process that yields one of other biochemical including biofuels in commercial quantities from fermentable carbohydrates using microbial biocatalysts. The fermentation process can be carried out under aerobic or microaerobic or anaerobic conditions based on the desired biochemical to be produced. The term “commercial quantities” as used in the present invention refers to the quantities of biochemical including biofuels that are produced in dedicated industrial plants for commercial sale as opposed to the quantities of the same biochemical produced in the lab scale for establishing the proof that DHA can be used as a source of organic carbon in the fermentative production of desired biochemical including biofuels.

The term “microbial biocatalyst” or “biocatalyst” as used in the present invention refers to the microbial organisms that can be useful in the fermentative production of biochemical including biofuels.

The present invention uses the term “aldose” to refer to monosaccharides that contains only one aldehyde group per molecule with the empirical formula Cₙ(H₂O)ₙ. The present invention uses the term “ketose” to refer to monosaccharides that contains only one ketone group per molecule with the empirical formula Cₙ(H₂O)ₙ. The present invention uses the term “triose” to refer to monosaccharide that contains 3 carbon atoms. The term “aldotriose” refer 3-carbon carbohydrates that contain one aldehyde group per molecule. The term “ketotriose” refer to 3-carbon compounds that contain one ketone group per molecule. DHA is a ketotriose carbohydrate and glyceraldehyde is an aldotriose carbohydrate. The present invention uses the term “tetrose” to refer to monosaccharide that contains 4 carbon atoms. The term “aldotetrose” refer 4-carbon carbohydrates that contain one aldehyde group per molecule. The term “ketotetrose” refer to 4-carbon carbohydrates that contain one ketone group per molecule. The present invention uses the term “pentose” to refer to monosaccharides that contains 5 carbon atoms. The term “aldopentose” refer to 5-carbon carbohydrates that contain one aldehyde group per molecule. The term “ketopentose” refer to 5-carbon carbohydrates that contain one ketone group per molecule. The present invention uses the term “hexose” to refer to monosaccharide that contains 6 carbon atoms. The term “aldohexose” refer to 6-carbon carbohydrates that contain one aldehyde group per molecule. The term “ketohexose” refer to 6-carbon carbohydrates that contain one ketone group per molecule. Aldoses and ketoses produced from formaldehyde according to the present invention are an isomeric mixture when produced using chemical catalyst or they can be in an isomerically pure form when produced through biological means.

The term “biofuels” as used in the present invention includes those biochemicals that can be manufactured using biological fermentation and are useful to replace or supplement gasoline in the transportation sector. The term biofuel includes but not limited to ethanol and butanol.

The term “titer” as used in the present invention refers to the quantity of specific biochemical produced in unit volume of the microbial culture medium in unit time (g/L/hr). The term “yield” as used in the present invention refers to the ratio of amount of a particular biochemical produced to the amount of feedstock material consumed. For example, in the fermentative production of ethanol using a bacterial biocatalyst using DHA as feedstock, the yield for ethanol production is a ratio of moles of ethanol produced to the moles of DHA consumed.

The term “host cell” is used interchangeably with “biocatalyst”, “microbial biocatalysts”, “microorganism” and “organism”. Generally, although not necessarily, the host cell is bacteria (including gram negative and gram positive bacteria), archaea, cyanobacterium, yeast, filamentous fungi or algae. Suitable eukaryotic host cells include, but are not limited to, fungal cells, algal cells, insect cells, and plant cells. Suitable fungal host cells include, but are not limited to, Ascomycota, Basidiomycota, Deuteromycetes, Fungi imperfecti, Saccharomyces cerevisiae, Saccharomyces sp., Schizosaccharomyces pombe, Pichia pastoris, Pichiajin-landica, Pichia trehalophila, Pichia kodamae, Pichia mem-branaefaciens, Pichia opuntiae, Pichia thermotolerans, Pichia salictaria, Pichia quercuum, Pichia pijperi, Pichia stipitis, Pichia methanolica, Pichia sp., Pichia angusta, Kluyveromyces sp., Kluyveromyces lactic, Candida albicans, Aspergillus nidulans, Aspergillus niger, Aspergillus oryzae, Trichoderma reesei, Chrysosporium lucknowense, Fusarium sp., Fusarium graminearum, Fusarium venenatum, and Neu-rospora crassa. Suitable algal host cells include, but are not limited to, Chlamydomonas reinhardtii and Phormidium sp. ATCC29409. Suitable bacterial hosts include, but are not limited to, any of a variety of gram-positive, gram-negative, or gram-variable bacteria such as microorganisms belonging to the genera Escherichia, Corynebacterium, Brevibacterium, Bacillus, Microbacterium, Enterobacterium, Serratia, Klebsiella, Erwinia, Pantoea, Pseudomonas, Agrobacterium, Alicyclobacillus, Anabaena, Anacystis, Arthrobacter, Azobacter, Chromatium, Envinia, Methylobacterium, Rhodobacter, Rhodopseudomonas, Rhodospirillum, Scenedesmun, Strepromyces, Synnecoccus, and Zymomonas. Examples of suitable host microorganisms used herein include Escherichia coli, LactoBacillus sp., Lactococcus lactis, Salmonella sp., Salmonella enterica, Salmonella typhi, Salmonella typhimu-rium, Shigella sp., Shigella.flexneri, Shigella sonnei, Shigella dysenteriae, Enterobacter sakazakii, Pseudomonas sp. D-0110, Pseudomonas pudica, Pseudomonas aeruginosa, Pseudomonas mevalonii, Rhodobacter sphaeroides, Rhodobacter capsulatus, Rhodospirillum rubrum; Rhodospirillum salexigens, Rhodospirillum salinarum, Rhodococcus sp., Mesorhizobium loti, Clostridium acetobutylicum, Clostridium tetani E88, Clostridium lituseburense, Clostridium saccharobutylicum, Clostridium perfringens, Clostridium beijerinckii, Fusobacterium nucleatum, Thermoanaerobacterium thermosaccharolyticum, Butyrivibrio jibrisolvens, Bacillus thuringiensis, Bacillus anthracis, Bacillus megaterium, Bacillus subtilis, Bacillus amylolique-facines, LactoBacillus johnsonii, Acinetobacter, Roseburia sp., Faecalibacterium prausnitzii, and Coprococcus sp., Sta-phylococcus epidermidis, Staphylococcus haemolyticus, Sta-phylococcus aureus, Brevibacterium ammoniagenes, Brevi-bacterium immariophilum ATCC14068, Brevibacterium saccharolyticum ATCC14066, Brevibacterium flavum ATCC14067, Brevibacterium lactofermentum ATCC13869, Corynebacterium glutamicum ATCC13032, Corynebacte-rium glutamicum ATCC14297, Corynebacterium acetoacidophilum ATCC 13870, Microbacterium ammoniaphilum ATCC15354, Serratia jicaria, Serratia fonticola, Serratia liquefaciens, Serratia marcescens, Agrobacterium radiobacter, Agrobacterium rhizogenes, Agrobacterium rubi, Anabaena cylindrica, Anabaena doliolum, Anbaena jlosaquae, Arthrobacter aurescens, Arthrobacter citreus, Arthrobacter globformis, Arthrobacter hydrocarboglutamicus, Arthro-bacter mysorens, Arthrobacter nicotianae, Arthrobacter parajfineus, Arthrobacte protophonniae, Arthrobacter roseoparajfinus, Arthrobacter sulfureus, Arthrobacter ureafaciens, Chromatium buderi, Chromatium tepidum, Chromatium vinosum, Chromatium warmingii, Chromatium fluviatile, Erwinia uredovora, Erwinia carotovora, Erwinia ananas, Er Ovinia herbicola, Erwinia punctata, Erwinia ferreus, Methylobacterium rhodesianum, Methylobacterium extorquens, Rhodopseudomonas blastica, Rhodopseudomonas marina, Rhodopseudomonas palustris, Streptomyces ambofaciens, Streptomyces aureofaciens, Streptomyces aureus, Streptomyces fungicidicus, Streptomyces griseochro-mogenes, Streptomyces griseus, Streptomyces lividans, Streptomyces olivogriseus, Streptomyces rameus, Streptomyces tanashiensis, Streptomyces vinaceus, Zymomonas mobilis. Suitable prokaryotic cells include, but are not limited to, any of a variety of laboratory strains of Escherichia coli, Lactobacillus sp., Salmonella sp., Shigella sp., and the like. Examples of Salmonella strains which can be employed in the present invention include, but are not limited to, Salmonella typhi and S. typhimurium. Suitable Shigella strains include, but are not limited to, Shigella jlexneri, Shigella sonnei, and Shigella disenteriae. Typically identified as oleaginous yeast include, but not limited to, Yarrowia, Candida, Rhodolorula, Rhodosporidium, Cryplococcus, Trichosporon and Lipomyces. More specifically, illustrative oleaginous yeasts include: Rhodosporidium Zoruloides, Lipomyces slarkeyii, L. lipoferus, Candida revkaufi, C. pulcherrima, C. Zropicalis, C. ulilis, T richosporon pullans, T culaneum, Rhodolorula glulinus, R. graminis and Yarrowia lipolylica Typically, the laboratory strain is one that is non-pathogenic. Non-limiting examples of other suitable bacteria include, but are not limited to, Bacillus subtilis, Pseudomonas pudita, Pseudomonas aeruginosa, Pseudomonas mevalonii, Rhodobacter, sphaeroides, Rhodobacter capsulatus, Rhodospirillum rubrum, Rhodococcus sp., and the like. In some embodiments host cell is Escherichia coli.

In the first stage of the process according to the present invention, suitable chemical catalysts are used to produce syngas from a fossil hydrocarbon, preferably methane. Methane useful for the present invention is derived from natural gas, shale gas, Town gas, liquefied refinery gas (LRG) and crystallized natural gas such as methane clathrate. In one aspect of the present invention, biogas derived from anaerobic digestion of biogenic waste can also be used as a source of methane. Syngas useful for the present invention can also be obtained directly from biomass through certain chemical conversion processes. However, syngas production from methane rich natural gas is highly preferred due to the lower cost and relative operational simplicity.

Two methods are available for the production of syngas from natural gas. Steam reforming method, an endothermic process, is widely used when hydrogen rich syngas is desired. For methanol related applications, an exothermic catalytic partial oxidation process is preferred due to its lower energy demand in large scale productions. CO₂, a common by-product of partial oxidation process, is also a precursor for methanol synthesis. NiO supported on various minerals such as silica, alumina, TiO₂ or CaO is generally used as a catalyst of choice in the partial oxidation process for the production of syngas from methane. Syngas production from natural gas is practiced in large scale by major oil companies for the gas to liquid (GTL) or methanol to gasoline (MTG) production processes.

As mentioned above, in another aspect of the present invention, syngas production from biogas is followed in the present invention. Biogas consists of methane and CO₂ along with some trace gases such as water vapor, H₂S, N₂, H₂ and oxygen. Purification methods are required to remove impurities to prevent catalyst poisoning. H₂S levels in the biogas needs to be reduced to ppm level before its use. H₂S is readily removed by adsorption onto the iron oxide catalyst or activated carbon. Iron sulfide, a by-product of the H₂S adsorption, is recycled by electrochemical methods for reuse. Sulfur, a by-product of the iron sulfide electrolysis, is processed to produce sulfuric acid or SO₂.

CO₂ reforming process known as Calcor process is a method of producing syngas from the reaction of CO₂ with hydrocarbons such as methane. Biogas produced from anaerobic digestion can be used as a feed for CO₂ reformation. CO₂ reforming utilizes both CO₂ and CH₄ in biogas to produce CO and H₂ useful for the methanol synthesis. CO₂ reforming of methane produces synthesis gas with a high CO/H₂ ratio, the ratio might vary based on the amount of CO₂ present in the biogas. High CO/H₂ ratio is desirable for oxo-synthesis and acetic acid production. However, methanol synthesis requires high H₂/CO ratio. The required hydrogen is supplied from an external source. Direct production of syngas with a wide range of H₂/CO ratio is efficiently accomplished by controlling the raw material compositions such as steam/methane ratios and CO₂/methane ratios. NiO supported on various minerals such as silica, alumina, TiO₂ or CaO is generally a catalyst of choice for many industrial scale CO₂ reforming process. Large scale production of syngas from CO₂ reforming is performed by the Chiyoda Corporation in Japan.

In the second stage of the process according to the present invention, syngas derived from methane or biomass is converted into methanol through a single step chemical process over a chemical catalyst. Methanol synthesis was first performed by BASF using ZnO/Cr₂O₃ catalyst at 250-350 bar and 350-420° C. (7). The ZnO/Cr₂O₃ catalyst is highly stable to sulfur and nitrogen impurities. However, chromium toxicity and high temperature/high pressure requirements for this catalyst limit its commercial viability. In 1996, a CuO/ZnO based catalyst was introduced for the methanol synthesis from sulfur free syngas containing high proportion of CO and hydrogen. Later, thermally stable CuO/ZnO/Al₂O₃ catalyst was developed and many variants of this catalyst are still being commercially used for the methanol synthesis. Copper based catalyst is extremely active and highly selective. Depending on the catalyst supplier, the reaction could be carried out at 220-250° C. at 50 bar, thereby avoiding premature aging of the catalyst. Most commercial methanol production facilities use CuO and ZnO with one or more stabilizing additives. The process is highly exothermic. Large portion of recycled H₂ is used to dilute the CO concentration at the inlet to about 10-15% to moderate the temperature rise. Methanol production from syngas is a commercially demonstrated technology, using both natural gas and coal as feedstock. The current world-class methanol plants are typically in the order of 2,000 to 2,500 metric tons per day. Large-scale (5,000 t/d) single train methanol process technologies are being offered. The list of major technology providers in this space include: (1) Toyo Engineering Corporation, (2) Lurgi Chemie GmbH, and (3) Foster Wheeler/Starchem.

Methanol useful for the present invention can also be produced from methane through partial oxidation process.

In another aspect of the present invention, methanol useful for the present invention can also be produced from hydrogenation of CO₂ generated either during syngas generation process or from the operation of industrial plants using fossil hydrocarbon as a source of energy. The chemical and petrochemical industry generates 18% of the direct industrial CO2 emission and steam cracking and ethylene production nearly accounts for 70% of CO2 emission from petrochemical industry.

CO₂ released from industrial plants is also useful as a source of carbon for manufacturing DHA useful in the present invention. Thus, the method for producing DHA from CO2 generated at the industrial plants using fossil hydrocarbons offers a novel carbon capture and utilization (CCU) technology. Large industrial plants such as power plants, steel mills and cement factories represent stationary single-point sources for large volume CO₂ suitable for manufacturing DHA useful in the present invention. Electricity generation contributes over 40% of CO₂ emissions from fossil fuels in U.S. Currently, U.S. power plants do not capture large volumes of CO₂ being released and the present invention introduces a novel carbon capture and utilization approach applicable for industrial plants.

First step in CCU is to capture CO₂ at the source and produce a concentrated clean stream for downstream processing. Currently, three main CCU methods are available. (1). Post-combustion capture involves extracting CO₂ from flue gas produced from coal based power plants. This method is expensive and accounts for 80-90% of the total cost of CCU. (2). Pre-combustion capture separates CO₂ from the fuel by combining the fuel with steam to produce hydrogen and CO₂. This process is only available from limited hydrogen producing facilities. (3). Oxy-fuel combustion capture uses oxygen instead of air for combustion and produces 85-90% pure CO2. However, use of oxygen instead of air is less preferred due to the higher cost.

CO₂ from exhaust gas contains small amounts of SO₂, CO, N₂ and NH₃. Purification methods are required to remove impurities to prevent catalyst poisoning. Currently, three main methods are available to remove the impurities in the CO₂ from exhaust gas. (1) Amine treatment methods are used trap CO₂ from flue gas and regenerate the CO₂ by thermal means. (2). Filtration membranes are designed to selectively let CO₂ pass through the membrane when the pore sizes are appropriate. (3). Condensation methods liquefy the CO₂ from impurities like CO and N₂. Pipelines are the most common method for transporting CO₂ in the United States. Currently over 4,100 miles of pipeline transport CO₂ predominately to oil and gas fields, where it is used for enhanced oil recovery. DHA production facility may be strategically located near an oil field to take advantage of the existing CO₂ pipeline infrastructure.

CO₂ to methanol conversion is achieved using chemical catalysts. Methanol synthesis was first performed by BASF using ZnO/Cr₂O₃ catalyst at 250-350 bar and 350-420° C. The catalyst was highly stable to sulfur and nitrogen impurities present in flue gas. However, chromium toxicity and high temperature, pressure requirements limit the commercial viability. In 1996, a CuO/ZnO based catalyst was introduced for the methanol synthesis for sulfur free CO2 containing high proportion of CO and hydrogen. Later, thermally stable CuO/ZnO/Al₂O₃ catalyst was developed and many variants of this catalyst are still being commercially used for the methanol synthesis. Copper based catalyst is extremely active and highly selective. Furthermore, the reaction could be carried out at 220-250° C. at 50 bar, thereby avoiding premature aging of the catalyst due to sintering of copper. Most commercial methanol production facilities use CuO and ZnO with one or more stabilizing additives. Carbon recycling International (CRI) has developed Emission to Liquid (ETL) technology that enables cost-effective conversion of CO₂ to methanol on pilot scale. Energy and hydrogen gas for the process is derived from renewable resources such as geothermal, solar, wind and hydro power.

In the third stage of the process according to the present invention, methanol is converted to formaldehyde through a single-step chemical process over a chemical catalyst. Formaldehyde has been produced commercially since 1889 by the catalytic oxidation of methanol following two different methods. In one method, methanol is subjected to partial oxidation and dehydrogenation reactions in the presence of silver crystals and steam at 680-720° C. under atmospheric pressure. Methanol conversion in this silver conversion process is 77-87% and formic acid, methyl formate, CO and CO₂ appear as typical byproducts. In the second process referred as Formox process, the oxidation of methanol is carried out with excess amount of air in the presence of Fe—Mo—V oxide catalyst at 250-400° C. under atmospheric pressure. Methanol conversion in the Formox process is 98-99% and the typical byproducts are formic acid, dimethyl ether and CO₂. The global production capacity of formaldehyde is 55 million metric tons a year. Major players in the formaldehyde markets are BASF, Dynea, Perstop, Georgia-Pacific Corp, Celanese and Ercros S.A. Major engineering and deployment firms such as Haldor Topsoe, Linde or Dynea provide engineering as well as operational support for building and operating pilot as well as demo scale methanol to formaldehyde production plants.

Formaldehyde useful for the present invention can also be produced from methane through partial oxidation process.

In the fourth stage of the process according to the present invention, formaldehyde is subjected to self-condensation reaction to yield DHA or glyceraldehyde. In the presence of bases, formaldehyde undergoes self-condensation reaction called formose reaction. However, formose reaction produces a complex mixture of racemic carbohydrates. DHA is a non-chiral three carbon sugar molecule. Chemical production of DHA from formaldehyde is a relatively simple and scalable process due to the lack of chirality. Moreover, the mild reaction conditions required for the formaldehyde self-condensation reaction required for the production of DHA prevents the formation of other complex sugar based byproducts. Thiazolium catalyst has been shown to catalyze the self-condensation of formaldehyde to DHA with up to 98% selectivity. From the economic stand point, formalin (37% formaldehyde in water) is a better feedstock than anhydrous paraformaldehyde. However, thiazolium based catalysts are deactivated in the presence of water. Several reports have recently appeared to solve this issue by continuous removal of water from the condensation reaction mixture. Using this process, 96% selectivity was achieved with the single pass conversion of around 30%. Similar DHA production methods are possible in industrial scale but were never practiced due to the lack of large market opportunity for DHA.

The conventional method for producing glycolaldehyde by the condensation of formaldehyde yields a mixture of DHA and glycolaldehyde. The reaction between hydridotetracarbonylcobalt and monomeric formaldehyde at 0° C. in the presence of 1 atm of carbon monoxide leads to stoichiometric hydroformylation with the formation of glycolaldehyde in high yield. (Roth et al, 1979, 1 organometallic chemistry. 172, C27-C28). A published international patent application (WO 2005/058788) provides rhodium based chemical catalyst capable of producing glycoldehyde from formaldehyde and syngas with up to 90% yield and selectivity.

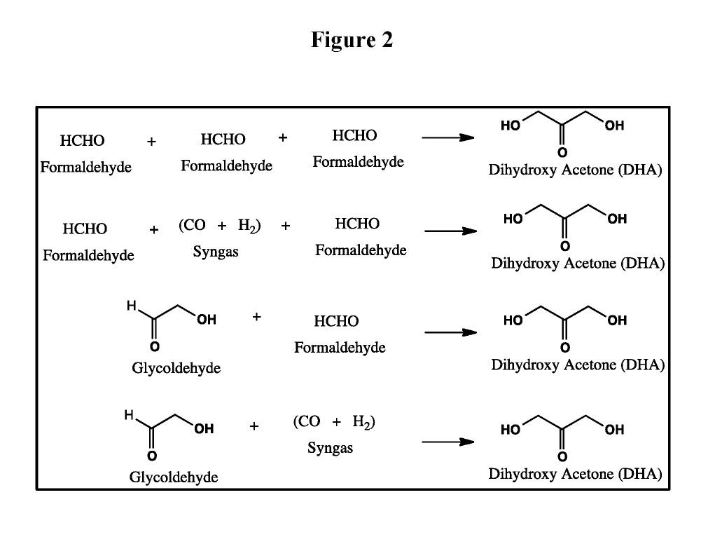

The production of DHA from syngas and formaldehyde can be achieved through four different pathways as illustrated in FIG. 2. Self-condensation of formaldehyde in the presence of a NHC catalyst is the most widely used method to produce DHA. Other methods such as cross-aldol condensation between glycolaldehyde and formaldehyde or glycolaldehdye and syngas can be used but are not well practiced in the industry. Matsumoto et al reported a condensation of paraformaldehyde catalyzed by thiazolium salt to produce DHA in high selectivity and yield (1 Am. Chem. Soc., 1984, 106, 4829-4832). A published European patent application (EU 1991/41063) from BP chemicals claims organic thiazolium based catalysts for the self-condensation of formaldehyde to DHA in a non-aqueous system containing formaldehyde and condensation catalyst. The reaction is carried out in the absence of the base to prevent the catalyst poisoning. Another European patent application (EU 1992/474387 claims the use of the thiazolium catalyst in aqueous system. The self-condensation reaction was promoted by the continuous removal of water from the reaction.

The production of glyceraldehyde from syngas and formaldehyde can be achieved through four different pathways. Cross-aldol condensation of glycolaldehyde with formaldehyde is the most widely practiced method to produce glyceraldehyde. This process can also be catalyzed by enzymes and other microorganisms to produce D-glyceraldehyde. Glyceraldehyde is also produced as mixture with glycolaldehyde by hydroformylation of formaldehyde and syngas. An enzymatic route to glyceraldehyde using D-Fructose-Phosphate aldolase (FSA) for direct syn cross-aldol addition of glycolaldehyde to formaldehyde in aqueous media has been developed (Garrabou et al, 2009, Angew. Chem. Int. Ed., 48, 5521-5525). It was found that glycolaldehyde has high affinity as a donor with formaldehyde and FSA. Cross-aldol reactions of glycolaldehyde with other less reactive aldehyde were also reported. A recent publication (Breslow et al, 2010, Proc Natl Acad Sci USA. 107, 5723-5725) discusses an amino acid based chemical catalyst for the synthesis of glyceraldehyde by reaction of formaldehyde with glycolaldehyde.

In another aspect of the present invention, two molecules of formaldehyde are condensed over a chemical catalyst to make glycolaldehyde. In another method to produce glycolaldehyde, syngas comprising carbon monoxide and hydrogen is passed over a catalyst and hydrogenated to produce glycloaldehyde as a single product or as a mixture with ethylene glycol. DHA useful for the present invention could also be made through condensation reaction of a molecule of glycolaldehyde with a molecule of formaldehyde. DHA could also be made through hydroformylation of formaldehyde using syngas over a chemical catalyst. In another method to produce DHA, syngas comprising carbon monoxide and hydrogen is passed over a chemical catalyst to produce DHA. In another aspect of this embodiment, three molecules of formaldehyde are condensed over a chemical catalyst to yield glyceraldehyde. Glyceraldehyde useful for the present invention could also be made through condensation reaction of a molecule of glycolaldehyde with a molecule of formaldehyde over a chemical catalyst. Glyceraldehyde could also be made through hydroformylation of formaldehyde using syngas over a chemical catalyst. In another method to produce glyceraldehyde, syngas is passed over a chemical catalyst to produce glyceraldehyde. In another method to produce glyceraldehyde, DHA is isomerized to produce glyceraldehyde by chemical or biological means.

In a preferred method, according to the present invention, the appropriate chemical catalysts with high selectivity for the production of DHA from syngas, methanol and formaldehyde are used.

Formose reaction is a self-condensation reaction of formaldehyde leading to the formation of mixture of sugars. Formose reaction is catalyzed by divalent base such as Ca, Ba, Tl and Pb. The intermediary steps taking place are aldol reactions, reverse aldol reactions, aldose-ketose isomerization. The reaction begins with two molecules of formaldehyde condensing to make glycolaldehyde, which further reacts with another equivalent of formaldehyde through aldol reaction to form glyceraldehyde. An aldose-ketose isomerization of glyceraldehyde forms DHA, which reacts with glycolaldehyde to form ketopentose. DHA can also react with formaldehyde to produce ketotetrose. The intermediates of formose reactions are a mixture of glycolaldehyde, glyceraldehyde, DHA, tetrose, pentose and hexose. Once the sugar has reached the size of C5 or C6 carbon atoms, they can form cyclic hemiacetals and deactivate the aldehyde from further reacting and thus formose reaction does not produce longer chain carbohydrates such as C7 and C8 sugars. Sugars produced from formose reactions are mixture of enantiomers and they are unsuitable for human consumption and other biological applications such as a feedstock in biochemical processes. Therefore, the preferred embodiment of the present invention aims at producing DHA as the main product in the formaldehyde self-condensation reaction.

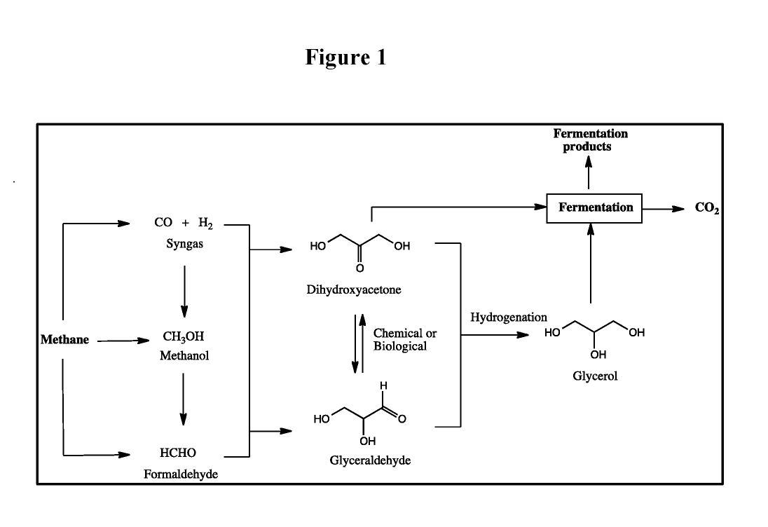

In another embodiment of present invention, glycerol is made from glyceraldehyde and DHA by hydrogenation reaction using hydrogen gas in the presence of chemical catalyst as illustrated in FIG. 1. Hydrogenation reaction can be performed using pure DHA, pure glyceraldehyde or a mixture of DHA and glyceraldehyde. In a preferred embodiment of the present invention, DHA is used as source of organic carbon and energy for the fermentative production of biochemicals including biofuels.

In yet another embodiment of the present invention, microbial biocatalysts with the ability to use DHA as a major source of organic carbon and energy in the production of value-added biochemicals including biofuels are provided. The microbial biocatalyst useful in the present invention includes microorganisms selected from a group consisting of gram negative bacterium, gram positive bacterium, algae, cyanobacterium, yeast and filamentous fungi. The microbial biocatalysts useful for the present invention are capable of growing in a medium comprising DHA as the major source of organic carbon and energy and produces value-added biochemicals including biofuels. The term “major source of organic carbon and energy” refers to the relative amount of a particular feedstock with reference to the amount of rest of the feedstocks in the fermentation medium. For example, if a fermentation medium contains a total of 10 grams/liter of feedstocks and the amount of dihydroxyacetone in the fermentation medium is 6 grams/liter, then dihydroxyacetone will be considered as the major source of organic carbon and energy in the fermentation medium when compared to other components of the feedstock. It is preferable to have dihydroxyacetone as the sole source of energy and carbon in the fermentation medium. However, in order to achieve proper redox balance within the biocatalyst to drive a particular metabolic pathway, it may be necessary to supplement the dihydroxyacetone in the fermentation medium with a small amount of other types of carbohydrates.

The biocatalysts useful in the present invention are either wild-type microorganisms obtained from Nature or microorganisms that have undergone certain genetic modifications that improves the utilization of DHA as a major source of organic carbon in the production of value-added biochemicals. In general, the wild-type microorganisms have multiple biochemical pathways leading to the production of a multiple biochemicals in small quantities simultaneously. In the industrial scale production of a value added biochemical, it is necessary to work with a biocatalyst that produces one particular desired biochemical in commercially significant quantities. This objective of producing a single value added biochemical in an industrial scale using a microbial biocatalyst is achieved by subjecting the selected biocatalyst to specific genetic modifications. Through specific genetic modifications, it is possible to block multiple biochemical pathways within a biocatalyst so that the production of those unwanted biochemicals are prevented and the microorganism is tailored to produce one specific biochemical in high-enough titer and yield. The term “titer” as used in the present invention refers to the quantity of specific biochemical produced in unit volume of the microbial culture medium in unit time (g/L/hr). The term “yield” as used in the present invention refers to the ratio of amount of a particular biochemical produced to the amount of feedstock material consumed. For example, in the fermentative production of ethanol using a bacterial biocatalyst using DHA, the yield for ethanol production is a ratio of moles of ethanol produced to the moles of DHA consumed.

Although it is desirable to use DHA as s sole source of organic carbon and energy in the fermentative production of a value-added biochemical using a selected biocatalyst in industrial scale, sometimes it would become necessary to use additional source of carbon such as glucose or glycerol for the purpose of balancing the redox potential within the selected biocatalyst so that the biochemical pathway leading to the production of desired biochemical functions within the biocatalyst are at the maximum efficiency. Under that situation, it becomes necessary to supplement the fossil fuel-derived DHA produced according to one of the method descried in the present invention with additional source of organic carbon such glucose and glycerol derived from renewable plant sources.

When natural gas-derived DHA is supplemented with plant-derived glucose or glycerol as an additional source of carbon it is possible to distinguish the contribution of DHA from the contribution of plant-derived additional carbon source such as glucose or glycerol in the production of desired value-added biochemical on the basis of the carbon 14 (¹⁴C) content of the resulting value-added biochemical following the method ASTM-D6866 provided by American Society of Testing and Materials. Cosmic radiation produces ¹⁴C (“radiocarbon”) in the stratosphere by neutron bombardment of nitrogen. ¹⁴C atoms combine with oxygen atom in the atmosphere to form heavy ¹⁴CO₂, which, except in the radioactive decay, is indistinguishable from the ordinary carbon dioxide. CO₂ concentration and the ¹⁴C/¹²C ratio is homogeneous over the globe and because it is used by the plants, the ratio ¹⁴C/¹²C in the atmosphere is retained by the biomass while the content of ¹⁴C in the fossil materials such as natural gas, originally derived from photosynthetic energy conversion, has decayed due to its short half-life of 5730 years. By means of analyzing the ratio of ¹⁴C to ¹²C, it is possible to determine the ratio of fossil fuel derived carbon to biomass-derived carbon. International Patent Application Publication No. WO2009/155085 A2 and U.S. Pat. No. 6,428,767 provide details about the use of ASTM-D6866 method for determining percent of biomass-derived carbon content in a chemical composition. The details related carbon dating disclosed in the U.S. Pat. No. 6,428,767 is incorporated herein by reference. An application note from Perkin Elmer entitled “Differentiation between Fossil and Biofuels by Liquid Scintillation Beta Spectrometry—Direct Method” provides details about the methods involving ASTM Standard D6866. Based on the information, it is desirable to select a biocatalyst which uses DHA as the primary or the sole source of carbon.

The genetic modifications conferring ability to a biocatalyst to produce one particular biochemical in commercially significant quantities using DHA as s major source of organic carbon can be carried out in multiple ways. In one aspect of the present invention, a biocatalyst already selected for producing a particular biochemical with high enough titer and yield at an industrial scale using conventional carbon sources such as glucose, glycerol or sucrose can be selected and subjected to specific genetic modifications that confer the ability to that particular biocatalyst to use DHA as s major or sole source of carbon to produce the desired biochemical with same titer and yield as in the growth medium containing conventional carbon source. For example, the bacterial biocatalysts already developed for the production of one or other desirable biochemical using glycerol or glucose or sucrose as the source of organic carbon can be subjected to specific genetic modification so that these biocatalysts gain the ability to use DHA as the sole or major source of organic carbon for the production of one or other desirable biochemical. The list of representative biocatalysts suitable for genetic modifications leading to the gain of function to use DHA as a sole or major source of organic carbon include but not limited to: (1) Escherichia coli strains genetically modified to produce ethanol at high titer and yield using glucose as a source of carbon. (2) Escherichia coli strains genetically modified to produce lactic acid at high titer and yield using glucose as a source of carbon. (3) Bacillus coagulans strains genetically modified to produce lactic acid at high titer and yield using glucose or cellulosic hydrolysate as a source of carbon. (4) Escherichia coli strains genetically modified to produce succinic acid at high titer and yield using glucose as a source of carbon. (5) Escherichia coli strains genetically modified to produce succinic acid at high titer and yield using sucrose as a source of carbon. (6) Escherichia coli strains genetically modified to produce succinic acid at high titer and yield using glycerol as a source of carbon. (7) Escherichia coli strains genetically modified to produce ethanol at high titer and yield using glycerol as a source of carbon. (8) Escherichia coli strains genetically modified to produce lactic acid at high titer and yield using glycerol as a source of carbon. (9) Escherichia coli strains genetically modified to produce butanol at high titer and yield using glucose as a source of carbon. (10) Escherichia coli strains genetically modified to produce butanol at high titer and yield using glucose as a source of carbon. (11) Escherichia coli strains genetically modified to produce 1,3 propanediol at high titer and yield using glucose as a source of carbon. It should be realized at this point that this list of ten biocatalysts and the associated fermentation product is provided only as examples to illustrate the spirit of the instant invention and the scope of the present invention is much broader. The present invention is suitable for use with a number of biocatalysts for producing broad range of biochemical using DHA as a feedstock as described in detail below.

DHA is the structurally simplest among all metabolizable carbohydrates useful as substrates in microbial fermentation. It is formed as intermediate in methanol assimilation by methylotrophic yeast and as the end product of dissimilative glycerol oxidation in the respiratory chain of Gluconobacter. DHA is taken up and metabolized by bacteria and eukaryotes. DHA entering into microbial cell is first reduced to glycerol. Glycerol is subsequently phosphorylated and the resulting glycerol phosphate enters into metabolic pathway. Alternatively, DHA can be phosphorylated to yield DHA phosphate (DHAP) which can enter into metabolic pathway. However, beyond certain concentration DHA is considered to be toxic to microbial growth. The present invention is based on the premises that by means of expressing DHA kinase at an increased level, it is possible to grow microbial organisms in a growth medium comprising DHA as a major source of carbon. There are two types of kinases namely glycerol kinase and DHA kinase that are responsible for the phosphorylation of DHA within the microbial cells. Glycerol kinase does not discriminate between glycerol and DHA. In contrast, DHA kinase is specific for DHA, D-glyceraldehyde and possibly other short chain aldehydes and ketones.

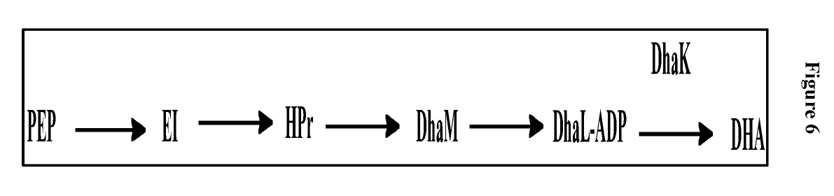

DHA kinases can be grouped in two structurally homologous but functionally different families namely ATP-dependent kinases and Phosphotransferase (PTS)-dependent kinases. The PTS is an energy-transducing system involved in carbohydrate uptake and control of carbon metabolism, which is ubiquitous in eubacteria but does not occur in archaebacterial and eukaryotes. The PTS-dependent kinase of Escherichia coli consists of three subunits: DhaK contains the DHA binding site; DhaL contains ADP as cofactor for the double displacement of phosphate from DhaM to DHA; and DhaM provides a phosphor-histidine relay between the PTS and DhaL::ADP (FIG. 6).

The present invention relates to the genetically engineered microorganisms utilizing DHA as a sole carbon energy source in the production of one or other value-added biochemical. It is presumed that DHA can enter the cell via the glycerol facilitator, GlpF, a non-specific channel protein capable of transporting straight-chain carbon compounds such as DHA. In 1984 Jin and Lin observed that a phosphoenolpyruvate:DHA phosphotransferase was induced in E. coli grown on DHA as a sole carbon source or in its presence. Subsequently in 2000, Paulsen et al showed that DHA can be utilized by E. coli as a sole exogenous source of carbon and energy. Paulsen et al employed Tn10 transposon mutagenesis to identify E. coli genes involved in the utilization of DHA. Tn10 insertion mutants were screened for loss of the ability to grow on DHA as sole carbon source and a single mutant Hfr7::Tn10, was obtained which was defective for growth on and phosphorylation of DHA, but retained the ability to grow on a variety of PTS sugars. The site of the transposon insertion in this mutant was mapped to the 26 min region of E. coli chromosome between the dadA and hemA genes. Two genes in this region, b1200 (ycg7) and b1199 (ycgS) encode proteins similar to the N- and C-terminal halves, respectively, of the DHA kinases from Citrobacter freundii, Saccharomyces cerevisiae and other organisms. Paulsen et al designated these genes dhaK1 and dhaK2 respectively. Further, Paulsen et al observed that the DHA phosphorylation defect in Hfr7::Tn10 could be complemented by the protein DhaK1 coded by dhaK1 gene alone. Therefore, Paulsen et al suggested that DhaK1 presumably plays a direct role in DHA phosphorylation, possibly functioning together with DhaK2 as a DHA kinase. Further insight into DHA kinase was provided by Gutknecht et al in 2001. According to Gutknecht et al DHA kinase (DhaK) of E. coli consists of three soluble protein subunits namely DhaK (YcgT; 39.5 kDa), DhaL (YcgS; 22.6 kDa) and homodimeric DhaM (YcgC; 51.6 kDa) consisting of three domains. The N-terminal dimerization domain has the same fold as the IIA domain of the mannose transporter of the bacterial phosphoenolpyruvate:sugar phosphotransferase system (PTS). The middle domain is similar to HPr and the C-terminus is similar to the N-terminal domain of enzyme I (EI) of the PTS. DhaM is phosphorylated three times by phosphoenolpyruvate in an E1- and HPr-dependent reaction. DhaK and DhaL are not phosphorylated. The IIA domain of the DhaM, instead of ATP, is the phosphoryl donor to DHA. It has also been suggested by Gutknecht et al that unlike the carbohydrate-specific transporters of the PTS, DhaK, DhaL and DhaM have no transport activity.

It has been demonstrated that DHA kinases in Saccharomyces cerevisiae are involved in detoxification of DHA. In S. cerevisiae, there are two genes namely dak1 and dak 2 coding for DHA kinase. Deletion of the both dak1 and dak2 genes makes the resulting yeast strain (dak1Δdak2Δ) highly sensitive to DHA. In the opposite case, overexpression of either DAK1 or DAK2 made the dak1Δdak2Δ strain highly resistant to DHA. Overexpression of either DAK protein provided cells with the capacity to grow efficiently on DHA as the only carbon and energy source.

In one aspect of the present invention, the genetically engineered microorganisms already known to produce one or other biochemical using conventional carbohydrate sources such as glucose, sucrose or glycerol is subjected to further genetic modification by means of transforming these microorganisms with the plasmids that code for the ATP or PEP dependent kinases that could phosphorylate DHA and facilitate its entry into the glycolytic cycle. The plasmids coding for the ATP or PEP dependent kinases can either be maintained as a self-replicating plasmid or stably integrated into the host chromosome. It is also possible to precisely integrate the gene coding for the ATP or PEP dependent kinases at precise location within the host chromosome.

It should be realized that certain biocatalyst already developed for use in industrial fermentation may be tolerant to high concentration of DHA in the growth medium due to a highly active endogenous DHA kinase enzymes which could efficiently phosphorylate DHA and reduce the toxicity associated with DHA. Under those circumstances there will not be any need to introduce exogenous kinases to phosphorylate the DHA to reduce the toxicity. It is also possible to subject the biocatalysts to metabolic evolution in the presence of increasing concentration of DHA in the growth medium so that the biocatalyst acquires required genetic changes and become resistant to DHA at higher concentration and becomes effective in using DHA as a sole source of carbon and energy.

In another aspect of the present invention, the genetically engineered microorganisms already selected to produce one or other biochemical using conventional carbohydrate source such as glucose, sucrose or glycerol is exposed to growth medium with increasing concentrations of DHA and subjected to metabolic evolution. Metabolic evolution as defined in this invention is a process for identifying those strains with the growth advantage over the rest of the population due to specific mutation that occurs when forced to grow in a growth medium comprising increasing concentrations of DHA. At the end of metabolic evolution those strains which have gained the ability to grow in the medium containing DHA as a major or the only source of organic carbon and still retain the original ability to produce desired biochemical at high enough titer and yield is identified and are subjected to whole genome sequencing. The genome sequence will be compared between the original parent strain and the metabolically evolved strains to identify the genetic modification in the metabolically evolved strain that confer the ability to use DHA as the major or sole source of organic carbon for the production of desired biochemical. Reverse genetic mutations are followed to validate the significance of the genetic modifications identified in the metabolically evolved strains through whole genome sequencing.

In another aspect of the present invention, the microbial strains selected for their ability to produce one or other biochemical in the growth medium comprising conventional carbon source such as glucose, sucrose or glycerol are subjected to standard chemical mutagenesis and the strains with the ability to grow and produce one or other desirable biochemical with high enough titer and yield in the growth medium comprising DHA as a sole or major source of carbon will be identified. Those microbial strains with the ability to grow in the growth medium with increasing concentrations of DHA and still retain the ability to produce desirable biochemical at high enough yield and titer will be subjected to whole genome sequencing and the identified mutations will be validated through reverse genetic engineering methods. Once a specific mutation associated with the ability to grow and produce biochemical in a medium comprising DHA is identified, such a mutation will be introduced into related microorganisms known to produce a different biochemical for the purpose of conferring the ability to use DHA as a sole or major source of organic carbon. For example, the genetic modification that confers the ability to use DHA as the sole or major source of organic carbon and energy in an ethanol producing biocatalyst will be incorporated into a lactic acid producing biocatalyst so that the lactic acid producing biocatalyst gains the ability to use DHA as s sole or major source of organic carbon and energy.

The biological fermentation according to the present invention is carried out using genetically modified biocatalysts including but not limited to gram-negative bacteria, gram-positive bacteria, yeast, filamentous fungi, and cyanobacteria. In general, the biocatalyst suitable for the present invention are subjected to genetic modifications that confer the ability to grow in a medium containing substantial amount of DHA as source of carbon for the production of one or other biochemical in commercially significant quantities. The genetically modified biocatalysts according to the present invention have the ability to grow and produce one or other biochemical in commercially significant quantities in a medium containing 1 g/L to 50 g/L of DHA as a source of carbon. In a preferred embodiment of the present invention, the genetically modified biocatalysts according to the present invention have the ability to grow and produce desired biochemical in commercially significant quantities in a medium containing 5 g/L to 25 g/L of DHA.

The biocatalysts useful in the present invention, besides having genetic modifications that confer ability to grow in a growth medium containing substantial amount of DHA, have additional genetic modifications that lead to the production of one or other biochemical in commercially significant quantities. In one embodiment of the present invention, in the first step, the wild type microorganisms are subjected to genetic modifications that confer the ability to grow in a growth medium containing substantial amount of DHA as a source of carbon. In the second step, those genetically modified microorganisms are subjected to further modifications to convert them into a biocatalyst with the ability to produce one or other biochemical in commercially significant quantity. In another embodiment of the present invention, in the first stage of the invention, microorganism with the ability to grow in a medium containing substantial amount of one or other convention carbon source including but not limited to glucose, glycerol and sucrose and ability to produce one or other biochemical in commercially significant quantity is generated or obtained from a microbial depository. In the second step of the invention, the microorganism from the first step is subjected to further genetic modification to confer the ability to grow in a growth medium comprising substantial quantity of DHA and produce one or other biochemical in commercially significant quantity.

In one embodiment, the present invention provides methods of producing organic acids such as malic acid, fumaric acid, succinic acid, citric acid, itaconic acid, lactic acid, malonic acid, 3-hydroxy butyric acid, 4-hydroxybutyric acid, 3-hydroxyisobutyric acid, 2-hydroxyisobutyric acid, mevalonic acid, pantothenic acid, 3-hydroxypropionic acid, fatty acid dicarboxylic acid, 3-hydroxyalkanoic acids, acetic acid, butyric acid and isobutyric acid using DHA as a major or sole source of carbon and energy.

In one aspect, the present invention provides genetically modified microorganisms producing specific organic acid are subjected to further genetic modifications to confer the ability to use DHA as a source of carbon. In another aspect of this invention, microorganisms that have ability to grow in a medium comprising DHA as a source of carbon is subjected to further genetic modifications to produce organic acids. Such a genetic modification would involve overexpressing one or more components of the malic acid, fumaric acid, succinic acid, citric acid, itaconic acid, lactic acid, malonic acid, 3-hydroxy butyric acid, 4-hydroxybutyric acid, 3-hydroxyisobutyric acid, 2-hydroxyisobutyric acid, mevalonic acid, pantothenic acid, 3-hydroxypropionic acid, fatty acid dicarboxylic acid and 3-hydroxyalkanoic acids biosynthetic pathway. The list of genetic manipulations required to enhance the organic acid biosynthesis within the microbial cell include: over expressing one or more of the enzymes in the malic acid biosynthetic pathway such as phosphoenol pyruvate carboxylase, pyruvate carboxylase and malate reductase; enhancing the activities of one or more enzymes in the citric acid biosynthetic pathway such as phosphoenol pyruvate carboxylase, pyruvate carboxylase and citrate synthase; enhancing the activities of one or more enzymes in the fumaric acid biosynthetic pathway such as phosphoenol pyruvate carboxylase, pyruvate carboxylase, malate reductase and fumarate hydratase; enhancing the activities of one or more enzymes in the succinic acid pathway such as phosphoenol pyruvate carboxylase, pyruvate carboxylase, malate reductase, fumarate hydratase and succinate dehydrogenase; enhancing the activities of one or more enzymes in the itaconic acid biosynthetic pathway such as phosphoenol pyruvate carboxylase, pyruvate carboxylase, citrate synthase and aconitase decarboxylate; enhancing the activities of one or more enzymes in the malonic acid pathway such as pyruvate dehydrogenase, acetyl-CoA carboxylase and malonate-CoA hydroxylase; enhancing the activities of one or more of the enzyme in the lactic acid biosynthetic pathway such as pyruvate kinase and lactate dehydrogenase; enhancing the activities of one or more of the enzymes in the 3-hydroxybutyric acid biosynthetic pathway such as acetyl-CoA acetyl transferase and 3-hydroxybutyryl-CoA reductase; enhancing the activities of one or more of the enzyme in the 2-hydroxyisobutyric acid biosynthetic pathways such as 3-hydroxybutyryl-CoA reductase and hydroxyisobutyryl-CoA mutase; enhancing the activities of one or more of the enzyme in the mevalonic acid biosynthetic pathway such as acetyl-CoA acetyl transferase and hydroxymethylglutaryl CoA synthase; enhancing the activities of one or more of the enzyme in the 4-hydroxybutyri acid biosynthetic pathway such as succinyl-CoA reductase and 4-hydroxybutyrate dehydrogenase; enhancing the activities of one or more of the enzymes in the 3-hydroxyisobutyric acid biosynthetic pathway such as 4-hydroxybutyrate dehydrogenase and 4-hydroxybutyryl-CoA mutase; enhancing the activities of one or more of the enzymes in the 3-hydroxypropionic acid pathway such as glycerol dehydratase and aldehyde dehydrogenase; enhancing the activities of one or more of the enzyme in the pantothenic acid biosynthetic pathway such as 2-dehydropantoate reductase and pantothenate-β-alanine ligase; and enhancing the activities of one or more of the enzyme in the fatty acid dicarboxylic acid biosynthetic pathway such as P450 hydroxylase and fatty alcohol oxidase.

Genetically modified microorganisms capable of producing “organic acids” using conventional sugars such as glucose, sucrose or glycerol are already known in the art. U.S. Pat. No. 8,900,835 provides genetically engineered thermotolerant microorganism with active lactate dehydrogenase (LDH) and inactive acetolactate synthase. U.S. Pat. No. 7,682,813 provides genetically engineered microorganism for producing lactic acid in high concentration and yield. U.S. Pat. No. 6,628,189 provides genetically engineered microorganism that has been altered with lactate dehydrogenase genes from Rhizopus oryzae. U.S. Pat. No. 6,258,587 provides genetically engineered L. johnsoni to selectively produce L-lactic acid. U.S. Pat. No. 8,137,953 provides genetically engineered yeast cells by reducing L or D lactate:ferricytochrome C oxidoreductase activity in the cell. This leads to reduced consumption of lactate in the cell and increased lactate yields. U.S. Pat. No. 7,534,597 provides genetically engineered microorganism where the exogenous LDH is integrated into the genome with deletion of native pyruvate decarboxylase (PDH) gene. U.S. Pat. No. 7,229,805 provides production of lactic acid using crabtree-negative yeast that has been transformed with LDH gene. U.S. Pat. No. 7,326,550 provides genetically engineered microorganism transformed with LDH gene and further modified for enhanced lactic acid production. U.S. Pat. No. 7,109,010 provides genetically engineered microorganism encoding heterologous LDH genes for producing lactate. U.S. Pat. No. 7,141,410 provides crabtree-negative cells comprising recombinant nucleic acid construct encoding proteins to produce lactic acid. U.S. Pat. No. 8,097,448 provides Issatchenkia orientalis transformed with exogenous LDH gene to produce lactate at low pH. U.S. Pat. No. 8,614,076 provides lactic acid producing genetically engineered microorganism where the racemic lactic acid is converted to either D or L lactic acid. U.S. Pat. No. 9,150,835 provides genetically engineered microorganism that express heterologous LDH enzymes for lactic acid production. U.S. Pat. No. 8,669,093 provides genetically engineered microorganism wherein Pfl, Dld genes are inactivated and ldhA gene is enhanced for the increased production of lactate. U.S. Pat. No. 8,871,489 provides alternative sugar transport system that decreases the activity of PEP dependent phosphotransferase system for the enhanced production of organic acids. U.S. Pat. No. 8,426,191 provides genetically engineered microorganism that has mgsA gene deleted or inactivated and produces chirally pure lactate. U.S. Pat. No. 7,629,162 provides genetically engineered microorganism that has inactivated mgsA, ackA, adhE, focA-pflB and frdA genes for the enhanced production of lactic acid. U.S. Pat. No. 5,416,020 provides genetically engineered Lactobacillus for the production of stereoisomerically pure lactic acid. U.S. Pat. No. 3,262,862 provides genetically engineered Sporolactobacillus for the production of lactic acid. U.S. Pat. No. 6,319,382 provides process in which lactic acid can be produced and isolated in a simple and inexpensive manner. U.S. Pat. No. 9,222,110 provides genetically engineered Enterococcus faecalis to produce lactic acid. U.S. Patent Application Publication No. 2014/0170719 reports recombinant microorganisms that has been transformed to produce lactate using heterologous LDH genes. U.S. Patent Application Publication No. 2010/0086980 reports genetically engineered microorganism producing lactic acid from glycerol. U.S. Patent Application Publication No. 2015/0240270 reports genetically engineered microorganism that are particularly tolerant to organic acids at low pH. U.S. Patent Application Publication No. 2005/0112737 reports genetically engineered microorganism producing high amounts of lactic acid and low-levels of byproduct impurities. U.S. Patent Application Publication No. 2007/0031950 reports genetically engineered microorganism encoding LDH functionally with deletion of PDH and PDC genes for lactate production. U.S. Patent Application Publication No. 2015/0299649 reports genetically engineered thermophilic Bacillus coagulans C106, J112, WCIPO-4 for highly efficient production of lactate. U.S. Patent Application Publication No. 2014/0128635 reports genetically engineered Zymomonas mobilis for the high optical pure and high yield lactate production. International PCT Application Publication No. WO2013/146557 provides genetically engineered microorganism to produce D-lactic acid from glycerol. International PCT Application Publication No. WO2010/143323 provides methods and processes to produce D-lactic acid. International PCT Application Publication No. WO2010/055363 provides acid resistant genetically engineered microorganism for the low-cost production of lactic acid. International PCT Application Publication No. WO2003/049525 provides genetically engineered Genera candida encoding LDH protein from Bacillus megaterum for lactate production. European Patent No. EP 2014/0178150 discloses genetically engineered microorganism comprising deletion of gene MgsA. Chinese Patent No. CN 2013/10293659 discloses recombinant microorganism comprising deletion of PflB, FrdABCD genes to produce lactic acid. Chinese Patent No. CN105062938 discloses recombinant microorganism wherein the replacement of AdhE gene with LdhA gene improves the production of lactate. Chinese Patent No. CN2013/10485910 discloses recombinant microorganism fto produce lactate from glycerol. Chinese Patent. No. CN2008/10024559 discloses recombinant microorganism to produce high optical purity lactate. A publication by Mazumdar et al (Escherichia coli strains engineered for homofermentative production of D-lactic acid from glycerol. Appl. Environ. Microbiol. 2010, 76, 4327-4336) discloses inactivation of PflB, FrdA, Pta, AdhE, Dld genes to produce lactate from glycerol.

U.S. Pat. No. 8,778,656 provides genetically engineered microorganism to produce succinic acid (SAC) in commercially significant quantities. U.S. Pat. No. 9,017,976 provides genetically engineered microorganism that are selected during metabolic evolution to contribute improved SAC productivity. U.S. Pat. No. 6,455,284 provides genetically engineered Escherichia coli where carbon flow is redirected towards overexpressed enzyme pyruvate carboxylase. U.S. Pat. No. 8,691,539 provides genetically engineered microorganism where LdhA, PflB, FocA, AckA and AdhE are deleted for the enhanced SAC production. U.S. Pat. No. 6,159,738 provides genetically engineered microorganism where the phosphotransferase system is altered for the increased production of SAC. U.S. Pat. No. 7,262,046 provides genetically engineered microorganism where reduced activity of phosphotransferase system due to a mutation in ptsG gene increased the SAC yield. U.S. Pat. No. 7,244,610 provides genetically engineered microorganism for SAC production under aerobic culture where genes PtsG, Ic1R and SdhAB are inactivated. U.S. Pat. No. 7,233,567 provides genetically engineered microorganism where genes IdhA, AdhE, Ic1R and Ack are inactivated for increased SAC production. U.S. Pat. No. 6,448,061 provides genetically engineered double mutant E. coli SS373 for improved SAC production. U.S. Pat. No. 7,763,447 provides genetically engineered microorganism comprising modified fumarate reductase and succinate dehydrogenase genes for SAC production. U.S. Pat. No. 5,770,435 provides genetically engineered microorganism that has been derived from parent lacking Pfl and Ldh genes. U.S. Pat. No. 8,497,104 provides genetically engineered microorganism with enhanced phosphoenolpyruvate carboxykinase activity and decreased glucose phosphotransferase activity. U.S. Pat. No. 5,504,004 provides genetically engineered bacterium 130Z for making SAC. U.S. Pat. No. 9,217,138 provides genetically engineered microorganism obtained by disrupting LdhA, Pta, AckA and Pfl for high growth rate and SAC productivity. U.S. Pat. No. 8,962,272 provides genetically engineered microorganism to produce SAC on low cost sugar source such as sucrose. U.S. Pat. No. 8,647,843 provides methods for adjusting oxygen transfer rate for high SAC production. U.S. Pat. No. 7,993,888 provides genetically engineered microorganism having high 2-oxoglutarate dehydrogenase activity for improved SAC production. U.S. Pat. No. 7,927,859 provides increasing the yield of SAC by increasing the intracellular availability of NADH. U.S. Pat. No. 7,790,416 provides genetically engineered microorganism obtained by disrupting Ldh, Adh, Ic1R and Ack genes for improved SAC production. U.S. Pat. No. 6,455,284 provides metabolic engineering approached to increase the carbon flow towards oxaloacetate to enhance to production of succinic acid. U.S. Pat. No. 7,829,316 provides genetically engineered microorganism obtained by increasing the activity of the protein coded by SucE1 gene to produce SAC. U.S. Pat. No. 7,972,823 provides genetically engineered microorganism obtained by decreasing acetyl-CoA hydrolase to improve SAC production. U.S. Pat. No. 7,368,268 provides genetically engineered microorganism obtained by disrupting Ldh gene and overexpressing pyruvate carboxylase gene to produce SAC at high yield. U.S. Pat. No. 8,877,466 provides genetically engineered microorganism having glyoxylate shunt for the production of SAC. U.S. Pat. No. 8,883,466 provides genetically engineered microorganism having formate dehydrogenase activity for the production of SAC. U.S. Pat. No. 9,023,632 provides genetically engineered microorganism using glycerol as a carbon source to produce SAC. U.S. Pat. No. 7,572,615 provides genetically engineered microorganism having FdhD and FdhE genes for the improved SAC production. U.S. Pat. No. 5,521,075 provides genetically engineered fluoroacetate variant of Anaerobiospirillum succiniciproducens for increased SAC production. U.S. Patent Application Publication No. 2007/0042481 reports genetically engineered microorganism encoding formate dehydrogenase D and E for enhanced SAC production. U.S. Patent Application Publication No. 2010/0297716 reports genetically engineered Enterobacteriaceae with enhanced phosphoenolpyruvate carboxykinase activity for improved SAC productivity. U.S. Patent Application Publication No. 2007/0154999 reports genetically engineered SAC producing microorganism where pyruvate oxidase activity is decreased. U.S. Patent Application Publication No. 2015/0284746 reports genetically engineered microorganism with increased activity of Pck gene and inactivation of AdhE, IdhA, FocA, PflA and Ack for increased SAC productivity. U.S. Patent Application Publication No. 2007/0111294 reports genetically engineered microorganism for the growth coupled production of SAC. U.S. Patent Application Publication No. 2003/0087381 reports genetically engineered microorganism for the enhanced production of oxaloacetate derived biochemical including SAC.

U.S. Pat. No. 8,192,965 provides genetically engineered yeast cells using glycerol as a carbon source to produce itaconic acid. U.S. Pat. Nos. 5,637,485 and 5,457,040 provide genetically engineered Aspergillus species to produce itaconic acid under aerobic microbial fermentation using glycerol as a part carbon source. U.S. Pat. No. 8,642,298 provides genetically engineered microorganism comprising itaconate transporting major facilitator superfamily transporter for the enhanced production of itaconate. U.S. Pat. No. 7,479,381 provides genetically engineered Pseudozyma antarctica NRRL Y-30980 for the high yield production of itaconate. U.S. Pat. No. 8,679,801 provides genetically engineered microorganism encoding Aspergillus mitochondrial tricarboxylic transporter for the production of itaconic acid. U.S. Pat. No. 5,231,016 provides economical production of itaconic acid using starch as one of the source of carbon. U.S. Pat. Nos. 2,657,173, 2,385,283, 2,462,981, 3,078,217 and 3,162,582 provides genetically engineered Aspergillus species for the enhanced itaconate production. U.S. Pat. No. 8,143,036 provides genetically engineered microorganism comprising enzymes cis-aconitate decarboxylase, phosphoenol pyruvate decarboxylase, citarate synthase and aconitase for the increased production of itaconic acid. U.S. Pat. No. 6,171,831 provides genetically engineered Aspergillus species for the fermentative production of itaconic acid. U.S. Pat. No. 8,440,436 provides genetically engineered A. niger comprising cis-aconitic acid decarboxylase derived from A. terreus for the enhanced production of itaconate. U.S. Patent Application Publication No. 2016/0060660 discloses cultivation of a genetically engineered host cell comprising mammalian Irg1 gene to produce itaconic acid. International PCT Application. Publication No. WO2015/181312 provides genetically engineered microorganism comprising activation of cis-aconitase decarboxylase activity and deactivation of trans-aconitase methyltransferase for the enhanced production of itaconic acid. International PCT Application. Publication No. WO2015/140314 provides genetically engineered microorganism encoding aconitase-delta-isomerase and trans-aconitase decarboxylase for the increased production of itaconic acid.

U.S. Pat. No. 5,100,789 provides genetically engineered Saccharomycopsis fibuligera to produce mevalonic acid. U.S. Pat. No. 5,116,757 provides genetically engineered Saccharomycopsis fibuligera that are resistant to ML-236B for the production of mevalonate. U.S. Pat. No. 5,149,641 provides genetically engineered Saccharomycopsis fibuligera IFO 0107 for the production of mevalonate. U.S. Pat. No. 3,617,447 provides genetically engineered Endomycopsis fibuliger NRRL Y-7069 for the enhanced production of mevalonate. U.S. Pat. No. 9,121,038 provides recombinant microorganism comprising heterologous expression of MvaE and MvaS genes for the increased production of mevalonate, isoprene and iosprenoids. U.S. Patent Application Publication No. 2005/0287655 discloses recombinant microorganism encoding 3-hydroxy-3-methylglutaryl CoA enzyme that to produce mevalonic acid in high yields.

U.S. Pat. Nos. 4,564,594 and 4,877,731 provides fermentation process to produce fumaric acid where the improvement comprises growing fungi of genus Rhizopus arrhizus. U.S. Pat. No. 8,735,112 provides fumaric acid producing recombinant microorganism comprising heterologous enzyme that catalyzes the conversion of malic acid to fumaric acid. U.S. Pat. Nos. 2,861,922, 2,327,191, 2,326,968, and 2,912,363 provides methods and processes for the production of fumaric acid by means of fumaric acid producing fungi. U.S. Patent Application Publication No. 2014/0045230 discloses recombinant fungus comprising an enzyme which catalyzes the conversion of malic acid to fumaric acid in the cytosol.

International PCT Application. Publication No. WO2013/134424 provides genetically engineered microorganism comprising heterologous malonyl-CoA hydrolase enzyme to produce malonic acid from malonyl-CoA. International PCT Application. Publication No. WO2015/200545 provides genetically engineered microorganism encoding MAE1 transport proteins that increases the production of malonate by the host cell.

U.S. Pat. No. 8,999,685 provides malic acid producing recombinant microorganism encoding heterologous malate dehydrogenase and pyruvate carboxylase enzyme. U.S. Pat. No. 3,063,910 provides recombinant Aspergillus species to produce malic acid. U.S. Pat. No. 9,062,330 provides recombinant microorganism comprising genes encoding enzymes imparting the production of malate and fumarate. U.S. Pat. No. 9,187,772 provides recombinant microorganism lacking fumarate reductase activity and increased phosphoenol pyruvate carboxykinase activity for the enhanced production of malate. U.S. Pat. No. 4,912,043 provides malic acid producing recombinant microorganism having resistant to 2-aminobutyric acid. U.S. Patent Application Publication No. 2008/0090273 discloses recombinant yeast comprising pyruvate carboxylase activity, malate dehydrogenase activity and malic acid transporter protein activity for the increased production of malic acid.

U.S. Pat. No. 8,883,464 provides genetically engineered microorganism comprising inhibitor of fatty acid synthase and increased malonyl-CoA reductase activity leading to increased utilization of malonyl-CoA to produce 3-hydroxypropionic acid (3HP). U.S. Pat. No. 8,809,027 provides genetically engineered microorganism comprising oxaloacetate alpha-decarboxylase and 3-hydroxypropionate dehydrogenase enzymatic activity for the increased 3HP production. U.S. Pat. No. 8,889,391 provides genetically engineered microorganism comprising beta-alanine/alpha-ketoglutarate aminotransferase and 3-HP dehydrogenase enzymatic activity for the increased 3HP production. U.S. Pat. No. 8,951,763 provides genetically engineered microorganism comprising glycerol dehydratase and aldehyde dehydrogenase enzymes to produce 3HP from glycerol. U.S. Pat. No. 8,124,388 provides genetically engineered microorganism comprising beta-alanine/alpha-ketoglutarate aminotransferase activity to produce 3HP from beta-alanine. U.S. Pat. No. 9,090,919 provides genetically engineered microorganism having ability to produce coenzyme B12 required for enzyme glycerol dehydratse and produce 3HP from glycerol. U.S. Pat. No. 9,090,918 provides genetically engineered yeast having active aspartate 1-decarboxylase enzyme and produce 3HP in high productivity from beta-alanine. U.S. Pat. No. 8,673,601 provides genetically engineered microorganism for the growth coupled production of 3-hydroxypropionic acid. U.S. Pat. No. 8,999,700 provides genetically engineered microorganism to produce 3HP from transformed cell comprising various engineered metabolic pathways. U.S. Pat. No. 7,186,541 provides genetically engineered microorganism to produce 3HP using several methods and metabolic pathways. U.S. Pat. No. 8,541,212 provides genetically engineered microorganism inhibiting the expression of lactate dehydrogenase, phosphotransacylase and alcohol dehydrogenase to produce 3HP using malonate semialdehyde reduction pathway. U.S. Pat. No. 6,852,517 provides genetically engineered microorganism encoding glycerol dehydratse and alcohol dehydrogenase for the production of 3HP from glycerol. U.S. Patent Application Publication No. 2012/0244588 discloses recombinant microorganism having activity of malonyl CoA reductase and malonate semialdehyde reductase to produce 3HP. U.S. Patent Application Publication No. 2015/0056684 discloses genetically modified microorganism that exhibits increased tolerance to 3HP. U.S. Patent Application Publication No. 2009/0325248 discloses genetically modified microorganism comprising increased activity of enzymes phosphoenol pyruvate carboxylase and aspartate 1-decarboxylase. U.S. Patent Application Publication No. 2011/0144377 discloses genetically modified microorganism comprising glycerol dehydratase and aldehyde dehydrogenase for the enhanced production of 3HP. U.S. Patent Application Publication No. 2010/0021978 discloses genetically modified microorganism comprising increased activities of enzymes 2-ketoacid decarboxylase, oxaloacetate dehydrogenase and malate decarboxylase to produce 3HP. U.S. Patent Application Publication No. 2015/0240269 discloses genetically engineered microorganism comprising increased activities of enzymes glycerol dehydratase and aldehyde dehydrogenase to produce 3HP from glycerol. U.S. Patent Application Publication No. 2014/0154760 discloses genetically modified microorganism with increased activities of enzymes glyceraldehyde-3-phosphate dehydrogenase, glycerate dehydratase and melonate semialdehyde dehydrogenase enzymes to produce 3HP. International PCT Application. Publication No. WO2013/095009 discloses succinate semialdehyde enzyme for the oxidation of 3-hydroxypropionaldehyde to 3-hydroxypropionic acid. International PCT Application. Publication No. WO2015/017721 provides genetically engineered microorganism comprising aspartate 1-decarboxylase from Insecta, Bivalvia, Branchiopoda for the production of 3HP. International PCT application. No. WO2013/137277 provides genetically engineered microorganism comprising enzymes malonate-CoA reductase and malonate-semialdehyde dehydrogenase to produce 3HP.

U.S. Pat. No. 6,316,262 provides genetically engineered microorganism encoding 4-hydroxybutyryl-CoA transferase or polyhydroxyalkanoate synthase to produce polyhydroxybutyric acid (PHB). U.S. Pat. Nos. 4,957,861 and 4,786,598 provide geneticallyengineered microorganism and culture conditions to produce poly-3-hydroxybutyric acid. U.S. Pat. No. 4,618,583 provides genetically engineered microorganism and methods to produce 3-hydroxyisobutyric acid. U.S. Pat. No. 8,900,837 provides genetically engineered microorganism having a 2-hydroxyisobutyricacid, 3-hydroxyisobutyric acid and methacrylic acid metabolic pathways. U.S. Pat. No. 4,310,635 provides genetically engineered microorganism for the fermentative production of 3-hydroxyisobutyric acid and methacrylic acid. U.S. Pat. No. 9,234,218 provides genetically engineered microorganism encoding enzymes methylmalonyl coenzyme A mutase for the production of 3-hydroxyisobutyric acid and polyhydroxyalkanoates. U.S. Pat. No. 4,211,846 provides genetically engineered microorganism for the fermentative production of 3-hydroxybutyric acid. U.S. Pat. No. 7,262,037 provides genetically engineered microorganism encoding genes PhbA, PhbB, Ptb and Buk to produce 3-hydroxybutyric acid. U.S. Pat. No. 4,540,665 provides genetically engineered microorganism to produce 3-hydroxyalkanoic acid from alkanoic acid, alkenoic acid and alcohols. U.S. Pat. No. 8,349,596 provides genetically engineered microorganism encoding enzyme hydroxyl-carboxylate-CoA mutase for the conversion of 3-hydroxybutyryl-CoA to 2-hydroxyisobutyryl-CoA. U.S. Patent Application Publication No. 2011/0151530 discloses fermentation methods with genetically modified microorganisms to favor the production of 2-hydroxyisobutyric acid.

U.S. Patent Application Publication No. 2009/0130731 discloses genetically engineered microorganisms obtained by introducing a polyhydroxyalkanoic acid synthase and 3-ketoacyl-ACP reductase genes to produce high molecular weight polyester polymers. U.S. Pat. No. 5,371,002 provides genetically engineered microorganism encoding polyhydroxybutyrate genes to produce polyhydroxyalkanoate. U.S. Pat. No. 6,593,116 provides transgenic microorganism encoding enzymes thiolase, reductase, PHB synthase and PHA synthase to produce polyhydroxyalkanoates. U.S. Pat. No. 7,968,325 provides genetically engineered microorganism comprising co-expression of polyhydroxyalkanoate synthase to a fattyacid:acyl-CoA transferase or an acyl-CoA synthase that enables the production of polyhydroxyalkanoates polyesters. U.S. Patent Application Publication No. 2016/0068463 discloses genetically engineered microorganisms to produce 4-hydroxybutyric acid from renewable carbohydrate based resources. U.S. Patent Application Publication No. 2005/0069995 discloses genetically engineered microorganisms comprising genes encoding intracellular polyhydroxyalkanoate depolymerase and polyhydroxyalkanoate biosynthesis related enzymes to produce 3-hydroxycarboxylic acids. U.S. Pat. No. 4,336,334 provides genetically engineered microorganism to produce polyhydroxybutyricacid using methanol as a sole source of carbon. U.S. Pat. No. 8,049,065 provides genetically engineered microorganism to produce poly(2-hydroxybutyrate-Co-3-hydroxyhexanoate) copolymer from carbohydrate based renewable resources. U.S. Pat. No. 7,455,999 provides genetically engineered microorganism to produce poly(3-hydroxybutyrate-Co-3-hydroxyhexanoate) copolymer from carbohydrate based renewable resources. U.S. Pat. No. 7,229,804 provides genetically engineered microorganism comprising gene encoding 4-hydroxybutyryl-CoA transferase enzyme to produce polyhydroxybutyrates. U.S. Pat. No. 5,512,456 provides genetically engineered microorganism and methods for the improved production and recovery of polyhydroxybutyrate. U.S. Pat. No. 6,117,658 provides genetically engineered microorganism for the PHA biosynthetic pathway combined with succinic semialdehdye metabolic pathway to produce polyhydroxyalkanoates. U.S. Pat. No. 4,138,291 provides genetically engineered microorganism for the conversion of variety of carbon sources to poly-3-hydroxybutyric acid. U.S. Pat. No. 9,273,331 provides genetically engineered microorganism and processes to produce 3-hydroxybutyricacid and their salts. U.S. Pat. No. 8,535,918 provides genetically engineered microorganism gene encoding enzymes beta-ketothiolase, 3-hydroxybutyryl-CoA dehydrogenase, acyl-coA hydrolase to produce 3-hydroxybutyric acid and 3-hydroxybutyrate ester. U.S. Pat. No. 4,443,053 provides genetically engineered microorganism for the continuous fermentation and accumulation of poly-3-hydroxybutyric acid under limited nutrient growth conditions. U.S. Pat. No. 6,472,188 provides genetically engineered microorganism to produce hydroxycarboxylic acid by the auto-degradation of polyhydroxyalkanoates.

U.S. Pat. No. 6,770,464 provides genetically engineered microorganisms encoding genes PhaE, PhaC components to produce polyhydroxy fatty acids. U.S. Patent Application Publication No. 2014/0256904 discloses genetically engineered microorganisms for the w-functionalization of aliphatic carboxylic acids and the production of long chain dicarboxylic acids. U.S. Patent Application Publication No. 2013/0267012 discloses genetically engineered microorganisms to produce fatty acid derived dicarboxylic acids ranging in chain length from C3 to C26. International PCT Application Publication No. WO2014/201474 provides genetically engineered microorganism and methods to produce w-hydroxylated aliphatic carboxylic acids, where the fatty acid is derived from carbohydrate based resources. U.S. Pat. No. 9,040,282 provides genetically engineered microorganisms comprising enzyme polyketide synthase capable of synthesizing dicarboxylic acids.

Citric acid is produced worldwide by the fermentation of carbohydrates followed by multi-step purification and recovery process. Aspergillus niger, a fungus and Candida sp, a yeast has been used for the commercial citric acid production using conventional carbohydrate sources such as molasses, dextrose from corn starch, cassava, sweet potatoes and cellulosic sugars. U.S. Pat. No. 3,622,455 provides production of citric acid from microbial strains Candida oleophila. U.S. Pat. No. 5,081,025 provides process for fermentative production of citric acid from carbohydrates by Aspergillus niger. U.S. Pat. No. 7,172,887 provides genetically engineered microorganisms having enhanced oxaloacetate hydrolase activity for the improved production of citric acid. U.S. Pat. No. 3,793,146 provides methods and processes for the production of citric acid from microbial strains Candida oleophila. U.S. Pat. Nos. 5,532,148, 3,809,611, 3,189,527, 2,739,923, 2,970,084 and 3,372,094 provide process for the production of citric acid in high yield and purity using Aspergillus niger. U.S. Pat. No. 2,993,838 provides method of producing citric acid by fermentation comprising strains of fungi from Trichoderma viride. U.S. Pat. No. 9,023,637 provides genetically engineered microorganisms having increased expression of LaeA gene and deletion of Alg3 gene for the enhanced citric acid production. U.S. Pat. No. 8,637,280 provides newly identified genes that encode proteins that are involved in the biosynthesis of citric acid. U.S. Pat. No. 3,717,549 provides process for producing citric acid by aerobic fermentation with yeast strains. U.S. Pat. Nos. 4,322,498 and 3,926,724 provide genetically engineered yeast strains which requires the presence of iron in the culture medium for the increased production of citric acid. U.S. Pat. No. 3,632,476 provides process for producing citric acid by Candida strains under aerobic fermentation methods. U.S. Pat. No. 3,886,041 provides increasing the yield of citric acid produced by the fermentation of sugars by Aspergillus niger comprising sodium hydroxide in sufficient amounts to maintain the pH. U.S. Pat. No. 5,827,700 provides improved processes and recovering methods to produce citric acid from impure process stream. U.S. Pat. No. 5,532,148 and U.S. Patent Application Publication No. 2011/0045558 disclose genetically engineered microorganisms to produce citric acid from glycerol with the yield of more than 70%.

U.S. Pat. No. 7,220,561 provides genetically engineered microorganisms having modified pantothenate biosynthetic enzyme activities with reduced byproduct formation and increased yield to produce pantothenate. U.S. Pat. No. 6,689,592 provides genetically engineered microorganisms of the family Enterobacteriaceae that already produce pantothenic acid for the fermentative production of pantothenate. U.S. Pat. No. 8,232,081 provides genetically engineered microorganisms comprising pantothenate biosynthetic pathway, isoleucine-valine biosynthetic pathway and CoenzymeA biosynthetic pathway for the enhanced production of pantothenate. U.S. Pat. No. 6,682,915 provides genetically engineered microorganisms overexpressing genes GlyA, IlvGM, PanB, PanE, PanD and PanC for the increased formation of pantothenate. U.S. Pat. No. 6,184,006 provides genetically engineered microorganisms of the family Enterobacteriaceae that already produce pantothenic acid where gene PanD is overexpressed for the enhanced production of pantothenate. U.S. Pat. No. 7,262,034 provides genetically engineered microorganisms of the family Bacillus that already produce pantothenic acid where genes SerA, SerC, YwpJ and GlyA are overexpressed for the enhanced production of pantothenate. U.S. Pat. No. 7,611,872 provides genetically engineered microorganisms of the family Bacillus where the supply of β-alanine is drastically reduced and produced internally for the synthesis of panthenic acid. U.S. Pat. No. 5,518,906 provides genetically engineered microorganisms of the family Enterobacteriaceae having resistance to salicyclic acid and capable of producing pantathenate in the presence of β-alanine. U.S. Pat. No. 5,932,457 provides genetically engineered microorganisms for the production of pantoic acid and further contacting the microorganism with β-alanine for the production of pantothenic acid. U.S. Pat. No. 6,171,845 provides genetically engineered microorganism overexpressing PanE and IlvC genes to produce pantothenic acid. U.S. Pat. No. 7,989,187 provides genetically engineered microorganisms having deregulated pantothenate biosynthetic pathway and deregulated methylenetetrahydrofolate biosynthetic pathway for the increased production of pantothenate. U.S. Pat. No. 7,244,593 provides genetically engineered microorganisms comprising overexpressed genes SerA and GlyA for the increased production of pantothenate. U.S. Pat. No. 6,623,944 provides genetically engineered microorganisms of the family Enterobacteriaceae that already produce pantothenic acid where gene PoxB is eliminated for the enhanced production of pantothenate. U.S. Pat. No. 6,667,166 provides genetically engineered microorganisms of the family Corneform where gene PfkA is overexpressed for the enhanced production of pantothenate. U.S. Pat. No. 6,184,007 provides genetically engineered microorganisms where gene PanD, PanB and PanC are overexpressed for the enhanced production of pantothenate. U.S. Pat. No. 6,787,334 provides genetically engineered microorganisms overexpressing enzyme ketopentoate reductase, gene PanE and gene IlvC to produce pantothenic acid. U.S. Pat. No. 7,338,792 provides genetically engineered microorganisms overexpressing genes YbbT, YwkA, YjmC, YtsJ, Mdh, CysK, IolJ, PdhD, YuiE and DhaS to produce pantothenate. U.S. Pat. No. 6,686,183 provides genetically engineered microorganisms overexpressing genes GcvT, GcvH and GcvP are overexpressed for the enhanced production of pantothenate. U.S. Pat. No. 6,913,912 provides genetically engineered microorganisms of the family Enterobacteriaceae that already produce pantothenic acid where gene Adk is enhanced for the increased production of pantothenate. U.S. Pat. No. 8,765,426 provides genetically engineered microorganisms of the family Zymomonas where enzymes 2-dehydropantoate reductase and aspartate 1-decarboxylase were overexpressed to produce pantothenate. U.S. Pat. No. 6,117,264 provides genetically engineered microorganisms overexpressing genes PanB, PanC and IlvD for the improved production of pantothenate. U.S. Pat. No. 6,911,329 provides genetically engineered microorganisms of the family Coryneform that already produce pantothenic acid where gene PoxB is eliminated for the enhanced production of pantothenate. U.S. Patent Application Publication No. 2015/0042104 discloses genetically engineered microorganisms where the gene encoding for phosphoenolpyruate carboxykinase is eliminate for the improved production of pantothenate. U.S. Patent Application Publication No. 2004/0048343 discloses genetically engineered microorganisms having modified pantothenate biosynthetic enzyme activities to produce 3-(2-hydroxy-3-methyl-butyrylamino)-propionic acid. International PCT Application. Publication No. WO2002/072855 provides genetically engineered microorganism of the family Enterobacteriaceae that already produce pantothenic acid where gene PoxB is eliminated for the increased production of pantothenate. International PCT Application. Publication No. WO2002/029020 provides genetically engineered microorganism of the family Coryneform that already produce pantothenic acid where gene poxB coding for enzyme pyruvate oxidase is eliminated for the enhanced production of pantothenate. International PCT Application. Publication No. WO2003/004673 provides genetically engineered microorganism of the family Bacillus that already produce pantothenic acid where the genes azlC, yzlD, ydaP and pckA are eliminated for the enhanced production of pantothenate. International PCT Application. Publication No. WO2003/029476 provides genetically engineered microorganism of the family Enterobacteriaceae that already produce pantothenic acid where the genes ytfP and yjfA are eliminated for the increased production of pantothenate. International PCT Application. Publication No. WO2002/024936 provides genetically engineered microorganism of the family Coryneform that already produce pantothenic acid where gene poxB coding for enzyme pyruvate oxidase is eliminated for the enhanced production of pantothenate.

In one method to enhance DHA uptake and utilization, the genetically modified host cells already known to produce organic acids, as described in patents above, are subjected to further genetic engineering by means of expressing plasmids that code ATP and PEP dependent kinases that could phosphorylate DHA and facilitate the entry of DHA into glycolytic cycle. In another method to enhance DHA uptake and utilization, genetically modified host cells already known to produce organic acids, as described in the patents above, is subjected to chemical mutagenesis and the strains with the ability to grow and produce desired organic acid with high enough titer and yield in a growth medium comprising DHA as a source of carbon are selected and subjected to whole genome sequencing to identify the specific mutations associated with the ability to grow and produce organic acid in a medium comprising DHA. Such specific mutations are subsequently introduced to the genetically modified host cells already known to produce organic acids for the purpose of conferring the ability to use DHA as a source of organic carbon. In yet another method to enhance DHA uptake and utilization, genetically modified host cells already known to produce organic acids, as described in patents above, is exposed to growth medium with increasing concentration of DHA and subjected to metabolic evolution. Strains that have gained ability to grow in the medium containing DHA as a major or sole source of carbon and still retain the ability to produce organic acids are selected and subjected to whole genome sequencing to identify specific mutations associated with the ability to grow and produce organic acids in a medium comprising DHA. Such specific mutations are introduced into the genetically modified host cells already known to produce organic acids, as described in patents above, for the purpose of conferring the ability to use DHA as a source of organic carbon.

In another embodiment, the present invention provides methods of producing C2-C3 alcohols such as ethanol, n-propanol and isopropanol. In one aspect, the present invention provides genetically modified microorganisms producing C2-C3 alcohols are subjected to further genetic modifications to confer the ability to use DHA as a source of carbon. In another aspect of this invention, microorganisms that have ability to grow in a medium comprising DHA as a source of carbon is subjected to further genetic modifications in the ethanol biosynthetic pathway, isopropanol biosynthetic pathway, lactate to n-propanol biosynthetic pathway, DHA to n-propanol biosynthetic pathway, succinyl-CoA to n-propanol biosynthetic pathway, threonine to n-propanol biosynthetic pathway and pyruvate to n-propanol biosynthetic pathway to produce C2-C3 alcohols. The list of the enzymes that are suitable to enhance the production of C2 and C3 alcohols includes: one or more enzymes involved in the ethanol biosynthetic pathway such as lactate dehydrogenase, pyruvate dehydrogenase, acetyl-CoA ligase, pyruvate decarboxylase, lactate-2-monooxygenase, acetaldehyde dehydrogenase, alcohol dehydrogenase, citrate lyase and formate acetyltransferase in the ethanol biosynthetic pathway; one more enzymes functional in the isoproponal biosynthetic pathway such as β-ketothiolase, acetoacetyl-CoA transferase, acetoacetate decarboxylase, alcohol dehydrogeanse and acetyl-CoA:butyryl-CoA transferase; one or more enzymes functional in the DHA to n-proponal biosynthetic pathway such as dihydroxyacetone kinase, methylglyoxal synthase, methylglyoxal reductase, acetol reductase, 1,2-propanediol oxidoreductase, propanediol dehydratase and propanol dehydrogenase; one or more enzymes functional in the lactate to n-propanol biosynthetic pathway such as pyruvate kinase, DHA-phosphotransferase system (pts), lactate dehydrogenase, lactoyl-CoA transferase, lactoyl-CoA reductase, 1,2-propanediol oxidoreductase, propanediol dehydratase, propanol dehydrogenase; one or more enzymes functional in the threonine to n-propanol biosynthetic pathway such phosphoenolpyruvate carboxylase, aspartate transaminase, aspartate kinase, aspartate semialdehyde dehydrogenase, homoserine dehydrogenase, homoserine kinase, threonine synthase, threonine dehydratase (IlvA), 2-ketoacid decarboxylase and propanol dehydrogenase; one or more enzymes functional in the succinyl-CoA to n-propanol biosynthetic pathway such as phosphoenolpyruvate carboxylase, malate dehydrogenase, fumarate hydratase, succinate dehydrogenase, isocitrate lyase, malate synthase, succinyl-CoA synthase, 2-ketoglutarate dehydrogenase, methylmalonyl-CoA mutase, methylmalonyl-CoA epimerase, methylmalonyl-CoA decarboxylase, aldehyde dehydrogenase and alcohol dehydrogenase; one or more enzymes functional in the pyruvate to n-propanol biosynthetic pathway such as citramalate synthase, citroconate hydrolase, methylmalate dehydratase, methylmalate dehydrogenase, 2-ketoacid decarboxylase and propanol dehydrogenase.

In some aspect, microorganisms that have ability to grow in DHA in a medium comprising DHA as a source of carbon is subjected to further genetic modifications to include one or more nucleic acid comprising a nucleotide sequence encoding ethanol pathway enzymes. ethanol is made from pyruvate by a series of enzyme catalyzed biochemical reactions. Microorganisms that already have an ability to produce high amounts of pyruvate or ethanol either natively or by one or more genetic modifications are a preferred host to produce ethanol. On the other hand, ethanol biosynthetic pathway can be directly engineered in to the host cell that previously lack the ability to produce pyruvate but can be engineered to selectively produce ethanol. Furthermore, in addition to the enzymes catalyze that catalyzes formation of pyruvate from DHA, one or more of the enzymes selected from a group of ethanol biosynthetic pathway such as lactate dehydrogenase, pyruvate dehydrogenase, acetyl-CoA ligase, pyruvate decarboxylase, lactate-2-monooxygenase, acetaldehyde dehydrogenase, alcohol dehydrogenase, citrate lyase and formate acetyltransferase are overexpressed for the enhanced production of ethanol from pyruvate. In addition, attenuation or deletion of methylglyoxal synthase, phosphoenolpyruvate carboxylase, pyruvate carboxylase, pyruvate kinase, malonate semi aldehyde dehydrogenase, succinate dehydrogenase, citrate synthase, acetyl-CoA carboxylase, acetyl-CoA acetyl transferase, hydroxymethyl-CoA glutaryl synthase and glycerol-3-phosphate dehydrogenase improve the carbon utilization in the ethanol synthesis. Blocking ethanol utilization pathway by attenuation or deletion of enzymes responsible for the degradation of ethanol to acetic acid and ethyl acetate improves ethanol accumulation. Furthermore, improving ethanol transport, reducing ethanol uptake from media and reducing feedback inhibition increases ethanol productivity.

In some embodiments, microorganisms that have ability to grow in DHA in a medium comprising DHA as a source of carbon is subjected to further genetic modifications to include one or more nucleic acid comprising a nucleotide sequence encoding isopropanol pathway enzymes. Isopropanol is made from acetyl-CoA by a series of enzyme catalyzed biochemical reactions. Microorganisms that already have an ability to produce high amounts of acetyl-CoA derived products either natively or by one or more genetic modifications are a preferred host to produce isopropanol. On the other hand, isopropanol biosynthetic pathway can be directly engineered in to the host cell that previously lack the ability to produce acetyl-CoA but can be engineered to selectively produce isopropanol. Furthermore, in addition to the enzymes that catalyze the formation of acetyl-CoA from DHA, one or more of the enzymes selected from a group of isopropanol biosynthetic pathway such as β-ketothiolase, acetoacetyl-CoA transferase, acetoacetate decarboxylase and alcohol dehydrogeanse are overexpressed to produce isopropanol from acetyl-CoA. Furthermore, gene encoding CoAT (acetyl-CoA:butyryl-CoA transferase) is introduced to enhance the conversion of acetoacetyl-CoA to acetoacetate by converting acetate and butyrate to acetyl-CoA and Butyryl-CoA respectively. In addition, attenuation or deletion of one or more of methylglyoxal synthase (MgsA), phosphoenolpyruvate carboxylate, pyruvate carboxylate, acetate kinase (AckA), aldehyde dehydrogenase (AldA), phosphotransacylase (Pta), pyruvate oxidase (PoxB), pyruvate-formate lyase (PflB), succinate dehydrogenase (FrdBC), malate dehydrogenase, 2-ketoglutarate dehydrogenase, lactate dehydrogenase (LdhA), acetyl-CoA carboxylase, hydroxymethyl-CoA glutaryl synthase, citrate synthase and improved acetyl-CoA utilization in isopropanol synthesis. Blocking isopropanol utilization pathway by attenuation or deletion of enzymes responsible for the degradation of isopropanol to acetic acid and isopropyl acetate improves isopropanol accumulation. Furthermore, improving isopropanol transport, reducing isopropanol uptake from media and reducing feedback inhibition increases isopropanol productivity.

In some embodiments, microorganisms that have ability to grow in DHA in a medium comprising DHA as a source of carbon is subjected to further genetic modifications to include one or more nucleic acid comprising a nucleotide sequence encoding n-propanol pathway enzymes. n-propanol is also made from 1,2-propanediol by a two-step enzyme catalyzed biochemical reactions. n-propanol is also made from L-threonine by a series of enzyme catalyzed biochemical reactions. In another method to produce n-propanol, succinyl-CoA is converted to n-propanol by a series of enzyme catalyzed biochemical reactions. In yet another method to produce n-propanol, metabolite pyruvate is converted to n-propanol by a series of enzyme catalyzed biochemical reactions. Microorganisms that already has an ability to produce high amounts of 1,2-propanediol, L-threonine or succinate either natively or by one or more genetic modifications are a preferred host for the production of n-propanol. On the other hand, n-propanol biosynthetic pathway can be directly engineered into the host cell that previously lack the ability to produce 1,2-propanediol, L-threonine or succinate but can be engineered to selectively produce n-propanol. Furthermore, in addition to the enzymes required to produce 1,2-propanediol from DHA, one or more of the enzymes selected from a group of DHA to n-propanol biosynthetic pathway such as propanediol dehydratase and propanol dehydrogenase are overexpressed for the enhanced production of n-propanol. In addition, attenuation or deletion of one or more of the enzyme triosephosphate isomerase, glycerol-3P-dehydrogenase and transketolase improves DHA utilization in the n-propanol production. In another method to produce n-propanol, lactate is used for the production of n-propanol. In addition to the enzymes that catalyze the production of lactate from DHA, one or more of the enzymes selected from the group of lactate to n-propanol biosynthetic pathway such as lactoyl-CoA transferase, lactoyl-CoA reductase, 1,2-propanediol oxidoreductase, propanediol dehydratase, propanol dehydrogenase are overexpressed for the enhanced production of n-propanol from lactate. Moreover, improvement of lactate production by overexpressing one or more enzymes in lactate biosynthetic pathway enhances the productivity of n-propanol. In addition, attenuation or deletion of one or more of methylglyoxal synthase, phosphoenolpyruvate carboxylate, pyruvate carboxylase, pyruvate dehydrogenase, acetate kinase, aldehyde dehydrogenase, phosphotransacylase, pyruvate oxidase, pyruvate-formate lyase), succinate dehydrogenase (FrdBC), acetyl-CoA carboxylase, hydroxymethyl-CoA glutaryl synthase, citrate synthase, alanine transaminase, lactate monooxigenase and β-ketothiolase (ThlAB) improves pyruvate utilization in n-propanol synthesis. In another method to produce n-propanol, amino acid L-threonine is used for the production of n-propanol. In addition to the enzymes that catalyze production of threonine from DHA, one or more of the enzymes selected from a group of L-threonine to n-propanol biosynthetic pathway such as threonine dehydratase (IlvA), 2-ketoacid decarboxylase, propanol dehydrogenase is overexpressed for the enhanced production of n-propanol from L-threonine. Moreover, improvement of threonine production by overexpressing one or more enzymes in threonine biosynthetic pathway enhances the productivity of n-propanol. In addition, attenuation or deletion of one or more of enzymes methylglyoxal synthase, acetate kinase, aldehyde dehydrogenase, phosphotransacylase, pyruvate oxidase, pyruvate-formate lyase, lactate dehydrogenase, acetyl-CoA carboxylase, hydroxymethyl-CoA glutaryl synthase, citrate synthase and β-ketothiolase (ThlAB) improves acetyl-CoA utilization in n-propanol synthesis. In addition, attenuation or deletion of one or more of enzymes isopropylmalate synthase (LeuA), isopropylmalate isomerase (LeuCD), isopropylmalate dehydrogenase (LeuB), acetolactate synthase (IlvB), keto-acidreducto isomerase (IlvC) improves 2-ketobutyrate utilization in n-propanol synthesis. In another method to produce n-propanol, succinyl-CoA is used for the production of n-propanol. In addition to the enzymes that catalyze production of succinyl-CoA from DHA, one or more of the enzymes selected from a group of succinate to n-propanol biosynthetic pathway such as succinyl-CoA synthase, 2-ketoglutarate dehydrogenase, methylmalonyl-CoA mutase, methylmalonyl-CoA epimerase, methylmalonyl-CoA decarboxylase, aldehyde dehydrogenase and alcohol dehydrogenase are overexpressed for the enhanced production of propanol from succinyl-CoA. Moreover, improvement of succinate production by overexpressing one or more enzymes in succinate biosynthetic pathway enhances the productivity of n-propanol. In addition, attenuation or deletion of one or more of enzymes methylglyoxal synthase (MgsA), acetate kinase (AckA), aldehyde dehydrogenase (AldA), phosphotransacylase (pta), pyruvate oxidase (PoxB), pyruvate-formate lyase (PflB), lactate dehydrogenase (LdhA), acetyl-CoA carboxylase, hydroxymethyl-CoA glutaryl synthase, β-ketothiolase (ThlAB), aspartate transaminase, glutamate dehydrogenase and aspartate ammonia-lyase enhances the carbon utilization in n-propanol synthesis. In yet another method to produce n-propanol pyruvate is used for the production of n-propanol. In addition to the enzymes that catalyze production of pyruvate from DHA, one or more of the enzymes selected from a group of pyruvate to n-propanol biosynthetic pathway such as citramalate synthase, citroconate hydrolase, methylmalate dehydratase, methaylmalate dehydrogenase, 2-ketoacid decarboxylase and propanol dehydrogenase are overexpressed for the enhanced production of propanol from pyruvate. In addition, attenuation or deletion of one or more of methylglyoxal synthase (MgsA), phosphoenolpyruvate carboxylate, pyruvate carboxylate, acetate kinase (AckA), aldehyde dehydrogenase (AldA), phosphotransacylase (pta), pyruvate oxidase (PoxB), pyruvate-formate lyase (PflB), succinate dehydrogenase (FrdBC), malate dehydrogenase, lactate dehydrogenase (LdhA), acetyl-CoA carboxylase, hydroxymethyl-CoA glutaryl synthase, citrate synthase and improves pyruvate utilization in n-propanol synthesis. Blocking n-propanol utilization pathway by attenuation or deletion of enzymes responsible for the degradation of n-propanol to propionic acid and propyl acetate improves n-propanol accumulation. Furthermore, improving n-propanol transport, reducing n-propanol uptake from media and reducing feedback inhibition increases n-propanol productivity.

Genetically modified microorganisms capable of producing C2-C3 alcohols using conventional sugars such as glucose, sucrose or glycerol are already known in the art. U.S. Pat. No. 5,028,539 provides mutant Escherichia coli with a gene coding for pyruvate decarboxylase for ethanol production. U.S. Pat. Nos. 5,482,846 and 5,916,787 provide gram-positive bacteria with heterologous genes which provide ability for the microbes to produce ethanol. U.S. Pat. No. 5,554,520 provides recombinant microorganism comprising genes coding for the alcohol dehydrogenase and pyruvate decarboxylase for the enhanced production of the ethanol. U.S. Pat. No. 5,000,000 provides plasmids comprising genes which code for the alcohol dehydrogenase and pyruvate decarboxylase activities of Zymomonas mobilis for the ethanol production. U.S. Pat. No. 8,623,622 provides genetically engineered microorganism optimized for the enhanced ethanol production. U.S. Pat. No. 4,403,034 and U.S. Pat. No. 4,443,544 provides culture conditions and methods to produce ethanol from Zymomonas mobilis. U.S. Patent Application Publication No. 2011/0020890 discloses thermophilic microorganism lacking lactate dehydrogenase activity producing ethanol through pyruvate-formate lyase pathway. U.S. Pat. No. 8,795,998 provides recombinant yeast lacking glycerol synthesis activity for the improved ethanol production. U.S. Pat. Nos. 8,394,622 and 8,980,617 provide novel yeast strains Saccharomyces cerevisae YE1358 and YE 1615 for the improved ethanol production. U.S. Pat. Nos. 8,216,816, 7,968,321, 7,981,647, 8,048,666, 8,227,237, 8,465,954, 8,986,964, 6,699,696, 6,306,639 and 9,150,888 provides engineered cyanobacterium comprising recombinant pyruvate decarboxylase gene to produce ethanol. U.S. Pat. No. 7,507,554 provides process to produce ethanol from algae. U.S. Pat. Nos. 8,790,911 and 9,045,779 provides increased ethanol production by genetic engineering of microorganisms to express hemoglobin. U.S. Pat. No. 5,424,202 provides recombinant host that has been transformed with pyruvate decarboxylase and alcohol dehydrogenase for ethanol production. U.S. Pat. Nos. 4,885,241 and 6,566,107 provide production of ethanol by Zymomonas fermentation. U.S. Pat. No. 8,268,600 provides production of ethanol by Kluyveromyces species IIPE453. U.S. Pat. No. 5,487,989 provides recombinant host comprising alcohol dehydrogenase and pyruvate decarboxylase for the ethanol production. U.S. Patent Application Publication No. 2014/0295515 discloses ethanol production from glycerol fermentation process. U.S. Pat. Nos. 7,691,620 and 8,192,977 provide thermophilic bacterium for the enhanced production of ethanol. U.S. Pat. No. 8,652,817 provides recombinant host to produce ethanol by degrading oligosaccharide. U.S. Pat. No. 4,393,136 provides ethanol production using immobilized bacterial cell. U.S. Patent Application Publication No. 2007/0141690 discloses ethanol producing xylose utilizing Saccharomyces cerevisiae. U.S. Pat. No. 8,932,841 provides thermophilic microorganism for ethanol production. U.S. Pat. No. 8,716,002 provides reengineered bacteria for the enhanced ethanol production. U.S. Patent Application Publication No. 2015/0072391 discloses engineered yeast for the enhanced ethanol production. U.S. Pat. No. 8,765,427 provides ethanol production from mannitol using yeast. U.S. Patent Application Publication No. 2009/0082600 discloses native homoethanol pathway for ethanol production in yeast. U.S. Pat. No. 4,652,526 provides ethanol producing mutant Clostridium thermosaccharolyticum. U.S. Pat. Nos. 5,677,154, 5,932,456 and 7,527,941 provide ethanol fermentation from biomass. U.S. Pat. No. 3,093,548 provides ethanol production from sugars using proteolytic enzymes. U.S. Pat. Nos. 4,393,136 and 4,731,329 provides methods for high-performance bacterial ethanol production. U.S. Pat. No. 4,560,659 provides ethanol production from fermentation of sugarcane. U.S. Pat. Nos. 8,574,879, 7,285,402, 8,642,301, 8,642,302 and 8,647,851 describe methods of increasing ethanol production from microbial fermentation. U.S. Pat. No. 5,173,429 provides Clostridium ljungdahlii for the anaerobic ethanol production. U.S. Pat. Nos. 5,514,583, 5,726,053 and 7,285,403 provides recombinant microorganism for the utilization of pentose sugar and ethanol production. U.S. Pat. Nos. 6,333,181 and 4,094,742 provide ethanol production from lignocellulose. U.S. Pat. No. 5,554,520 provides recombinant host for the production of ethanol. U.S. Patent Application Publication No. 2009/0042265 discloses thermophilic microorganism with inactivated Ldh for ethanol production. U.S. Pat. Nos. 8,021,865, 8,143,038, 8,852,906 and 8,932,841 provides thermophilic microorganism for ethanol production. U.S. Pat. No. 8,486,687 provides sporulation-deficient thermophilic microorganism for the production of ethanol. International PCT Application. Publication No. WO1998/045425 provides development of high-ethanol resistant Escherichia coli for ethanol production. U.S. Pat. Nos. 7,691,620 and 8,192,977 provide fermentative production of ethanol by heterologous expression of pyruvate decarboxylase gene. U.S. Pat. Nos. 7,226,776 and 7,026,152 provide methods and recombinant microorganisms for the simultaneous saccharification and fermentation for the ethanol production. U.S. Pat. Nos. 8,093,037, 8,227,236, and 8,114,974 provide engineered microorganism with enhanced fermentation activity. U.S. Patent Application Publication No. 2014/0356921 discloses engineered microorganism to increase ethanol production by metabolic redirection. U.S. Patent Application Publication No. 2015/0299736 discloses modified bacteria capable of fermenting both hexose and pentose to produce ethanol. U.S. Patent Application Publication No. 2011/0230682 discloses microorganism with inactivated Ldh for ethanol production. U.S. Pat. No. 6,849,434 discloses ethanol production in recombinant microorganisms. U.S. Pat. Nos. 6,102,690 and 5,100,791 disclose recombinant organisms capable of fermenting cellobiose. U.S. Pat. No. 6,280,986 discloses ethanol production in bacterial strains lacking lactate dehydrogenase and pyruvate lyase activities. U.S. Pat. No. 8,097,460 discloses ethanol production in recombinant Bacillus. U.S. Pat. No. 7,598,063 discloses process for producing ethanol by using recombinant Coryneform bacterium. U.S. Pat. No. 8,338,149 discloses yeast for ethanol production process. U.S. Pat. No. 8,716,002 discloses reengineered Escherichia coli for the ethanol production. U.S. Pat. No. 9,034,619 discloses recombinant Deinococcus bacterium to produce ethanol. U.S. Pat. No. 4,472,501 discloses process of manufacturing ethanol by culturing Kluyveromyces species. International PCT Application Publication No. WO2012/109578 provides Clostridium thermocellum strains for enhanced ethanol production. U.S. Pat. No. 4,368,268 provides direct fermentation of xylose for the ethanol production. U.S. Pat. Nos. 4,663,284, 4,359,534 and 4,511,656 disclose enhanced fermentation of xylose to ethanol. U.S. Pat. No. 4,840,903 discloses process for producing ethanol from plant biomass using fungus Paecilomyces sp. U.S. Patent Application Publication No. 2014/0342424 discloses novel yeast designated as Candida intermedia for the ethanol production.

U.S. Pat. No. 8,715,976 provides recombinant microorganism having enhanced n-propanol biosynthetic activity from threonine. U.S. Pat. No. 8,715,971 provides recombinant microorganism and methods for co-producing isopropanol and n-propanol by various metabolic pathways. U.S. Patent Application Publication No. 2009/0246842 discloses engineered microorganism to produce isopropanol from acetone. U.S. Patent Application Publication No. 2015/0064760 discloses modified microorganism and methods to produce n-propanol via 1,2-propanediol metabolic intermediate. U.S. Patent Application Publication No. 2015/0064759 discloses enzymes and metabolic pathways to produce isopropanol and 1-propanol via 1,2-propanediol metabolic intermediate. U.S. Patent Application Publication No. 2015/0152440 discloses modified microorganisms and methods for co-producing butadiene, n-propanol via 1,2-propanediol metabolic intermediate. U.S. Patent Application Publication No. 2014/0134691 discloses recombinant microorganism comprising lactate dehydrogenase and lactate dehydratase activity for the n-propanol production. U.S. Patent Application Publication No. 2013/0280775 discloses methods of co-producing n-propanol with isopropanol using recombinant microorganism. U.S. Patent Application Publication No. 2014/0134690 discloses metabolically engineered microorganism to produce n-propanol from 1,2-propanediol intermediate. U.S. Patent Application Publication No. 2013/0095542 discloses engineered microorganism and integrated process for producing n-propanol and propylene. U.S. Patent Application Publication No. 2009/063963 discloses microorganism producing n-propanol through 1,2-propanediol from dihydroxyacetonephosphate metabolite. U.S. Patent Application Publication No. 2014/099707 discloses process for producing n-propanol from propionic acid pathway using metabolically engineered Propionobacteria. U.S. Patent Application Publication No. 2014/102180 discloses n-propanol production by Lactobacillus bacterium.

In one method to enhance DHA uptake and utilization, the genetically modified host cells already known to produce C2-C3 alcohols as described in patents above are subjected to further genetic engineering by means of expressing plasmids that code ATP and PEP dependent kinases that could phosphorylate DHA and facilitate its entry into glycolytic cycle. In another approach to enhance DHA uptake and utilization, genetically modified host cells already known to produce C2-C3 alcohols as described in patents above are subjected to chemical mutagenesis and the strains with the ability to grow and produce desired C2-C3 alcohols with high enough titer and yield in a growth medium comprising DHA as a source of carbon are selected and subjected to whole genome sequencing to identify specific mutations associated with the ability to grow and produce C2-C3 alcohols in a medium comprising DHA. Such specific mutations are introduced into the genetically modified host cells already known to produce C2-C3 alcohols to confer the ability to use DHA as a source of organic carbon to produce C2-C3 alcohols. In yet another approach to enhance DHA uptake and utilization, genetically modified host cells already known to produce C2-C3 alcohols, as described in patents above, are exposed to growth medium with increasing concentration of DHA and subjected to metabolic evolution. Strains that have gained ability to grow in the medium containing DHA as a major or sole source of carbon and still retain the ability to produce C2-C3 alcohols are selected and subjected to whole genome sequencing to identify specific mutation associated with the ability to grow and produce C2-C3 alcohols in a medium comprising DHA. Such specific mutations are introduced into the genetically modified host cells already known to produce C2-C3 alcohols as described in patents above for the purpose of conferring the ability to use DHA as a source of organic carbon.

In another embodiment, the present invention provides methods of producing C4-C10 alcohols such as n-butanol, isobutanol, 2-butanol, n-pentanol, n-hexanol, n-heptanol, 2-methyl-1-butanol, 3-methyl-1-pentanol, 4-methyl-1-hexanol, 5-methyl-1-heptanol, 3-methyl-1-butanol, 4-methyl-1-pentanol, 5-methyl-1-hexanol and 6-methyl-1-heptanol. In one aspect, genetically modified microorganisms producing C4-C10 alcohols are subjected to further genetic modifications to confer the ability to use DHA as a source of carbon. In another aspect of this invention, microorganisms that have ability to grow in DHA in a medium comprising DHA as a source of carbon is subjected to further genetic modifications involving overexpression in one or more components of the acetyl-CoA to n-butanol biosynthetic pathway, pyruvate to n-butanol biosynthetic pathway, threonine to n-butanol biosynthetic pathway, succinyl-CoA to 2-butanol biosynthetic pathway, pyruvate to 2-butanol biosynthetic pathway, isobutanol biosynthetic pathway, n-pentanol biosynthetic pathway, n-hexanol biosynthetic pathway, n-heptanol biosynthetic pathway, 2-methyl-1-butanol biosynthetic pathway, 3-methyl-1-pentanol biosynthetic pathway, 4-methyl-1-hexanol biosynthetic pathway, 5-methyl-1-heptanol biosynthetic pathway, 3-methyl-1-butanol biosynthetic pathway, 4-methyl-1-pentanol biosynthetic pathway, 5-methyl-1-hexanol biosynthetic pathway and 6-methyl-1-heptanol biosynthetic pathway to produce C4-C10 alcohols.

The list of genetic manipulations required to enhance the C4-C10 alcohol biosynthesis within the microbial cell include: enhancing the activities of one or more of the enzymes involved in the acetyl-CoA to n-butanol biosynthetic pathway such as pyruvate decarboxylase, acetyl-CoA synthase, β-ketothiolase, 3-hydroxybutyryl-CoA reductase, crotonase, Butyryl-CoA dehydrogenase, butaraldehyde dehydrogenase and butanol dehydrogenase; enhancing activities of one or more of the enzymes in the pyruvate to n-butanol biosynthetic pathway such as pyruvate kinase, DHA:PEP phosphotransferase system (pts), acetolactate synthase, keto-acidreducto isomerase, dihydroxyacid dehydratase, enzyme which converts 2-oxoisovalerate to isobutyryl-CoA, isobutyryl-CoA mutase, butyraldehdye dehydrogenase and butanol dehydrogenase; enhancing activities of one or more of the enzymes functional in the threonine to n-butanol biosynthetic pathway such as phosphoenolpyruvate carboxylase, aspartate transaminase, aspartate kinase, aspartate semialdehyde dehydrogenase, homoserine dehydrogenase, homoserine kinase, threonine synthase, threonine dehydratase, isopropylmalate synthase, isopropylmalate isomerase, isopropylmalate dehydrogenase that converts 2-oxobutyrate to 2-oxovalerate, 2-ketoacid decarboxylase, alcohol dehydrogenase, citramalate synthase, citraconate hydrolase, methylmalate dehydratase and methylmalate dehydrogenase; enhancing the activities of one or more of the enzymes functional in the n-pentanol biosynthetic pathway such as phosphoenolpyruvate carboxylase, aspartate transaminase, aspartate kinase, aspartate semialdehyde dehydrogenase, homoserine dehydrogenase, homoserine kinase, threonine synthase, threonine dehydratase (IlvA), isopropylmalate synthase, isopropylmalate isomerase, isopropylmalate dehydrogenase that converts 2-oxobutyrate to 2-oxovalerate, isopropylmalate synthase, isopropylmalate isomerase, isopropylmalate dehydrogenase that converts 2-oxovalerate to 2-oxocaproate, 2-ketoacid decarboxylase and alcohol dehydrogenase; enhancing the activities of one or more of the enzymes functional in the in the n-hexanol biosynthetic pathway such as phosphoenolpyruvate carboxylase, aspartate transaminase, aspartate kinase, aspartate semialdehyde dehydrogenase, homoserine dehydrogenase, homoserine kinase, threonine synthase, threonine dehydratase, isopropylmalate synthase, isopropylmalate isomerase, isopropylmalate dehydrogenase that converts 2-oxobutyrate to 2-oxoheptanoate, 2-ketoacid decarboxylase and alcohol dehydrogenase; enhancing the activities of one or more of the enzymes functional in the in the n-heptanol biosynthetic pathway such as phosphoenolpyruvate carboxylase, aspartate transaminase, aspartate kinase, aspartate semi aldehyde dehydrogenase, homoserine dehydrogenase, homoserine kinase, threonine synthase, threonine dehydratase, isopropylmalate synthase, isopropylmalate isomerase, isopropylmalate dehydrogenase that converts 2-oxobutyrate to 2-oxooctanoate, 2-ketoacid decarboxylase and alcohol dehydrogenase; enhancing the activities of one or more of the enzymes functional in the 2-methyl-1-butanol biosynthetic pathway phosphoenolpyruvate carboxylase, aspartate transaminase, aspartate kinase, aspartate semialdehyde dehydrogenase, homoserine dehydrogenase, homoserine kinase, threonine synthase, threonine dehydratase (IlvA), acetolactate synthase, keto-acid reducto isomerase, dihydroxyacid dehydratase, 2-ketoacid decarboxylase and alcohol dehydrogenase; enhancing the activities of one or more of the enzymes functional in the 2-methyl-1-butanol biosynthetic pathway such as phosphoenolpyruvate carboxylase, aspartate transaminase, aspartate kinase, aspartate semi aldehyde dehydrogenase, homoserine dehydrogenase, homoserine kinase, threonine synthase, threonine dehydratase, acetolactate synthase, keto-acid reducto isomerase, dihydroxyacid dehydratase, isopropylmalate synthase, isopropylmalate isomerase, isopropylmalate dehydrogenase that converts 2-keto-3-methylvalerate to 2-keto-4-methylvalerate, 2-ketoacid decarboxylase and alcohol dehydrogenase; enhancing the activities of one or more of the enzymes functional in the in the 3-methyl-1-pentanol biosynthetic pathway such as phosphoenolpyruvate carboxylase, aspartate transaminase, aspartate kinase, aspartate semi aldehyde dehydrogenase, homoserine dehydrogenase, homoserine kinase, threonine synthase, threonine dehydratase, acetolactate synthase, keto-acid reducto isomerase, dihydroxyacid dehydratase, isopropylmalate synthase, isopropylmalate isomerase, isopropylmalate dehydrogenase that converts 2-keto-3-methylvalerate to 2-keto-5-methylvalerate, 2-ketoacid decarboxylase and alcohol dehydrogenase; enhancing the activities of one or more of the enzymes functional in the 4-methyl-1-hexanol biosynthetic pathway such as phosphoenolpyruvate carboxylase, aspartate transaminase, aspartate kinase, aspartate semi aldehyde dehydrogenase, homoserine dehydrogenase, homoserine kinase, threonine synthase, threonine dehydratase, acetolactate synthase, keto-acid reducto isomerase, dihydroxyacid dehydratase, isopropylmalate synthase, isopropylmalate isomerase, isopropylmalate dehydrogenase that converts 2-keto-3-methylvalerate to 2-keto-5-methylheptanoate, 2-ketoacid decarboxylase and alcohol dehydrogenase; enhancing the activities of one or more of the enzymes functional 5-methyl-1-heptanol biosynthetic pathway such as phosphoenolpyruvate carboxylase, aspartate transaminase, aspartate kinase, aspartate semi aldehyde dehydrogenase, homoserine dehydrogenase, homoserine kinase, threonine synthase, threonine dehydratase, acetolactate synthase, keto-acid reducto isomerase, dihydroxyacid dehydratase, isopropylmalate synthase, isopropylmalate isomerase, isopropylmalate dehydrogenase that converts 2-keto-3-methylvalerate to 2-keto-6-methyloctanoate, 2-ketoacid decarboxylase and alcohol dehydrogenase in the 5-methyl-1-heptanol biosynthetic pathway; enhancing the activities of one or more of the enzymes functional in the in the 3-methyl-1-butanol biosynthetic pathway such as acetolactate synthase, keto-acid reducto isomerase, dihydroxyacid dehydratase, isopropylmalate synthase, isopropylmalate isomerase, isopropylmalate dehydrogenase that converts 2-ketoisovalerate to 2-ketoisocaproate, 2-ketoacid decarboxylase and alcohol dehydrogenase; enhancing the activities of one or more of the enzymes functional acetolactate synthase, keto-acid reducto isomerase, dihydroxyacid dehydratase, gene LeuABCD encoding for enzymes isopropylmalate synthase, isopropylmalate isomerase, isopropylmalate dehydrogenase that converts 2-ketoisovalerate to 2-keto-5-methylcaproate, 2-ketoacid decarboxylase, alcohol dehydrogenase in the 4-methyl-1-pentanol biosynthetic pathway; enhancing the activities of one or more of the enzymes functional in the 5-methyl-1-hexanol biosynthetic pathway such as acetolactate synthase, keto-acid reducto isomerase, dihydroxyacid dehydratase, isopropylmalate synthase, isopropylmalate isomerase, isopropylmalate dehydrogenase that converts 2-ketoisovalerate to 2-keto-6-methylheptanoate, 2ketoacid decarboxylase and alcohol dehydrogenase; enhancing the activities of one or more of the enzymes functional in the 6-methyl-1-heptanol biosynthetic pathway such as acetolactate synthase, keto-acid reducto isomerase, dihydroxyacid dehydratase, isopropylmalate synthase, isopropylmalate isomerase, isopropylmalate dehydrogenase that converts 2-ketoisovalerate to 2-keto-7-methyloctanoate, 2-ketoacid decarboxylase and alcohol dehydrogenase; enhancing the activities of one or more of the enzymes functional in the isobutanol biosynthetic pathway such as acetolactate synthase, keto-acid reducto isomerase, dihydroxyacid dehydratase, 2-ketoacid decarboxylase, isobutaraldehyde dehydrogenase, valine transaminase, valine decarboxylase, omega-transaminase and alcohol dehydrogenase; enhancing the activities of one or more of the enzymes functional in the pyruvate to 2-butanol biosynthetic pathway such as acetolactate synthase, acetolactate decarboxylase, diacetyl reducatse, acetoin reductase, butanediol dehydrotase, 2-butanol dehydrogenase; enhancing the activities of one or more of the enzymes functional in the succinyl-CoA to 2-butanol biosynthetic pathway such as methylmalonyl-CoA mutase, methylmalonyl-CoA epimerase, methylmalonyl-CoA decarboxylase, β-ketovalerylthiolase, β-ketovalerate-CoA transferase, β-ketovalerate decarboxylase, 2-methyl acetoacetyl-CoA thiolase, 2-methyl acetoacetyl-CoA transferase, 2-methyl acetoacetate decarboxylase and alcohol dehydrogenase.

Genetically modified microorganisms capable of producing C4-C10 alcohols using conventional sugars such as glucose, sucrose or glycerol are already known in the art. U.S. Pat. No. 8,518,678 provides methods of increasing cyclopropane fatty acid synthase enzyme for the improved butanol tolerance. U.S. Pat. No. 8,426,173 provides method for reducing fermentation temperature for more robust tolerance of the butanol producing microorganism. U.S. Pat. No. 8,652,823 provides increased butanol tolerance when the membrane content of the unsaturated fatty acid is increased. U.S. Pat. No. 8,420,359 provides butanol producing microorganism using sugar as a source of carbon in the presence of lactic acid and acetic acid. U.S. Pat. No. 4,443,542 provides butanol producing microorganism using cellulose as a carbon source. U.S. Pat. No. 4,539,293 provides co-culturing methods to convert butyric acid in the medium to increased butanol production. U.S. Pat. No. 6,358,717 provides genetically engineered Clostridium bijerinckii to produce butanol. U.S. Pat. No. 5,192,673 provides asporogenic mutant of Clostridium acetobutylicum for the enhanced production of butanol. U.S. Pat. No. 8,940,511 provides increased butanol production in yeast cells with reduced general control response to amino acid starvation. U.S. Pat. No. 7,541,173 provides Lactobacillus bacteria having enhanced tolerance to butanol. U.S. Pat. No. 9,096,872 provides recombinant microorganism obtained by manipulating the metabolic flux of microorganism to selectively produce butanol. U.S. Pat. No. 9,005,953 provides recombinant Escherichia coli having butanol production pathways and butanol production capacity. U.S. Pat. No. 4,757,010 provides recombinant microorganism having high butanol and acetone productivity for the production of acetone and butanol mixture. U.S. Pat. No. 7,659,104 provides Enterococcus bacteria having enhanced tolerance to butanol for the production of butanol. U.S. Pat. No. 7,659,105 provides butanol export proteins OmrA or LmrA for the enhanced production of butanol. U.S. Pat. No. 9,284,580 provides recombinant Clostridium tyrobutyricum for the production of butanol and ethanol. U.S. Pat. No. 8,372,672 provides recombinant microorganism having reduced accumulation of (p)ppGpp for the enhanced butanol tolerance. U.S. Patent Application Publication No. 2015/0010984 discloses adaptive recombinant microorganism to produce butanol. U.S. Patent Application Publication No. 2009/0111154 discloses recombinant microorganism with the overexpressing genes encoding for the proteins Thl, Hbd, Bcd and AdhE2 for the improved butanol production. U.S. Patent Application Publication No. 2010/0062505 discloses genetically engineered microorganism having increased cytosolic acetyl-CoA for the butanol production. U.S. Patent Application Publication No. 2015/0299740 discloses recombinant microorganism having suppressed acetyl-CoA to acetate pathway for the enhanced butanol production. U.S. Patent Application Publication No. 2015/0004664 discloses recombinant microorganism comprising deletion of gene coding for Rex, the redox sensing transcriptional repressor for the improved butanol production. U.S. Patent Application Publication No. 2014/0377825 discloses eliminating acetate pathway increases butanol production. U.S. Patent Application Publication No. 2013/0149757 discloses a new Clostridium beijerinckii strain to produce primarily isopropanol and butanol. U.S. Patent Application Publication No. 2011/0097775 discloses a thermophilic Bacillaceae comprising high butanol tolerance for the enhanced butanol production. U.S. Patent Application Publication No. 2010/0143985 discloses recombinant yeast having butanol producing ability in which CoA-transferase capable of converting butyric acid to butyryl-CoA. U.S. Patent Application Publication No. 2010/0151544 discloses a method for enhancing butanol production by enhancing butryl-CoA activity and diminishing acetyl-CoA activity. U.S. Patent Application Publication No. 2010/0330636 discloses enhanced butanol production by eliminating acetate, butyrate and acetone metabolic pathways. U.S. Patent Application Publication No. 2015/0031102 discloses methods of modifying quorum sensing proteins for the improved butanol production. U.S. Patent Application Publication No. 2011/0020888 discloses recombinant microorganism containing gene AdhE and CoA-transferase (CoAT) for the conversion of butyrate to butanol. U.S. Patent Application Publication No. 2010/0159546 discloses genetically engineered microorganism having active metabolic pathways from pyruvate to 1-butanol for the enhanced butanol production. U.S. Patent Application Publication No. 2015/0376655 discloses recombinant microorganism having inhibited acetyl-CoA to acetate pathway and enhanced acetyl-CoA to butyryl-CoA pathway. U.S. Patent Application Publication No. 2009/0155869 discloses inactivation of one or more pathways that compete with NADH dependent heterologous pathway to produce butanol. International PCT Application. Publication No. WO2014/135633 provides recombinant microorganism in which overexpression of genes coding for Crt, Bcd and Hbd resulted increased butanol production. International PCT Application. Publication No. WO2014/006203 provides improved thiolase variants for the enhanced butanol production. International PCT Application. Publication No. WO2014/160050 provides recombinant microorganism having deletion or disruption of glycerol-3-phosphate dehydrogenase for the butanol production. International PCT Application Publication No. WO2012/136826 provides genetically modified Arxula adeninivorans to produce butanol. International PCT Application Publication No. WO2008/074794 provides genetic modification of prokaryotic cell to produce butanol. International PCT Application. Publication No. WO2010/031772 provides alternative butanol production from 2-ketoisovalerate catalyzed by isobutyryl-CoA mutase. International PCT Application. Publication No. WO2012/099934 provides methods for controlling NADH utilization by deleting competing pathways and the genes such as FrdBC, LdhA, Pta and AdhE. International PCT Application Publication No. WO2012/033334 provides gene VorABC encoding enzyme that converts 2-ketoisovalerate to isobutyryl-CoA and butyryl-CoA for the production of butanol.

U.S. Pat. No. 7,501,268 provides recombinant microorganism comprising prenyl diphosphate synthase gene to produce prenyl alcohol. U.S. Patent Application Publication No. 2004/0063182 discloses recombinant microorganism producing prenyl alcohol comprising mutant cell that has been mutated to have reduced squalene synthase activity. U.S. Patent Application Publication No. 2004/0029239 discloses recombinant microorganism having transcription promotor such as PGK1, TEF2, GAL1 and transcription terminator ADH1, CYC1 to produce prenyl alcohol. U.S. Pat. Nos. 9,080,188 and 8,114,641 provide genes, metabolic pathways, microbial strains and methods to produce methyl butanols such as 2-methyl-1-butanol and 3-methyl-2-butanol. U.S. Pat. No. 8,975,049 provides recombinant microorganism useful for producing higher alcohol biofuels such as 1-butanol, 1-propanol, 2-methyl-1-butanol, 3-methyl-1-butanol and 2-phenylethanol. U.S. Pat. No. 8,298,798 provides recombinant microorganism for the production of C5-C8 biofuels such as 3-methyl-1-pentanol, 3-methyl-1-butanol, 2-isopropyl-1-butanol, 4-methyl-1-hexanol, 5-methyl-1-heptanol, 4-methyl-1-pentanol. U.S. Pat. No. 7,985,567 provides recombinant microorganism to produce isopentenol and 3-methyl-1-butanol. U.S. Pat. No. 9,181,566 provides recombinant microorganism comprising promotor nucleic acid to produce 2-butanol. U.S. Pat. No. 8,426,174 employs reduction in temperature during the fermentation that results increased robustness of the recombinant microorganism to the butanol product. U.S. Pat. No. 8,980,612 provides recombinant microorganism and biosynthetic pathways for the production of 2-butanone and 2-butanol. U.S. Pat. No. 8,206,970 provides recombinant microorganism comprising aminobutanol phosphate phosphorylase to produce of 2-butanol. U.S. Pat. No. 9,273,330 provides recombinant microorganism comprising reduced pyruvate carboxylase activity and modified adenylate cyclase activity for the improved production of isobutanol. U.S. Pat. No. 8,828,704 provides recombinant microorganism that uses coenzyme B12 independent butanediol dehydratase that catalyze the conversion of 2,3-butanediol to 2-butanone. U.S. Patent Application Publication No. 2010/0184173 discloses recombinant microorganism to produce 2-butanone and 2-butanol by various biosynthetic pathways. U.S. Patent Application Publication No. 2009/0162911 discloses recombinant microorganism comprising genetic modifications that results in a reduced production of AcrA and AcrB. U.S. Pat. No. 8,158,404 provides recombinant microorganism comprising biosynthetic pathways for the production of isobutanol from 3-ketoacid. U.S. Pat. No. 8,153,415 provides recombinant microorganism for the reduced by-product accumulation and improved formation of isobutanol. U.S. Pat. No. 8,133,715 provides recombinant microorganism having reduce glycerol-3-phosphate dehydrogenase and pyruvate decarboxylase activity for improved isobutanol production. U.S. Pat. No. 7,851,188 provides recombinant microorganism comprising heterologous DNA molecules encoding polypeptides that catalyze pyruvate to isobutanol. U.S. Pat. No. 8,718,328 provides recombinant microorganism comprising inactivated aldehyde dehydrogenase for the improved production of isobutanol. U.S. Pat. No. 8,283,144 provides recombinant microorganism comprising inactivated genes thereby reducing yield loss from competing pathways for carbon flow. U.S. Pat. No. 8,735,114 provides recombinant microorganism further comprising acetolactate synthase, keto-acid reductoisomerase enzymes for the isobutanol production. U.S. Pat. No. 8,889,385 provides recombinant microorganism further comprising dihydroxyacid dehydratase and alcohol dehydrogenase enzymes for the isobutanol production. U.S. Pat. No. 8,951,774 provides recombinant microorganism comprising valine transaminase and valine decarboxylase for the production of isobutanol. U.S. Pat. No. 8,828,694 provides recombinant yeast mitochondria for the production of isobutanol. U.S. Pat. No. 9,249,420 provides recombinant yeast in which gene encoding for OPT1 is deleted for the enhanced production of isobutanol. U.S. Pat. No. 9,234,217 provides recombinant microorganism in which overexpression of NADPH dependent isocitrate dehydrogenase improves the productivity of the isobutanol. U.S. Pat. No. 8,945,859 provides recombinant microorganism encoding biosynthetic pathways for the production of isobutanol. U.S. Pat. No. 8,530,226 provides recombinant microorganism where in increased metabolic flow of materials from pyruvate to isobutanol is achieved for higher isobutanol production. U.S. Pat. No. 8,232,089 provides recombinant microorganism where in cytosolically active dihydroxyacid dehydratase enzyme is used for higher isobutanol production. U.S. Pat. Nos. 7,910,342 and 9,284,612 provide recombinant microorganism using highly active keto-acid reductoisomerase for the fermentative production of isobutanol. U.S. Pat. Nos. 9,068,190 and 7,993,889 provide recombinant microorganism to produce isobutanol from sugar substrates. U.S. Pat. No. 8,455,239 provides recombinant yeast microorganism to produce isobutanol. U.S. Pat. No. 8,017,375 provides recombinant yeast microorganism to produce isobutanol. U.S. Pat. No. 8,785,166 provides recombinant yeast microorganism for the increased production of isobutanol with reduced mitochondrial aminoacid biosynthesis. U.S. Pat. No. 9,012,190 provides the use of thiamine and nicotine adenine dinucleotide for the improved production of isobutanol. U.S. Patent Application Publication No. 2009/0186910 discloses recombinant microorganism comprising increased dihydroxyacid dehydratase activity for the improved isobutanol production. U.S. Patent Application Publication No. 2009/0305369 discloses recombinant microorganism wherein genes coding for AdhE, LdhA, FrdB, PflB are disrupted for the enhanced production of isobutanol. U.S. Patent Application Publication No. 2011/0244536 discloses recombinant microorganism wherein keto-acid reductoisomerase efficiency is improved for the enhanced butanol production. U.S. Patent Application Publication No. 2013/0071898 discloses recombinant microorganism and methods for production of isobutanol. U.S. Patent Application Publication No. 2008/0274526 discloses fermentative conditions in which reduction in fermentation temperature improves butanol tolerance. International PCT Application Publication No. WO2014/037912 provides architecture of energy distribution that can sustain the increased formation of NADH/NADPH for improved isobutanol production.

In one method to enhance DHA uptake and utilization, the genetically modified host cells already known to produce C4-C10 alcohols, as described in patents above, are subjected to further genetic engineering by means of expressing plasmids that code ATP and PEP dependent kinases that could phosphorylate DHA and facilitate its entry into glycolytic cycle. In another method to enhance DHA uptake and utilization, genetically modified host cells already known to produce C4-C10 alcohols, as described in patents above, are subjected to chemical mutagenesis and the strains with the ability to grow and produce desired C4-C10 alcohols with high enough titer and yield in a growth medium comprising DHA as a source of carbon are selected and subjected to whole genome sequencing to identify specific mutations associated with the ability to grow and produce C4-C10 alcohols in a medium comprising DHA. Such specific mutations are introduced into the genetically modified host cells already known to produce C4-C10 alcohols for the purpose of conferring the ability to use DHA as a source of organic carbon. In yet another method to enhance DHA uptake and utilization, genetically modified host cells already known to produce C4-C10 alcohols, as described in patents above, are exposed to growth medium with increasing concentration of DHA and subjected to metabolic evolution. Strains that have gained ability to grow in the medium containing DHA as a major or sole source of carbon and still retain the ability to produce C4-C10 alcohols are selected and subjected to whole genome sequencing to identify specific mutations associated with the ability to grow and produce C4-C10 alcohols in a medium comprising DHA. Such specific mutations are introduced into the genetically modified host cells already known to produce C4-C10 alcohols, as described in patents above, for the purpose of conferring the ability to use DHA as a source of organic carbon.

In another embodiment, the present invention provides methods of producing diols such as 1,4-butanediol, 1,3-propanediol, 2,3-butanediol, 1,3-butanediol, 1,2-propanediol, ethylene glycol, 1,5-pentanediol, and 1,6-hexanediol. In one aspect, genetically modified microorganisms producing diols are subjected to further genetic modifications to confer the ability to use DHA as a source of carbon. In another aspect of this invention, microorganisms that have ability to grow in a medium comprising DHA as a source of carbon is subjected to further genetic modifications to produce diols. The list of genetic modification that are required to increase the production of diol in a microbial cell would involve overexpressing one or more components of the glycerol to 1,3-propanediol biosynthetic pathway, malate to 1,3-propanediol biosynthetic pathway, pyruvate to 1,2-propanediol biosynthetic pathway, dihydroxyacetone to 1,2-propanediol biosynthetic pathway, pyruvate to 2,3-butanediol biosynthetic pathway, succinate to 1,4-butanediol biosynthetic pathway, L-glutamate to 1,4-butanediol biosynthetic pathway, acetyl-CoA to 1,4-butanediol biosynthetic pathway, acetyl-CoA to 1,3-butanediol biosynthetic pathway and succinate to 1,3-butanediol biosynthetic pathway. More specifically, the genetic modifications required to achieve the production of a diol in a microbial cell would involve enhancing the activities of one or more of the enzymes selected from a group consisting of glycerol dehydratase encoded by dhaB gene, 1,3-propanediol oxidoreductase encoded by dhaT gene, vitamin B12 receptor precursor encoded by btuB gene, vitamin B12 transport system permease protein encoded by btuC gene, vitamin B12 transport ATP-binding protein coded by btuD gene, dehydratase reactivation factor for glycerol dehydratase in the glycerol to 1,3-propanediol biosynthetic pathways encoded by orfX, orfY, orfW and orfZ genes; enhancing the activities of one or more enzymes functional in the malate to 1,3-propanediol biosynthetic pathway such as malate kinase, malate semialdehyde dehydrogenase, malate semialdehyde reductase, 2,4-dihydroxybutyrate dehydrogenase, 2-oxo-4-hydroxybutyrate decarboxylase, phosphoenolpyruvate carboxylase, pyruvate carboxylase, malate dehydrogenase and isocitrate lyase; enhancing the activities of one or more enzymes in the acetyl-CoA to 1,3-butanediol biosynthetic pathway including -ketothiolase, acetoacetyl-CoA reductase, 3-oxobutyraldehyde reductase, 3-hydroxybutaraldehyde reductase, 3-hydroxybutyryl-CoA reductase, acetoacetyl-CoA reductase (CoA dependent alcohol forming), acetoacetyl-CoA reductase (ketone reducing), 3-hydroxybutyryl-CoA reductase (aldehyde forming), 3-hydroxybutyryl-CoA reductase (alcohol forming) and 4-hydroxy-2-butanone reductase; enhancing the activities of one or more enzymes functional in the succinate to 1,3-butanediol biosynthetic pathway including α-ketoglutarate dehydrogenase, α-ketoglutarate decarboxylase, succinyl-CoA synthase, Succinate reductase, succinic semialdehyde dehydrogenase, 4-hydroxybutyrate dehydrogenase, 4-hydroxybutyryl-CoA dehydratase, vinylacetyl-CoA-6-isomerase, 3-hydroxybutyryl-CoA dehydratase, 3-hydroxybutyryl-CoA reductase (aldehyde forming) and 3-hydroxybutyryl-CoA reductase (alcohol forming); enhancing the activities of one or more enzymes functional in the in the 2,3-butanediol biosynthetic pathway including acetolactate synthase, acetolactate decarboxylase, acetoin reductase and diacetyl reductase; enhancing the activities of one more enzymes functional in the in the pyruvate to 1,2-propanediol biosynthetic pathway including lactate dehydrogenase, lactoyl-CoA transferase, loctoyl-CoA reductase and 1,2-propanediol oxidoreductase; enhancing the activities of one or more enzymes functional in the in the dihydroxyacetone to 1,2-propanediol biosynthetic pathway including dihydroxyacetone kinase, methylglyoxal synthase, methyl glyoxal reductase (ketone forming), methyl glyoxal reductase (aldehyde forming), acetol reductase and 1,2-propanediol oxidoreductase; enhancing the activities of one or more enzymes functional in the in the acetyl-CoA to 1,4-butanediol biosynthetic pathway including β-ketothiolase, 3-hydroxybutyryl-CoA reductase, 3-hydroxybutyryl-CoA dehydratase, 4-hydroxybutyryl-CoA dehydratase, vinylacetyl-CoA-6-isomerase, aldehyde dehydrogenase and alcohol dehydrogenase; enhancing the activities of one or more enzymes functional in the in the succinate to 1,4-butanediol biosynthetic pathway including α-ketoglutarate dehydrogenase, α-ketoglutarate decarboxylase, succinyl-CoA synthase, Succinate reductase, succinic semialdehyde dehydrogenase, succinic semialdehyde transaminase, 4-hydroxybutyrate dehydrogenase, 4-hydroxybutyryl-CoA transferase, butyrate kinase, phosphotransbutyrylase, aldehyde dehydrogenase and alcohol dehydrogenase; enhancing the activities of one or more enzymes in the L-glutamate to 1,4-butanediol biosynthetic pathway including glutamate-5-kinase, glutamate-5-semialdehyde dehydrogenase, oxidoreductase, aminotransferase, decarboxylase, alcohol dehydrogenase.

Genetically modified microorganisms capable of producing diols using conventional sugars such as glucose, sucrose or glycerol are already known in the art. U.S. Pat. Nos. 8,470,582 and 7,947,483 provide genetically engineered microorganisms where one or more gene disruptions confers stable growth-coupled to the production of 1,4-butanediol (BDO). U.S. Pat. No. 8,129,169 provides exogeneous nucleic acid encoding a BDO pathway enzyme expressed in a sufficient amount to produce BDO. U.S. Pat. Nos. 8,357,520, 8,969,054, 8,889,399 and 8,067,214 provides genetically engineered microorganisms having 4-hydroxybutyric acid biosynthetic pathway further modified to produce BDO. U.S. Pat. Nos. 8,377,666 and 377,667 provide genetically engineered microorganisms to produce BDO, 4-hydroxybutanal, putrescine, 4-hydroxybutyryl-CoA and related compounds. U.S. Pat. No. 9,175,297 provides genetically engineered microorganisms for the conversion of 4-aminobutyric acid and 4-aminobutanol to BDO. U.S. Pat. No. 9,200,288 provides genetically engineered microorganisms for the conversion of D-xylose and D-xylonic acid to BDO. U.S. Pat. No. 8,921,083 provides genetically engineered microorganisms comprising disruptions or deletions of gene encoding transcription regulatory factors NCg12886, NCg12090, NCg10224, and NCg12956. U.S. Pat. No. 9,096,860 and U.S. Patent Application Publication No. 2015/0353964 provide methods for further mutating genetically engineered succinic acid producing microorganism by introducing and amplifying genes encoding enzymes converting succinate to BDO. U.S. Patent Application Publication No. 2015/0376657 discloses production of BDO through acetyl-CoA, acetoacetyl-CoA, 3-hydroxybutyryl-CoA, crotonyl-CoA and 4-hydroxybutyryl-CoA by using genetically engineered microorganisms. U.S. Patent Application Publication No. 2011/0014669 discloses biological methods for the conversion of L-glutamate to BDO using genetically engineered microorganisms that avoids the production of 4-hydroxybutyrate. U.S. Patent Application Publication No. 2014/0030781 discloses genetically engineered Corynebacterium glutamicum with the genes encoding for proteins ThiL, Hbd, Crt, AbfD and DhaT for the production of BDO. U.S. Patent Application Publication No. 2013/0034844 discloses genetically engineered microorganisms comprising BDO, 4-hydroxybutyryl-CoA, 4-hydroxybutanal and putrescine pathway for the increased production of BDO. U.S. Patent Application Publication No. 2015/0148513 discloses genetically engineered microorganisms to produce 4-hydroxybutyrate and BDO.

U.S. Pat. No. 8,658,408 provides genetically engineered microorganisms to produce 2,3-butanediol by anaerobic fermentation using CO as a feedstock. U.S. Pat. No. 9,080,179 and U.S. Pat. No. 8,455,224 provides genetically engineered Lactobacillus plantarum for the enhanced production of 2,3-butanediol from pyruvate through substantial elimination of lactate dehydrogenase. U.S. Patent Application Publication No. 2013/0316419 discloses genetically engineered 2,3-butanediol producing microorganisms that has acetolactate decarboxylase activity substantially higher than the non-modified strain. U.S. Patent Application Publication No. 2013/0330794 discloses genetically engineered 2,3-butanediol producing microorganisms that has acetolactate synthase activity substantially higher than the non-modified strain. U.S. Patent Application Publication No. 2015/0259711 discloses genetically engineered cyanobacteria having acetolactate synthase and acetolactate decarboxylase activity to produce 2,3-butanediol. International PCT Application. Publication No. WO2014/148754 provides genetically engineered 2,3-butanediol producing microorganism where the pathways converting pyruvate to acetyl-CoA, formic acid and lactate is inhibited. U.S. Patent Application No. 2015/0191752 discloses genetically engineered Raoultella planticola for the industrial production of 2,3-butanediol from glycerol. U.S. Patent Application Publication No. 2015/0167027 discloses genetically engineered yeast having a 2,3-butanediol pathway wherein enzymatic activity of pyruvate decarboxylase is inhibited and genes associated with 2,3-butanediol biosynthesis is introduced. U.S. Patent Application Publication No. 2014/0342419 discloses genetically engineered microorganism wherein said microorganism overexpress at least one gene encoding the conversion of pyruvate into 2,3-butanediol. U.S. Patent Application Publication No. 2013/0316418 discloses genetically engineered 2,3-butanediol producing microorganism that has acetoin reductase activity 35 times higher than the unmodified strain.

U.S. Pat. No. 8,497,102 provides genetically engineered microorganisms that comprises of mutant methylglyoxal synthase enzyme for the production 1,2-propanediol. U.S. Pat. No. 8,980,604 provides genetically engineered microorganisms that comprises deletion or attenuation of glycerol dehydrogenase for the enhanced production 1,2-porpanediol. U.S. Pat. No. 8,969,053 provides genetically engineered microorganisms that comprises deletion or attenuation of gene YqhD to produce 1,2-propanediol. U.S. Pat. No. 6,303,352 provides genetically engineered microorganisms that express recombinant methylglyoxal synthase activity for the enhanced production of 1,2-propanediol. U.S. Pat. No. 6,087,140 provides genetically engineered microorganisms that express glycerol dehydrogenase, methylglyoxal synthase and aldose reductase activities for the enhanced production 1,2-propanediol. U.S. Pat. No. 8,298,807 provides genetically engineered microorganisms where the expression of TpiA gene is reduced or eliminate for the enhanced production of 1,2-propanediol. U.S. Pat. No. 7,049,109 provides genetically engineered Klebsiella pneumoniae for the enhanced production of 1,2-propanediol from sugars. U.S. Pat. No. 9,051,588 provides genetically engineered 1,2-propanediol producing microorganisms that has increased methylglyoxal reductase activity by overexpressing one of the YqhD, YafB, YdhF, YcdW, YqhE, YeaE and YghZ genes. U.S. Pat. No. 8,252,579 provides genetically engineered microorganisms where the expression of TpiA, GloA, AldA and AldB gene is reduced or eliminated for the enhanced production of 1,2-propanediol. U.S. Patent Application Publication No. 2015/0159181 discloses genetically engineered microorganism that utilize sucrose as substrate for the fermentative production of 1,2-propanediol. U.S. Patent Application Publication No. 2010/0261239 discloses genetically engineered microorganism that has improved activity of the biosynthetic pathway from dihydroxyacetone phosphate to 1,2-propanediol and attenuated activity of the glyceraldehyde-3-phosphate to produce 1,2-propanediol. U.S. Patent Application Publication No. 2014/0178953 discloses genetically engineered microorganism having lactoyl-CoA reductase activity to produce 1,2-propanediol. International PCT Application. Publication No. WO1999/028481 provides genetically engineered yeast which ferments sugar into hydroxyacetone or 1,2-propanediol. International PCT Application Publication No. WO2015/173247 provides new microorganism and method to produce 1,2-propanediol based on NADPH dependent hydroxyacetone reductase.

U.S. Pat. No. 9,017,983 provides genetically engineered microorganisms having 1,3-butanediol pathways and the production of 1,3-butanediol from acetyl-CoA through acetoacetyl-CoA and 3-hydroxybutyryl-CoA. U.S. Patent Application Publication No. 2016/0076060 discloses genetically engineered microorganism having 1,3-butanediol pathways and production of 1,3-propanediol from L-alanine through 2-amino-4-oxopentanoate, 3-hydroxybutaraldehyde, 3-oxobutyraldehyde and 4-hydroxy-2-butanone. U.S. Patent Application Publication No. 2012/0276606 discloses genetically engineered microorganism to produce 1,3-butanediol where the activity of 3-hydroxybutyryl-CoA reductase and 3-hydroxybutaraldehyde reductase in enhanced. U.S. Patent Application Publication No. 2013/0066035 discloses genetically engineered microorganism having enhanced 1,3-butanediol production that is engineered to produce and increase the availability of cytosolic acetyl-CoA.

U.S. Pat. No. 7,267,972 provides methods for preparing 1,3-propnaediol (PDO) by a genetically engineered microorganism in the absence of Coenzyme B12 or one of its precursor. U.S. Pat. No. 7,371,558 provides genetically engineered microorganisms that comprises genetic disruption of phosphoenolpyruvate-glucose phosphotransferase system for the enhanced production of PDO. U.S. Pat. No. 7,629,161 provides genetically engineered microorganisms that comprises genes encoding enzyme glycerol or diol dehydratse for the improved production of PDO. U.S. Pat. No. 7,135,309 provides genetically engineered microorganisms that has transformed with genes coding for the proteins DhaB1, DhaB2, DhaB3 and DhaT for the improved production of PDO. U.S. Patent Application Publication No. 2015/0147795 discloses genetically engineered microorganism to produce PDO from malate through malate semialdehyde, 2-oxo-4-hydroxybutyrate and 3-hydroxypropionaldehyde. U.S. Pat. No. 7,074,608 provides genetically engineered microorganisms comprising genes for Coenzyme B12 synthesis for the production of PDO. U.S. Pat. No. 7,582,457 provides genetically engineered microorganisms comprising genes encoding cob(II)alamin reductase, cob(I)alamin adenosyltransferase, glycerol dehydratase and 1,3-propanediol oxidoreductase to produce PDO. U.S. Pat. No. 6,432,686 provides genetically engineered microorganisms with genes encoding for BtuB, BtuC, BtuD, glycerol dehydratase and 1,3-propanediol oxidoreductase to produce PDO. U.S. Pat. No. 8,338,148 provides genetically engineered microorganisms in which glycerol oxidative pathway has been blocked to enhance the sugar utilization towards PDO production. U.S. Pat. No. 8,486,673 provides genetically engineered microorganisms for producing 1,3-propanediol using crude glycerol, a by-product from biodiesel production. U.S. Pat. No. 7,745,184 provides genetically engineered microorganisms that has upregulated genes coding for proteins GalP and Glk, and downregulated genes coding for the proteins GapA and ArcA to produce PDO. U.S. Pat. No. 5,633,362 provides genetically engineered microorganisms having glycerol dehydratase enzyme for the production of PDO from glycerol. U.S. Pat. No. 5,821,092 provides bioconversion of glycerol to PDO in which genes from bacteria known to possess diol dehydratase enzyme for the 1,2-propanediol degradation are cloned into a bacterial host for PDO production. U.S. Pat. No. 8,236,994 provides genetically engineered Clostridium sp. with reduced butyrate, butanol and ethanol production and increased production of PDO. U.S. Pat. Nos. 7,067,300, 6,514,733 and 7,452,710 provides genetically engineered microorganisms with the genes encoding for DhaR, OrfY, DhaT, OrfX, OrfW, DhaB1, DhaB2, DhaB3 and OrfZ for the improved production of PDO. U.S. Pat. No. 6,136,576 provides genetically engineered microorganisms with the genes encoding for OrfY, OrfX, OrfW and enzymes glycerol or diol dehydratase for the enhanced production of PDO. U.S. Pat. No. 6,013,494 and U.S. Pat. No. 6,953,684 provides genetically engineered microorganisms comprising genes encoding glycerol-3-phosphate dehydrogenase, glycerol-3-phosphatase, glycerol dehydratase and 1,3-propanediol oxidoreductase to produce PDO. U.S. Patent Application Publication No. 2016/0040195 discloses continuous culture for PDO production using high glycerol concentration. U.S. Patent Application Publication No. 2013/0177956 discloses genetically engineered Clostridium acetobutylicum useful to produce PDO from glycerol. U.S. Patent Application Publication No. 2015/0072388 discloses genetically engineered Halanaerobium sp. for the fermentative production of PDO from glycerol. International PCT Application. Publication No. WO2010/079500 provides genetically engineered Klebsiella pneumoniae for the aerobic production of PDO from crude glycerol from biodiesel process.

In one method to enhance DHA uptake and utilization, the genetically modified host cells already known to produce diols, as described in patents above, is subjected to further genetic engineering by means of expressing plasmids that code ATP and PEP dependent kinases that could phosphorylate DHA and facilitate its entry into glycolytic cycle. In one method to enhance DHA uptake and utilization, genetically modified host cells already known to produce diols, as described in patents above, are subjected to chemical mutagenesis and the strains with the ability to grow and produce desired diol with high enough titer and yield in a growth medium comprising DHA as a source of carbon are selected and subjected to whole genome sequencing to identify specific mutation associated with the ability to grow and produce diol in a medium comprising DHA. Such specific mutations are introduced into the genetically modified host cells already known to produce diols for the purpose of conferring the ability to use DHA as a source of organic carbon. In another method to enhance DHA uptake and utilization, genetically modified host cells already known to produce diols, as described in patents above, are exposed to growth medium with increasing concentration of DHA and subjected to metabolic evolution. Strains that have gained ability to grow in the medium containing DHA as a major or sole source of carbon and still retain the ability to produce diols are selected and subjected to whole genome sequencing to identify specific mutations associated with the ability to grow and produce diols in a medium comprising DHA. Such specific mutations are introduced into the genetically modified host cells already known to produce diols, as described in patents above, for the purpose of conferring the ability to use DHA as a source of organic carbon.

In one embodiment, the present invention provides methods of producing isoprenoids in the form of carotenoids, monoterpenoids, diterpenoids, sesquiterpenoids and prenyl compounds. In one aspect, genetically modified microorganisms producing isoprenoids are subjected to further genetic modifications to confer the ability to use DHA as a sole or major source of carbon. In another aspect of this invention, microorganisms that have ability to grow in a medium comprising DHA as a source of carbon is subjected to further genetic modifications to produce one or other isoprenoids. The genetic modifications required to produce one or other isoprenoids would involve overexpressing one or more components of the carotenoid biosynthetic pathway, monoterpenoids biosynthetic pathway, diterpenoids biosynthetic pathway, sesquiterpenoids biosynthetic pathway and prenyl biosynthetic pathway. The list of genetic manipulations required to enhance the isoprenoid biosynthesis within the microbial cell includes but not limited to: enhancing the activities of one or more of the enzymes involved in the carotenoid biosynthetic pathway such as phytoene synthase, phytoene dehydrogenase, lycopene cyclase, beta-carotene dioxygenase, retinol dehydrogenase, zeaxanthin epoxidase, carotenoid 1,2-hydratase, beta-carotene ketolase, beta-carotene 3-hydroxylase, hydroxycarotenoid 3,4-desaturase, demethylspheroidene O-methyl transferase, carotene epsilon monooxygenase, beta-ring hydroxylase, lycopene epsilon cyclase and lycopene beta-cyclase; enhancing the activities of one or more of the enzymes involved in the monoterpenoid biosynthetic pathways such as geraniol hydrolase, linalool synthase, myrcene synthase, limonene synthase, pinene synthase, fenchol synthase, sabinene synthase, terpinol synthase, carvol dehydrogenase, limonene monooxygenase, isopiperitenol dehydrogenase, isopiperitenone reductase, isopulegone dehydrogenase, carveol dehydrogenase, pulegone reductase, methol dehydrogenase, neomenthol dehydrogenase, menthofuran synthase, comphene synthase, bornyl synthase in the monoterpenoids biosynthetic pathways; enhancing the activities of one or more of the enzymes involved in the sesquiterpenoid biosynthetic pathway such as alpha-farnesene synthase, beta-farnesene synthase, nerolidol synthase, isozizaene synthase, cedrol synthase, bisbolene synthase, macrocarpene synthase, trichodiene synthase, amorphadiene synthase, santelene synthase, barbatene synthase, chamigrene synthase, thujopsene synthase, germacrene synthase, germacradienol synthase, aristolochene synthase, epiaristolochene synthase, valencene synthase, vetispiradiene synthase, selinenesynthase, episelinene synthase, patchoulol synthase, avermitilol synthase, caryophyllene synthase, longifolene synthase, humulene synthase, pentalene synthase, cadinene synthase and muuroladiene synthase; enhancing the activities of one or more of the enzymes involved in the in the diterpenoid biosynthetic pathway including casbene synthase, taxadiene synthase, levopimaradiene synthase, abietadiene synthase, isopemaradiene synthase, neosbietadiene synthase, paulstradiene synthase, miltiradiene synthase, copalyl synthase, cembrene synthase, labdadiene synthase, fusicocca-2, 10(14)-diene synthase, ent-sandaracopimaradiene synthase, pimara-7,15-diene synthase, ent-pimara-8(14)-15-diene synthase, ent-kaur-15-ene synthase, ent-kaur-16-ene synthase, syn-stemar-13 (17)-ene synthase, entcopalyl synthase, syn-stemar-13-ene synthase; enhancing the activities of one or more of the enzymes involved in the prenyl biosynthetic pathway including geranyl diphosphate synthase, farnesyl diphosphate synthase, geranylgeranyl diphosphate synthase, hexaprenyl diphosphate synthase, heptaprenyl diphosphate synthase, octaprenyl diphosphate synthase, nonaprenyl diphosphate synthase, solanesyl diphosphate synthase, decaprenyl diphosphate synthase, chicle synthase, gutta-percha synthase, Z-nonaprenyl diphosphate synthase, Z-undecaprenyl diphosphate synthase, Z-dihydrodolichyl diphosphate synthase, Z-eicosaprenyl diphosphate synthase and rubber ci s-polyprenyl ci stransferase.

In another aspect of this embodiment, microorganisms that have ability to grow in DHA in a medium comprising DHA as a source of carbon is subjected to further genetic modifications to include one or more nucleic acid molecules comprising one or more nucleotide sequences encoding one or more of either mevalonate pathway or DXP pathway enzymes and nucleic acid molecules comprising nucleotide sequence that encodes prenyl biosynthetic pathway enzymes. Suitable prenyl biosynthetic pathway enzymes include enzymes that catalyze the conversion of metabolite dimethylallyl pyrophosphate and isopentyl pyrophosphate to form isoprenoid compounds with from 2 isoprene units to about 6000 isoprene units or more. Microorganisms that already has an ability to produce high amounts of dimethylallyl pyrophosphate or isopentyl pyrophosphate either natively or by one or more genetic modifications are a preferred host to produce prenyl compounds. On the other hand, prenyl biosynthetic pathway can be directly engineered in the host cell that previously lack the ability to produce dimethylallyl pyrophosphate and isopentyl pyrophosphate but can be engineered to selectively produce prenyl compounds. Furthermore, one or more of the enzymes functional in the prenyl biosynthetic pathway such as geranyl diphosphate synthase, farnesyl diphosphate synthase, geranylgeranyl diphosphate synthase, hexaprenyl diphosphate synthase, heptaprenyl diphosphate synthase, octaprenyl diphosphate synthase, nonaprenyl diphosphate synthase, solanesyl diphosphate synthase, decaprenyl diphosphate synthase, chicle synthase, gutta-percha synthase, Z-nonaprenyl diphosphate synthase, Z-undecaprenyl diphosphate synthase, Z-dihydrodolichyl diphosphate synthase, Z-eicosaprenyl diphosphate synthase, and rubber cis-polyprenylcistransferase are overexpressed for the enhanced production of prenyl compounds. Moreover, improvement of dimethylallyl pyrophosphate and isopentyl pyrophosphate production by overexpressing one or more enzymes in mavolonyl pathway or DXP pathway enhances the productivity of prenyl compounds. Some non-limiting examples of a prenyl compound thus produced, include geraniol, farnesol, geranylgeraniol, hexaprenyl alcohol, heptaprenyl alcohol, octaprenyl alcohol, decaprenyl alcohol and nonaprenyl alcohol. In addition, attenuation or deletion of one or more of enzymes including methylglyoxal synthase, acetate kinase, pyruvate carboxylase, phosphoenolpyruvate carboxylase, succinate reductase, citrate synthase, aldehyde dehydrogenase, pyruvate oxidase, pyruvate-formate lyase, alanine transaminase, lactate dehydrogenase, acetolactate synthase, acetyl-CoA carboxylase and beta-ketothiolase improves pyruvate utilization in prenyl biosynthesis. Furthermore, blocking one or more of prenyl degradation pathways such as the formation of monoterpenoids, diterpenoids, sesquiterpenoids and carotenoids and utilization of prenyl compounds to produce other downstream metabolites improves prenyl accumulation. Furthermore, improving prenyl transport, reducing uptake from media and reducing feedback inhibition increases prenyl productivity.

In some embodiments, microorganisms that have ability to grow in DHA in a medium comprising DHA as a source of carbon is subjected to further genetic modifications to include one or more nucleic acid molecules comprising one or more nucleotide sequences encoding diterpenoid biosynthetic pathway enzymes. Suitable diterpenoid synthesis enzymes catalyze the conversion of metabolite geranylgeranyl pyrophosphate to one or more of the diterpenoid compounds. Microorganisms that already has an ability to produce high amounts of geranylgeranyl pyrophosphate either natively or by one or more genetic modifications are a preferred host to produce diterpenoids. On the other hand, diterpenoid biosynthetic pathway can be directly engineered in the host cell that previously lack the ability to produce geranylgeranyl pyrophosphate but can be engineered to selectively produce diterpenoid. Furthermore, one or more of the enzymes selected from a group of diterpenoid biosynthetic pathway such as casbene synthase, taxadiene synthase, levopimaradiene synthase, abietadiene synthase, isopemaradiene synthase, neosbietadiene synthase, paulstradiene synthase, miltiradiene synthase, copalyl synthase, cembrene synthase, labdadiene synthase, fusicocca-2, 10(14)-diene synthase, ent-sandaracopimaradiene synthase, pimara-7,15-diene synthase, ent-pimara-8(14)-15-diene synthase, ent-kaur-15-ene synthase, ent-kaur-16-ene synthase, syn-stemar-13(17)-ene synthase, entcopalyl synthase and syn-stemar-13-ene synthase are overexpressed for the enhanced production of diterpenoids from geranylgeranyl pyrophosphate. Moreover, improvement of geranylgeranyl pyrophosphate production by overexpressing one or more enzymes in prenyl biosynthetic pathway enhances the productivity of diterpenoid. Some non-limiting examples of a diterpenoid compound thus produced, include geranylgeraneol, gibberellin, taxol, taxadiene, copalol, aconitine, veatchine, kauroic acid, hydroxyisokaurene, hydroxycassadiene, sandracopimaradiene, pimaradiene, abietate, levopimaric acid, neoabietic acid, paulstric acid, isopimaric acid, miltiradiene, stemarene, stemedenic acid, aphidicolin, momilactone, pimaradiene, labdatriene, cembrene, casbene, lab dadieneol, sclareol, fusicoccadiene, plaunotol, geranyllinalool, abietadiene, levopimaradiene, neoabietadiene, palustradiene, isopimaradiene, citronellol, cubebol, nootkatone, cineol, limonene, eleutherobin, sarcodictyin, pseudoterosins, labdenediol, ginkgolides, stevioside, rebaudioside A, sclareol, levopimaradiene, sandracopimaradiene, isopemaradiene, arbrusideE and stemodene. In addition, attenuation or deletion of one or more of enzymes including methylglyoxal synthase, acetate kinase, pyruvate carboxylase, phosphoenolpyruvate carboxylase, succinate reductase, citrate synthase, aldehyde dehydrogenase, pyruvate oxidase, pyruvate-formate lyase, alanine transaminase, lactate dehydrogenase, acetolactate synthase, acetyl-CoA carboxylase and beta-ketothiolase improves pyruvate utilization in diterpenoid biosynthesis. Furthermore, blocking one or more of dipterpenoid degradation pathways such as the formation of glycoside catalyzed by glucosyl transferases and utilization of diterpenoids to produce other downstream metabolites improves diterpenoid accumulation. Furthermore, improving diterpenoid transport, reducing uptake from media and reducing feedback inhibition increases diterpenoid productivity.

In some aspect of this embodiment, microorganisms that have ability to grow in a medium comprising DHA as a source of carbon is subjected to further genetic modifications to include one or more nucleic acid molecules comprising one or more nucleotide sequences encoding sesquiterpenoid biosynthetic pathway enzymes including the enzymes that catalyze the conversion of metabolite farnesyl pyrophosphate to form one or more of the sesquiterpenoid compounds. Microorganisms that already have an ability to produce high amounts of farnesyl pyrophosphate either natively or by one or more genetic modifications are a preferred host for the production of sesquiterpenoids. On the other hand, sesquiterpenoid biosynthetic pathway can be directly engineered in to the host cell that previously lack the ability to produce farnesyl pyrophosphate but can be engineered to selectively produce sesquiterpenoid. Furthermore, one or more of the enzymes selected from a group of sesquiterpenoid biosynthetic pathway such as alpha-farnesene synthase, beta-farnesene synthase, nerolidol synthase, isozizaene synthase, cedrol synthase, bisbolene synthase, macrocarpene synthase, trichodiene synthase, amorphadiene synthase, santelene synthase, barbatene synthase, chamigrene synthase, thujopsene synthase, germacrene synthase, germacradienol synthase, aristolochene synthase, epiaristolochene synthase, valencene synthase, vetispiradiene synthase, selinenesynthase, episelinene synthase, patchoulol synthase, avermitilol synthase, caryophyllene synthase, longifolene synthase, humulene synthase, pentalene synthase, cadinene synthase and muuroladiene synthase are overexpressed for the enhanced production of sesquiterpenoids from farnesyl pyrophosphate. Moreover, improvement of farnesyl pyrophosphate production by overexpressing one or more enzymes in prenyl biosynthetic pathway enhances the productivity of sesquiterpenoid. Some non-limiting examples of a sesquiterpenoid compound thus produced, include farnesol, alpha-farnesene, beta-farnesene, S-nerolidol, R-nerolidol, epi-isozizaene, albaflavenone, epi-cedrol, alpha-bisbolene, beta-bisbolene, gamma-bisbolene, macrocarpene, trichodiene, nivalenol, amorphadiene, alpha-santelene, beta-santelene, beta-chamigrene, thujopsene, germacreneA, germacreneB, germacreneC, germacreneD, geosmin, aristolochene, epiaristolochene, valencene, vetispiradiene, solavetivone, bete-selinene, alpha-selinene, delta-selinene, patchoulol, avermitilol, caryophyllene, caryolanol, longifolene, alpha-humulene, delta-humulene, pentalenene, taxadiene, citronellol, cubebol, nootkatone, cineol, limonene, eleutherobin, sarcodictyin, pseudoterosins, labdenediol, ginkgolides, stevioside, rebaudioside A, sclareol, levopimaradiene, sandracopimaradiene, isopemaradiene, arbrusideE, muuroladiene, alpha-zingiberne, sesquiphellandrene, curcumene, gossonorol, alpha-, gamma-, delta-cadinene, caryophyllene, vetivazulene, guaiazulene, longifolene, copaene, patchoulol, dictyophorine A, dictyophorine B, eudesman, ermophilan, valeran, cadinan, driman, guajan, himachalan, longipinan, icrotoxan, daucan, illudan, prezizaan, marasman, acoran, chamigran, cedarn, thujopsan, hirsutan, limonene, terpinolene, terpinene, sabinene, myrcene, ocimene, germacrenes, elemenes, copaene, cadiene and bourbonene. In addition, attenuation or deletion of one or more of enzymes including methylglyoxal synthase, acetate kinase, pyruvate carboxylase, phosphoenolpyruvate carboxylase, succinate reductase, citrate synthase, aldehyde dehydrogenase, pyruvate oxidase, pyruvate-formate lyase, alanine transaminase, lactate dehydrogenase, acetolactate synthase, acetyl-CoA carboxylase and beta-ketothiolase improves pyruvate utilization in sesquiterpenoid biosynthesis. Furthermore, blocking one or more of sesquiterpenoid degradation pathways such as the formation of glycoside catalyzed by glucosyl transferases and utilization of sesquiterpenoids to produce other downstream metabolites improves sesquiterpenoid accumulation. Furthermore, improving sesquiterpenoid transport, reducing uptake from media and reducing feedback inhibition increases sesquiterpenoid productivity.

In some embodiments, microorganisms that have ability to grow in a medium comprising DHA as a source of carbon is subjected to further genetic modifications to include one or more nucleic acid molecules comprising one or more nucleotide sequence encoding monoterpenoid biosynthetic pathway enzymes. Suitable monoterpenoid synthesis enzymes include enzymes that catalyze the conversion of metabolite geranyl pyrophosphate to form one or more of the monoterpenoid compounds. Microorganisms that already has an ability to produce high amounts of geranyl pyrophosphate either natively or by one or more genetic modifications are a preferred host for the production of monoterpenoids. On the other hand, monoterpenoid biosynthetic pathway can be directly engineered in to the host cell that previously lack the ability to produce geranyl pyrophosphate but can be engineered to selectively produce monoterpenoids. Furthermore, one or more of the enzymes selected from monoterpenoid biosynthetic pathway such as geraniol hydrolase, linalool synthase, myrcene synthase, limonene synthase, pinene synthase, fenchol synthase, sabinene synthase, terpinol synthase, carvol dehydrogenase, limonene monooxygenase, isopiperitenol dehydrogenase, isopiperitenone reductase, isopulegone dehydrogenase, carveol dehydrogenase, pulegone reductase, methol dehydrogenase, neomenthol dehydrogenase, menthofuran synthase, comphene synthase and bornyl synthase are overexpressed for the enhanced production of monoterpenoids from geranyl pyrophosphate. Moreover, improvement of geranyl pyrophosphate production by overexpressing one or more enzymes in prenyl biosynthetic pathway enhances the productivity of monoterpenoid. Some non-limiting examples of a monoterpenoid compound thus produced, including geraniol, linalool, myrecene, linalool, oxolinalool, hydroxylinolool, limonene, carvol, carvone, perillyl alcohol, isopiperitenol, isopiperitenone, isopulegone, pulegone, menthone, menthofuran, isomenthone, menthol, neomenthol, pinene, endo-fenchol, sabinene, terpineol, cineole, oxocineole, hydroxycineole, comphene and bornylol. In addition, attenuation or deletion of one or more of enzymes methylglyoxal synthase, acetate kinase, pyruvate carboxylase, phosphoenolpyruvate carboxylase, succinate reductase, citrate synthase, aldehyde dehydrogenase, pyruvate oxidase, pyruvate-formate lyase, alanine transaminase, lactate dehydrogenase, acetolactate synthase, acetyl-CoA carboxylase and beta-ketothiolase improves pyruvate utilization in monoterpenoid biosynthesis. Furthermore, blocking one or more of monoterpenoid degradation pathways such as the formation of glycoside catalyzed by glucosyl transferases and utilization of monoterpenoids to produce other downstream metabolites improves monoterpenoid accumulation. Furthermore, improving monoterpenoid transport, reducing uptake from media and reducing feedback inhibition increases monoterpenoid productivity.

In some embodiments, microorganisms that have ability to grow in DHA in a medium comprising DHA as a source of carbon is subjected to further genetic modifications to include one or more nucleic acid molecules comprising one or more nucleotide sequences encoding carotenoid biosynthetic pathway enzymes. Suitable carotenoid biosynthesis pathway enzymes include enzymes that catalyze the conversion of metabolite geranylgeranyl pyrophosphate to form one or more of the carotenoid compounds. Microorganisms that already has an ability to produce high amounts of geranylgeranyl pyrophosphate either natively or by one or more genetic modifications are a preferred host to produce carotenoids. On the other hand, carotenoid biosynthetic pathway can be directly engineered in the host cell that previously lack the ability to produce geranylgeranyl pyrophosphate but can be engineered to selectively produce carotenoids. Furthermore, one or more of the enzymes selected from a group of carotenoid biosynthetic pathway such as phytoene synthase, phytoene dehydrogenase, lycopene cyclase, beta-carotene dioxygenase, retinol dehydrogenase, zeaxanthin epoxidase, carotenoid 1,2-hydratase, beta-carotene ketolase, beta-carotene 3-hydroxylase, hydroxycarotenoid 3,4-desaturase, demethylspheroidene O-methyl transferase, carotene epsilon monooxygenase, beta-ring hydroxylase, lycopene epsilon cyclase, and lycopene beta-cyclase are overexpressed for the enhanced production of carotenoids from geranylgeranyl pyrophosphate. Moreover, improvement of geranylgeranyl pyrophosphate production by overexpressing one or more enzymes in prenyl biosynthetic pathway enhances the productivity of carotenoids. Some non-limiting examples of a carotenoid compound thus produced, include myxobacton, spheroidene, spheroidenone, lutein, violaxanthin, 4-ketorulene, myxoxanthrophyll, echinenione, canthaxanthin, phytoene, α-, γ-, β-, δ-, ϵ-carotene lycopene, β-cryptoxanthin monoglucoside and neoxanthin. In another embodiment, some non-limiting examples of carotenoids may include antheraxanthin, adonixanthin, astaxanthin, canthaxanthin, capsorubrin, β-cryptoxanthin, echinenone, zeta-carotene, alpha-cryptoxanthin, diatoxanthin, 7,8-didehy droastaxanthin, fucoxanthin, fucoxanthinol, isorenieratene, lactucaxanthin, lutein, lycopene, neoxanthin, neurosporene, hydroxy-neurosporene, peridinin, phytoene, rhodopin, rhodopin glucoside, siphonaxanthin, spheroidene, spheroidenone, spirilloxanthin, uriolide, uriolide acetate, violaxanthin, zeaxanthin-β-diglucoside and zeaxanthin. This list of carotenoids are to be considered as part of this invention. In addition, attenuation or deletion of one or more of enzymes methylglyoxal synthase, acetate kinase, pyruvate carboxylase, phosphoenolpyruvate carboxylase, succinate reductase, citrate synthase, aldehyde dehydrogenase, pyruvate oxidase, pyruvate-formate lyase, alanine transaminase, lactate dehydrogenase, acetolactate synthase, acetyl-CoA carboxylase and beta-ketothiolase improves pyruvate utilization in carotenoid biosynthesis. Furthermore, blocking one or more of carotenoid degradation pathways such as the formation of glycoside catalyzed by glucosyl transferases and utilization of carotenoids to produce other downstream metabolites improves carotenoid accumulation. Furthermore, improving carotenoid transport, reducing uptake from media and reducing feedback inhibition increases carotenoid productivity.

Genetically microbial cells capable of producing isoprenoids and isoprenoid precursors using conventional sugars such as glucose, sucrose or glycerol are already known in the art. For example, U.S. Patent Application Publication No. 2012/8257957 provides details about genetically modified host cells producing isoprenoids and terpenoids. The U.S. Pat. No. 7,670,825 provides genetically modified host cells and methods for the enhanced production of isoprenoids, terpenoids and carotenoids. The U.S. Pat. No. 8,062,878 provides recombinant expression of terpenoid synthase enzymes and geranylgeranyl diphosphate synthase enzymes in genetically modified microbial cells to produce diterpenoid. The U.S. Pat. No. 8,114,645 provides methods for increasing isoprenoid production in a genetically modified host cell by modulating fatty acid levels in the cell. The U.S. Pat. No. 8,512,988 provides methods for increasing the production of pharmaceutical products from the isoprenoid pathway in a genetically modified host cell. The U.S. Pat. Nos. 7,741,070 & 7,695,932 describe the genetic modifications useful for the enhanced production of carotenoids. The U.S. Patent Application Publication No. 2013/0298861 describes the production of sesquiterpenes in a genetically modified host cell. U.S. Patent Application Publication No. 2015/0044747 describes the production of isoprenol in a genetically modified host cell comprising a phosphatase capable of catalyzing the dephosphorylation of dimethylallyl diphosphate. U.S. Patent Application Publication No. 2010/0180491 describes a method of culturing genetically modified host cell which expresses an enzyme capable of catalyzing the esterification of an isoprenol and straight chain fatty acid to produce isoprenyl alkanoates. The U.S. Pat. Nos. 5,429,939, 5,530,118, 5,530,189 and 5,656,472 describe the synthesis of carotenoid in a genetically modified host cell. U.S. Patent Application Publication No. 2012/0164678 describes synthesis of natural sweetener steviol glycosides by recombinant expression of steviol or steviol glycosides biosynthetic pathway enzymes in a genetically modified host cell. U.S. Pat. Nos. 6,531,303 and 6,972,191 describe biological methods of producing geranylgeraniol. U.S. Pat. No. 6,689,593 describes biological methods of producing farnesol and geranylgeraniol. U.S. Pat. No. 7,718,417 describes biological methods of producing isoprenoids by DXP metabolic pathway. U.S. Pat. No. 7,732,161 describes biological methods of producing mevalonic acid. U.S. Pat. Nos. 7,838,279, 7,842,497, 7,927,861, 8,828,684 and 7,927,862 describe a recombinant yeast having mevalonate pathway to produce isoprenoids. U.S. Pat. Nos. 8,236,552, 7,622,283 and 8,241,888 describe biological methods of producing mevalonic acid. U.S. Pat. No. 9,102,954 describes a recombinant yeast and biological methods to produce isoprenoids. U.S. Pat. No. 7,192,751 describes genetically engineered microorganism to produce amorpha-4,11-diene. U.S. Pat. Nos. 6,072,045, 8,354,504, 8,741,651, 5,824,744 and 8,106,260 describes biological methods of producing chimeric isoprenoids using genetically engineered microorganism comprising isoprenoid synthase. U.S. Pat. Nos. 6,114,160, 6,610,527, 7,273,755 and 5,994,114 describe genetically engineered microorganisms for the production of Taxol. U.S. Pat. No. 6,284,506 describes process to produce isoprenoids and terpenoids using genetically engineered microorganism. U.S. Pat. Nos. 6,989,257 and 7,232,679 describes isolated polypeptides used for the production of carotenoids. U.S. Pat. Nos. 7,659,097 and 9,200,296 describes genetically engineered microorganism to produce isoprenoids. U.S. Pat. No. 8,114,645 describes increased isoprenoid precursor production by modulating fatty acid levels. U.S. Pat. No. 8,257,957 provides genetically modified host cells for producing isoprenoids. U.S. Pat. No. 8,828,684 provides genetically engineered microorganism with increased levels of prenyltransferase and decreased levels of squalene synthase for isoprenoid production. U.S. Pat. No. 9,200,298 describes the microbial production of isoprenyl alkanoates. U.S. Pat. Nos. 7,172,886, 7,622,282, 7,667,017, 7,736,882, 7,915,026 and 8,288,147 describe microbial production of isopentyl pyrophosphate. U.S. Pat. No. 5,871,988 describes DNA encoding limonene synthase from Mentha spicata for the production of limonene. U.S. Pat. Nos. 8,124,811, 8,362,309, 7,622,614 and 8,642,615 describes methods of producing fragrances using genetically modified organisms comprising isoprenoid pathways. U.S. Pat. Nos. 8,192,950, 6,265,639, 7,790,426, 7,273,735 and 6,451,576 describe sesquiterpene gene for the production of sesquiterpenes. U.S. Pat. Nos. 8,481,286, 8,609,371, 8,835,139, 7,442,785, 7,790,413 and 8,753,842 describe the deletion of squalene synthase genes and its variants for the improved production of isoprenoids. U.S. Patent Application Publication Nos. 2014/022434 2014/0073020 describe polypeptides comprising labdenediol, sclareol and ambroxide synthase enzymes for the production there of. U.S. Pat. Application No. 2015/0007368 and U.S. Pat. Application No. 2012/0246767 describes polypeptide comprising valencene synthase to produce valencene. U.S. Pat. Nos. 6,468,772, 6,495,354, 6,559,297, 6,569,656, 6,645,762 and 6,890,752 describes nucleic acid encoding novel terpene synthases. U.S. Patent Application No. 2015/0361469 describes the biosynthesis of cannabinoids. U.S. Pat. No. 9,297,004 and U.S. Patent Application Publication No. 2011/0008836 describe the production of santalene by genetically modified microorganism. U.S. Pat. No. 8,629,321 describes farnesyl diphosphate synthase and sesquiterpene synthase to produce sesquiterpenoids. U.S. Pat. No. 6,342,380 describes germacrene C synthase gene to produce germacrenes. U.S. Pat. Nos. 7,541,172 and 7,091,027 describes polypeptide encoding amorpha-4,11-diene synthase. U.S. Pat. Nos. 8,288,149, 8,691,555, 9,297,031 and 7,851,199 describe system for producing engineered oleaginous yeast or fungi that express carotenoids. U.S. Pat. Nos. 7,129,392, 7,618,819, 6,586,202, 7,132,268, 8,951,764 and 8,999,682 describes materials and methods for increasing isoprenoids production in microbial cells. U.S. Pat. No. 7,238,514 describes unicellular organism such as yeast to produce diterpenes. U.S. Pat. Nos. 8,617,860, 8,586,328 and 9,267,155 describes polypeptide with sclarol synthase activity for a method of producing sclareol. U.S. Pat. Nos. 4,245,109, 5,182,208, 5,356,809, 5,356,810, 5,466,599, 5,599,711, 5,648,261, 5,679,567, 5,656,472, 5,709,856, 5,712,110, 5,879,927, 5,916,791, 5,922,560, 5,972,642, 6,015,684, 6,022,701, 6,413,736, 7,063,957, 7,074,604, 7,723,066, and 7,999,151, and U.S. Pat. Application Publication Nos. 2005/0124032, 2004/0078846, 2013/0012594 describe the production of astaxanthin from a genetically engineered microorganism. U.S. Pat. Nos. 2,959,521, 2,959,522, 3,095,357, 3,128,236, 3,226,302, 3,235,467, 3,242,054, 3,378,460, 3,421,980, 3,492,202, 5,328,845, 5,691,190, 4,318,987, 5,530,188, 8,859,228, 6,372,479 and 5,422,247 describe genetically engineered microorganism to produce high beta-carotene. U.S. Pat. Nos. 3,891,504, 3,841,967, 3,951,742, 3,951,743, 5,308,759, 5,427,783, 8,883,443, 5,684,238 and 5,360,730 and U.S. Patent. Application Publication No. 2006/0121557 describe zeaxanthin producing genetically engineered microorganisms. U.S. Pat. Nos. 2,974,044, 5,607,839, 5,858,761, 5,935,808, 6,087,152, 6,124,133, 6,207,409, 6,291,204, 6,627,795, 6,613,543, 6,677,134, 7,745,170, 6,696,293, 6,821,749, 6,869,773, 6,291,204, 6,677,134, 7,063,956, 6,642,021, 7,217,537, 7,504,236, 8,030,022, 7,687,246, 8,846,374, 8,691,555, 7,205,123, 6,329,141, and 8,252,489 and U.S. Patent Application Publication Nos. 2009/0197321, 2005/0003474, 2006/0234333 and U.S. Pat. No. describes genetically engineered microorganism to produce carotenoids. U.S. Pat. Nos. 5,811,273, 6,150,130 and 5,972,690 describes DNA strands useful for the synthesis of xanthophylls and the process for producing it. U.S. Pat. Nos. 6,551,807 and 7,425,625 describe carotenoid ketolase gene for the production of ketocarotenoids. U.S. Pat. Nos. 7,183,089 and 7,670,825 describes method for enhancing the production of isoprenoid compounds. U.S. Pat. No. 9,121,038 describes recombinant microorganism for the enhanced production of mevalonate, isoprene and isoprenoids. U.S. Pat. Nos. 3,467,579, 8,232,083, 8,828,697, 3,097,146, 3,369,174, 5,350,189 and 6,696,282, U.S. Patent Application Publication No. 2005/0059134 describes methods of producing lycopene by recombinant microorganism. U.S. Pat. Nos. 8,030,022 and 8,569,014 and U.S. Patent Application Publication No. 2006/0121556 describe recombinant microorganism to produce canthaxanthin. U.S. Pat. Application Publication No. 2003/0207947 and U.S. Pat. No. 3,280,502 describe recombinant microorganism to produce lutein. U.S. Pat. No. 7,098,000 describes methods to produce C30-aldehyde carotenoids. U.S. Pat. No. 5,545,816 describes the production of phytoene by recombinant microorganism. U.S. Patent Application Publication Nos. 2007/0161712 and 2002/0086380 describe genes encoding epsilon lycopene synthase and methods of producing epsilon carotene. U.S. Pat. No. 8,889,381 describes enzyme diterpene synthase and the methods of producing diterpenes. U.S. Pat. No. 5,189,187 describes microbial production of clerodane type diterpenes. U.S. Pat. No. 7,132,257 describes production of aromatic carotenoids in gram negative bacteria. U.S. Patent Application Publication No. 2014/0248668 describes method and materials for the recombinant production of saffron compounds. U.S. Patent Application Publication Nos. 2014/0329281 and 2015/0361476 describe recombinant production of steviol glycosides. International PCT application. No. WO2015/028324 provides recombinant microorganisms to produce resveratrol. U.S. Pat. Application Publication No. 2006/0053513 describes methods of producing ketocarotenoids by genetically modified organisms. Recently, retinoid production has been achieved using a genetically modified E. Coli (Microbial Cell Factories, 2011, 10, 59-71 and Appl. Microbiol. Biotechnol., 2015, 99, 7813-7826).

In one method to enhance DHA uptake and utilization, the genetically modified host cells already known to produce isoprenoids, as described in patents above, are subjected to further genetic engineering by means of expressing plasmids that code ATP and PEP dependent kinases that could phosphorylate DHA and facilitate its entry into glycolytic cycle. In one method to enhance DHA uptake and utilization, genetically modified host cells already known to produce isoprenoids, as described in patents above, are subjected to chemical mutagenesis and the strains with the ability to grow and produce desired isoprenoid with high enough titer and yield in a growth medium comprising DHA as a source of carbon are selected and subjected to whole genome sequencing to identify specific mutations associated with the ability to grow and produce isoprenoid in a medium comprising DHA. Such specific mutations introduced to the genetically modified host cells already known to produce isoprenoid to confer the ability to use DHA as a source of organic carbon. In another method to enhance DHA uptake and utilization, genetically modified host cells already known to produce isoprenoids, as described in patents above, is exposed to growth medium with increasing concentration of DHA and subjected to metabolic evolution. Strains that have gained ability to grow in the medium containing DHA as a major or sole source of carbon and still retain the ability to produce desired isoprenoids are selected and subjected to whole genome sequencing to identify specific mutations associated with the ability to grow and produce isoprenoid in a medium comprising DHA. Such specific mutations are introduced to the genetically modified host cells already known to produce isoprenoid, as described in patents above, for the purpose of conferring the ability to use DHA as a source of organic carbon.

In another embodiment, the present invention provides methods of producing fatty acids in the form of saturated fatty acids, monounsaturated fatty acids, polyunsaturated fatty acids (PUFA), fatty alcohols, iso-branched fatty acid, anteiso-branched fatty acid and odd chain fatty acid.

The fatty acids may be in a free acid state or in an esterified form such as part of a triglyceride, diacylglyceride, monoacylglyceride, acyl-CoA bound or other bound form. The fatty acid may be esterified as a phospholipid such as phosphatidylcholine, phosphatidylethanolamine, phosphatidylserine, phosphatidylglycerol, phosphatidylinositol or diphosphatidylglycerol forms. In one aspect, genetically modified fatty acid producing oleaginous microorganism is subjected to further genetic modifications to confer the ability to use DHA as a source of carbon. In another aspect of this invention, microorganisms that have ability to grow in DHA in a medium comprising DHA as a source of carbon is subjected to further genetic modifications to produce fatty acids. Such a genetic modification would involve overexpressing one or more components of the saturated fatty acid biosynthetic pathway, unsaturated fatty acid biosynthetic pathway, iso-branched fatty acid biosynthetic pathway, anteiso-branched fatty acid biosynthetic pathway. The list of genetic manipulations required to enhance the fatty acid biosynthesis within the microbial cell include but not limited to: enhancing the activities of one or more of the enzymes involved in the saturated fatty acid biosynthetic pathway such as acetyl-CoA carboxylase (AccABCD), acetyl CoA:ACP transacylase, malonyl CoA:ACP transacylase (FabD), 3-ketoacyl-ACP synthase (FabBF), 3-ketoacyl-ACP reductase (FabG), 3-hydroxyacyl ACP dehydrase (FabAG), enoyl-ACP reductase (FabIKL), myristic-ACP thioesterase, palmitoyl-ACP thioesterase, stearoyl-ACP thioesterase, oleoyl-ACP thioesterase, glycerol kinase, glycerol-3P-acyltransferase, 1-acylglycerol-3P-acyltransferase, phosphatidic acid phosphatase, diacylglycerol acyltransferase, CDP-DAG synthase; enhancing the activities of one or more of the enzymes involved in the unsaturated fatty acid biosynthetic pathway such as C_{14/16} elongase, C_{16/18} elongase, 49 desaturase, 412 desaturase, 415 desaturase, 46 desaturase, 49 elongase, 48 desaturase, C_{18/20} elongase, 45 desaturase, 417 desaturase C₂₀₁₂₂ elongase, 44 desaturase, glycerol kinase, glycerol-3P-acyltransferase, 1-acylglycerol-3P-acyltransferase, phosphatidic acid phosphatase, diacylglycerol acyltransferase and CDP-DAG synthase; enhancing activities of one or more of the enzymes involved in the fatty alcohol biosynthetic pathway such as polyunsaturated fatty acid synthase, thioesterase, acyl-CoA synthase, acyl-CoA reductase, alcohol dehydrogenase, fatty alcohol-CoA reductase and wax synthase; enhancing activities of one or more of the enzymes involved in the iso-branched fatty acid biosynthetic pathway such as α-keto acid dehydrogenase, β-ketoacyl ACP synthase, β-ketoacyl-ACP-reductase, β-hydroxyacyl-ACP-dehydratase and enoyl-ACP-reductase; enhancing activities of one or more of the enzymes involved in the anteiso-branched fatty acid biosynthetic pathway such as α-keto acid dehydrogenase, β-ketoacyl ACP synthase, β-ketoacyl-ACP-reductase, β-hydroxyacyl-ACP-dehydratase and enoyl-ACP-reductase; enhancing activities of one or more of the enzymes involved in the odd chain fatty acid biosynthetic pathway such as 2-keto dehydrogenase, acetyl-CoA carboxylase (AccABCD), malonyl CoA:ACP transacylase (FabD), 3-ketoacyl-ACP synthase (FabBF), 3-ketoacyl-ACP reductase (FabG), 3-hydroxyacyl ACP dehydrase (FabAG), enoyl-ACP reductase (FabIKL), glycerol kinase, glycerol-3P-acyltransferase, 1-acylglycerol-3P-acyltransferase, phosphatidic acid phosphatase, diacylglycerol acyltransferase and CDP-DAG synthase.

In one aspect of the present invention, microorganisms that have ability to grow in DHA in a medium comprising DHA as a source of carbon is subjected to further genetic modifications to produce polyunsaturated fatty acids (PUFA). Such genetic modifications would involve overexpressing one or more components of the PUFA biosynthesis pathway and further extending the carbon chain length with elongases and introducing unsaturation with desaturases.

In another aspect of this embodiment, microorganisms that have ability to grow in a medium comprising DHA as a source of carbon is subjected to further genetic modifications to include one or more nucleic acid molecules comprising one or more nucleotide sequence encoding saturated fatty acid biosynthetic pathway enzymes. The list of the enzymes functional in the saturated fatty acid biosynthetic pathway includes the enzymes that catalyze the condensation of acetyl-CoA and malonyl-CoA to form long-chain saturated fatty acids with carbon chain lengths from C14 to C16 and C18 or more. Microorganisms that already has an ability to produce high amounts of acetyl-CoA and malonyl-CoA either natively or by one or more genetic modifications are a preferred host for the production of saturated fatty acids. On the other hand, saturated fatty acid biosynthetic pathway can be directly engineered in the host cell that previously lack the ability to produce acetyl-CoA or malonyl-CoA but can be engineered to selectively produce saturated fatty acids. Furthermore, one or more of the enzymes selected from a group of saturated fatty acid biosynthetic pathway such as acetyl-CoA carboxylase (AccABCD), acetyl CoA:ACP transacylase, malonyl CoA:ACP transacylase (FabD), 3-ketoacyl-ACP synthase (FabBF), 3-ketoacyl-ACP reductase (FabG), 3-hydroxyacyl ACP dehydrase (FabAG), enoyl-ACP reductase (FabIKL), myristic-ACP thioesterase, palmitoyl-ACP thioesterase, stearoyl-ACP thioesterase, oleoyl-ACP thioesterase, glycerol kinase, glycerol-3P-acyltransferase, 1-acylglycerol-3P-acyltransferase, phosphatidic acid phosphatase, diacylglycerol acyltransferase and CDP-DAG synthase are overexpressed for the enhanced production of saturated fatty acid from acetyl-CoA and malonyl-CoA. Some non-limiting examples of saturated fatty acids includes caprylic acid, capric acid, lauric acid, myristic acid, palmitic acid, stearic acid, arachidic acid, behenic acid, lignoceric acid, cerotic acid and their respective monoacyl glyceride, diacyl glyceride, triacyl glyceride, phosphatidic acid, phosphatidylethanolamine, phosphatidylcholine, phosphatidylserine, phosphatidylinositol, phosphatidylinositol phosphate, phosphatidylinositol bisphosphate, and phosphatidylinositol triphosphate. In addition, attenuation or deletion of one or more of enzymes methylglyoxal synthase, acetate kinase, pyruvate carboxylase, phosphoenolpyruvate carboxylase, succinate reductase, citrate synthase, aldehyde dehydrogenase, pyruvate oxidase, pyruvate-formate lyase, alanine transaminase, lactate dehydrogenase, acetolactate synthase and HMG-CoA synthase improves pyruvate utilization in saturated fatty acid biosynthesis. Furthermore, blocking one or more of saturated fatty acid degradation pathways such as formation of alcohol catalyzed by aldehyde dehydrogenase and alcohol dehydrogenase, fatty acid oxidation reaction catalyzed by acetyl-CoA dehydrogenases improves saturated fatty acid accumulation. Furthermore, improving saturated fatty acid transport, reducing uptake from media and reducing feedback inhibition increases saturated fatty acid productivity.

In yet another aspect of this embodiment, microorganisms that have ability to grow in DHA in a medium comprising DHA as a source of carbon is subjected to further genetic modifications to include one or more nucleic acid molecules comprising one or more nucleotide sequence encoding enzymes functional in unsaturated fatty acid biosynthetic pathway. The list of the enzymes functional in the unsaturated fatty acid synthesis includes enzymes that catalyze the elongation and desaturation of saturated fatty acid to form long-chain monounsaturated and polyunsaturated fatty acids with carbon chain lengths from C18 to C22. Microorganisms that already has an ability to produce high amounts of saturated fatty acids either natively or by one or more genetic modifications are a preferred host to produce unsaturated fatty acids. On the other hand, unsaturated fatty acid biosynthetic pathway can be directly engineered in the host cell that previously lack the ability to produce unsaturated fatty acid but can be engineered to selectively produce unsaturated fatty acids. Furthermore, one or more of the enzymes selected from a group of unsaturated fatty acid biosynthetic pathway such as C_{14/16} elongase, C_{16/18} elongase, Δ9 desaturase, Δ12 desaturase, Δ15 desaturase, Δ6 desaturase, Δ9 elongase, Δ8 desaturase, C_{18/20} elongase, Δ5 desaturase, Δ17 desaturase C_{20/22} elongase, Δ4 desaturase, glycerol kinase, glycerol-3P-acyltransferase, 1-acylglycerol-3P-acyltransferase, phosphatidic acid phosphatase, diacylglycerol acyltransferase and CDP-DAG synthase are overexpressed for the enhanced production of unsaturated fatty acids from saturated fatty acids. Moreover, improvement of saturated fatty acid production by overexpressing one or more enzymes in saturated fatty acid biosynthetic pathway enhances the productivity of unsaturated fatty acids. Some non-limiting examples of monounsaturated fatty acids include myristoleic acid, palmitoleic acid, sapienic acid, oleic acid, elaidic acid vaccenic acid and their respective monoacyl glyceride, diacyl glyceride, triacyl glyceride, phosphatidic acid, phosphatidylethanolamine, phosphatidylcholine, phosphatidylserine, phosphatidylinositol, phosphatidylinositol phosphate, phosphatidylinositol bisphosphate, and phosphatidylinositol triphosphate. Some non-liming examples of polyunsaturated fatty acids include, linoleic acid, eicosadienoic acid, eicosatrienoic acid, α-linoleic acid, γ-linoleic acid, stearidonic acid, eicosatetraenoic acid, dihomo-γ-linoleic acid, arachidonic acid, eicosapentaenoic acid, docosapentaenoic acid, docosahexaenoic acid and their respective monoacyl glyceride, diacyl glyceride, triacyl glyceride, phosphatidic acid, phosphatidylethanolamine, phosphatidylcholine, phosphatidylserine, phosphatidylinositol, phosphatidylinositol phosphate, phosphatidylinositol bisphosphate, and phosphatidylinositol triphosphate. In addition, attenuation or deletion of one or more of enzymes methylglyoxal synthase, acetate kinase, pyruvate carboxylase, phosphoenolpyruvate carboxylase, succinate reductase, citrate synthase, aldehyde dehydrogenase, pyruvate oxidase, pyruvate-formate lyase, alanine transaminase, lactate dehydrogenase, acetolactate synthase and HMG-CoA synthase improves pyruvate utilization in unsaturated fatty acid biosynthesis. Furthermore, blocking one or more of unsaturated fatty acid degradation pathways such as formation of alcohol catalyzed by aldehyde dehydrogenase and alcohol dehydrogenase, fatty acid oxidation reaction catalyzed by acetyl-CoA dehydrogenases improves unsaturated fatty acid accumulation. Furthermore, improving unsaturated fatty acid transport, reducing uptake from media and reducing feedback inhibition increases unsaturated fatty acid productivity.

In another aspect of this embodiments, microorganisms that have ability to grow in DHA in a medium comprising DHA as a source of carbon is subjected to further genetic modifications to include one or more nucleic acid molecules comprising one or more nucleotide sequence encoding enzymes functional in the fatty alcohol biosynthetic pathway and wax biosynthetic pathway. Suitable fatty alcohol synthesis enzymes catalyze the reduction of saturated and unsaturated fatty acids to form long-chain fatty alcohols with carbon chain lengths from C14 to C16 and C18 or more. Microorganisms that already has an ability to produce high amounts of fatty acids either natively or by one or more genetic modifications are preferred host to produce fatty alcohols. On the other hand, fatty alcohol biosynthetic pathway can be directly engineered in the host cell that previously lack the ability to produce fatty acid but can be engineered to selectively produce fatty alcohols. Furthermore, one or more of the enzymes selected from a group of fatty alcohol biosynthetic pathway such as polyunsaturated fatty acid synthase, thioesterase, acyl-CoA synthase, acyl-CoA reductase, alcohol dehydrogenase, fatty alcohol-CoA reductase and wax synthase are overexpressed to enhance the production of fatty alcohol and wax biosynthesis. Moreover, improvement of fatty acid production by overexpressing one or more enzymes in saturated fatty acid biosynthetic pathway or unsaturated fatty acid biosynthetic pathway enhances the productivity of fatty alcohols. Some non-liming examples of fatty alcohols include, linoleic alcohol, eicosadienoic alcohol, eicosatrienoic alcohol, α-linoleic alcohol, γ-linoleic alcohol, stearidonic alcohol, eicosatetraenoic alcohol, dihomo-γ-linoleic alcohol, arachidonic alcohol, eicosapentaenoic alcohol, docosapentaenoic alcohol, docosahexaenoic alcohol, myristoleic alcohol, palmitoleic alcohol, sapienic alcohol, oleic alcohol, elaidic alcohol, vaccenic alcohol, caprylic alcohol, capric alcohol, lauric alcohol, myristic alcohol, palmitic alcohol, stearic alcohol, arachidic alcohol, behenic alcohol, lignoceric alcohol and cerotic alcohol. In addition, attenuation or deletion of one or more of enzymes methylglyoxal synthase, acetate kinase, pyruvate carboxylase, phosphoenolpyruvate carboxylase, succinate reductase, citrate synthase, aldehyde dehydrogenase, pyruvate oxidase, pyruvate-formate lyase, alanine transaminase, lactate dehydrogenase, acetolactate synthase and HMG-CoA synthase improves pyruvate utilization in fatty alcohol biosynthesis. Furthermore, blocking one or more of unsaturated fatty alcohol degradation pathways such as oxidation reaction to fatty acid improves unsaturated fatty alcohol accumulation. Furthermore, improving fatty alcohol transport, reducing uptake from media and reducing feedback inhibition increases unsaturated fatty alcohol productivity.

In yet another aspect of this embodiments, microorganisms that have ability to grow in DHA in a medium comprising DHA as a source of carbon is subjected to further genetic modifications to include one or more nucleic acid molecules comprising one or more nucleotide sequence encoding iso fatty acid biosynthetic pathway enzymes. Iso branched chain fatty acids and anteiso branched chain fatty acids are carboxylic acid with a methyl branch in the n−1 and n−2 carbon, respectively. Iso branched chain fatty acids are made from 2-ketoisocaproate or 2-ketoisovalerate. Microorganisms that already have an ability to produce high amounts of 2-ketoisocaproate or 2-ketoisovalerate either natively or by one or more genetic modifications are preferred host to produce Iso branched chain fatty acids. Iso branched chain fatty acid biosynthetic pathway can be directly engineered in the host cell that previously lack the ability to produce 2-ketoisocaproate or 2-ketoisovalerate but can be engineered to selectively produce iso branched chain fatty acid. In addition to the enzymes catalyzing the formation of 2-ketoisocaproate or 2-ketoisovalerate from DHA, one or more of the enzymes selected from a group of iso branched chain fatty acid biosynthetic pathway such as α-keto acid dehydrogenase, β-ketoacyl ACP synthase, β-ketoacyl-ACP-reductase, β-hydroxyacyl-ACP-dehydratase and enoyl-ACP-reductase are overexpressed for the enhanced production of iso branched chain fatty acid from 2-ketoisocaproate or 2-ketoisovalerate. Moreover, improvement of 2-ketoisocaproate or 2-ketoisovalerate production by overexpressing one or more enzymes in their metabolic pathways enhances the productivity of iso branched chain fatty acids. In another method, anteiso branched chain fatty acids are made from 2-keto-3-methylvalerate. Microorganisms that already has an ability to produce high amounts of 2-keto-3-methylvalerate either natively or by one or more genetic modifications are a preferred host to produce anteiso branched chain fatty acids. On the other hand, anteiso branched chain fatty acid biosynthetic pathway can be directly engineered in to the host cell that previously lack the ability to produce 2-keto-3-methylvalerate but can be engineered to selectively produce anteiso branched chain fatty acid. In addition to the enzymes catalyzing the formation of 2-keto-3-methylvalerate from DHA, one or more of the enzymes selected from a group of anteiso branched chain fatty acid biosynthetic pathway such as α-keto acid dehydrogenase, β-ketoacyl ACP synthase, β-ketoacyl-ACP-reductase, β-hydroxyacyl-ACP-dehydratase and enoyl-ACP-reductase are overexpressed for the enhanced production of anteiso branched chain fatty acid from 2-keto-3-methylvalerate. Moreover, improvement of 2-keto-3-methylvalerate production by overexpressing one or more enzymes in 2-keto-3-methylvalerate metabolic pathway enhances the productivity of anteiso branched chain fatty acids. In addition, attenuation or deletion of one or more of enzymes methylglyoxal synthase, acetate kinase, aldehyde dehydrogenase, pyruvate oxidase, pyruvate-formate lyase, alanine transaminase, lactate dehydrogenase and HMG-CoA synthase improves pyruvate utilization in iso fatty acid biosynthesis. Furthermore, blocking one or more of fatty acid degradation pathways such as formation of alcohol catalyzed by aldehyde dehydrogenase and alcohol dehydrogenase, fatty acid oxidation reaction catalyzed by acetyl-CoA dehydrogenases improves iso fatty acid accumulation. Furthermore, improving iso fatty acid transport, reducing uptake from media and reducing feedback inhibition increases iso fatty acid productivity.

In yet another aspect of this embodiments, microorganisms that have ability to grow in a medium comprising DHA as a source of carbon is subjected to further genetic modifications to include one or more nucleic acid molecules comprising one or more nucleotide sequences encoding odd chain fatty acid biosynthetic pathway enzymes. In contrast to the saturated fatty acids, where the number of carbon in the fatty acids are even number, an odd chain fatty acids are long chain carboxylic acid, where the number of carbon in the fatty acid is an odd number. Odd chain fatty acids are made from propionyl-CoA. Microorganisms that already has an ability to produce high amounts of propionyl-CoA either natively or by one or more genetic modifications are a preferred host to produce odd chain fatty acids. On the other hand, odd chain fatty acid biosynthetic pathway can be directly engineered in the host cell that previously lack the ability to produce propionyl-CoA but can be engineered to selectively produce odd chain fatty acid. In addition to the enzymes catalyzing the formation of propionyl-CoA from DHA, one or more of the enzymes selected from a group of odd chain fatty acid biosynthetic pathway such as 2-keto dehydrogenase, acetyl-CoA carboxylase (AccABCD), malonyl CoA:ACP transacylase (FabD), 3-ketoacyl-ACP synthase (FabBF), 3-ketoacyl-ACP reductase (FabG), 3-hydroxyacyl ACP dehydrase (FabAG), enoyl-ACP reductase (FabIKL), glycerol kinase, glycerol-3P-acyltransferase, 1-acylglycerol-3P-acyltransferase, phosphatidic acid phosphatase, diacylglycerol acyltransferase, and CDP-DAG synthase are overexpressed for the enhanced production of odd chain fatty acids from propionyl-CoA. Moreover, improvement of 2-ketobutyrate production by overexpressing one or more enzymes in 2-ketobutyrate metabolic pathway enhances the productivity of odd chain fatty acids. In addition, attenuation or deletion of one or more of enzymes methylglyoxal synthase, acetate kinase, aldehyde dehydrogenase, pyruvate oxidase, pyruvate-formate lyase, alanine transaminase, lactate dehydrogenase and HMG-CoA synthase improves pyruvate utilization in odd chain fatty acid biosynthesis.

Furthermore, blocking one or more of fatty acid degradation pathways such as formation of alcohol catalyzed by aldehyde dehydrogenase and alcohol dehydrogenase, fatty acid oxidation reaction catalyzed by acetyl-CoA dehydrogenases improves odd chain fatty acid accumulation. Furthermore, improving odd chain fatty acid transport, reducing uptake from media and reducing feedback inhibition increases odd chain fatty acid productivity.

Genetically modified host cells capable of producing fatty acid precursors using conventional sugars such as glucose, sucrose or glycerol are already known in the art. For example, U.S. Pat. No. 7,736,884 provides genetically engineered Saccharomyces cerevisiae to produce polyunsaturated fatty acids. The U.S. Patent Application Publication No. 2012/0142979 describes methods for producing fatty alcohols from fatty acids in a genetically engineered microorganism with enhanced fatty alcohol forming activity. U.S. Pat. No. 8,816,106 describes enzymes which possess desatuase, conjucase, epoxidase and hydroxylase activity that can be used to synthesize fatty acids. U.S. Pat. No. 8,765,404 provides genetically engineered microorganism to produce fatty acid and derivatives such as triacyl glycerides. U.S. Pat. No. 7,901,928 describes acyltransferase enzymes for the production of phospholipids and acylglycerides. U.S. Pat. No. 7,736,884 describes enzyme 49 desaturase for the introduction of monounsaturation in saturated fatty acids. U.S. Pat. No. 7,749,703 describes enzyme 412 desaturase for the conversion of oleic acid to linoleic acid. U.S. Pat. No. 7,465,564 describes the introduction enzymes 46 desaturase, 412 desaturase for the production of γ-linolenic acid. U.S. Pat. No. 5,663,068 describes cyanobacterial enzyme 46 desaturase for the conversion of linoleic acid to γ-linolenic acid. U.S. Pat. No. 8,772,384 describes methods of modulating the levels of desaturase to produce long chain PUFA. U.S. Pat. No. 5,552,306 describes the production of γ-linolenic acid from linolenic acid using 46 desaturase. U.S. Pat. No. 5,057,419 describes genetically engineered yeast organism with 49 desaturase for the production of PUFA. U.S. Pat. No. 6,136,574 describes the production of γ-linolenic acid from linolenic acid in a genetically modified yeast cell with morierella alpina 46 desaturase. U.S. Pat. No. 8,951,776 describes genetically engineered microorganisms with greatly enhanced triacyl glyceride synthesis properties. U.S. Pat. No. 7,273,746 describes genetically engineered microorganism with diacylglycerol acyltransferase enzyme for the enhanced production of PUFA triacyl glycerides. U.S. Patent Application Publication No. 2013/0344548 describes the production of microbial oil from cellulosic materials. U.S. Pat. Nos. 5,583,019, 5,204,250, 6,749,849, 5,658,767, 5,882,703, 6,245,365, 6,319,698, 6,541,049, 7,195,791, 7,601,523, 7,666,657, 7,736,885, 6,958,229 and 7,585,651 and International PCT Application Publication No. WO1992/013086 describe genetically engineered microorganism for the production of arachidonic acid. U.S. Pat. Nos. 7,091,244, 7,863,024, 6,461,839 8,841,097, 8,945,886, and 9,023,616 and U.S. Patent Application Publication No. 2005/0129739, describe process to produce polyunsaturated fatty acid containing oils. U.S. Pat. Nos. 6,150,144 and 6,812,020 describe process for producing omega-9 highly unsaturated fatty acid from recombinant microorganism. U.S. Pat. Nos. 6,977,166, 6,509,178, 6,582,941, 7,259,006, 7,863,026, 5,407,957, 7,163,811, 7,252,979, 7,745,183, 7,824,892, 7,829,129, 7,871,809, 8,008,050, 8,663,953, 8,669,090, 9,249,434 and U.S. Pat. Nos. 7,514,244 and 6,207,441 describe the production of docosahexanoic acid from recombinant microorganism. U.S. Pat. Nos. 7,238,482, 7,553,628, 8,399,242 and 9,133,465 describe the production of polyunsaturated fatty acids using recombinant microorganism. U.S. Pat. Nos. 7,588,931, 7,550,286, 7,932,077, 8,518,674, 8,685,682, 7,364,883 and 8,815,566 describes high unsaturated fatty acid producing Yarrowia lipolytica strains. U.S. Pat. Nos. 8,124,384, 6,607,900, 7,579,174, 7,732,170, 8,124,385, 8,133,706, 8,187,845, 8,187,846, 8,206,956, 8,216,812, 8,288,133 and 8,288,134 describes eukaryotic microbes for the production of polyenenoic fatty acids. U.S. Pat. Nos. 7,470,527 and 6,117,905 describes microbial oil comprising high arachidonic acid. U.S. Pat. No. 4,783,408 describes fungal body for the preparation of gamma-linolenic acid. U.S. Pat. Nos. 4,916,066, 5,401,646, 5,034,321, 8,871,808, 6,280,982, 6,602,690 and 5,093,249 describes the production of bishomo-gamma-linolenic acid. U.S. Pat. Nos. 5,244,921, 5,567,732, 5,246,841, 5,246,842, 8,822,185, 8,323,935, 8,735,108 and 9,222,112 and U.S. Patent Application Publication Nos. 2011/0177031, 2015/0313861 and 2015/0237888 and describe the production of eicosapentaenoic acid from recombinant microorganism. U.S. Pat. Nos. 7,645,604, 8,048,653, 8,049,062, 420,892, 9,150,874, 8,465,787, 8,168,858, 8,377,673, 7,794,701, 8,119,860 and 8,298,797 describes delta-9-elongases to produce polyunsaturated fatty acids. U.S. Pat. Nos. 7,943,823, 7,256,033, 7,550,651, 8,058,517, 8,470,571, 6,825,017, 402,735, 8,338,152, 7,790,156, 8,188,338, 8,318,463, 8,026,089, 7,709,239, 8,859,849, 7,863,502 and 8,124,838 describes delta-8-desaturase for the production of polyunsaturated fattyacids. U.S. Pat. No. 8,828,690 describes multienzymes comprising delta-9-elongase and delta-8-desaturase and their use in making polyunsaturated fatty acids. U.S. Pat. No. 7,842,852 describes methods for increasing polyunsaturated long-chain fatty acids in transgenic organism. U.S. Pat. Nos. 7,807,849, 8,106,226, 8,288,572, 8,575,377, 8,071,341, 7,834,250, 7,932,438, 8,158,392, 8,535,917, 8,778,644, 7,736,884, 8,790,901 and 8,853,432 describes the synthesis of long-chain polyunsaturated fatty acids by recombinant microorganisms. U.S. Pat. Nos. 7,214,491, 7,504,259 and 7,749,703 describe delta-12 desaturase gene to produce polyunsaturated fatty acid in recombinant microorganisms. U.S. Pat. Nos. 7,659,120, 8,273,957 and 9,150,836, describe delta-15-desaturase gene to produce polyunsaturated fatty acid in recombinant microorganisms. U.S. Pat. No. 5,322,780 describes process to produce omega-9-polyunsaturated fatty acids. U.S. Pat. No. 5,128,259 describes the production of polyunsaturated fatty acids having odd number of carbon atoms. U.S. Pat. No. 5,376,541 describes the production of 8,11-eicosadienoic acid using recombinant microorganism. U.S. Pat. Nos. 5,130,242 and 8,877,465 describes the heterotrophic production process for polyunsaturated fatty acids. U.S. Pat. Nos. 5,552,306, 5,663,068, 6,683,232, 5,689,0507,456,270, 8,778,632, 6,355,861, 7,189,894, 5,614,393, 5,789,220, and 7,282,623, U.S. Patent Application Publication Nos. 2007/0130654, 2009/0077692, describe the production of gamma-linolenic acid by a delta-6 desaturase. U.S. Pat. No. 5,314,812 describes microbiological process to produce high unsaturated fatty acids. U.S. Pat. Nos. 7,695,950 7,678,560, 8,962,917 and 8,049,070 describes delta-5-desaturase for the production of polyunsaturated fatty acids. U.S. Pat. No. 7,465,793 describes delta-17-desaturase to produce polyunsaturated fatty acids. U.S. Pat. No. 7,470,532 describes C16/C18 fatty acid elongase to produce polyunsaturated fatty acids. U.S. Pat. Nos. 7,883,882, 7,935,515, 8,187,860, U.S. Pat. Nos. 8,222,010, 8,268,610, 8,435,767, 8,674,180, 8,697,427, 8,772,575 and 9,062,294 provides recombinant microorganism that are producing novel triglyceride oils. U.S. Patent Application Publication No. 2012/0156717 describes biofuel production from recombinant oleaginous algae. U.S. Pat. Nos. 8,476,059, 8,497,116, 8,512,999, 8,518,689, 8,647,397, 8,697,402, 8,790,914, 8,802,422, 8,889,401 and 8,889,402 describe the use of sucrose based feedstock utilization for triglyceride-oil based fuel production using microalgae grown under heterotrophic conditions. U.S. Patent Application Publication No. 2010/0303957 describes the production of microalgal based triglyceride oil suitable for human consumption lacking in microalgal toxins. U.S. Pat. Nos. 8,852,885, 8,633,012 and 9,249,436 describe method for using sucrose to produce an oil comprising triglycerides that comprise ricinoleic acid or other hydroxylated fatty acids from the fatty acids. U.S. Pat. Nos. 8,592,188, 8,765,424, 9,109,239, 9,255,282 and 9,279,136 recombinant heterotrophic microorganism for the production of tailored triglyceride based oil containing mono and di-unsaturated fatty acids. U.S. Pat. Nos. 8,119,583, 8,450,083, 8,278,261, 8,822,176 and 8,822,177 describe soap comprising fatty acid salts of saponified microalgal lipid and the method of producing the lipids from sugar based carbon sources. U.S. Pat. No. 9,249,252 describes the production of heterotrophic microalgae based low polyunsaturated fatty acid oils and their uses in industrial and cosmetic applications. U.S. Pat. Nos. 9,200,307, 8,846,375, 8,945,908, 9,068,213, 9,102,973 and 9,249,441 describes recombinant microorganism to produce tailored triglyceride oil having altered fatty acid profile when compared to an oil produced by a non-recombinant microalga. U.S. Pat. Nos. 5,518,918, 5,340,742, 5,688,500, 5,908,622, 6,103,225, 6,566,123, 7,381,558 and 8,129,172 describes microfloral biomass having high omega-3-unsaturated fatty acids. U.S. Patent Application Publication Nos. 2010/0071259 and 2015/0259714 describes systems and methods for the mixed fatty esters. U.S. Patent Application Publication Nos. 2010/0257777, 2010/0257778 and 2015/0267134 describe production of fatty acid derivatives and their use as biodiesel. U.S. Pat. No. 9,133,406 describes the production of fatty acid derivatives. U.S. Patent Application Publication Nos. 2010/0170148 and 2013/0115668 describes host cells and methods for producing fatty acid derived compounds. U.S. Pat. Nos. 8,110,670, 8,283,143 and 9,017,984, describe genetically engineered cells and microorganism for the enhanced production of fatty acid derivatives. U.S. Pat. Application Publication Nos. 2010/0242345, 2012/0142979, 2013/0245339, 2015/0133698, 2015/0064782, 2014/0179940, and 2013/0197248 and U.S. Pat. Nos. 9,068,201, 999,686, 8,097,439, 8,216,815, 8,574,877 and 8,883,467 describe production of fatty acid derivatives such as fatty alcohols, wax and triglycerides. U.S. Pat. Application Publication No. 2010/0274033 describes methods of producing fatty acid esters. U.S. Patent Application Publication No. 2011/0146142 describes systems and methods for the production of mixed fatty esters. International PCT Application. Publication No. WO2015/085271 provides microbial production of fatty amines. U.S. Pat. Nos. 8,268,599, 8,323,924, 8,633,022, 8,658,404 and 8,846,371 and U.S. Patent Application Publication Nos. 2013/0035513 and 2010/0298612 describe methods and genetically engineered microorganism to produce fatty alcohol, fatty aldehydes, alkanes, alkenes and hydrocarbons. U.S. Patent Application Publication Nos. 2011/0151526 and 2011/0166370, International PCT Application. Publication No. WO2012/096686 and U.S. Pat. No. 8,530,221 describe recombinant microorganism to produce branched chain fatty acids. International PCT Application Publication No. WO2013/082309 describes production of oil, fuels and oleochemicals from glycerol and other waste products. U.S. Pat. No. 8,372,610 describes recombinant microorganism to produce odd chain fatty acids. U.S. Patent Application No. 2014/0178948 describes the production of omega-amino fatty acids. U.S. Patent Application No. 2015/0064757 describes the improved production of fatty alcohols using novel carboxylic acid reductase enzyme. U.S. Pat. No. 9,284,566 describes recombinant microorganism for the production of biofuels and biochemical. U.S. Patent Application Publication No. 2016/0002681 describes recombinant microorganism for the production of fatty acid mediated by acyl carrier protein. U.S. Patent Application Publication No. 2016/0361454 describes acyl-ACP reductase enzyme variants that results in the improved production of fatty acids and fatty aldehydes. U.S. Patent Application Publication No. 2015/0059295 describes host cells and methods for producing fatty acids. U.S. Pat. No. 6,982,155 describes methods of producing fatty acids lower alcohol ester. U.S. Patent Application Publication No. 2014/0051136 describes microorganisms and methods for the production of fatty acids and fatty esters. U.S. Pat. Nos. 9,017,975 and 8,921,090 describe production and secretion of fatty acids and fatty acid derivatives. U.S. Patent Application Publication No. 2016/0060663 describes recombinant microorganism for the production of fatty acid esters.

In one method to enhance DHA uptake and utilization, the genetically modified host cells already known to produce fatty acids, as described in patents above, is subjected to further genetic engineering by means of expressing plasmids that code ATP and PEP dependent kinases that could phosphorylate DHA and facilitate its entry into glycolytic cycle. In another method to enhance DHA uptake and utilization, genetically modified host cells already known to produce fatty acids, as described in patents above, is subjected to chemical mutagenesis and the strains with the ability to grow and produce desired fatty acid with high enough titer and yield in a growth medium comprising DHA as a source of carbon will be selected and subjected to whole genome sequencing to identify specific mutation associated with the ability to grow and produce fatty acid in a medium comprising DHA. Such a mutation will be introduced to the genetically modified host cells already known to produce fatty acid to confer the ability to use DHA as a source of organic carbon. In another method to enhance DHA uptake and utilization, genetically modified host cells already known to produce fatty acids, as described in patents above, is exposed to growth medium with increasing concentration of DHA and subjected to metabolic evolution. Strains that have gained ability to grow in the medium containing DHA as a major or sole source of carbon and still retain the ability to produce desired fatty acids is selected and subjected to whole genome sequencing to identify specific mutation associated with the ability to grow and produce fatty acids in a medium comprising DHA. Such a mutation will be introduced to the genetically modified host cells already known to produce fatty acid, as described in patents above, for the purpose of conferring the ability to use DHA as a source of organic carbon.

In yet another embodiment, the present invention provides methods of producing L-glutamic acid and “L-glutamine”. In one aspect, genetically modified L-glutamic acid and L-glutamine producing microorganisms are subjected to further genetic modifications to confer the ability to use DHA as a source of carbon in a medium comprising dihydroxyacetone as a carbon source. In another aspect of this invention, microorganisms that have ability to grow in a medium comprising DHA as a source of carbon is subjected to further genetic modifications to produce L-glutamic acid and L-glutamine. Such a genetic modification would involve overexpressing one or more enzymes functioning in the glutamate biosynthesis pathway such as isocitrate dehydrogenase (ICDH), pyruvate carboxylase and glutamate dehydrogenase (GDH) and decreasing the activities of 2-oxoglutarate dehydrogenase complex (ODHC) and isocitrate lyase. In addition, triggering operations such as depletion of biotin and addition of detergent polyoxyethylene sorbitan monopalmitate (Tween 40) and β-lactam antibiotics such as penicillin is performed to enhance the glutamate production.

In some embodiments, microorganisms that have ability to grow in a medium comprising DHA as a source of carbon is subjected to further genetic modifications to include one or more nucleic acid molecules comprising one or more nucleotide sequences encoding glutamic acid biosynthetic pathway enzymes. Glutamic acid is made from 2-oxoglutarate by a single step biochemical reaction catalyzed by glutamate dehydrogenase. Microorganisms that already has an ability to produce high amounts of 2-oxoglutarate by one or more genetic modifications are preferred hosts to produce glutamic acid. On the other hand, glutamic acid biosynthetic pathway can be directly engineered in the host cell that previously lack the ability to produce 2-oxoglutarate but can be engineered to selectively produce glutamic acid. Furthermore, one or more of the genes selected from a group of glutamic acid biosynthetic pathway such as glutamate dehydrogenase, pyruvate carboxylase and citrate synthase are overexpressed for the enhanced production of glutamic acid. In addition, attenuation or deletion of one or more of enzymes methylglyoxal synthase, acetate kinase, aldehyde dehydrogenase, pyruvate oxidase, pyruvate-formate lyase, alanine transaminase, lactate dehydrogenase, acetyl-CoA carboxylase, beta-ketothiolase and HMG-CoA synthase improves pyruvate utilization in glutamic acid biosynthesis. Furthermore, decreased activities of 2-oxo-glutarate dehydrogenase, isocitrate lyase, and malate dehydrogenase improves glutamate yield. Mutation or deletion in the gene encoding aspartate ammonium lyase (aspA), decreasing the expression of gluX gene and increasing the expression of fasR gene improve the glutamate production. When glutamate export gene yhfK is enhanced or overexpressed, there is an accumulation of glutamate in the medium. Furthermore, blocking one or more of glutamic acid degradation pathways such as formation of succinyl-CoA catalyzed by 2-oxoglutarate dehydrogenase and formation of 2-hydroxyglutarate catalyzed by malate dehydrogenase improves glutamic acid accumulation. Furthermore, improving glutamate transport, reducing uptake from media and reducing feedback inhibition increases glutamate productivity.

In some embodiments, microorganisms that have ability to grow in a medium comprising DHA as a source of carbon is subjected to further genetic modifications to include one or more nucleic acid molecules comprising one or more nucleotide sequence encoding glutamine biosynthetic pathway enzymes. Glutamine is made from glutamic acid by a single step biochemical reaction catalyzed by glutamate ammonia ligase. Microorganisms that already has an ability to produce high amounts of glutamate by one or more genetic modifications are a preferred host to produce glutamine. On the other hand, glutamine biosynthetic pathway can be directly engineered in the host cell that previously lack the ability to produce glutamate but can be engineered to selectively produce glutamine. Furthermore, one or more of the genes selected from a group of glutamine biosynthetic pathway such as glutamate dehydrogenase, pyruvate carboxylase, glutamate ammonia ligase and citrate synthase are overexpressed for the enhanced production of glutamine. Furthermore, blocking one or more of glutamine degradation pathways such as formation of 2-oxoglutaramate catalyzed by glutamine-oxoacid transaminase improves glutamine accumulation. Furthermore, improving glutamine transport, reducing uptake from media and reducing feedback inhibition increases glutamine productivity.

Genetically modified host cells capable of producing glutamate using conventional sugars such as glucose, sucrose or glycerol are already known in the art. For example, U.S. Pat. No. 6,852,516 provides genetically engineered Corneform bacterium that has enhanced pyruvate carboxylase activity. U.S. Pat. No. 5,492,818 provides reduced 2-oxoglutarate dehydrogenase activity and U.S. Pat. No. 7,205,132 provides mutated odhA gene for the increase glutamate production. U.S. Pat. Nos. 7,785,845 and 7,344,874 and U.S. Patent Application Publication No. 2005/0196846 identified and overexpressed a glutamate export gene yhfK for the improved accumulation of glutamate. U.S. Pat. No. 7,867,735 identified and overexpressed fasR gene for the improved glutamate accumulation. U.S. Pat. Nos. 5,573,945 and 5,378,616 provide genetically engineered Escherichia with reduced 2-ketoglutarate dehydrogenase activity and amplified pyruvate carboxylase and glutamate dehydrogenase. U.S. Pat. No. 8,110,381 identified and deactivated gluX gene and U.S. Pat. No. 7,307,160 identified and deleted otsA for improved accumulation of glutamate. U.S. Pat. No. 7,256,021 provides genetically engineered Escherichia with deletion in the gene coding aspartate lyase (aspA) and an improved production of amino acids such as lysine, valine, threonine and homoserine. U.S. Pat. No. 5,250,434 provides genetically engineered Corynebacterium thermoaminogenes having improved heat performance in the glutamate production. U.S. Pat. Nos. 5,908,768 and 5,378,616 provides genetically modified Escherichia having resistance to aspartate antimetabolite and reduced 2-ketoglutarate dehydrogenase activity for improved glutamate productivity. U.S. Pat. No. 5,846,790 provides mutant strains having ability to produce glutamate in absence of any biotin action-suppressing agent. U.S. Pat. No. 4,393,135 provides a genetically modified organism for glutamate production. U.S. Pat. No. 6,319,696 provides genetically engineered organism which overexpressed phosphoenolpyruvate synthase for the enhanced production of amino acids. U.S. Pat. No. 6,197,559 describes Klebsiella, Erwinia, Pantoea having ability to produce glutamate. U.S. Pat. No. 7,247,459 provides Klebsiella, Erwinia, Pantoea, Enterobacter, Serattia encoded by citrate synthase gene derived from coryneform bacterium for glutamate production. U.S. Pat. No. 8,080,396 provides production of glutamate family of L-aminoacids including L-proline, L-hydroxyproline, L-ornithine, L-glutamine, L-glutamate, L-citrulline and L-arginine by increasing 2-ketoglutarate synthase activity. U.S. Pat. No. 8,129,151 describes Pantoea, Serattia with enhanced glutamate productivity and reduced formation of other glutamate family L-aminoacids. U.S. Pat. No. 7,955,823 provides genetically engineered Corynebacterium glutamicum mutant strains for the enhanced glutamate accumulation. European Patent No. EP1038970 describes the deletion of one or more of glutamate uptake system genes gluABCD from Coryneform bacterium. U.S. Pat. No. 8,211,688 describes the production of glutamine from glutamate biosynthesis pathway by reducing the activities of glutamine synthetase adenylyltransferase (GlnE) and PII regulatory protein for glutamine synthetase in genetically engineered Escherichia. U.S. Pat. No. 8,067,211 describes genetically engineered Coryneform bacterium for glutamine production by deleting, adding and substituting amino acids in the amino acid sequence of glutamine synthetase enzyme. U.S. Pat. No. 7,262,035 describes genetically engineered Coryneform bacterium with enhanced glutamine synthetase activity for glutamine production. U.S. Pat. No. 7,943,364 describes genetically engineered Coryneform bacterium with enhanced glutamine synthetase activity and reduced glutaminase activity for glutamine production.

In one method to enhance DHA uptake and utilization, the genetically modified host cells already known to produce glutamate and glutamine, as described in patents above, is subjected to further genetic engineering by means of expressing plasmids that code ATP and PEP dependent kinases that could phosphorylate DHA and facilitate its entry into glycolytic cycle. In another method to enhance DHA uptake and utilization, genetically modified host cells already known to produce glutamate, as described in patents above, is subjected to chemical mutagenesis and the strains with the ability to grow and produce desired glutamate with high enough titer and yield in a growth medium comprising DHA as a source of carbon will be selected and subjected to whole genome sequencing to identify specific mutation associated with the ability to grow and produce glutamate in a medium comprising DHA. Such a mutation will be introduced to the genetically modified host cells already known to produce glutamate to confer the ability to use DHA as a source of organic carbon. In another method to enhance DHA uptake and utilization, genetically modified host cells already known to produce glutamate, as described in patents above, is exposed to growth medium with increasing concentration of DHA and subjected to metabolic evolution. Strains that have gained ability to grow in the medium containing DHA as a major or sole source of carbon and still retain the ability to produce desired glutamate is selected and subjected to whole genome sequencing to identify specific mutation associated with the ability to grow and produce glutamate in a medium comprising DHA. Such a mutation will be introduced to the genetically modified host cells already known to produce glutamate, as described in patents above, for the purpose of conferring the ability to use DHA as a source of organic carbon.

In another embodiment, the present invention provides methods of producing glutamate derived compounds such as L-arginine, L-ornithine, L-citrulline, putrescine, L-proline, L-4-hydroxy proline. In one aspect, genetically modified microorganisms producing glutamate derived compounds are subjected to further genetic modifications to confer the ability to use DHA as a source of carbon. In another aspect of this invention, microorganisms that have ability to grow in a medium comprising DHA as a source of carbon is subjected to further genetic modifications to produce glutamate derived compounds. Such a genetic modification would involve overexpressing one or more components of the glutamate derivative biosynthetic pathway involving the enzyme N-acetylglutamate synthase, acetlyglutamate kinase, N-acetyl-γ-phospahte reductase, acetlyornithine aminotransferase, ornithine carbamoyltransferase, argininosuccinate synthase and argininosuccinate lyase.

In one aspect of this embodiment, microorganisms that have ability to grow in a medium comprising DHA as a source of carbon is subjected to further genetic modifications to include one or more nucleic acid comprising one or more nucleotide sequences encoding arginine biosynthetic pathway enzymes. Arginine, a glutamic acid derivative, is made from glutamic acid by a series of enzyme catalyzed biochemical reactions. Microorganisms that already have an ability to produce high amounts of glutamic acid natively or by one or more genetic modifications are the preferred host to produce arginine. On the other hand, arginine biosynthetic pathway can be directly engineered in the host cell that previously lack the ability to produce glutamic acid but can be engineered to selectively produce arginine. Furthermore, one or more of the enzymes functional in the arginine biosynthetic pathway such as N-acetylglutamate synthase, acetylglutamate kinase, N-acetyl-gamma-glutamyl phosphate reductase, acetylornithine aminotransferase, glutamate acyltransferase, ornithine carbamoyltransferase, argininosuccinate synthase and argininosuccinate lyase are overexpressed for the enhanced production of arginine from glutamic acid. In addition, attenuation or deletion of one or more of enzymes such as methylglyoxal synthase, acetate kinase, aldehyde dehydrogenase, pyruvate oxidase, pyruvate-formate lyase, alanine transaminase, lactate dehydrogenase, acetyl-CoA carboxylase, beta-ketothiolase and HMG-CoA synthase improves pyruvate utilization in arginine biosynthesis. Furthermore, increasing the expression of the lysE gene and argJ gene encoding the ornithine acetyl transferase would improve the arginine production. In addition, microorganisms having enhanced aspartate ammonia-lyase and argininosuccinate synthase activity cause an accumulation of arginine in the medium. Furthermore, blocking one or more of arginine degradation pathways such as formation of agmatine catalyzed by arginine decarboxylase, formation of 4-guanidinobutanamide catalyzed by arginine 2-monooxigenase, formation of ornithine catalyzed by arginase and formation of citruline catalyzed by arginine deiminase improves arginine accumulation. Furthermore, improving arginine transport, reducing uptake from media and reducing feedback inhibition increases arginine productivity.

In another aspect of this embodiment, microorganisms that have ability to grow in a medium comprising DHA as a source of carbon is subjected to further genetic modifications to include one or more nucleic acid molecules comprising one or more nucleotide sequence encoding ornithine biosynthetic pathway enzymes. Ornithine, a glutamic acid derivative, is made from glutamic acid by a series of enzyme catalyzed biochemical reactions.

Microorganisms that already has an ability to produce high amounts of glutamic acid natively or by one or more genetic modifications are preferred hosts to produce ornithine. On the other hand, ornithine biosynthetic pathway can be directly engineered in the host cell that previously lack the ability to produce glutamic acid but can be engineered to selectively produce ornithine. Furthermore, one or more of the enzymes functional in the ornithine biosynthetic pathway such as N-acetylglutamate synthase, acetylglutamate kinase, N-acetyl-gamma-glutamyl phosphate reductase, acetylornithine aminotransferase, acetylornithine deacetylase and glutamate acyltransferase are overexpressed for the enhanced production of ornithine from glutamic acid. In addition, attenuation or deletion of one or more of enzymes methylglyoxal synthase, acetate kinase, aldehyde dehydrogenase, pyruvate oxidase, pyruvate-formate lyase, alanine transaminase, lactate dehydrogenase, acetyl-CoA carboxylase, beta-ketothiolase and HMG-CoA synthase improves pyruvate utilization in ornithine biosynthesis. Furthermore, increasing the expression of the polynucleotide encoding ornithine exporter polypeptide improves the ornithine excretion. In addition, attenuating one or more of the genes selected from the group consisting of odhA, sucA, dapA, dapB, ddh, lysA, argR, argF, argG, argH, lysC and asd improves the production of ornithine. Furthermore, blocking one or more of ornithine degradation pathways such as formation of putrescine catalyzed by ornithine decarboxylase, formation of citrulline catalyzed by ornithine carbomyl transferase, and formation of arginine catalyzed by glycine amidino transferase improves ornithine accumulation. Furthermore, improving ornithine transport, reducing uptake from media and reducing feedback inhibition increases ornithine productivity.

In yet another aspect of this embodiment, microorganisms that have ability to grow in a medium comprising DHA as a source of carbon is subjected to further genetic modifications to include one or more nucleic acid molecules comprising one or more nucleotide sequence encoding citrulline biosynthetic pathway enzymes. Citrulline, a glutamic acid derivative, is made from glutamic acid by a series of enzyme catalyzed biochemical reactions.

Microorganisms that already has an ability to produce high amounts of glutamic acid natively or by one or more genetic modifications are preferred host to produce citrulline. On the other hand, citrulline biosynthetic pathway can be directly engineered in the host cell that previously lack the ability to produce glutamic acid but can be engineered to selectively produce citrulline. Furthermore, one or more of the enzymes selected from a group of citrulline biosynthetic pathway such as N-acetylglutamate synthase, acetylglutamate kinase, N-acetyl-gamma-glutamyl phosphate reductase, acetylornithine aminotransferase, acetylornithine deacetylase, glutamate acyltransferase and ornithine carbamoyltransferase are overexpressed for the enhanced production of citrulline from glutamic acid. In addition, attenuation or deletion of one or more of enzymes methylglyoxal synthase, acetate kinase, aldehyde dehydrogenase, pyruvate oxidase, pyruvate-formate lyase, alanine transaminase, lactate dehydrogenase, acetyl-CoA carboxylase, beta-ketothiolase and HMG-CoA synthase improves pyruvate utilization in citrulline biosynthesis. Furthermore, attenuating the expression of one or more of genes pepA, pepB, pepD, argG, and argR and overexpressing of argF, argJ and argB (feedback resistance N-acetyl L-glutamate kinase) genes improves the production of citrulline. Furthermore, blocking one or more of citrulline degradation pathways such as formation of arginine catalyzed by arginine deiminase, formation of arginino succinate catalyzed by arginino succinate synthase improves citrulline accumulation. Furthermore, improving citrulline transport, reducing uptake from media and reducing feedback inhibition increases citrulline productivity.

In one aspect of this embodiments, microorganisms that have ability to grow in a medium comprising DHA as a source of carbon is subjected to further genetic modifications to include one or more nucleic acid molecules comprising one or more nucleotide sequences encoding proline biosynthetic pathway enzymes. Proline, a glutamic acid derivative, is made from glutamic acid by a series of enzyme catalyzed biochemical reactions. Proline is also made from ornithine catalyzed by ornithine cyclo deaminase. Microorganisms that already has an ability to produce high amounts of glutamic acid or ornithine natively or by one or more genetic modifications are a preferred host to produce proline. On the other hand, proline biosynthetic pathway can be directly engineered in the host cell that previously lack the ability to produce glutamic acid or ornithine but can be engineered to selectively produce proline. Furthermore, one or more of the enzymes functional in the proline biosynthetic pathway such as glutamate kinase, glutamyl phosphate reductase, pyrroline-5-carboxylate reductase and ornithine cyclo deaminase are overexpressed for the enhanced production of proline from glutamic acid. In addition, attenuation or deletion of one or more of enzymes methylglyoxal synthase, acetate kinase, aldehyde dehydrogenase, pyruvate oxidase, pyruvate-formate lyase, alanine transaminase, lactate dehydrogenase, acetyl-CoA carboxylase, beta-ketothiolase and HMG-CoA synthase improves pyruvate utilization in proline biosynthesis. Furthermore, overexpression of proB gene containing nucleotide sequence for gamma-glutamyl kinase activity improves the production of proline and hydroxyproline. Furthermore, blocking one or more of proline degradation pathways such as formation of pyrroline-5-carboxylate catalyzed by proline dehydrogenase, formation of 2-ketoaminovalerate catalyzed by proline reductase improves proline accumulation. Furthermore, improving proline transport, reducing uptake from media and reducing feedback inhibition increases proline productivity.

In some embodiments, microorganisms that have ability to grow in a medium comprising DHA as a source of carbon is subjected to further genetic modifications to include one or more nucleic acid molecules comprising one or more nucleotide sequences encoding hydroxyproline biosynthetic pathway enzymes. Hydroxproline is made from proline by a single step biochemical reaction catalyzed by hydroxylase. Microorganisms that already has an ability to produce high amounts of proline by one or more genetic modifications are a preferred host for the production of hydroxyproline. On the other hand, hydroxyproline biosynthetic pathway can be directly engineered in the host cell that previously lack the ability to produce proline but can be engineered to selectively produce hydroxyproline. Furthermore, one or more of the genes selected from a group of hydroxyproline biosynthetic pathway such as glutamate kinase, glutamyl phosphate reductase, pyrroline-5-carboxylate reductase, ornithine cyclo deaminase and hydroxylase are overexpressed for the enhanced production of hydroxyproline. Furthermore, blocking one or more of hydroxyproline degradation pathways such as formation of pyrroline-3-hydroxy-5-carboxylate catalyzed by proline dehydrogenase improves hydroxyproline accumulation. Furthermore, improving hydroxyproline transport, reducing uptake from media and reducing feedback inhibition increases hydroxyproline productivity.

In some embodiments, microorganisms that have ability to grow in a medium comprising DHA as a source of carbon is subjected to further genetic modifications to include one or more nucleic acid molecules comprising one or more nucleotide sequences encoding putrescine biosynthetic pathway enzymes. Putrescine, a glutamic acid derivative, is made from arginine by a series of enzyme catalyzed biochemical reactions. Putrescine is also made from ornithine catalyzed by ornithine decarboxylase. Microorganisms that already has an ability to produce high amounts of arginine or ornithine natively or by one or more genetic modifications are a preferred host to produce putrescine. On the other hand, putrescine biosynthetic pathway can be directly engineered in to the host cell that previously lack the ability to produce arginine or ornithine but can be engineered to selectively produce putrescine. Furthermore, one or more of the enzymes selected from a group of putrescine biosynthetic pathway such as ornithine decarboxylase, arginine decarboxylase and agmatinase are overexpressed for the enhanced production of putrescine from arginine and ornithine. In addition, attenuation or deletion of one or more of enzymes methylglyoxal synthase, acetate kinase, aldehyde dehydrogenase, pyruvate oxidase, pyruvate-formate lyase, alanine transaminase, lactate dehydrogenase, acetyl-CoA carboxylase, beta-ketothiolase and HMG-CoA synthase improves pyruvate utilization in putrescine biosynthesis. Furthermore, inactivation or deletion of one or more gene selected from speE encoding spermidine synthase, speG encoding spermidine N-acetyltransferase, argl encoding ornithine carbamoyltransferase and puuP gene encoding putrescine importer improves putrescine productivity. In addition, overexpression of argCDE encoding ornithine biosynthesis, speA encoding arginine decarboxylase, speB encoding agmatinase and speC gene encoding ornithine decarboxylase enhances the putrescine productivity.

Genetically modified host cells capable of producing “glutamate derived compounds” using conventional sugars such as glucose, sucrose or glycerol are already known in the art. For example, U.S. Pat. No. 8,647,838 provides genetically engineered Escherichia and Pantoea that has been modified to attenuate expression of several genes encoding arginine transporter. U.S. Patent Application Publication No. 2003/0124686 provides increased arginine productivity on genetically engineered organism that has enhanced argininosuccinate synthase activity. U.S. Pat. No. 6,897,048 provides genetically engineered arginine producing organism with recombinant DNA containing argJ gene encoding ornithine acyltransferase activity. U.S. Pat. No. 7,252,978 provides enhanced expression of lysE gene for arginine production. U.S. Pat. Nos. 4,430,430, 4,775,623, 3,849,250, 5,034,319 and 5,017,482 provides genetically engineered Corneform, Corynebacterium and Brevibacterium for the production arginine. U.S. Patent Application Publication No. 2003/0153058 provides genetically engineered microbial cells having high glutamate dehydrogenase activity. U.S. Pat. No. 5,217,888 provides genetically engineered Kluyveromyces polysporus for the high arginine production. U.S. Pat. No. 4,086,137 provides genetically engineered Bacillus for high arginine production. European Patent No. EP2990475 provides enhanced aspartate ammonia lyase activity for increased arginine production. Chinese Patent Nos. CN105018515 CN103173466 provides methods and gene knock outs for enhanced arginine production. In addition, recent publication by Park et al (Metabolic engineering of Corynebacterium glutamicum for L-arginine production. Nat. Commun. 2014, 5, 4618) provides metabolically engineered Corynebacterium for high arginine production. In another publication from Ginesy et al (Metabolic engineering of Escherichia Coli for enhanced L-arginine biosynthesis. Microbial Cell Factories, 2015, 14, 29) reported enhanced arginine productivity when argR, speC, speE, adiA were knocked out and feedback resistance argA214 and argA215 were introduced. U.S. Pat. No. 5,188,947 provides genetically engineered Arthrobacter, Corynebacterium and Brevibacterium for high ornithine production. U.S. Pat. Nos. 3,374,150 and 2,988,489 provides fermentative production of ornithine by wild type and genetically engineered Micrococcus glutamicus. U.S. Pat. No. 3,574,061 provides process for preparing L-ornithine using mutant microorganisms. U.S. Pat. No. 8,951,759 increased the production of L-ornithine by expressing polynucleotide encoding a L-ornithine exporter. U.S. Pat. No. 8,741,608 increased ornithine production by attenuating one or more genes in the group consisting of odhA, sucA, dapA, dapB, ddh, lysA, argR, argF, argG, argH and lysC genes. U.S. Patent Application Publication. No. 2013/0023016 provides Corynebacteria transformant strains for producing ornithine and arginine. In addition, a recent publication from Jiang et al (Metabolic evolution of Corynebacterium glutamicum for increased production of L-ornithine. BMC Biotechnology, 2013, 13, 47) reported C glutamicum (ΔargF, ΔproB, ΔargR, ΔspeE) producing up to 25 g/L ornithine. In another publication from Qin et al (Modular pathway rewiring of Saccharomyces cerevisiae enables high-level production of ornithine. Nat. Commun. 2015, 6, 8224) reported Saccharomyces cerevisiae for the high-level production of ornithine. In another publication, Dongmei et al reported (Engineering Corynebacterium glutamicum to enhance L-ornithine production by gene knockout and comparative proteomic analysis. Chin. J. Chem. Eng., 2012, 20(4), 731-739) C. glutamicum (ΔargF, ΔproB, Δkgd) producing up to 5 g/L ornithine. In another publication from Kim et al (Metabolic engineering of Corynebacterium glutamicum for the production of L-ornithine. Biotechnology and Bioengineering, 2015, 112, 416) reported genetically engineered C. glutamicum (ΔargF, ΔproB, ΔagrR) allowed production of 52 g of ornithine. U.S. Pat. No. 8,722,370 provides genetically engineered Enterobacteriaceae spp. that has been modified to attenuate the expression of pepA, pepB and pepD genes for the production of L-citrulline. U.S. Pat. No. 3,282,794 provides genetically engineered Bacillus subtilis to produce citrulline. In addition, a recent publication from Ikeda et al (Reengineering of a Corynebacterium glutamicum L-arginine and L-citruline producer. Appl. Environ. Microbiol. 2009, 75, 1635-1641) reported genetically engineered C. glutamicum (ΔargR) for the production of citrulline. In another recent publication from Eberhardt et al (L-citurlline production by metabolically engineered Corynebacterium glutamicum from glucose and alternative carbon sources. AMB Express, 2014, 4, 85) reported C. glutamicum (ΔargR) and overexpressed argB and argF for the production of citrulline. Hao et al have reported (Improvement of L-citrulline production in Corynebacterium glutamicum by ornithine acetyltransferase J Ind. Microbiol. Biotechnol. 2015, 42, 307-313) that genetically engineered C. glutamicum (ΔargR, ΔargG) and overexpressed argJ for the production of citrulline. U.S. Pat. No. 4,444,885 provides genetically engineered Arthrobacter, corynebacterium, Saccharomyces and Brevibacterium for high proline production. U.S. Pat. No. 8,883,459 provides genetically engineered microorganisms for the conversion of 4-hydroxy-2-oxoglutaric acid to 4-hydroxy-L-proline. U.S. Pat. No. 3,650,899 provides genetically engineered microorganisms for the production of proline and hydroxyproline. U.S. Pat. No. 3,819,483 provides genetically engineered Corynebacterium and Brevibacterium to produce proline. U.S. Pat. No. 8,343,734 provides genetically engineered host microorganism having mutated variants of proB gene for the increased proline production. U.S. Pat. No. 5,334,517 provides genetically engineered Clonostachys, Gliocladium, Nectria for the conversion of sugars to hydroxyl proline. Chinese Patent No. CN104561072 discloses method of producing L-proline by expressing gamma-glutamate kinase activity in the genetically engineered organisms. Chinese Patent Nos. CN103509813 and CN104894152 discloses method of producing 4-hydroxy proline by overexpressing proline-4-hydroxylase activity in the genetically engineered organism. Chinese Patent No. CN104928311 discloses genetically engineered microorganism that co-expresses glutamyl kinase and proline-4-hydroxylase for the enhanced production of 4-hydroxyproline. In addition, a publication from Katz et al (Biosynthesis of trans-4-hydroxy-L-proline by Streptomyces griseoviridus. J. Biol. Chem., 1979, 254, 6684-6690) reported synthesis of 4-hydroxyproline from proline under microbial fermetative conditions. In another recent paper, Yi et al (Biosynthesis of trans-4-hydroxy-L-proline by recombinant strains of Corynebacterium glutamicum and Escherichia coli. BMC Biotechnology, 2014, 14, 44) disclosed heterologous expression of proline-4-hydrolase in genetically engineered microorganisms for the 4-hydroxy proline production. In another publication, Jensen et al (Ornithine cyclodeaminase-based proline production by Corynebacterium glutamicum. Microbial Cell Factories, 2013, 12, 63) disclosed a direct conversion of ornithine to proline by ornithine cyclodeaminase using Corynebacterium glutamicum. In another recent publication, Falcioni et al (Efficient hydroxyproline production from glucose in minimal media by Corynebacterium glutamicum. Biotechnology and Bioenginnering, 2015, 112, 2, 322-330) reported a C. glutamicum whole cell biocatalyst for the efficient production of 4-hydroxyproline. U.S. Patent Application Publication. No. 2009/0275093 provides genetically engineered microorganism having overexpressed levels of speA, speB, speC, and speF for putrescine productivity. U.S. Patent Application Publication. No. 2014/0363859 provides recombinant microorganism having enhanced ability to produce putrescine. U.S. Patent Application Publication. No. 2004/0086985 provides recombinant microorganism wherein an arginine decarboxylase gene is amplified and expressed for agmatine production. U.S. Pat. No. 8,481,293 provides mutant microorganism having enhanced ability to produce putrescine wherein gene involved in the putrescine degradation is inactivated or deleted. International PCT Application Publication No. WO2014/148743 provides recombinant microorganism for producing putrescine. International PCT Application Publication No. WO2012/077995 provides recombinant microorganism for producing putrescine wherein ornithine carbamoyl transferase and the activity of the protein NCg11221 is weakened. A recent publication by Nguyen et al (Fermentative production of the diamine putrescine: system metabolic engineering of Corynebacterium glutamicum. Metabolites, 2015, 5, 211-231) discloses overexpression of feedback-resistant N-acetylglutamate kinase and deleting gene snaA for improved putrescine production. Another publication from Schneider et al (Putrescine production by engineered Corynebacterium glutamicum. Appl. Microbiol. Biotechnol. 2010, 88, 859-868) discloses overexpression of speC and deletion of argR and argF for enhanced putrescine productivity. Another publication from Qian et al (Metabolic engineering of Escherichia coli for the production of putrescine: A four carbon diamine. Biotechnology and Bioengineering, 2009, 104, 651-662) discloses overexpression of argCDE and deletion of argl for enhanced putrescine productivity. Another publication from Schneider et al (Improving putrescine production by engineered Corynebacterium glutamicum by finetuning Ornithine transcarbomoylase activity using plasmid addiction system. Appl. Microbiol. Biotechnol. 2012, 95, 169-178) discloses overexpression of speC and fine-tuned argF activity for enhanced putrescine productivity. Another publication from Meiswinkel et al (Crude glycerol-based production of amino acids and putrescine by Corynebacterium glutamicum. Bioresource Technology. 2013, 145, 254-258) discloses expression of glpF, glpK, glpD for enhanced glycerol uptake for the production of putrescine and amino acids.

In one method to enhance DHA uptake and utilization, the genetically modified host cells already known to produce glutamate derived compounds, as described in patents above, is subjected to further genetic engineering by means of expressing plasmids that code ATP and PEP dependent kinases that could phosphorylate DHA and facilitate its entry into glycolytic cycle. In an another method to enhance DHA uptake and utilization, genetically modified host cells already known to produce glutamate derived compounds, as described in patents above, is subjected to chemical mutagenesis and the strains with the ability to grow and produce desired glutamate with high enough titer and yield in a growth medium comprising DHA as a source of carbon is selected and subjected to whole genome sequencing to identify specific mutation associated with the ability to grow and produce glutamate derived compounds in a medium comprising DHA. Such a mutation will be introduced to the genetically modified host cells already known to produce glutamate derived compounds to produce conferring the ability to use DHA as a source of organic carbon. In another method to enhance DHA uptake and utilization, genetically modified host cells already known to produce glutamate derived compounds, as described in patents above, is exposed to growth medium with increasing concentration of DHA and subjected to metabolic evolution. Strains that have gained ability to grow in the medium containing DHA as a major or sole source of carbon and still retain the ability to produce desired glutamate derived compounds is selected and subjected to whole genome sequencing to identify specific mutation associated with the ability to grow and produce glutamate derived compounds in a medium comprising DHA. Such a mutation is introduced to the genetically modified host cells already known to produce glutamate derived compounds, as described in patents above, for the purpose of conferring the ability to use DHA as a source of organic carbon.

In this embodiment, the present invention provides methods of producing L-aspartic acid. In one aspect of this invention, genetically modified L-aspartic acid producing microorganisms are subjected to further genetic modifications to confer the ability to use DHA as a source of carbon. In another aspect of this invention, microorganisms that have ability to grow in a medium comprising DHA as a source of carbon is subjected to further genetic modifications to produce L-aspartic acid. Such a genetic modification would involve overexpressing one or more components of the aspartate biosynthesis pathway such as enhancing the activities of the enzyme pyruvate carboxylase, phosphoenolpyruvate carboxylase, phosphoenol pyruvate carboxykinase and aspartate transaminase and attenuating the activities of the enzymes citrate synthase, malate dehydrogenase, aspartate kinase and aspartate decarboxylase. In addition, protein functionalities are modified to build aspartic acid feedback resistance and enhance aspartate productivity. Cell structure is modified to increase aspartic acid release and reduce aspartic acid based intracellular toxicity.

In some embodiments, microorganisms that have ability to grow in DHA in a medium comprising DHA as a source of carbon is subjected to further genetic modifications to include one or more nucleic acid molecules comprising one or more nucleotide sequences encoding aspartic acid biosynthetic pathway enzymes. Aspartic is synthesized from TCA metabolites by two methods. In one method, fumaric acid, a metabolite in TCA cycle, is aminated by aspartate ammonia lyase. In another method, oxaloacetate is transformed to aspartic acid through reductive amination reaction by the action of aspartate transaminase. Suitable aspartic acid biosynthesis includes enzymes pyruvate carboxylate, phosphoenolpyruvate carboxylase, aspartate transaminase, phosphoenolpyruvate carboxykinase and aspartate ammonia lyase. Furthermore, inactivation or deletion of one or more gene selected from SdhABCD encoding succinate dehydrogenase, Cit123 encoding citrate synthase, AceE and Lpd encoding pyruvate decarboxylase, PanD encoding aspartate decarboxylase and Ask gene encoding aspartate kinase improves aspartic acid productivity. In addition, overexpression of AspC encoding aspartate transaminase, AspA encoding aspartate ammonia lyase, phosphoenolpyruvate carboxylase and pyruvate carboxylase enhances the aspartic acid productivity.

Genetically modified host cells capable of producing aspartate using conventional sugars such as glucose, sucrose or glycerol are already known in the art. For example, U.S. Pat. No. 9,051,591 provides genetically engineered bacterium of Enterobacteriaceae family that has enhanced aspartase transaminase and decreased citrate synthase and glutamate dehydrogenase activity for the increase aspartic acid production. U.S. Pat. No. 4,000,040 provides genetically engineered Corynebacterium and Brevibacterium for the enhanced aspartic acid productivity. International PCT Application Publication No. WO2010/038905 provides methods for the enhanced aspartic acid production from Pantoea which has been modified to have decreased 2-ketoglutarate dehydrogenase, citrate synthase and increased PEP carboxylase and pyruvate carboxylase activity. Chinese Patent No. CN105296411 discloses genetically engineered Escherichia coli obtained by knocking out malate synthase and isocitrate synthase and inserting aspartase gene for the enhanced production of aspartic acid.

In one method to enhance DHA uptake and utilization, the genetically modified host cells already known to produce aspartate, as described in patents above, is subjected to further genetic engineering by means of expressing plasmids that code ATP and PEP dependent kinases that could phosphorylate DHA and facilitate its entry into glycolytic cycle. In another method to enhance DHA uptake and utilization, genetically modified host cells already known to produce aspartate, as described in patents above, is subjected to chemical mutagenesis and the strains with the ability to grow and produce desired glutamate with high enough titer and yield in a growth medium comprising DHA as a source of carbon is selected and subjected to whole genome sequencing to identify specific mutation associated with the ability to grow and produce aspartate in a medium comprising DHA. Such a mutation is introduced to the genetically modified host cells already known to produce aspartate to confer the ability to use DHA as a source of organic carbon. In another method to enhance DHA uptake and utilization, genetically modified host cells already known to produce aspartate, as described in patents above, is exposed to growth medium with increasing concentration of DHA and subjected to metabolic evolution. Strains that have gained ability to grow in the medium containing DHA as a major or sole source of carbon and still retain the ability to produce aspartate is selected and subjected to whole genome sequencing to identify specific mutation associated with the ability to grow and produce aspartate in a medium comprising DHA. Such a mutation will be introduced to the genetically modified host cells already known to produce aspartate, as described in patents above, for the purpose of conferring the ability to use DHA as a source of organic carbon.

In one aspect, the present invention provides methods of producing aspartate derived compounds such as L-lysine, L-threonine, L-methionine, cadaverine, L-serine, L-cystine. In one aspect, genetically modified microorganisms producing aspartate derived compounds are subjected to further genetic modifications to confer the ability to use DHA as a source of carbon. In another aspect of this invention, microorganisms that have ability to grow in a medium comprising DHA as a source of carbon is subjected to further genetic modifications to produce aspartate derived compounds. Such a genetic modification would involve overexpressing one or more enzymes functional in the the aspartate derivative biosynthetic pathway including but not limited to aspartate kinase, aspartate semialdehyde dehydrogenase, dihydrodipicolinate synthase, dihydrodipicolinate reductase, tetrahydrodipicolinate succinylase, succinyl-diaminopimelate-aminotransferase, succinyl-diaminopimelate desuccinylase, diaminopimelate epimerase, diaminopimelate decarboxylase, homoserine dehydrogenase, homoserine kinase, threonine synthase, homoserine succinyltransferase, O-succinylhomoserine-(thiol)-lyase, adenosyl homocysteinase, methionine adenosyl transferase and lysine decarboxylase.

In one aspect of this embodiment, microorganisms that have ability to grow in a medium comprising DHA as a source of carbon is subjected to further genetic modifications to include one or more nucleic acid molecules comprising one or more nucleotide sequences encoding L-threonine biosynthetic pathway enzymes. L-threonine, an aspartate derivative, is made from aspartate by several biochemical steps that includes enzymes such as aspartate kinase (Ask), aspartate semialdehyde dehydrogenase (Asd), homoserine dehydrogenase (ThrA), homoserine kinase (ThrB) and threonine synthase (ThrC). Furthermore, one or more of the genes selected from ThrABC encoding homoserine dehydrogenase, homoserine kinase and threonine synthase and Pyc gene encoding pyruvate carboxylase, Pps gene encoding phosphoenol pyruvate synthase, Ppc gene encoding phophoenolpyruvate carboxylase, RhtB gene imparting homoserine resistance, RhtC imparting threonine resistance and ThrE gene encoding threonine export is overexpressed for the enhanced production of threonine. In addition, attenuating the expression of threonine dehydratase, homoserine succinyl transferase, dihydropicolinate synthase, threonine aldolase, threonine-3-dehydrogenase improves the production of L-threonine.

In some embodiments, microorganisms that have ability to grow in a medium comprising DHA as a source of carbon is subjected to further genetic modifications to include one or more nucleic acid molecules comprising one or more nucleotide sequences encoding L-methionine biosynthetic pathway enzymes. L-methionine, an aspartate derivative, is made from aspartate by several biochemical steps. Aspartate is converted through multiple steps to homoserine. Homoserine is converted to O-acetyl-homoserine or O-succinyl-homoserine by MetA or MetX. Some microorganisms utilize MetA polypeptide to make O-succinyl-homoserine while other organisms utilize MetX to make O-acetly-homoserine. O-acetly and O-succinyl-homoserine can be directly converted to homocysteine through transsulfuration. Enzymes included in methionine productions are aspartate kinase (Ask), aspartate semialdehyde dehydrogenase (Asd), homoserine dehydrogenase (ThrA), homoserinesuccinyl transferase (MetA), O-Succinylhomoserine thiol-lyase (MetBC or MetZ), Vitamin B₁₂-dependent methionine synthase (MetH), Vitamin B₁₂ independent methionine synthase (MetE), Adenoylmethionine synthase (MetK). Furthermore, one or more of the genes selected from a group of methionine biosynthetic pathway such as MetA, MetB, MetC, MetE, MetY, MetZ, MetX, MetH, MetF, MetK and MetQ are overexpressed for the enhanced production of methionine. In addition, attenuation or deletion of methionine S-oxide reductase, methionine tRNA ligase and threonine aldolase further improves the productivity of methionine.

In some embodiments, microorganisms that have ability to grow in DHA in a medium comprising DHA as a source of carbon is subjected to further genetic modifications to include one or more nucleic acid molecules comprising one or more nucleotide sequences encoding L-lysine biosynthetic pathway enzymes. L-lysine, an aspartate derivative, is made by two methods. In one method, aspartate is converted to lysine via 2,6-diamino pimelate. In 2,6-diamino pimelate pathway, Aspartate is converted through multiple biochemical steps to aspartate-4-semialdehyde. Aspartate-4-semialdehyde is converted to tetrahydro dipicolinate by DapAB. Tetrahydropicolinate is converted to 2,6-diaminopimelate by DapL or Ddh. In another method, tetrahydro dipicolinate is converted to 2,6-diaminopimelate in multiple biochemical steps by DapCDE. Meso-2,6-diaminopimelate is converted to L-lysine by LysA. In another method, L-lysine is produced from 2-Oxoglutarate via 2-aminoadipic acid. In 2-aminoadipic acid pathway, 2-oxoglutarate is converted to homocitrate by the action of homocitrate synthase; homocitrate is converted to cis-homo-aconitate by homocitrate dehydratase; cis-homo-aconitate is converted to homoisocitrate by homoaconitase hydrate; homoisocitrate is converted to oxaloglutarate by homoisocitrate dehydrogenase; oxaloglutarate is converted to 2-oxoadipate by oxaloglutarate decarboxylase; oxoadipate is converted to 2-aminoadipate by aminoadipate transaminase; 2-aminoadipate is converted to 2-aminoadipate semialdehyde by aminoadipate reductase; 2-aminoadipate semialdehyde is converted to saccharopine by saccharopine reducatse; saccharopine is converted to L-lysine by saccharopine dehydrogenase. Furthermore, one or more of the genes selected from a group of 2,6-diaminopimelate biosynthetic pathway such as AspC, Ask, Asd, DapA, DapB, DapD, DapC, DapE, DapF, DapL, Ddh and LysA are overexpressed for the enhanced production of lysine. In addition, attenuation or deletion of lysine decarboxylase, lysine acetyl transferase, lysine RNA ligase and lysine oxygenase further improves the productivity of lysine.

In some embodiments, microorganisms that have ability to grow in DHA in a medium comprising DHA as a source of carbon is subjected to further genetic modifications to include one or more nucleic acid molecules comprising one or more nucleotide sequences encoding cadaverine biosynthetic pathway enzymes. Cadaverine, an aspartate derivative, is made by decarboxylation of lysine catalyzed by enzyme lysine decarboxylase. Microorganisms that already has an ability to produce high amounts of lysine by one or more genetic modifications are a preferred host to produce cadaverine. On the other hand, cadaverine biosynthetic pathway can be directly engineered in the host cell that previously lack the ability to produce lysine but can be engineered to selectively produce cadaverine. Furthermore, one or more of the genes selected from a group of 2,6-diaminopimelate biosynthetic pathway such as AspC, Ask, Asd, DapA, DapB, DapD, DapC, DapE, DapF, DapL, Ddh, LysA and CadA are overexpressed for the enhanced production of cadaverine. In addition, attenuation or deletion of SpeE gene encoding putrescine/cadaverine aminopropyl transferase; SpeG gene encoding spermidine N-acyltransferase; YgjG gene encoding putrescine/cadaverine amino transferase; PuuP gene encoding putrescine importer; PuuA gene encoding glutamate-putrescine/glutamate-cadaverine ligase further improves the productivity of cadaverine.

Genetically modified microorganisms capable of producing aspartate derived compounds using conventional sugars such as glucose, sucrose or glycerol are already known in the art. For example, U.S. Pat. No. 9,080,190 provides genetically engineered Corynebacterium and Brevibacterium that have resistance to 2,2′-thiobis(ethylamine) for the increased production of cadaverine. U.S. Pat. No. 8,741,623 provides recombinant Corynebacterium and Escherichia for the enhanced cadaverine production. U.S. Pat. No. 7,189,543 provides methods for the production cadaverine from lysine, where the lysine is produced from conventional sugars. U.S. Pat. No. 8,871,477 provides genetically engineered microorganism that extracellularly secretes lysine decarboxylase to increase the yield of cadaverine. U.S. Patent Application Publication. No. 2012/0295317 provides recombinant microorganism that has intracellular and an extracellular lysine decarboxylase activity. U.S. Patent Application Publication. No. 2013/0157323 provides mutant organism that has high ability to produce cadaverine wherein cadaverine degradation pathways are inactivated or deleted. U.S. Patent Application Publication. No. 2011/0039313 provides recombinant microorganisms with enhanced lysine decarboxylate activity. U.S. Patent Application Publication. No. 2014/0134682 provides recombinant microorganisms with intracellular lysine decarboxylate activity and deregulated cadaverine export activity for enhanced cadaverine production. Chinese Patent No. CN102424811 provides genetically engineered microorganism with CadA and CadB gene activity for increased cadaverine production. International PCT Application. Publication No. WO2015/197014 provides genetically engineered Escherichia coli overexpressing CadA and CadB cell to produce cadaverine from lysine. A recent publication from Li et al (Cadaverine production by heterologous expression of Klebsiella oxytoca lysine decarboxylase, Biotechnology and Bioprocess engineering, 2014, 19, 965-972) disclosed methods for cadaverine production. Another publication from Kim et al (Optimization of direct lysine decarboxylase biotransformation for cadaverine production with whole-cell biocatalyst at high lysine concentration, J. Microbiol. Biotechnol, 2015, 25(7), 1108-1113) discloses a recombinant Escherichia coli overexpressing E. coli MG1655 cadA gene for the whole-cell production of cadaverine. In another recent publication, Kind et al (Identification and elimination of the competing N-acetlydiamonioentane pathway for improved production of diamino pentane by Corynebacterium glutamicum, Appl. Environ. Microbiol 2010, 76(15), 5175-5180) discloses genetically engineered Corynebacterium glutamicum and deletion of N-acetyldiaminopentane pathway for the increased production of cadaverine. Metabolic engineering of cadaverine cellular transport process (Metabolic engineering of cellular transport for overproduction of the platform chemical 1,5-diaminopentane in Corynebacterium glutamicum. Metabolic Engineering, 2011, 13, 617-627) and system wide engineering for cadaverine production (Systems-wide metabolic pathway engineering in Corynebacterium glutamicum for bio-based production of diaminopentane, Metabolic Engineering, 2010, 12, 341-351) have also been reported. In another publication by Mimitsuka et al (Metabolic engineering of Corynebacterium glutamicum for cadaverine fermentation, Biosci. Biotechnol. Biochem, 2007, 71(9), 2130-2135) discloses genetically engineered C. glutamicum with higher cadaverine production. In another publication, Li et al (Improving the secreation of cadaverine in Corynebacterium glutamicum by cadaverine-lysine antiporter, J. Ind. Microbiol. Biotechnol. 2014, 41, 701-709) have disclosed a cadB encoded antiporter for cadaverine secretion. Publications by Tateno et al (Direct production of cadaverine from soluble starch using Corynebacterium glutamicum coexpressing alpha-amylase and lysine decarboxylase, Appl. Microbiol. Biotechnol 2009, 82, 115-121) and Buschke et al (Metabolic engineering of Corynebacterium glutamicum for production of 1,5-diaminopentane from hemicellulose, Biotechnol. J. 2011, 6, 306-317 and Systems metabolic engineering of xylose-utilizing Corynebacterium glutamicum for production of 1,5-diaminopentane, Biotechnol. J. 2013, 8, 557-570) disclose the use of alternative sugars for cadaverine production. In another publication, Ma et al (Enhanced cadaverine production from L-lysine using recombinant Escherichia coli co-overexpressing CadA and CadB, Biotechnol. Lett., 2015, 37, 799-806) disclose co-expression of CadA and CadB for cadaverine production. In another publication, Qian et al (Metabolic engineering of Escherichia coli for the production of cadaverine: A five carbon diamine, Biotechnology and Bioengineering, 2011, 108, 11, 93-103) disclose metabolically engineered microorganisms for the production of cadaverine.

U.S. Pat. No. 7,790,424 provides genetically engineered microorganism overexpressing proteins involved in L-methionine synthesis. U.S. Pat. No. 8,551,742 provides microorganisms genetically engineered to overexpress MetABCEYXZH, CysDNCHIJGKM genes for production of methionine. U.S. Pat. No. 8,163,532 provides microorganism genetically engineered to produce methionine by reactivation of MetH. U.S. Pat. No. 8,871,477 provides genetically engineered microorganism. U.S. Pat. No. 8,148,117 provides genetically engineered microorganism that overexpress MetBCE and deleted McbR and McrB for the enhanced methionine production. U.S. Pat. No. 8,389,251 provides genetically engineered microorganism with increased expression of metabolic regulator RXN02910 and suppressing the activity of regulator RXA00655 for the enhanced methionine production. U.S. Pat. No. 3,729,381 provides genetically engineered microorganism and process for methionine production. U.S. Pat. No. 7,611,873 provides genetically engineered microorganism with enhanced intracellular homoserine transsuccinylase and deficient in methionine repressor for high methionine production. U.S. Pat. No. 8,389,250 provides genetically engineered microorganism wherein enhanced cysteine productivity improved methionine productivity. U.S. Pat. No. 8,017,362 provides genetically engineered microorganism that is deficient in a repressor of methionine biosynthesis system. U.S. Patent Application Publication. No. 2015/0247175 provides recombinant microorganism wherein MetE is attenuated for higher methionine production. U.S. Patent Application Publication. No. 2009/0298136 provides recombinant microorganisms that produce increased levels of methionine compared to their wild-type counterparts. U.S. Patent Application Publication. No. 2010/0009416 reported increased methionine productivity by increasing the amount of serine available for the metabolism. U.S. Patent Application Publication. No. 2009/0191610 reported increased methionine production by optimizing metabolic flux for organisms with respect to methionine synthesis. U.S. Patent Application Publication. No. 2011/0117614 disclosed recombinant microorganism with reduced isocitrate dehydrogenase activity for the production of methionine. U.S. Patent Application Publication. No. 2012/0288901 reported recombinant microorganism with higher methionine productivity by over expressing pentose pathway. U.S. Patent Application Publication. No. 2012/0288904 discloses modified strains with attenuated transformation of threonine to glycine or 2-keto butarate for higher methionine productivity. U.S. Patent Application Publication. No. 2009/0298135 provides a recombinant microorganism suitable for methionine production that comprises increased activity of YjeH gene. International PCT Application Publication No. WO2015/028674 provides genetically engineered microorganism wherein cobaltamin-dependent methionine synthase activity and methionine efflux are enhanced.

U.S. Pat. No. 7,319,026 provides genetically engineered microorganism with enhancement of RhtB, RhtC, and YfiK genes and attenuation of acetic acid formation for the higher threonine production. U.S. Pat. No. 7,598,062 provides genetically engineered microorganism in which FruR gene is switched off and ThrABC, PntA, PntB, RhtB, RhtC is overexpressed for higher threonine production. U.S. Pat. No. 4,347,318 and U.S. Pat. No. 4,452,890 provides genetically engineered microorganism resistant to 2-amino-3-hydroxy valeric acid resulting in higher threonine production. U.S. Pat. No. 7,256,021 provides genetically engineered microorganism which has mutation or deletion in the gene encoding aspartate ammonium lyase (AspA) resulting in greater threonine productivity. U.S. Pat. No. 7,638,313 provides genetically engineered microorganism in which YjgF gene is inactivated resulting in higher threonine productivity. U.S. Pat. No. 6,132,999 provides genetically engineered microorganism that bears threonine biosynthesis genes for 70 g/L threonine productivity. U.S. Pat. Nos. 8,389,251 and 5,188,949 provides genetically engineered microorganism that has resistance to mycophenolic acid and is capable of producing threonine. U.S. Pat. No. 5,077,207 provides genetically engineered microorganism from which dihydrodipicolinate synthase has been removed accumulating threonine as a product of cultivation. U.S. Pat. No. 6,297,031 and U.S. Pat. No. 7,138,266 provides genetically engineered microorganism that have inactivated threonine dehydrogenase activity useful for producing threonine. U.S. Pat. No. 5,661,012 provides genetically engineered microorganism that releases the feedback inhibition by lysine on aspartokinase III leading to higher threonine production. U.S. Pat. No. 4,996,147 provides genetically engineered microorganism having resistance to rifampicin, lysine, methionine, aspartic acid and homoserine accumulating threonine in the culture. U.S. Pat. No. 3,375,173 provides genetically engineered microorganism for fermentative production of threonine. U.S. Pat. No. 5,376,538 provides genetically engineered threonine producing microorganism that is resistant to phenylalanine and leucine. U.S. Pat. No. 5,153,123 provides process for producing threonine from aspartic acid with ethanol or glucose in a biotin free aqueous solution. U.S. Pat. No. 5,939,307 provides genetically engineered microorganism for the enhanced threonine productivity. U.S. Pat. No. 4,463,094 provides methionine metabolizing genetically engineered threonine producing organism. U.S. Pat. No. 4,321,325 provides genetically engineered microorganism for threonine production. U.S. Pat. No. 5,264,353 provides genetically engineered threonine producing microorganism having resistance to isoleucine. U.S. Pat. No. 7,220,571 provides genetically engineered microorganism that has resistance to threonine raffinate and borrelidin for threonine production. U.S. Pat. No. 5,342,766 provides genetically engineered microorganism having resistance to methionine and capable of producing threonine. U.S. Pat. Nos. 8,101,386 and 7,767,431 provides genetically engineered microorganism for the overproduction of threonine. U.S. Pat. No. 7,368,266 provides genetically engineered microorganism that has overexpressed phosphoenolpyruvate carboxylase for threonine production. U.S. Pat. No. 5,631,157 provides Escherichia coli VNII genetika 472T23 for threonine productivity. U.S. Patent Application Publication. No. 2005/0221448 reports a Enterobacteriaceae family where AceB gene is attenuated for threonine production. U.S. Patent Application Publication. No. 2010/0159537 reports genetically engineered threonine producing microorganism in which Asd gene is operably associated with at least one non-native promoter. U.S. Patent Application Publication. No. 2004/0132165 reports genetically engineered microorganism with enhanced aspartate aminotransferase activity for threonine production. International PCT application. No. WO2008/111708 provides genetically engineered microorganism with site-specific mutation for high threonine production. European Patent No. EP1,285,075 reports genetically engineered Enterobacteriaceae bacteria with ThrE gene from Coryneform for higher threonine productivity. International PCT Application. Publication Nos. WO2000/009660 and WO2002/031172 provides genetically engineered microorganism for threonine production.

U.S. Pat. No. 8,268,597 provides genetically engineered E. coli comprising a DapA gene from B. subtilis to produce lysine. U.S. Pat. No. 8,062,869 provides genetically engineered organism with enhanced expression of dihydropicolinate reductase, tetrahydropicolinate succinylase, aspartate semialdehyde dehydrogenase and phosphoenolpyruvate carboxylase for the production of lysine. U.S. Pat. No. 8,048,650 provides enhanced lysine productivity by inactivating endogeneous NCgI 1090 gene from a recombinant Corynebacterium. U.S. Pat. No. 7,981,640 provides recombinant microorganism with enhanced LysE, DapA, LysC genes to produce lysine. U.S. Pat. No. 5,268,293 provides lysine producing microorganism which is resistant to 1-amino-2-hydroxyvaleric acid, S-(2-aminoethyl)-L-cysteine and methyl lysine for enhanced lysine production. U.S. Pat. No. 6,090,597 provides Coryneform bacterium in which DNA encoding diaminopimelate decarboxylase and diaminopimelate dehydrogenase are enhanced to produce lysine. U.S. Pat. No. 5,766,925 provides genetically engineered microorganism with desensitized feedback inhibition by lysine or threonine. U.S. Pat. No. 6,040,160 provides lysine producing genetically modified microorganism with enhanced dihydropicolinate synthase, aspartokinase III having mutation to desensitize feedback inhibition from lysine. U.S. Pat. No. 6,004,773 provides lysine producing genetically engineered microorganism having an aspartokinase in which feedback inhibition is substantially desensitized. U.S. Pat. No. 4,066,501 provides genetically engineered Brevibacterium or Corynebacterium that are resistant to 2-aminolauryllactam, 3-methyl-lysine, N-carbobenzyloxy lysine for the increased production of lysine. U.S. Pat. No. 4,411,997 provides genetically engineered Brevibacterium or Corynebacterium that are resistant to ethylene glycol and capable of producing lysine. U.S. Pat. No. 4,657,860 provides genetically engineered Brevibacterium or Corynebacterium having resistant to purine and pyrimidine analog. U.S. Pat. No. 3,929,571 provides a method for better lysine production by supplementing nutrient medium with an antibiotic, surface active agent and an anti-oxidant. U.S. Pat. No. 3,920,520 provides genetically engineered microorganism for the substantial lysine product tolerance. U.S. Pat. No. 3,907,637 provides genetically engineered lysine producing microorganism having a resistance to 2-aminoethyl-L-cysteine and threonine. U.S. Pat. Nos. 3,871,960, 4,560,654 and 3,825,472 provides genetically engineered lysine producing microorganism that has resistant to feedback inhibition by 2-aminoethyl-L-cysteine. U.S. Pat. Nos. 3,708,395 and 3,707,441 provides genetically engineered lysine producing microorganism with substantial nutritional requirements for the increased productivity of lysine. U.S. Pat. No. 3,687,810 provides genetically engineered lysine producing microorganism capable of resisting antibiotics. U.S. Pat. No. 4,954,441 provides genetically engineered microorganism that contains a gene involved in the synthesis of dihydropicolinic acid synthetase. U.S. Pat. No. 4,861,722 provides genetically engineered lysine producing microorganism having the activity of diaminopimelic acid decarboxylase. U.S. Pat. No. 4,346,170 provides genetically engineered lysine producing microorganism that has resistant to a lysine analogue. U.S. Pat. No. 4,169,763 provides genetically engineered lysine producing microorganism resistant to aspartic acid analogs and sulfa drugs in the nutrient medium. U.S. Pat. No. 3,959,075 provides method for increasing lysine yield by culturing lysine producing microorganism in a nutrient medium supplemented by the culture liquor of a leucine producing microorganism. U.S. Pat. No. 8,058,036 provides genetically engineered microorganism showing enhanced lysine productivity by inactivating endogenous NCgI2534. U.S. Pat. No. 6,221,636 provides genetically engineered lysine producing microorganism in which feedback inhibition lysine and threonine is substantially desensitized. U.S. Pat. No. 8,067,210 provides genetically engineered microorganism for increased production of lysine by amplifying amino acid pathway genes in a host cell and further reducing feedback resistance. U.S. Pat. No. 8,932,861 provides genetically engineered lysine producing microorganism transformed with vector comprising fragments of a gene transposase. U.S. Pat. No. 9,109,242 provides genetically engineered microorganism having activity greater than the endogenous activity of lysine producing microorganisms. U.S. Pat. No. 6,740,742 provides a microbial cell with n-succimylaminokeptopimelate transaminase activity for the enhanced lysine production. U.S. Pat. No. 4,954,441 provides genetically engineered lysine producing microorganism that contains dihydropicolinic acid synthetase. U.S. Pat. Nos. 6,844,176 and 7,135,313 reports alleles of the LysC gene from Corynebacteria that code for desensitized aspartokinase and preparation of lysine using organism that contains these alleles. U.S. Pat. No. 7,083,942 reports alleles of the AceA gene from Coryneform encoding isocitrate lyases and producing lysine using organism that contains these alleles. U.S. Pat. Nos. 6,861,246, 7,981,640 and 6,200,785 provide genetically engineered microorganism containing enhanced Pyc, DapA, LysC, LysE, and DapB genes for the increased lysine production. U.S. Pat. No. 6,927,046 provides increased production of lysine by amplification of lysine biosynthetic pathway in genetically engineered microorganism. U.S. Pat. No. 7,205,131 provides genetically engineered microorganism to produce lysine via overexpression of the PtsH gene. U.S. Patent Application Publication No. 2010/0190217 reports lysine production in a genetically modified microorganism that has decreased activities of meso-diaminopimelic acid synthesis pathway and increased activities of diaminopimelate dehydrogenase. U.S. Patent Application Publication No. 2005/0266536 reports genetically modified organism for the enhanced production of lysine that has attenuated enzymes such as trehalose phosphate, maltooligosyl-trehalose synthase, maltooligosyl-trehalose trehalohydrolase. U.S. Patent Application Publication No. 2014/0045228 reports genetically engineered microorganism for the production of lysine from crude glycerol.

In one method to enhance DHA uptake and utilization, the genetically modified host cells already known to produce aspartate derived compounds, as described in patents above, is subjected to further genetic engineering by means of expressing plasmids that code ATP and PEP dependent kinases that could phosphorylate DHA and facilitate its entry into glycolytic cycle. In another method to enhance DHA uptake and utilization, genetically modified host cells already known to produce aspartate derived compounds, as described in patents above, is subjected to chemical mutagenesis and the strains with the ability to grow and produce desired glutamate with high enough titer and yield in a growth medium comprising DHA as a source of carbon is selected and subjected to whole genome sequencing to identify the specific mutation associated with the ability to grow and produce aspartate derived compounds in a medium comprising DHA. Such a mutation will be introduced to the genetically modified host cells already known to produce aspartate derived compounds to confer the ability to use DHA as a source of organic carbon. In yet another method to enhance DHA uptake and utilization, genetically modified host cells already known to produce aspartate derived compounds, as described in patents above, is exposed to growth medium with increasing concentration of DHA and subjected to metabolic evolution. Strains that have gained ability to grow in the medium containing DHA as a major or sole source of carbon and still retain the ability to produce aspartate derived compounds is selected and subjected to whole genome sequencing to identify the specific mutation associated with the ability to grow and produce aspartate derived compounds in a medium comprising DHA. Such a mutation will be introduced to the genetically modified host cells already known to produce aspartate derived compounds, as described in patents above, for the purpose of conferring the ability to use DHA as a source of organic carbon.

In another embodiment, the present invention provides methods of producing branched chain amino acids such as L-leucine, L-isoleucine and L-valine using DHA as a source of carbon. In one aspect, genetically modified microorganisms producing branched chain amino acids are subjected to further genetic modifications to confer the ability to use DHA as a source of carbon in a medium containing dihydroxyacetone as a carbon source. In another aspect of this invention, microorganisms that have ability to grow in a medium comprising DHA as a source of carbon is subjected to further genetic modifications to produce branched chain amino acids. Such a genetic modification would involve overexpressing one or more components of the valine, leucine, isoleucine derivative biosynthetic pathway, including but not limited to acetolactate synthase, keto-acid reductoisomerase, dihydroxy acid dehydratase, branched-chain amino acid aminotransferase, threonine dehydratase, isopropylmalate synthase, isopropylmalate isomerase, isopropylmalate dehydrogenase and tyrosine transaminase.

In one aspect of this embodiment, microorganisms that have ability to grow in DHA in a medium comprising DHA as a source of carbon is subjected to further genetic modifications to include one or more nucleic acid molecules comprising one or more nucleotide sequences encoding shikimic acid biosynthetic pathway enzymes. Shikimic acid, an aromatic amino acid pathway product, is made from D-erythrose-4P by a series of enzyme catalyzed biochemical reactions. Microorganisms that already has an ability to produce one or more aromatic amino acids are a preferred host to produce shikimic acid. On the other hand, shikimic acid biosynthetic pathway can be directly engineered in to the host cell that previously lack the ability to produce shikimic acid but can be engineered to selectively produce shikimic acid. Furthermore, one or more of the genes selected from a group of shikimic acid biosynthetic pathway such as TktA, TalB, AroG, AroF, AroH, AroB, AroD and AroE is overexpressed for the enhanced production of shikimic acid. Blocking shikimic acid degradation pathway by attenuation or deletion of AroK and AroL gene encoding shikimate kinase reduces the production of chorismate. Substantial reduction of the activity of the negative regulator TyrR improves the productivity of shikimic acid. Overexpressing pyruvate kinase to increase the production of phosphoenol pyruvate, attenuation or deletion of phosphoenolpyruvate carboxylase and pyruvate carboxylase increases the availability of phosphoenol pyruvate for shikimic acid synthesis. Furthermore, improving shikimic acid transport, reducing shikimic acid uptake from media and blocking feedback inhibition increases shikimic acid productivity. Shikimic acid biosynthetic pathway can be further extended to produce chrismate from shikimic acid by means of expressing AroK, AroL, AroA and AroC genes.

In another aspect of this embodiment, microorganisms that have ability to grow in DHA in a medium comprising DHA as a source of carbon is subjected to further genetic modifications to include one or more nucleic acid molecules comprising one or more nucleotide sequence encoding phenylalanine biosynthetic pathway enzymes. Phenylalanine, an aromatic amino acid, is made from chorismate by a series of enzyme catalyzed biochemical reactions.

Microorganisms that already has an ability to produce high amounts of chorismate by one or more genetic modifications are a preferred host for the production of phenylalanine. On the other hand, phenylalanine biosynthetic pathway can be directly engineered in to the host cell that previously lack the ability to produce chorismate but can be engineered to selectively produce phenylalanine. Furthermore, one or more of the genes selected from a group of phenylalanine biosynthetic pathway such as TyrA, PheA and TyrB is overexpressed for the enhanced production of phenylalanine. In addition, attenuation or deletion of TrpE gene encoding anthraniline synthase, TrpD gene encoding anthranilate phosphoribosyl transferase and AroE gene encoding shikimate dehydrogenase further improves the productivity of phenylalanine. Furthermore, improving phenylalanine transport, reducing phenylalanine uptake from media and reducing feedback inhibition increases phenylalanine productivity.

In some embodiments, microorganisms that have ability to grow in DHA in a medium comprising DHA as a source of carbon is subjected to further genetic modifications to include one or more nucleic acid molecules comprising one or more nucleotide sequence encoding tyrosine biosynthetic pathway enzymes. Tyrosine, an aromatic amino acid, is made from chorismate by a series of enzyme catalyzed biochemical reactions. Microorganisms that already has an ability to produce high amounts of chorismate by one or more genetic modifications are a preferred host to produce tyrosine. On the other hand, phenylalanine biosynthetic pathway can be directly engineered in the host cell that previously lack the ability to produce chorismate but can be engineered to selectively produce tyrosine. Furthermore, one or more of the genes selected from a group of tyrosine biosynthetic pathway such as TyrA gene encoding chorismate mutase/prephenate dehydrogenas and TyrB encoding tyrosine transaminase are overexpressed for the enhanced production of tyrosine. In addition, attenuation or deletion of PheA gene encoding chorismate mutase/prephenate dehydratase, TrpE gene encoding anthraniline synthase, TrpD gene encoding anthranilate phosphoribosyl transferase and AroE gene encoding shikimate dehydrogenase further improves the productivity of tyrosine. Furthermore, improving tyrosine transport, reducing tyrosine uptake from media, desensitizing prephenate dehydrogenase for the feedback inhibition from tyrosine and other metabolic engineering methods to reduce tyrosine feedback inhibition increases tyrosine productivity.

In some embodiments, microorganisms that have ability to grow in DHA in a medium comprising DHA as a source of carbon is subjected to further genetic modifications to include one or more nucleic acid molecules comprising one or more nucleotide sequences encoding tryptophan biosynthetic pathway enzymes. Tryptophan, an aromatic amino acid, is made by a series of enzymatic biochemical reactions starting from chorismate. Microorganisms that already has an ability to produce high amounts of chorismate by one or more genetic modifications are a preferred host to produce tryptophan. On the other hand, tryptophan biosynthetic pathway can be directly engineered in to the host cell that previously lack the ability to produce chorismate but can be engineered to selectively produce tryptophan. Furthermore, one or more of the genes selected from a group of tryptophan biosynthetic pathway such as TrpE, TrpD, TrpC, TrpA and TrpB is overexpressed for the enhanced production of tryptophan. In addition, attenuation or deletion of gene TyrA encoding chorismate mutase/prephenate dehydrogenase and PheA encoding chorismate mutase/prephenate dehydratase, TrpR gene encoding protein TrpR necessary for the regulation of tryptophan operon, AroE gene encoding shikimate dehydrogenase and anthranilate 1,2-dioxygenase improves the tryptophan productivity. Mutating serine production by overexpressing SerA, SerB, SerC genes and further removing serine degradation pathway by deleting serine deaminase improves tryptophan productivity. Furthermore, improving tryptophan transport from intracellular to the medium, reducing tryptophan uptake from media by deletion of Mtr gene and TnaB gene, reducing tryptophan degradation by deleting TnaA gene and other metabolic engineering methods to reduce tryptophan feedback inhibition increases tryptophan productivity.

In some embodiments, microorganisms that have ability to grow in DHA in a medium comprising DHA as a source of carbon is subjected to further genetic modifications to include one or more nucleic acid molecules comprising one or more nucleotide sequences encoding muconic acid biosynthetic pathway enzymes. Muconic acid, an aromatic amino acid pathway product, is made by a series of enzymatic biochemical reactions starting from 3-dehydroshikimate. Microorganisms that already has an ability to produce high amounts of aromatic amino acids by one or more genetic modifications are a preferred host to produce muconic acid. On the other hand, muconic acid biosynthetic pathway can be directly engineered in to the host cell that previously lack the ability to produce aromatic acid but can be engineered to selectively produce muconic acid. Furthermore, one or more of the genes selected from a group of muconic acid biosynthetic pathway such as TktA, TalB, AroG, AroF, AroH, AroB, AroD, AroZ, AroY and CatA is overexpressed for the enhanced production of muconic acid. Overexpressing pyruvate kinase to increase the production of phosphoenol pyruvate, attenuation or deletion of phosphoenolpyruvate carboxylase and pyruvate carboxylase increases the availability of phosphoenol pyruvate for muconic acid synthesis. In addition, attenuation or deletion of gene AroE encoding shikimate dehydrogenase enhances the production of muconic acid. Furthermore, improving muconic acid transport, reducing muconic acid uptake from media and blocking feedback inhibition increases muconic acid productivity.

Genetically modified microorganisms capable of producing aromatic amino acid pathway products using conventional sugars such as glucose, sucrose or glycerol are already known in the art. U.S. Pat. No. 5,030,567 provides optimal temperature for the production of phenylalanine using genetically engineered microorganism. U.S. Pat. No. 7,700,328 provides genetically engineered microorganism that has strong expression of TyrA gene and inactivation of PheA gene for the high levels of tyrosine production. U.S. Pat. No. 7,709,242 provides genetically engineered microorganism that has mutant prephenate dehydrogenase desensitized for feedback inhibition by tyrosine. U.S. Pat. No. 8,569,066 provides genetically engineered tryptophan producing microorganism wherein tryptophan auxotrophy is released, phenylalanine biosynthesis is blocked and tryptophan productivity is enhanced by reinforcing gene involved tryptophan biosynthesis. U.S. Pat. Nos. 5,484,716 and 5,595,906 provides genetically engineered aromatic amino acid producing microorganism that has decreased or lacked phosphoenolpyruvate carboxylase activity. U.S. Pat. No. 4,742,007 provides genetically engineered tryptophan producing microorganism that has resistant to glyphosate and paraquat. U.S. Pat. No. 4,271,267 provides genetically engineered microorganism producing tryptophan using ethanol as a main source of carbon. U.S. Pat. No. 4,707,449 provides production of single-cell protein from yeast Pichia Pastoris with high tryptophan content. U.S. Pat. No. 3,296,090 describes microorganism and process for production of tryptophan. U.S. Pat. No. 4,588,687 provides genetically engineered tryptophan producing microorganism that is resistant to tryptophan antagonists. U.S. Pat. No. 4,885,245 provides genetically engineered tryptophan producing microorganism that has overexpression of tryptophan synthase enzyme. U.S. Pat. No. 5,017,481 provides genetically engineered microorganism to produce aromatic amino acid that has overexpression of shikimate kinase. U.S. Pat. No. 5,304,475 provides genetically engineered microorganism to produce high yields phenylalanine that contains overexpression of PheA and AroF genes. U.S. Pat. No. 5,776,736 provides efficient production of aromatic amino acid pathway products by the enhanced expression of a selected group of structural genes of the common aromatic amino acid pathway. U.S. Pat. No. 5,624,828 provides genetically engineered serine auxotrophic microorganisms to produce tryptophan and threonine. U.S. Pat. No. 5,034,318 provides genetically engineered tryptophan producing microorganism that contains overexpression of an operably linked anthranilic acid phosphoribosyl transferase gene. U.S. Pat. Nos. 5,563,052, 5,407,824 and 5,447,857 provide genetically engineered tryptophan producing microorganism that composes vector DNA and DNA fragments bearing all of genetic information relating to the synthesis of anthranilate synthase, anthranilate phosphoribosyl transferase, N-phosphoribosyl anthranilate isomerase, indole-3-glycerol phosphate synthase, and tryptophan synthase. U.S. Pat. No. 4,968,609 provides genetically engineered microorganism producing aromatic amino acids that contains overexpression of 3-deoxy-D-arabino-heptulonic acid-7-phophate synthase. U.S. Pat. No. 3,594,279 provides for producing tryptophan from genetically engineered microorganisms using inexpensive carbon sources. U.S. Pat. No. 3,385,762 provides genetically engineered microorganism for the production of tryptophan from anthranilic acid or indole. U.S. Pat. No. 6,180,373 provides genetically engineered tryptophan producing microorganisms that is both tryptophan feedback resistant and serine feedback resistant. U.S. Pat. Nos. 4,363,875 and 3,801,457 provide production of tryptophan from anthranilic acid using genetically engineered microorganism. U.S. Pat. No. 3,700,558 provides genetically engineered microorganism to produce tryptophan. U.S. Pat. Nos. 4,560,652 and 4,601,982 provide genetically engineered tryptophan producing microorganism that is resistant to azaserine and a tryptophane antagonist. U.S. Pat. No. 3,700,559 provides genetically engineered tryptophan producing microorganism that is resistant to 5-methyl-DL-tryptophan. U.S. Pat. Nos. 8,067,210 and 4,618,580 provides genetically engineered microorganism that is resistant to sulfaguanidine and capable of producing tryptophan. U.S. Pat. No. 5,275,940 provides genetically engineered tryptophan producing microorganism that is resistant to aminoquinoline and phenothiazine derivatives. U.S. Pat. No. 3,849,251 provides genetically engineered tryptophan producing microorganism that is resistant to the inhibitory actions of the analogues of tryptophan, phenylalanine and tyrosine. U.S. Pat. No. 3,709,785 provides genetically engineered tyrosine producing microorganism containing m-fluorophenylalanine to suppress the production of other aromatic amino acid products. U.S. Pat. No. 9,267,118 provides genetically engineered microorganism that has been modified by the heterologous expression of bacterial tyrosinase for the production of tyrosine. U.S. Pat. No. 7,531,345 provides genetically engineered microorganism that has been modified by the introduction of chorismate mutase/prephenate dehydrogenase for the production of tyrosine. U.S. Pat. No. 8,735,132 provides methods and processes to identify genetically engineered microorganism that shows enhanced ability to produce tyrosine. U.S. Pat. No. 5,272,073 provides genetically engineered microorganism for the production of catechol by inducing divergent pathway comprising DHA dehydratase, protocatechuate decarboxylase. U.S. Pat. No. 7,638,312 provides genetically engineered microorganism that contains overexpression of AroF, AroG, TrpR and TryR genes for the increased production of tryptophan. U.S. Pat. No. 5,906,925 provides genetically engineered microorganism for the overproduction of 3-deoxy-D-arabino-heptulosonate-7-phosphate where the overexpression of phosphoenol pyruvate synthase increases the product production. U.S. Pat. No. 5,939,295 provides genetically engineered microorganism to produce tryptophan that contains overexpression of AroG and SerA genes. U.S. Pat. No. 5,756,345 provides genetically engineered microorganism to produce tryptophan where the transport system encoded by AroP, Mtr, TnaB are being transformed with a plasmid encoding anthranilate synthase freed from tryptophan feedback inhibition. U.S. Pat. No. 5,605,818 provides genetically engineered microorganism that overexpresses transketolase activity for the increased production of aromatic aminoacids. U.S. Pat. No. 4,371,614 provides genetically engineered microorganism deficient in the enzyme tryptophanase responsible for degradation of tryptophan to indole for the increased production of tryptophan. U.S. Pat. No. 5,168,056 provides genetically engineered microorganism for the production of aromatic amino acids that encodes transketolase utilized to enhance diversion of carbon sources into the common aromatic pathway. U.S. Pat. No. 4,874,698 provides genetically engineered microorganism that contains anthranilic acid synthase gene for the increased accumulation of tryptophan. U.S. Pat. No. 3,787,287 provides genetically engineered Corynebacterium glutamicum to produce tyrosine.

U.S. Pat. No. 4,783,403 provides genetically engineered microorganism to produce phenylalanine. U.S. Pat. No. 4,591,562 provides genetically engineered microorganism for the fermentative production of phenylalanine that contains resistance to phenylalanine antagonist. U.S. Pat. No. 4,403,033 provides genetically engineered microorganism that have been given sensitivity to decoyinine showed improved productivity to phenylalanine. U.S. Pat. No. 4,407,952 provides genetically engineered phenylalanine producing microorganism that has been expressed with phenylalanine biosynthetic pathway. U.S. Pat. No. 4,908,312 provides genetically engineered microorganism that has been transformed with chorismate mutase or prephenate dehydratase for the enhanced production of phenylalanine. U.S. Pat. No. 3,660,235 provides genetically engineered phenylalanine producing microorganisms that are tolerant to higher amounts of phenylalanine analogues. U.S. Pat. Nos. 3,759,790 and 3,917,511 provides genetically engineered phenylalanine producing microorganisms that show resistance to aromatic amino acids. U.S. Pat. No. 3,909,353 provides tyrosine requiring mutant strains that exhibit high growth in phenylalanine containing media. Chinese Patent No. CN103074292 describes a recombinant microorganism that contains overexpression of prephenate dehydratase, transketolase, phosphoenolpyruviac acid synthase for the increased phenylalanine production. Chinese Patent No. CN102399835 describes the overexpression of AroG in recombinant microorganism for the enhanced production of phenylalanine. Chinese Patent No. CN103484508 describes the method of enhancing the citric acid absorption capability to improve output of phenylalanine. Chinese Patent No. CN102604882 discloses recombinant microorganism capable of producing phenylalanine that contains overexpression of AroD, AroE, PheA and TalA genes. European Patent No. EP0263515 discloses recombinant microorganism to produce tyrosine that contains enzyme chorismate mutase and 3-deoxy-D-arabino-hepturosonate-7-phophate synthase. European Patend No. EP0138526 discloses phenylalanine auxotrophic mutant strains for the enhanced production of phenylalanine. European Patent No. EP2147972 discloses recombinant microorganisms to produce tryptophan containing 3-deoxy-D-arabino-hepturosonate-7-phophate synthase. European Patent No. EP0077196 discloses genetically modified microorganism to produce aromatic amino acids that expresses 3-deoxy-D-arabino-hepturosonate-7-phophate synthase and resistant to feedback inhibition by aromatic amino acids. European Patent No. EP0745671 discloses genetically modified microorganism to produce aromatic amino acids where the AroF and AroG genes are overexpressed. U.S. Pat. No. 6,613,552 provides genetically engineered microorganism wherein the expression of AroB, AroE and AroF genes were performed for the enhanced production of shikimic acid. U.S. Pat. No. 6,436,664 provides genetically engineered microorganism which is deficient in shikimate kinase activity. U.S. Pat. No. 8,372,621 provides genetically engineered microorganism containing KDPGal aldolase, 3-dehydroquinone synthase and DHQ dehydratase enzymes to produce shikimic acid. U.S. Pat. No. 8,809,583 provides genetically engineered microorganism for the production of muconic acid where 3-dehydroshikimate dehydratase, protocatechuate decarboxylase and catechol 1,2-dioxygenase enzymes were overexpressed. U.S. Pat. No. 5,616,496 provides genetically engineered microorganism that expresses heterologous genes encoding the enzymes 3-dehydroshikimate dehydratase, protocatechuate decarboxylase and catechol 1,2-dioxygenase for the production of muconic acid. U.S. Patent Application Publication No. 2015/0044755 reports muconic acid production in a genetically modified microorganism using renewable carbon resources.

In one method to enhance DHA uptake and utilization, the genetically modified host cells already known to produce aromatic amino acid pathway products, as described in patents above, are subjected to further genetic engineering by means of expressing plasmids that code ATP and PEP dependent kinases that could phosphorylate DHA and facilitate its entry into glycolytic cycle. In another method to enhance DHA uptake and utilization, genetically modified host cells already known to produce aromatic amino acid pathway products, as described in patents above, is subjected to chemical mutagenesis and the strains with the ability to grow and produce desired aromatic amino acid pathway products with high enough titer and yield in a growth medium comprising DHA as a source of carbon will be selected and subjected to whole genome sequencing. to identify the specific mutation associated with the ability to grow and produce aromatic amino acid pathway products in a medium comprising DHA. Such a mutation will be introduced to the genetically modified host cells already known to produce aromatic amino acid pathway products for the purpose of conferring the ability to use DHA as a source of organic carbon. In yet another method to enhance DHA uptake and utilization, genetically modified host cells already known to produce aromatic amino acid pathway products, as described in patents above, is exposed to growth medium with increasing concentration of DHA and subjected to metabolic evolution. Strains that have gained ability to grow in the medium containing DHA as a major or sole source of carbon and still retain the ability to produce aromatic amino acid pathway products is selected and subjected to whole genome sequencing to identify the specific mutation associated with the ability to grow and produce aromatic amino acid pathway products in a medium comprising DHA. Such a mutation will be introduced to the genetically modified host cells already known to produce aromatic amino acid pathway products, as described in patents above, for the purpose of conferring the ability to use DHA as a source of organic carbon.

In another embodiment, the present invention provides methods of producing branched chain amino acids such as L-leucine, L-isoleucine and L-valine. In one aspect, genetically modified microorganisms producing branched chain amino acids are subjected to further genetic modifications to confer the ability to use DHA as a source of carbon in a medium containing dihydroxyacetone as a carbon source. In another aspect of this invention, microorganisms that have ability to grow in a medium comprising DHA as a source of carbon is subjected to further genetic modifications to produce branched chain amino acids. Such a genetic modification would involve overexpressing one or more components of the valine, leucine, isoleucine derivative biosynthetic pathway, including but not limited to acetolactate synthase, keto-acid reductoisomerase, dihydroxy acid dehydratase, branched-chain amino acid aminotransferase, threonine dehydratase, isopropylmalate synthase, isopropylmalate isomerase, isopropylmalate dehydrogenase and tyrosine transaminase.

In one aspect of this embodiments, microorganisms that have ability to grow in DHA in a medium comprising DHA as a source of carbon is subjected to further genetic modifications to include one or more nucleic acid molecules comprising one or more nucleotide sequence encoding branched chain amino acid pathway enzymes. Valine, a branched chain amino acid, is made from pyruvic acid by a series of enzyme catalyzed biochemical reactions. Microorganisms that already has an ability to produce high amounts of pyruvate by one or more genetic modifications are a preferred host to produce valine. On the other hand, valine biosynthetic pathway can be directly engineered in the host cell that previously lack the ability to produce pyruvic acid but can be engineered to selectively produce valine. Furthermore, one or more of the genes selected from a group of valine biosynthetic pathway such as IlvB, IlvC, IlvD and IlvE is overexpressed for the enhanced production of valine. In addition, attenuation or deletion of pyruvate carboxylase, pyruvate kinase, phosphoenolpyruvate carboxylase, lactate dehydrogenase enhances the pyruvate utilization in valine synthesis. Moreover, attenuation or deletion of IlvA gene encoding threonine dehydratase, LeuA gene encoding isopropylmalate synthase reduces the formation of isoleucine and leucine. Blocking valine utilization pathway by reducing the synthesis of pantothenate by means of deletion of PanB, PanC, PanD and PanE genes improves valine accumulation. Furthermore, improving valine transport, reducing valine uptake from media and reducing feedback inhibition increases valine productivity.

In some embodiments, microorganisms that have ability to grow in DHA in a medium comprising DHA as a source of carbon is subjected to further genetic modifications to include one or more nucleic acid molecules comprising one or more nucleotide sequence encoding branched chain amino acid pathway enzymes. Leucine, a branched chain amino acid, is made from pyruvic acid by a series of enzyme catalyzed biochemical reactions. Microorganisms that already has an ability to produce high amounts of pyruvate by one or more genetic modifications are a preferred host to produce leucine. On the other hand, leucine biosynthetic pathway can be directly engineered in the host cell that previously lack the ability to produce pyruvic acid but can be engineered to selectively produce leucine. Furthermore, one or more of the genes selected from a group of leucine biosynthetic pathway such as IlvB, IlvC, IlvD, LeuA, LeuC, LeuD, LeuB, TyrB is overexpressed for the enhanced production of leucine. In addition, attenuation or deletion of pyruvate carboxylase, pyruvate kinase, phosphoenolpyruvate carboxylase, lactate dehydrogenase enhances the pyruvate utilization in leucine synthesis. Moreover, attenuation or deletion of IlvA gene encoding threonine dehydratase, IlvE gene encoding branched-chain amino acid aminotrasferase reduces the formation of isoleucine and valine. Blocking leucine utilization pathway by reducing the production of isovaleric acid by means of deletion of 2-oxoisocaproate dehydrogenase improves leucine accumulation. Furthermore, improving leucine transport, reducing leucine uptake from media and reducing feedback inhibition increases leucine productivity.

In some embodiments, microorganisms that have ability to grow in DHA in a medium comprising DHA as a source of carbon is subjected to further genetic modifications to include one or more nucleic acid molecule comprising one or more nucleotide sequence encoding branched chain amino acid pathway enzymes. Isoleucine, a branched chain amino acid, is made from threonine by a series of enzyme catalyzed biochemical reactions. Microorganisms that already has an ability to produce high amounts of threonine by one or more genetic modifications are a preferred host to produce isoleucine. On the other hand, isoleucine biosynthetic pathway can be directly engineered in to the host cell that previously lack the ability to produce threonine but can be engineered to selectively produce isoleucine. Furthermore, one or more of the genes selected from a group of isoleucine biosynthetic pathway such as LysC, Asd, Horn, ThrB, ThrC, IlvA, IlvB, IlvC, IlvD and IlvE is overexpressed for the enhanced production of isoleucine. Moreover, reducing pyruvate available for valine synthesis by overexpression of pyruvate carboxylase, phosphoenolpyruvate carboxylase improves isoleucine formation and reduces valine formation. Attenuation or deletion of LeuA gene encoding isopropylmalate synthase reduces the formation of leucine. In another method to produce isoleucine, pyruvate is converted to isoleucine by a series of enzyme catalyzed biochemical reactions. In addition to the enzymes catalyze production of pyruvate from DHA, one or more of the enzymes selected from a group of pyruvate to isoleucine biosynthetic pathway such as citramalate synthase, citroconate hydrolase, methylmalate dehydratase, methylmalate dehydrogenase, acetolactate synthase (IlvB), keto-acid reductoisomerase (IlvC), dihydroxyacid dehydratase (IlvD) and branchedchain amino acid amino transferase (IlvE) are overexpressed for the enhanced production of isoleucine from pyruvate. In addition, attenuation or deletion of one or more of methylglyoxal synthase, phosphoenolpyruvate carboxylate, pyruvate carboxylate, acetate kinase, aldehyde dehydrogenase, phosphotransacylase, pyruvate oxidase, pyruvate-formate lyase, succinate dehydrogenase, malate dehydrogenase, lactate dehydrogenase, acetyl-CoA carboxylase, hydroxymethyl-CoA glutaryl synthase, citrate synthase and improves pyruvate utilization in isoleucine synthesis. Blocking isoleucine utilization pathway by reducing the production of 2-methylnutyric acid by means of deletion of 2-oxo-3-methylvalerate dehydrogenase improves isoleucine accumulation. Furthermore, improving leucine transport, reducing leucine uptake from media and reducing feedback inhibition increases isoleucine productivity.

Genetically modified microorganisms capable of producing branched chain amino acids using conventional sugars such as glucose, sucrose or glycerol are already known in the art. U.S. Pat. No. 6,214,591 provides genetically engineered microorganism capable of producing valine and leucine wherein it requires lipoic acid for growth and/or it is deficient in H⁺-ATPase. U.S. Pat. No. 3,970,519 provides genetically engineered microorganism which resist feedback inhibition by leucine or its analogs for the enhanced production of leucine. U.S. Pat. No. 5,534,421 provides genetically engineered microorganism for the improved production of isoleucine that has no negative feedback inhibition of isoleucine production. U.S. Pat. No. 6,124,121 provides genetically engineered microorganism that has an ability to produce leucine and is resistant to leucine in the fermentation medium. U.S. Pat. No. 7,179,623 provides genetically engineered microorganism that contains genes encoding metabolic pathways for sucrose utilization to produce isoleucine, valine and lysine. U.S. Pat. No. 7,220,572 provides genetically engineered microorganism to produce valine, isoleucine and homoleucine that has an inactivation of IlvE gene for the reduced production of leucine. U.S. Pat. No. 6,737,255 provides genetically engineered microorganism containing acetohydroxy acid synthase isozyme III to produce acetolactate and acetohydroxy butyrate for the production of valine. U.S. Pat. No. 5,658,766 provides aminoacyl-tRNA synthetase mutations for preparing genetically engineered organisms possessing enhanced capability of producing isoleucine and valine. U.S. Pat. No. 4,946,781 provides genetically engineered microorganism having operationally inserted homoserine kinase for the production of isoleucine or threonine in large quantities. U.S. Pat. No. 6,107,063 provides genetically engineered microorganism for the increase production of isoleucine that has decreased feedback inhibition from isoleucine and increased expression of threonine dehydratase. U.S. Pat. No. 4,391,907 provides genetically engineered microorganism for the enhanced production of valine that has reduced feedback resistance from valine and valine analogues. U.S. Pat. No. 4,601,983 provides genetically engineered microorganism having homoserine dehydrogenase activity for the production of threonine and isoleucine. U.S. Pat. No. 7,632,663 provides genetically engineered microorganism that has reduced pantothenate production and increased expression of IlvD, IlvB, IlvN, IlvC gene for the enhanced production of valine. U.S. Pat. No. 5,362,637 provides genetically engineered microorganism to produce isoleucine that has resistant to isoleucine analogue such as thiaisoleucine or isoleucine hydroxamate. U.S. Pat. No. 5,629,180 provides genetically engineered microorganism that has resistance to 2-ketobutyric acid and has an ability to produce valine, leucine and isoleucine. U.S. Pat. No. 8,158,390 provides genetically engineered microorganism that has an enhanced transaminase C activity to produce valine higher than that of non-modified organism. U.S. Pat. No. 4,329,427 provides analogue resistant genetically engineered microorganism to produce isoleucine from threonine. U.S. Pat. No. 5,629,180 provides genetically engineered microorganism. U.S. Pat. Nos. 3,231,478, 3,058,888, 3,262,861, 3,086,918, 3,502,544 and 3,041,247 provides genetically engineered microorganism for the production of isoleucine by fermentative methods and processes in a medium containing carbon source and nitrogen source. U.S. Pat. No. 5,695,972 provides production of isoleucine in a medium containing glucose as a carbon source and homoserine as the single nitrogen source. U.S. Pat. No. 3,671,396 provides genetically engineered microorganism to produce isoleucine using hydrocarbon substances as a source of carbon. U.S. Pat. No. 5,474,918 provides genetically engineered microorganism that has resistance to purine analogues and has an ability to produce threonine and isoleucine. U.S. Pat. No. 3,767,529 provides genetically engineered microorganism to produce isoleucine directly from glucose and acetic acid in the culture medium. U.S. Pat. No. 6,403,342 provides genetically engineered microorganism that is desensitized in feedback inhibition of leucine and has an overexpression of isopropylmalate synthase enzyme for the production of leucine. U.S. Pat. No. 5,744,331 provides genetically engineered microorganism having resistance to leucine analogue and have an ability to produce leucine in the fermentation medium. U.S. Pat. Nos. 4,421,853 and 4,421,854 provides genetically engineered microorganism and fermentation methods, processes and downstream isolation processes to produce leucine. U.S. Pat. No. 4,237,228 provides genetically engineered microorganism for the production of isoleucine in a nutrient medium containing nitrogen, carbon, mineral salts and vitamins under aerobic conditions. U.S. Pat. No. 3,865,690 provides genetically engineered microorganism that are resistant to leucine antagonists and produce leucine in the culture medium. U.S. Pat. No. 3,668,073 provides genetically engineered microorganism containing promoters selected from the group consisting of isoleucine, methionine, phenylalanine, valine in a culture medium containing sugar source, nitrogen source to produce leucine. U.S. Pat. No. 5,763,231 provides genetically engineered microorganism developed by incubating microorganism in a culture medium and reacting the resulting cells with sugars and acetic acid to produce leucine. U.S. Pat. No. 5,118,619 provides genetically engineered microorganism, which utilize D-lactate for the production of isoleucine from D, L-2-hydroxybutyrate. U.S. Pat. No. 4,656,135 provides genetically engineered microorganism capable of producing isoleucine that has methyllysine and 2-ketomalonic acid resistance. U.S. Pat. No. 4,442,208 provides genetically engineered microorganism obtained by isolating a transformed strain resistant to 2-amino-3-hydroxy valeric acid for the production of isoleucine. U.S. Pat. No. 5,521,074 provides genetically engineered microorganism that exhibits resistance to valine in the medium, sensitivity to pyruvic acid analog in a medium containing glucose as a sole carbon source for the production of valine. U.S. Pat. Nos. 7,635,579 and 7,323,321 provides genetically engineered microorganism obtained by mutagenizing a parent strain to be auxotrophic or bradytrophic for branched chain amino acid synthesis for the production of amino acids. U.S. Pat. No. 8,465,962 provides genetically engineered microorganism that has resistance to valine and derivatives for the enhanced production of valine. U.S. Pat. No. 3,893,888 provides genetically engineered microorganism having resistance to 2-thiazolalanine to produce valine in the culture medium. U.S. Pat. No. 5,188,948 provides genetically engineered microorganism having resistance to polyketide to produce valine and then recovering accumulated valine in the culture medium. U.S. Pat. No. 7,202,060 provides genetically engineered microorganism that has enhanced alanine-valine transaminase activity for the production of amino acids. U.S. Patent Application Publication No. 2010/0151449 reports genetically engineered microorganism that has inhibited alanine transaminase activity leading to the enhanced production of valine, leucine and isoleucine. U.S. Patent Application Publication No. 2004/0091980 reports method of producing leucine using genetically engineered microorganism that has inactivated IlvE gene coding for the branched chain amino acid transaminase and increased activity of aromatic amino acid transaminase encoded by TyrB gene. European Patent No. EP 0204326 discloses process for producing threonine and isoleucine using genetically engineered microorganism containing homoserine dehydrogenase and homoserine kinase. European Patent No. EP 2060636 discloses methods for manufacturing amino acids using genetically engineered microorganism containing overexpressed glgS gene. European Patent No. EP 0356739 discloses production of valine, leucine and isoleucine using a genetically engineered microorganism comprising the gene coding for actohydroxyacid synthase. European Patent No. EP 0233581 discloses production of isoleucine and threonine using recombinant DNA bearing genetic information of homoserine dehydrogenase, homoserine kinase, threonine synthase. European Patent No. EP 0436886 discloses production of isoleucine using recombinant DNA bearing genetic information of threonine dehydratase and acetohydroxy acid synthase. European Patent No. EP 28110828 discloses production of leucine, valine and isoleucine by a recombinant microorganism containing overexpression of genes IlvB, IlvN. European Patent No. EP 0287123 discloses enhanced production of valine by a recombinant microorganism that are resistant to polyketides. Chinese Patent No. CN1100146 discloses recombinant microorganisms that are resistant to leucine analogue and has the ability to produce leucine. Chinese Pat. No. CN101962662 discloses method of leucine production in which addition of citric acid reduces the formation of by-products such as valine and alanine. International PCT Application. Publication, No. WO2014/200126 provides genetically engineered microorganism that are resistant to derivatives of isoleucine and threonine to produce isoleucine.

In one method to enhance DHA uptake and utilization, the genetically modified host cells already known to produce branched chain amino acids, as described in patents above, is subjected to further genetic engineering by means of expressing plasmids that code ATP and PEP dependent kinases that could phosphorylate DHA and facilitate its entry into glycolytic cycle. In another method to enhance DHA uptake and utilization, genetically modified host cells already known to produce branched chain amino acids, as described in patents above, is subjected to chemical mutagenesis and the strains with the ability to grow and produce desired branched chain amino acid with high enough titer and yield in a growth medium comprising DHA as a source of carbon is selected and subjected to whole genome sequencing to identify the specific mutation associated with the ability to grow and produce branched chain amino acid in a medium comprising DHA. Such a mutation will be introduced to the genetically modified host cells already known to produce branched chain amino acids to confer the ability to use DHA as a source of organic carbon. In yet another method to enhance DHA uptake and utilization, genetically modified host cells already known to produce branched chain amino acids, as described in patents above, is exposed to growth medium with increasing concentration of DHA and subjected to metabolic evolution. Strains that have gained ability to grow in the medium containing DHA as a major or sole source of carbon and still retain the ability to produce branched chain amino acids is selected and subjected to whole genome sequencing to identify specific mutation associated with the ability to grow and produce branched chain amino acids in a medium comprising DHA. Such a mutation will be introduced to the genetically modified host cells already known to produce branched chain amino acids, as described in patents above, for the purpose of conferring the ability to use DHA as a source of organic carbon.

In yet another embodiment, the present invention provides methods of producing vitamins such as vitamin C, biotin, Vitamin D2, vitamin D3, vitamin K 1, vitamin K2, vitamin B2, vitamin B12, coenzyme Q10, vitamin A and Vitamin E through biological fermentation using DHA as a source of carbon. In one aspect of the present invention, genetically modified microorganisms producing vitamins are subjected to further genetic modifications to confer the ability to use DHA as a source of carbon. In another aspect of this invention, microorganisms that have ability to grow in DHA in a medium comprising DHA as a source of carbon is subjected to further genetic modifications to produce vitamins. Such a genetic modification would involve overexpressing one or more components of the glucose to vitamin C biosynthetic pathway, fructose to vitamin C biosynthetic pathway, biotin biosynthetic pathway, vitamin D2 biosynthetic pathway, vitamin D3 biosynthetic pathway, vitamin B2 biosynthetic pathway, vitamin B12 biosynthetic pathway, vitamin K1 biosynthetic pathway, vitamin K2 biosynthetic pathway, vitamin E biosynthetic pathway and coenzyme Q10 biosynthetic pathway. The list of genetic manipulations required to enhance the vitamin biosynthesis within the microbial cell include: enhancing the activities of one or more of the enzymes involved in the glucose to vitamin C biosynthetic pathway such as UTP-glucose-1-phosphate uridylyl transferase, UDP-glucose dehydrogenase, Glucuronate-1-phosphate uridylyl transferase, D-glucurono kinase, urono lactonase, glucurono lactone reductase, gulonolactone oxidase, aldono lactonase and D-glucuronate reductase; enhancing the activities of one or more enzymes involved in the fructose to vitamin C biosynthetic pathway such as mannose-6P-isomerase, phosphomannomutase, mannose-1P-guanylyl transferase, GDP-mannose-3,5-epimerase, hydrolase, sugar phosphatase, L-galactose dehydrogenase and galactono-1,4-lactone dehydrogenase; enhancing the activities of one or more enzymes involved in the biotin biosynthetic pathway such as enzymes facilitating the conversion of pimelic acid to pimelyl-CoA, enzymes pimeloyl-CoA synthase, 7-keto-8-amino nonanoate synthase, 7,8-diamino pelargonic acid amino transferase, dethiobiotin synthetase and biotin synthetase; enhancing the activities of one or more enzymes involved in the in the vitamin D biosynthetic pathway such as squalene monooxygenase, lanosterol synthase, sterol-14-demethylase, methyl sterol monooxygenase, sterol-4α-carboxylase-3-dehydrogenase, 3-keto steroid reductase, zymosterol demethylase, Δ24-sterol reductase, cholestenolΔisomerase, Δ7sterol-5-desaturase, 7-dehydro cholesterol reductase, stero124C methyl transferase, C8 sterol isomerase, Δ7-sterol-5-desaturase, sterol-22-desaturase and Δ24-sterol reductase; enhancing the activities of one or more of the enzymes involved in the vitamin E biosynthetic pathway such as geranylgeranyl reductase, tyrosine amino transferase, 4-hydroxy phenyl pyruvate dioxygenase, homogentisate phytyl transferase, tocopherol cyclase, tocopherol-O-methyl transferase, MPBQ/MSBQ methyltransferase and tocopherol cyclase; enhancing the activities of one or more enzymes involved in the vitamin K biosynthetic pathway such as isochorismate synthase, 2-succinyl-5-enol-pyruvyl-6-hydroxy-3-cyclohexene-1-carboxylate synthase, 2-succinyl-6-hydroxy-2,4-cyclohexadiene-1-carboxylate synthase, O-succinylbenzoate synthase, O-succinylbenzoateCoA ligase, naphthoate synthase, 1,4-dihydroxy-2-naphthoyl-CoA hydrolase, 1,4-dihydroxy-2-napthoate octaprenyl transferase and 2-demethyl menaquinone methyl transferase; enhancing the activities of one or more enzymes involved in the coenzyme Q10 biosynthetic pathway such as chrorismate pyruvate lyase, 4-hydroxybenzoate decaprenyl transferase, 3-decaprenyl-4-hydroxybenzoate decarboxylase, octaprenylphenol hydroxylase, 6-hydroxyphenyl-2-decaprenyl methylase, 6-methoxyphenyl-2-decaprenyl hydroxylase, 6-methoxy-2-decaprenyl 1,4-benzoquinol methylase, 6-methoxy-2-decaprenyl-3-methyl-1,4-benzoquinol hydroxylase, 6-hydroxyphenyl-2-decaprenyl methylase and decaprenyl dihydroxybenzoate methyl transferase; enhancing the activities of one or more enzymes in the vitamin B2 biosynthetic pathway such as riboflavin synthase, 6,7-dimethyl-8-ribityllumazine synthase, 3,4-dihydroxy-2-butanone-4-phosphate synthase, 5-amino-6-(5-ribosylamino) uracil reductase, 5-amino-6-(5-ribosylamino) uracil phosphatase and diaminohydroxyphosphoribosylaminopyrimidine; enhancing the activities of one or more enzymes in the vitamin B12 biosynthetic pathway such as uroporphyrinogen III methyl transferase, precorrin-2-C20 methyltransferase, enzymes catalyzing the conversion of precorrin 3 to cobyrinate diamide and enzymes catalyzing the conversion of cobyrinate diamide to vitamin B12.

In one method of the present invention, microorganisms that have ability to grow in DHA in a medium comprising DHA as a source of carbon is subjected to further genetic modifications to include one or more nucleic acid comprising a nucleotide sequence encoding vitamin C biosynthetic pathway enzymes. Vitamin C is made from glucose by a series of enzyme catalyzed biochemical reactions. In another method, vitamin C is made from fructose by a series of enzyme catalyzed biochemical reactions. Microorganisms that already has an ability to utilize glucose or fructose either natively or by one or more genetic modifications are a preferred host to produce vitamin C. On the other hand, vitamin C biosynthetic pathway can be directly engineered in to the host cell that previously lack the ability to utilize glucose or fructose but can be engineered to selectively produce vitamin C. Furthermore, in addition to the enzymes that catalyze the formation of glucose-1P from DHA, one or more of the enzymes selected from a group of glucose to vitamin C biosynthetic pathway such as UTP-glucose-1-phosphate uridylyl transferase, UDP-glucose dehydrogenase, Glucuronate-1-phosphate uridylyl transferase, D-glucurono kinase, urono lactonase, glucurono lactone reductase, gulonolactone oxidase, aldono lactonase and D-glucuronate reductase are overexpressed for the enhanced production of vitamin C from glucose. In addition, attenuation or deletion of one or more of methylglyoxal synthase (MgsA), glycerol-3P-dehydrogenase, transketolase, transaldolase, glyceraldehyde-P-dehydrogenase, glucose-6P-dehydrogenase and glutamine fructose-6P-transaminase improves DHA utilization in vitamin C synthesis. When fructose is used to produce vitamin C, in addition to the enzymes catalyze production of fructose-6P from DHA, one or more of the enzymes selected from a group of fructose to vitamin C biosynthetic pathway such as mannose-6P-isomerase, phosphomannomutase, mannose-1P-guanylyl transferase, GDP-mannose-3,5-epimerase, hydrolase, sugar phosphatase, L-galactose dehydrogenase, galactono-1,4-lactone dehydrogenase are overexpressed for the enhanced production of vitamin C from fructose. In addition, attenuation or deletion of one or more of methylglyoxal synthase (MgsA), glycerol-3P-dehydrogenase, transketolase, transaldolase, glyceraldehyde-P-dehydrogenase, hexulose-6P-isomerase, glucosamine-P-isomerase and glutamine fructose-6P-transaminase improves DHA utilization in vitamin C synthesis. Blocking vitamin C utilization pathway by attenuation or deletion of enzymes ascorbate oxidase and glutathione dehydrogenase responsible for the oxidation of vitamin C and ascorbate-2,3-dioxygenase responsible for the degradation of vitamin C to oxalate improves vitamin C accumulation. Furthermore, improving vitamin C transport, reducing vitamin C uptake from media and reducing feedback inhibition increase vitamin C productivity.

In another aspect of the present invention, microorganisms that have ability to grow in a medium comprising DHA as a source of carbon is subjected to further genetic modifications to include one or more nucleic acid comprising a nucleotide sequence encoding biotin biosynthetic pathway enzymes. Biotin is made from pimelic acid by a series of enzyme catalyzed biochemical reactions. Microorganisms that already has an ability to produce high amounts of pimelic acid either natively or by one or more genetic modifications are a preferred host to produce biotin. On the other hand, biotin biosynthetic pathway can be directly engineered in the host cell that previously lack the ability to produce pimelic acid but can be engineered to selectively produce biotin. Furthermore, in addition to the enzymes that catalyze the formation of pimelic acid from DHA, one or more of the enzymes selected from a group of biotin biosynthetic pathway such as genes BioC and BioH encoding for enzymes facilitating the conversion of pimelic acid to pimelyl-CoA, enzymes pimeloyl-CoA synthase, 7-keto-8-amino nonanoate synthase (BioF), 7,8-diamino pelargonic acid amino transferase (BioA), dethiobiotin synthetase (BioD) and biotin synthetase (BioB) are overexpressed for the enhanced production of biotin from pimelic acid. In addition, attenuation or deletion one or more of enzymes such as methylglyoxal synthase (MgsA), phosphoenolpyruvate carboxylate, pyruvate carboxylate, acetate kinase (AckA), aldehyde dehydrogenase (AldA), pyruvate oxidase (PoxB), succinate dehydrogenase (FrdBC), malate dehydrogenase, 2-ketoglutarate dehydrogenase, lactate dehydrogenase (LdhA), p-ketothiolase, hydroxymethyl-CoA glutaryl synthase and citrate synthase improves acetyl-CoA utilization in pimelic acid synthesis. Blocking biotin utilization pathway by attenuation or deletion of enzymes biotin sulfoxide reductase and biotinyl-CoA synthetase responsible for the degradation of biotin to bisnorbiotin via biotinyl-CoA improves biotin accumulation. Furthermore, improving biotin transport, reducing biotin uptake from media and reducing feedback inhibition increases biotin productivity.

In yet another aspect of the present invention, microorganisms that have ability to grow in a medium comprising DHA as a source of carbon is subjected to further genetic modifications to include one or more nucleic acid comprising a nucleotide sequence of genes encoding proteins functional in the vitamin D biosynthetic pathway. Vitamin D includes but not limited to vitamin D2 and vitamin D3. Vitamin D2, is produced from its provitamin precursor ergosterol and vitamin D3 is produced from its provitamin precursor 7-dehydrocholestrol. Vitamin D biosynthetic pathway includes but not limited to ergosterol biosynthetic pathway and 7-dehydrocholestrol biosynthetic pathway. Vitamin D is made from squalene by a series of enzyme catalyzed biochemical reactions. Microorganisms that already has an ability to produce high amounts of squalene either natively or by one or more genetic modifications are a preferred host to produce vitamin D. On the other hand, vitamin D biosynthetic pathway can be directly engineered in to the host cell that previously lack the ability to produce squalene and can be used to selectively produce vitamin D. Furthermore, in addition to the enzymes that catalyze the formation of squalene from DHA, one or more of the enzymes selected from a group of vitamin D biosynthetic pathway such as squalene monooxigenase, lanosterol synthase, sterol-14-demethylase, methyl sterol monooxygenase, sterol-4α-carboxylase-3-dehydrogenase, 3-keto steroid reductase, zymosterol demethylase, Δ24-sterol reductase, cholestenolΔisomerase, Δ7sterol-5-desaturase, 7-dehydro cholesterol reductase, sterol24C methyl transferase, C8 sterol isomerase, Δ7-sterol-5-desaturase, sterol-22-desaturase and Δ24-sterol reductase are overexpressed for the enhanced production of vitamin D from squalene. Moreover, improvement of squalene production by overexpressing one or more enzymes in squalene biosynthetic pathway enhances the productivity of vitamin D. In addition, attenuation or deletion of one or more of methylglyoxal synthase (MgsA), phosphoenolpyruvate carboxylate, pyruvate carboxylase, pyruvate dehydrogenase, acetate kinase (AckA), aldehyde dehydrogenase (AldA), phosphotransacylase (pta), pyruvate oxidase (PoxB), pyruvate-formate lyase (PflB), succinate dehydrogenase (FrdBC), acetyl-CoA carboxylase, citrate synthase, alanine transaminase, lactate dehydrogenase (Ldh) and β-ketothiolase (ThlAB) improves acetyl-CoA utilization in vitamin D synthesis. Furthermore, blocking one or more of prenol utilization pathways such as monoterprene synthesis catalyzed by monoterpene cyclase, sequiperpene synthesis by sesquiperpene cyclase and geranylgeranyl-PP synthesis by GGPP synthase improves prenol utilization in the vitamin D synthesis. Blocking vitamin D utilization pathway by attenuation or deletion of enzyme vitamin D-25-hydroxylase responsible for the production of calcidiol from vitamin D3 improves vitamin D accumulation. Furthermore, improving ergosterol and 7-dehydrocholestrol transport, reducing ergosterol and 7-dehydrocholestrol uptake from media and reducing feedback inhibition increases vitamin D productivity.

In some embodiments, microorganisms that have ability to grow in DHA in a medium comprising DHA as a source of carbon is subjected to further genetic modifications to include one or more nucleic acid comprising a nucleotide sequence encoding vitamin E biosynthetic pathway enzymes. Vitamin E is made from geranylgeranyl-PP and tyrosine by a series of enzyme catalyzed biochemical reactions. Microorganisms that already has an ability to produce high amounts of geranylgeranyl-PP either natively or by one or more genetic modifications are a preferred host for the production of vitamin E. On the other hand, vitamin E biosynthetic pathway can be directly engineered in to the host cell that previously lack the ability to produce geranylgeranyl-PP but can be engineered to selectively produce vitamin E. Furthermore, in addition to the enzymes that catalyze the formation of geranylgeranyl-PP from DHA, one or more of the enzymes selected from a group of vitamin E biosynthetic pathway such as geranylgeranyl reductase, tyrosine amino transferase, 4-hydroxy phenyl pyruvate dioxygenase, homogentisate phytyl transferase, tocopherol cyclase, tocopherol-O-methyl transferase, MPBQ/MSBQ methyltransferase and tocopherol cyclase are overexpressed for the enhanced production of vitamin E from geranylgeranyl-PP. In addition, attenuation or deletion of one or more of methylglyoxal synthase (MgsA), phosphoenolpyruvate carboxylate, pyruvate carboxylase, pyruvate dehydrogenase, acetate kinase (AckA), aldehyde dehydrogenase (AldA), phosphotransacylase (pta), pyruvate oxidase (PoxB), pyruvate-formate lyase (PflB), succinate dehydrogenase (FrdBC), acetyl-CoA carboxylase, citrate synthase, alanine transaminase, lactate dehydrogenase (Ldh) and β-ketothiolase (ThlAB) improves acetyl-CoA utilization in vitamin E synthesis. Furthermore, blocking one or more of prenol utilization pathways such as monoterprene synthesis catalyzed by monoterpene cyclase, sequiperpene synthesis by sesquiperpene cyclase, squalene synthesis by squalene synthase, diterpenes synthesis by diterpene cyclase and phytoene synthesis by phytoene synthase improves geranylgeranyl-PP utilization in the vitamin E synthesis. Blocking vitamin E utilization pathway by attenuation or deletion of enzymes facilitating the degradation of vitamin E through oxidation process improves vitamin E accumulation. Furthermore, improving tocopherol transport, reducing tocopherol uptake from media and reducing feedback inhibition increases vitamin E productivity.

In some embodiments, microorganisms that have ability to grow in a medium comprising DHA as a source of carbon is subjected to further genetic modifications to include one or more nucleic acid comprising a nucleotide sequence encoding vitamin K biosynthetic pathway enzymes. Vitamin K includes but not limited to vitamin K1 and vitamin K2. Vitamin K biosynthetic pathway includes but not limited to phylloquinone biosynthetic pathway and menaquinone biosynthetic pathway. Vitamin K is made from chorismate and polyprenyl-PP by a series of enzyme catalyzed biochemical reactions. Microorganisms that already has an ability to produce high amounts of chorismate and polyprenyl-PP either natively or by one or more genetic modifications are a preferred host to produce vitamin K. On the other hand, vitamin K biosynthetic pathway can be directly engineered into the host cell that previously lack the ability to produce chorismate or polyprenyl-PP but can be engineered to selectively produce vitamin K. Furthermore, in addition to the enzymes that catalyze the formation of chorismate and polyprenyl-PP from DHA, one or more of the enzymes selected from a group of vitamin K biosynthetic pathway such as isochorismate synthase, 2-succinyl-5-enol-pyruvyl-6-hydroxy-3-cyclohexene-1-carboxylate synthase, 2-succinyl-6-hydroxy-2,4-cyclohexadiene-1-carboxylate synthase, O-succinylbenzoate synthase, O-succinylbenzoateCoA ligase, naphthoate synthase, 1,4-dihydroxy-2-naphthoyl-CoA hydrolase, 1,4-dihydroxy-2-napthoate octaprenyl transferase and 2-demethyl menaquinone methyl transferase are overexpressed for the enhanced production of vitamin K from chorismate. In addition, attenuation or deletion of one or more of enzymes methylglyoxal synthase (MgsA), phosphoenolpyruvate carboxylate, pyruvate carboxylase, pyruvate dehydrogenase, acetate kinase (AckA), aldehyde dehydrogenase (AldA), phosphotransacylase (pta), pyruvate oxidase (PoxB), pyruvate-formate lyase (PflB), succinate dehydrogenase (FrdBC), acetyl-CoA carboxylase, citrate synthase, alanine transaminase, lactate dehydrogenase (Ldh), acetate carboxylase and β-ketothiolase (ThlAB) improves acetyl-CoA utilization in vitamin K synthesis. Furthermore, blocking one or more of prenol utilization pathways such as monoterprene synthesis catalyzed by monoterpene cyclase, sequiperpene synthesis catalyzed by sesquiperpene cyclase, squalene synthesis catalyzed by squalene synthase, diterpenes synthesis catalyzed by diterpene cyclase and phytoene synthesis catalyzed by phytoene synthase improves geranylgeranyl-PP utilization in the vitamin K synthesis. In addition, blocking one or more chorismate utilization pathways such as prephenate synthesis catalyzed by chorsmate mutase, anthranilate synthesis by anthranilate synthase improves chorismate utilization in vitamin K synthesis. Blocking vitamin K utilization pathway by attenuation or deletion of enzymes menaquinone reductase and phylloquinone reductase catalyzing the transformation of menaquione and phylloquinone to menaquinol and phylloquinol improves vitamin K accumulation. Furthermore, improving menaquinone and phylloquinone transport, reducing menaquinone and phylloquinone uptake from media and reducing feedback inhibition increases vitamin K productivity.

In some embodiments, microorganisms that have ability to grow in a medium comprising DHA as a source of carbon is subjected to further genetic modifications to include one or more nucleic acid comprising a nucleotide sequence encoding coenzyme Q10 biosynthetic pathway enzymes. Coenzyme Q10 is made from chorismate and polyprenyl-PP by a series of enzyme catalyzed biochemical reactions. Microorganisms that already has an ability to produce high amounts of chorismate and decaprenyl-PP either natively or by one or more genetic modifications are a preferred host for the production of coenzyme Q10. On the other hand, coenzyme Q10 biosynthetic pathway can be directly engineered in the host cell that previously lack the ability to produce chorismate or decaprenyl-PP but can be engineered to selectively produce coenzyme Q10. Furthermore, in addition to the enzymes catalyze the formation of chorismate and decaprenyl-PP from DHA, one or more of the enzymes selected from a group of coenzyme Q10 biosynthetic pathway such as chrorismate pyruvate lyase, 4-hydroxybenzoate decaprenyl transferase, 3-decaprenyl-4-hydroxybenzoate decarboxylase, octaprenylphenol hydroxylase, 6-hydroxyphenyl-2-decaprenyl methylase, 6-methoxyphenyl-2-decaprenyl hydroxylase, 6-methoxy-2-decaprenyl 1,4-benzoquinol methylase, 6-methoxy-2-decaprenyl-3-methyl-1,4-benzoquinol hydroxylase, 6-hydroxyphenyl-2-decaprenyl methylase and decaprenyl dihydroxybenzoate methyl transferase are overexpressed for the enhanced production of coenzyme Q10 from chorismate. In addition, attenuation or deletion of one or more of enzymes such as methylglyoxal synthase (MgsA), phosphoenolpyruvate carboxylate, pyruvate carboxylase, pyruvate dehydrogenase, acetate kinase (AckA), aldehyde dehydrogenase (AldA), phosphotransacylase (pta), pyruvate oxidase (PoxB), pyruvate-formate lyase (PflB), succinate dehydrogenase (FrdBC), acetyl-CoA carboxylase, citrate synthase, alanine transaminase, lactate dehydrogenase (Ldh), acetate carboxylase and β-ketothiolase (ThlAB) improves acetyl-CoA utilization in coenzyme Q10 synthesis. Furthermore, blocking one or more of prenol utilization pathways such as monoterprene synthesis catalyzed by monoterpene cyclase, sesquiterpene synthesis catalyzed by sesquiterpene cyclase, squalene synthesis catalyzed by squalene synthase, diterpenes synthesis catalyzed by diterpene cyclase and phytoene synthesis catalyzed by phytoene synthase improves geranylgeranyl-PP utilization in the coenzyme Q10 synthesis. In addition, blocking one or more chorismate utilization pathways such as prephenate synthesis catalyzed by chorismate mutase, anthranilate synthesis by anthranilate synthase improves chorismate utilization in coenzyme Q10 synthesis. Blocking coenzyme Q10 utilization pathway by attenuation or deletion of enzymes catalyzing the oxidative phosphorylation of ubiquinone improves coenzyme Q10 accumulation. Furthermore, improving coenzyme Q10 transport, reducing coenzyme Q10 uptake from media and reducing feedback inhibition increases coenzyme Q10 productivity.

In yet another aspect of the present invention, microorganisms that have ability to grow in a medium comprising DHA as a source of carbon is subjected to further genetic modifications to include one or more nucleic acid comprising a nucleotide sequence encoding riboflavin biosynthetic pathway enzymes. Riboflavin is made from guanosine triphosphate and ribulose-5-phosphate by a series of enzyme catalyzed biochemical reactions. Furthermore, in addition to the enzymes catalyze the formation of guanosine triphosphate and ribulose-5-phophate from DHA, one or more of the enzymes selected from a group of riboflavin biosynthetic pathway such as riboflavin synthase, 6,7-dimethyl-8-ribityllumazine synthase, 3,4-dihydroxy-2-butanone-4-phosphate synthase, 5-amino-6-(5-ribosylamino) uracil reductase, 5-amino-6-(5-ribosylamino) uracil phosphatase, diaminohydroxyphosphoribosylaminopyrimidine are overexpressed for the enhanced production of riboflavin from quanosine triphosphate. In addition, attenuation or deletion of one or more of enzymes methylglyoxal synthase (MgsA), acetate kinase (AckA), aldehyde dehydrogenase (AldA), phosphotransacylase (pta), pyruvate oxidase (PoxB), pyruvate-formate lyase (PflB), alanine transaminase, lactate dehydrogenase (Ldh) and glyceraldehyde-P-dehydrogenase improves DHA utilization in riboflavin synthesis. Furthermore, blocking one or more of D-ribulose-5P utilization pathways such as xylulose synthesis catalyzed by ribose-P-isomerase, D-ribulose synthesis catalyzed by D-ribulose kinase improves ribulose-5P utilization in the riboflavin synthesis. Blocking riboflavin utilization pathway by attenuation or deletion of enzyme riboflavin kinase catalyzing the phosphorylation of riboflavin to riboflavin-5-phosphate improves riboflavin accumulation. Furthermore, improving riboflavin transport, reducing riboflavin uptake from media and reducing feedback inhibition increases riboflavin productivity.

In yet another aspect of the present invention, microorganisms that have ability to grow in DHA in a medium comprising DHA as a source of carbon is subjected to further genetic modifications to include one or more nucleic acid comprising a nucleotide sequence encoding vitamin-B12 biosynthetic pathway enzymes. Vitamin-B12 is made from succinyl-CoA via 5-amino levulinate and uroporphyringogen III by a series of enzyme catalyzed biochemical reactions. Microorganisms that already has an ability to produce high amounts of 5-amino levulinate either natively or by one or more genetic modifications are a preferred host for the production of vitamin-B12. On the other hand, vitamin-B12 biosynthetic pathway can be directly engineered in the host cell that previously lack the ability to produce 5-aminolevulinate but can be engineered to selectively produce vitamin-B12. Furthermore, in addition to the enzymes that catalyze the formation of uroporphyrinogen III from DHA, one or more of the enzymes selected from a group of vitamin-B12 biosynthetic pathway such as uroporphyrinogen III methyl transferase, precorrin-2-C20 methyltransferase, enzymes that catalyze the conversion of precorrin 3 to cobyrinate diamide and encoded by the genes CobG, CogJ, CobM, CobF, CobK, CobH, CobB, CobNST and enzymes that catalyze the conversion of cobyrinate diamide to vitamin B12 and encoded by the genes CobO, CobQ, CobC, CobD, CobP, CobU, CobV and CobS are overexpressed for the enhanced production of vitamin B12 from uroporphyrinogen III. In addition, attenuation or deletion of one or more of enzymes methylglyoxal synthase (MgsA), acetate kinase (AckA), aldehyde dehydrogenase (AldA), phosphotransacylase (pta), pyruvate oxidase (PoxB), pyruvate-formate lyase (PflB), alanine transaminase, lactate dehydrogenase (Ldh), acetyl-CoA carboxylase, β-ketothiolase, aspartate transaminase, glutamate dehydrogenase and hydroxymethylglutararyl-CoA synthase improves DHA utilization in vitamin B12 synthesis. Furthermore, blocking one or more of 5-aminolevulinate utilization pathways such as glutamate semialdehyde synthesis catalyzed by glutamate semialdehyde-2,1-aminomutase and succinyl-CoA utilization pathway such as succinate semialdehyde synthesis catalyzed by succinyl-CoA semialdehyde synthase, methylmalonyl-CoA synthesis catalyzed by methyl-malonyl-CoA mutase improves 5-aminolevulinate utilization in the vitamin B12 synthesis. Blocking vitamin B12 utilization pathway by attenuation or deletion of enzyme catalyzing the degradation of vitamin B12 to other products improves vitamin B12 accumulation. Furthermore, improving vitamin B12 transport, reducing vitamin B12 uptake from media and reducing feedback inhibition increases vitamin B12 productivity.

Genetically modified microorganisms capable of producing vitamins using conventional sugars such as glucose, sucrose or glycerol are already known in the art. U.S. Pat. No. 8,765,421 provides method for producing coenzyme Q10 by solid state fermentation. U.S. Pat. No. 3,769,170 provides yeast and bacteria that produce large amounts of intracellular coenzyme Q10. Chinese Patent Nos. CN101333509 CN103509728 provides genetically engineered Rhodobacter sphaeroides for the production of coenzyme Q10. U.S. Pat. No. 8,815,567 provides genetically engineered oleaginous yeast Yarrowia lipolytica for the production of coenzyme Q10. U.S. Patent application publication No. 2011/0269196 discloses a new isolate Sporidobolus johnsonii capable of producing coenzyme Q10. International PCT Application. Publication Nos. WO2008/063020 and WO2008/100782 provide Rhodobacter sphaeroides sk2 h2 strain for the enhanced production of coenzyme Q10. U.S. Pat. No. 4,367,288 provide methods for producing coenzyme Q10 in good yield by using Aureobasidium sp. International PCT Application Publication No. WO2008/023264 provides coenzyme Q10 production from marine bacteria. U.S. Pat. No. 4,367,289 provides production of coenzyme Q10 in 4-hydroxy benzoic acid containing culture media using Rhodotorula sp. U.S. Pat. No. 6,461,842 provides recombinant Escherichia coli for the production of coenzyme Q10 by hetreologously expressing Rhizobiaceae genes. U.S. Pat. No. 6,762,037 provides microbial production of coenzyme Q10 by expressing enzyme synthesizing coenzyme Q10. U.S. Pat. No. 3,066,080 provides fermentative production of coenzyme Q10. U.S. Patent Application Publication Nos. 2005/0181490 and 2008/0261282 disclose fermentation process for preparing coenzyme Q10 by the recombinant Agrobacterium tumefaciens. U.S. Patent Application Publication No. 2004/0209368 discloses process for producing Sporidiobolus ruineniae strains with improved coenzyme Q10 production. U.S. Pat. No. 7,320,883 provides process for producing coenzyme Q10 with fungal species belong to Aspergillus and Leucosporidium genus. U.S. Patent Application Publication No. 2008/0064074 discloses production of coenzyme Q10 wherein the genes derived from Rhodotorula is used. U.S. Patent Application Publication No. 2004/0234975 discloses production of coenzyme Q10 where in the genes derived from Bulleromyces is used. U.S. Pat. No. 7,422,878 provides process for microbial production of ubiquinone-10 using Rhodobacter. International PCT Application. Publication No. WO2004/047763 provides bacteria for the enhanced production of ubiquinone. U.S. Pat. No. 4,070,244 provides microorganism belonging to the genus Sporidiobolus and Oosporidium to produce ubiquinone-10. U.S. Pat. No. 4,205,125 provides process for production of coenzyme Q8-Q10 using microorganism belonging to genus Pseudomonas. U.S. Pat. No. 4,220,719 provides process for the production of coenzyme Q10 by cultivating microorganism belonging to genus Cryptococcus, Aspergillus, Sporobolomyces, Torulopsis, Sporidiobolus, Ooporidium, Rhodotorula and Cladosporium. U.S. Pat. No. 4,245,048 provides novel microorganism belonging to genus Trichosporon for the production of coenzyme Q10. U.S. Pat. No. 3,658,648 provides coenzyme-Q10 producing microorganism belonging to the Candida genus. Chinese Patent No. CN102994409 provides new bacterial strain Proteus penneri CA8 for coenzyme Q10 production. U.S. Pat. No. 6,103,488 provides production of ubiquinone-10 in Escherichia coli. U.S. Patent Application Publication No. 2009/0226986 discloses improved processes for the fermentative production of the coenzyme Q10 using microorganism of the genus Rhodobacter. U.S. Pat. Nos. 6,492,141 and 3,013,948 provide production of vitamin B12 by culturing the strains of the genus Propionibacterium. U.S. Pat. Nos. 4,544,633 and 2,715,602 provides production of vitamin B12 by culturing vitamin B12 producing microorganism belonging to the genus Propionibacterium. U.S. Pat. Nos. 2,643,213 and 2,595,159 provides production of vitamin B12 by Streptomyces olivaceus. U.S. Pat. No. 2,595,499 provides production of vitamin B12 by means of a vitamin B12 producing strain of Streptomyces griseus. U.S. Pat. No. 3,018,225 provides production of vitamin B12 under fermentative nutrient medium containing a source of cobalt ion. International PCT Application Publication No. WO2013/084052 provides vitamin B12 producing Lactobacillus reuteri strains. U.S. Pat. No. 2,798,840 provides the production of vitamin B12 by Agrobacterium radiobacter. U.S. Patent Application Publication No. 2006/0105432 discloses method to produce vitamin B12 using genetically modified Bacillus megaterium. U.S. Pat. Nos. 5,545,538 and 5,538,888 provide method of producing vitamin B12 using Rhizobium cobalaminogenum BP-4429. U.S. Pat. No. 4,119,492 provides fermentative production of vitamin B12 using microorganism of the species Arthrobacter hyalinus. U.S. Pat. Nos. 2,816,856, 2,753,289, 2,795,602, 3,411,991 and 2,956,932 provides improvement in production of vitamin B12 by Propionibacterium freudenreichii. U.S. Patent Application Publication Nos. 2004/0235120, 2004/0241809 and 2006/0105432 and U.S. Pat. No. 2,576,982 provide vitamin B12 producing strains of Bacillus megaterium. International PCT Application. Publication No. WO2003/083123 provides improved production of vitamin B12 wherein at least one enzyme of the Sirohaemsynthese is used. U.S. Pat. No. 3,085,049 provides process for producing vitamin B12 and antibiotics. U.S. Pat. No. 2,561,364 provides Flavobacterium devorans for the production of vitamin B12. U.S. Pat. No. 4,210,720 provides production of vitamin B12 from microorganism belonging to genus of Arthrobacter and Propionibacterium. U.S. Pat. No. 8,633,009 and U.S. Patent Application Publication No. 2014/0315279 provide genetically engineered oleaginous yeast for the production of quinone derived compounds. U.S. Pat. No. 4,978,617 provides plant tissue culturing process for the production of vitamin E. U.S. Patent Application Publication No. 2006/0021085 provides transgenic plants having elevated vitamin E content by modifying the serine-acetyl transferase content. U.S. Patent Application Publication No. 2003/0182679 provides improved methods for the biosynthesis of vitamin E. U.S. Pat. No. 4,978,617 provides Lactococcus lactic strain with high vitamin K2 production. U.S. Pat. No. 6,677,143 provides culturing Bacillus subtilis for the increased production of vitamin K. U.S. Pat. No. 8,871,195 provides bacteria from group consisting of genera Lactococcus, Leuconostoc and Bacillus that are ThyA(−) mutant that produce increased amounts of vitamin K2. U.S. Pat. No. 8,361,525 provides culture methods favoring the production of vitamin K2 by lactic bacteria. U.S. Pat. No. 7,718,407 provides recombinant Bacillus subtilis for the enhanced production of vitamin K2. International PCT Application Publication No. WO2016/0008411 provides bacterial strains having an outstanding ability to produce menaquinone. U.S. Pat. Nos. 8,114,642 and 8,603,552 provides production of vitamin K2 from culture of Bacillus natto. U.S. Pat. No. 4,649,354 provides process for the production of menaquinone-4 from Corynebacterium, Brevibacterium, Microbacterium, Cutobacterium, Auteobacterium and Flavobacterium. U.S. Pat. Nos. 8,765,118 and 8,673,616 provides Lactococcus lactis strain for the production of vitamin K2. U.S. Patent Application Publication No. 2011/0195467 provides fermentative production of menaquinone-7 using Escherichia coli. U.S. Patent Application Publication No. 2012/0020927 provides bacterial strains having outstanding ability to produce menaquinone. International PCT Application Publication No. WO2011/158998 provides Bacillus amyloliquefaciens for the improved production of vitamin K2. Publication by Morishita et al (Journal of Dairy Science, 1999, 82(9), 1897-1903) reports the production of menaquinones by lactic acid bacteria. A publication from Sato et al (Journal of Industrial Microbiology & Biotechnology, 2001, 25, 115-120) reports menadione resistant mutant of Bacillus subtilis for the efficient production of menaquinone. Another publication from Sato et al (Journal of Bioscience and Engineering, 2001, 91(1), 16-20) reports the production of menaquinone by Bacillus subtillis.

U.S. Pat. No. 2,363,227 provides process to produce riboflavin from yeast species Candida guilliermondia. U.S. Pat. No. 8,551,732 provides process for the increased production of riboflavin by the stabilization of mRNA. International PCT Application Publication No. WO2008/056535 provides high vitamin B2 producing Bacillus natto. U.S. Pat. No. 2,445,128 provides biological process to produce riboflavin. International PCT Application Publication No. WO1992/001060 provides fermentation process for the riboflavin producing organism. U.S. Pat. No. 2,537,148 provides fermentation process for the production of riboflavin using Mycocandida riboflavina. U.S. Patent Application Publication No. 2011/0312025 provides improved production of riboflavin using genetically engineered microorganism. U.S. Pat. No. 2,424,003 provides methods for high yield riboflavin production using Candida flareri. U.S. Patent Application Publication No. 2005/0239161 provides genetic strain optimization for the improved riboflavin production. U.S. Pat. Nos. 2,449,141, 2,581,419 and 2,477,812 provides increased riboflavin production using Clostridium acetobutylicum. U.S. Pat. Nos. 5,334,510, 7,166,456, and 7,078,222 and International PCT Application Publication No. WO2004/046347 provide Bacillus subtilis for the production of riboflavin. U.S. Pat. Nos. 5,231,007, 5,120,655 and 5,164,303 provide production of riboflavin by the strains isolated from Candida famata. U.S. Pat. Nos. 6,376,222 and 5,589,355 provides isolated DNA molecule having riboflavin biosynthetic activities and the expression its expression in the host cell for the riboflavin production. U.S. Pat. No. 5,976,844 provides enhanced riboflavin production by means of increasing isocitrate lyase activity. U.S. Pat. Nos. 2,702,265, 2,666,014, 2,445,128, 2,631,120, 2,578,738, 2,876,169, 5,821,090 and 2,667,445 provide production of riboflavin Ashbya gossypii. Chinese Patent No. CN1033172 provides riboflavin production by genetically altered Candida yeast. U.S. Pat. Nos. 8,759,024 and 3,900,368 provides riboflavin production process by culturing a riboflavin producing microorganism of the genus Bacillus. U.S. Pat. Nos. 2,483,855, 2,615,829, 2,948,549, 2,493,274, 2,647,074, 2,374,503, 2,400,710, 2,473,817, 2,473,818, 2,605,210, 2,498,549 and 3,475,274 provide riboflavin production process by an active culture of the Eremothecium ashbyii. U.S. Pat. No. 6,322,995 provides recombinant bacterium to produce riboflavin. U.S. Pat. Nos. 5,126,248, 6,929,933 and 4,794,081 provides riboflavin production process using Saccharomyces cerevisiae. U.S. Pat. Nos. 5,837,528, 5,925,538, 6,551,813 and 7,091,000 provides bacterial strains for the overproduction of the riboflavin. U.S. Pat. No. 3,433,707 provides riboflavin production culturing yeast belonging to Pichia genus.

U.S. Pat. No. 3,006,932 provides production of ergosterol from yeast. Chinese Patent No. CN102911883 provides production of ergosterol from Saccharomyces cerevisiae Scut-B6305. U.S. Patent Application Publication No. 2004/0235088 provides recombinant yeast for the production of ergosterol. U.S. Patent Application Publication No. 2012/0231495 and U.S. Pat. No. 2,817,624 provides production of non-yeast sterols in yeast such as Saccharomyces cerevisiae. Chinese Patent No. CN1683517 provides production of ergosterol from Saccharomyces cerevisiae ZGFH-88. U.S. Pat. No. 3,884,759 provides production of ergosterol from Trichoderma, Fusarium, Cephalosporium. U.S. Pat. No. 8,367,395 provides sterols production in oleaginous yeast and fungi. U.S. Pat. No. 3,965,130 provides methods for preparing ergosterol and ubiquinone-9 in a single process from a biomass of yeast such as Candida. U.S. Pat. No. 2,794,035 provides process of producing ergosterol and cerebrin. U.S. Pat. No. 7,608,421 provides transgenic organisms for the production 7-dehydrocholesterol. U.S. Patent Application Publication No. 2002/0115126 provides enzyme cholesterol desaturases from Ciliates for the use and production of 7-dehydrocholesterol. Chinese Patent No. CN103275997 provides Saccharomyces cerevisiae to produce 7-dehydrocholesterol. A published article by Avruch et al (Canadian Journal of Biochemistry, 1976, 54(7), 657-665) discloses induced biosynthesis of 7-dehydrocholesterol in Saccharomyces cerevisiae.

U.S. Pat. Nos. 9,115,378 and 8,318,462 provide production of vitamin C by cultivating host-cell selected from group of Gluconobacter and Acetobacter. U.S. Patent Application Publication No. 2002/0012979 provides biosynthetic method to produce vitamin C using microorganisms. U.S. Pat. No. 7,670,814 provides production of vitamin C using microorganisms belonging to the genus Ketogulonicigenium. U.S. Pat. No. 4,945,052 provides production of vitamin C precursor using genetically modified organisms. U.S. Pat. Nos. 7,341,854 and 9,079,951 provide microbial production of vitamin C using Gluconobacter oxydans. U.S. Pat. Nos. 8,338,144 and 7,700,723 provides microbial production of vitamin C wherein the host organism encodes polypeptide having L-sorbosone dehydrogenase activity. U.S. Pat. Nos. 7,579,171 and 6,630,330 provides recombinant Zygosaccharomyces sp. or Kluyveromyces sp. To produce vitamin C. U.S. Pat. No. 5,900,370 provides process for the production of ascorbic acid with Prototheca. U.S. Pat. No. 5,032,514 provides metabolic pathway engineering to increase production of vitamin C. Chinese Patent No. CN101208428 provides recombinant Gluconobacter or Acetobacter for the improved vitamin C production. U.S. Patent Application Publication No. 2006/0234360 provides recombinant yeast to produce vitamin C. U.S. Pat. Nos. 5,001,059 and 5,521,090 provide vitamin C production employing Chlorella pyrenoidosa as microorganism. U.S. Pat. No. 5,792,631 provides ascorbic acid production from Chlorella protothecoides. U.S. Pat. No. 5,922,581 provides process to produce biotin by cultivating recombinant microorganism of the genus Kurthia. U.S. Pat. No. 3,393,129 provides production of biotin by cultivating strain belonging to the genus Sporobolomyces. U.S. Pat. No. 7,423,136 provides production process of biotin by fermentation using genetically engineered microorganisms including Escherichia coli and Pseudomonas mutabilis. U.S. Pat. No. 5,432,067 provides biotin production process using bacteria belonging to the genus Sphingnmonas. U.S. Pat. No. 6,284,500 provides microorganism resistant to β-hydroxynorvaline and having a nucleic acid sequence for the biosynthesis of biotin. U.S. Pat. No. 3,859,167 provides microbial production of biotin using strains belonging to Pseudomonas and Cornybacterium. U.S. Pat. No. 5,445,952 provides recombinant Escherichia coli to produce biotin. U.S. Pat. No. 6,277,609 provides recombinant Escherichia coli that has been transformed with gene BioH for the biotin production. U.S. Pat. No. 6,361,978 provides process for making biotin using recombinant microorganism containing genes BioB, NifU and NifS. U.S. Pat. No. 5,693,504 provides microorganism resistant to 6-aminonicotinamide and having a nucleic acid sequence for the biosynthesis of biotin. U.S. Pat. Nos. 6,083,712 and 5,096,823 provides an isolated DNA molecule comprising biotin-synthesis structural genes BioB, BioF, BioC, BioD and BioA for the biosynthesis of biotin. U.S. Pat. Nos. 6,436,681, 6,955,906, 6,117,669, 6,365,388, and 6,723,544 provide process for preparing biotin using a host organism comprising expression of biotin synthase gene. U.S. Pat. No. 5,179,011 provides recombinant microorganism belongs to Escherichia, Bacillus, Pseudomonas or Serratia to produce biotin. U.S. Pat. Nos. 6,303,377, 6,841,366 and 6,057,136 provides biotin biosynthesis in Bacillus subtilis. U.S. Pat. No. 5,374,554 provides process for the production of biotin using host microorganism of the genus Serratia. U.S. Pat. No. 7,033,814 provides genetically modified yeast with biotin producing activity. U.S. Patent Application Publication No. 2011/0262976 provides production of lipoic acid by a recombinant Gluconobacter oxydans. U.S. Patent application publication No. 2006/0234359 provides method to produce lipoic acid by fermentation.

In one method to enhance DHA uptake and utilization, the genetically modified host cells already known to produce vitamins, as described in patents above, is subjected to further genetic engineering by means of expressing plasmids that code ATP and PEP dependent kinases that could phosphorylate DHA and facilitate its entry into glycolytic cycle. In another method to enhance DHA uptake and utilization, genetically modified host cells already known to produce vitamins, as described in patents above, is subjected to chemical mutagenesis and the strains with the ability to grow and produce desired vitamins with high enough titer and yield in a growth medium comprising DHA as a source of carbon will be identified and subjected to whole genome sequencing. Specific mutation associated with the ability to grow and produce vitamins in a medium comprising DHA is identified and such a mutation will be introduced to the genetically modified host cells already known to produce vitamins to confer the ability to use DHA as a source of organic carbon. In yet another method to enhance DHA uptake and utilization, genetically modified host cells already known to produce vitamins, as described in patents above, is exposed to growth medium with increasing concentration of DHA and subjected to metabolic evolution. Strains that have gained ability to grow in the medium containing DHA as a major or sole source of carbon and still retain the ability to produce vitamins is identified and subjected to whole genome sequencing. Specific mutation associated with the ability to grow and produce vitamins in a medium comprising DHA is identified, such a mutation will be introduced to the genetically modified host cells already known to produce vitamins, as described in patents above to confer the ability to use DHA as a source of organic carbon.

In another embodiment, the present invention provides methods of producing steroids such as cholesterol, pregnenolone, progesterone, cortisol, cortisone, testosterone and estrone. In one aspect of the present invention genetically modified microorganisms producing steroids are subjected to further genetic modifications to confer the ability to use DHA as a source of carbon. In another aspect of the present invention, microorganisms that have ability to grow in DHA in a medium comprising DHA as a source of carbon is subjected to further genetic modifications to produce steroids. The genetic modifications required to produce one or other steroids would involve overexpressing one or more enzymes functional in the of the pregnenolone biosynthetic pathway, progesterone biosynthetic pathway, cortisone biosynthetic pathway, testosterone biosynthetic pathway and estrone biosynthetic pathway such as cholesterol-22β-hydroxylase, 22β-hydroxycholestrol-20α-hydroxylase, 20α, 22β-dihydroxy cholesterol lyase, pregnenolone-17α-hydroxylase, 17α hydroxylase pregnenolone-17,20-lyase, DHA dehydrogenase, androstenedione isomerase, androstenedione reductase, androstenedione 19 hydroxylase, 19-oxoandrostenedione-19-hydrogenase, 19-oxoandrostenedione-2β-hydroxylase, progensterone isomerase, 17-hydroxylase, 21-hydroxylase, 11-deoxycortisol oxidase and dehydrogenase.

In one aspect of the present invention, microorganisms that have ability to grow in DHA in a medium comprising DHA as a source of carbon is subjected to further genetic modifications to include one or more nucleic acid encoding steroid biosynthetic pathway enzymes. Steroids as in the present invention includes but not limited to pregnenolone, progesterone, cortisol, cortisone, testosterone, estrone, estriol, aldosterone and corticosterone. Steroid biosynthetic pathway as used in the present invention includes but not limited to progestrone biosynthetic pathway, testosterone biosynthetic pathway, estrone biosynthetic pathway, aldosterone biosynthetic pathway and cortisone biosynthetic pathway. Steroids are made from cholesterol by a series of enzyme catalyzed biochemical reactions. Microorganisms that already has an ability to produce high amounts of cholesterol either natively or by one or more genetic modifications are a preferred host to produce steroids. On the other hand, steroid biosynthetic pathway can be directly engineered in the host cell that previously lack the ability to produce cholesterol but can be engineered to selectively produce desired steroid. Furthermore, in addition to the enzymes catalyze the formation of cholesterol from DHA, one or more of the enzymes selected from a group of steroid biosynthetic pathway such as cholesterol-22β-hydroxylase, 22β-hydroxycholestrol-20α-hydroxylase, 20α, 22β-dihydroxy cholesterol lyase, pregnenolone-17α-hydroxylase, 17α hydroxylase pregnenolone-17,20-lyase, DHA dehydrogenase, androstenedione isomerase, androstenedione reductase, androstenedione 19 hydroxylase, 19-oxoandrostenedione-19-hydrogenase, 19-oxoandrostenedione-2β-hydroxylase, progensterone isomerase, 17-hydroxylase, 21-hydroxylase, 11-deoxycortisol oxidase and dehydrogenase are overexpressed for the enhanced production of steroids from cholesterol. In addition, attenuation or deletion of one or more of enzymes methylglyoxal synthase (MgsA), phosphoenolpyruvate carboxylate, pyruvate carboxylase, pyruvate dehydrogenase, acetate kinase (AckA), aldehyde dehydrogenase (AldA), phosphotransacylase (pta), pyruvate oxidase (PoxB), pyruvate-formate lyase (PflB), succinate dehydrogenase (FrdBC), acetyl-CoA carboxylase, citrate synthase, alanine transaminase, lactate dehydrogenase (Ldh), acetate carboxylase and β-ketothiolase (ThlAB) improves acetyl-CoA utilization in steroid biosynthesis. Furthermore, blocking one or more of prenol utilization pathways such as monoterprene synthesis catalyzed by monoterpene cyclase, sequiterpene synthesis catalyzed by sesquiterpene cyclase, diterpenes synthesis catalyzed by diterpene cyclase, vitamin K and ubiquinone synthesis catalyzed by polyprenyl transferases and phytoene synthesis catalyzed by phytoene synthase improves farnesyl-PP utilization in the cholesterol synthesis. Blocking cholesterol utilization to bile acid synthesis by deleting one or more of enzymes cholesterol oxidase, cholesterol acyltransferase and cholesterol 7a-monooxygenase improves cholesterol utilization in the steroid synthesis. Blocking steroid utilization pathway by attenuation or deletion of enzymes facilitating the degradation of desired steroid through oxidation process improves steroid accumulation. Furthermore, improving steroid transport, reducing steroid uptake from media and reducing feedback inhibition increases steroid productivity.

Genetically modified microorganisms capable of producing steroids using conventional sugars such as glucose, sucrose or glycerol are already known in the art. U.S. Pat. Nos. 6,333,172 and 6,107,462 provides genes and proteins controlling cholesterol biosynthesis. U.S. Pat. No. 3,526,576 provides production of steroid compounds by microbial conversion. U.S. Pat. No. 3,741,870 provides fermentation method to produce Δ9(11)-estrone. U.S. Pat. Nos. 8,685,705 and 8,211,676 provide cholesterol producing yeast strains and its application in the production of cholesterol. U.S. Pat. Nos. 7,977,065 and 7,670,829 provide yeast strains autonomously producing steroids. U.S. Pat. No. 7,033,779 provides method for preparing steroids by yeast strain transformed to express the Cyp7b gene. U.S. Pat. Nos. 7,879,592 and 8,173,402 provide modified yeast to produce steroid derivatives. U.S. Pat. Nos. 5,989,881 and 5,759,801 and 5,965,417 provides nucleic acid molecules encoding Δ5,7-srerol, 47 reductase to produce steroids. U.S. Pat. Nos. 6,218,139 and 6,503,749 provide yeast strains possessing the interrupted ATF2 gene for the production of steroids. Chinese Patent No. CN 103,756,940 provides Mycobacterium fortuitum for the fermentative production of estrone lactone. U.S. Pat. No. 3,125,495 provides microbial preparation of 7 and 15 hydroxy steroids. Chinese Pat. No. CN1670185 provides Mycobacterium fortuitum for the fermentative production of testosterone. U.S. Pat. No. 2,982,694 provides steroid production with Streptomyces and Cladosporium. U.S. Pat. No. 2,954,326 provides steroid production with Dactylium dendroides. U.S. Pat. No. 2,977,286 provides synthesis of steroids with Kabatiella phoradendri. U.S. Pat. No. 3,005,017 provides synthesis of steroids by Streptomyces roseochromogenus. U.S. Pat. No. 2,753,290 provides microbiological production of 7 and 15 hydroxy progesterone. U.S. Pat. No. 2,840,579 provides 9-alpha hydroxyl-6-oxygenated progesterones. A publication from Xu et al (Biochem. Biophys Res. Commun. 1988, 155(1), 509-517) discloses biosynthesis of cholesterol in the yeast mutant Erg6. Another recent publication from Szczebara et al (Nat. Biotechnol. 2003, 21(2), 143-149) discloses total biosynthesis of hydrocortisone from a sugar based carbon sources in yeast.

In one method to enhance DHA uptake and utilization, the genetically modified host cells already known to produce steroids, as described in patents above, is subjected to further genetic engineering by means of expressing plasmids that code ATP and PEP dependent kinases that could phosphorylate DHA and facilitate its entry into glycolytic cycle. In another method to enhance DHA uptake and utilization, genetically modified host cells already known to produce steroids, as described in patents above, is subjected to chemical mutagenesis and the strains with the ability to grow and produce desired sterols with high enough titer and yield in a growth medium comprising DHA as a source of carbon will be identified and subjected to whole genome sequencing. Specific mutation associated with the ability to grow and produce sterols in a medium comprising DHA is identified and such a mutation will be introduced to the genetically modified host cells already known to produce steroids to confer the ability to use DHA as a source of organic carbon. In another method to enhance DHA uptake and utilization, genetically modified host cells already known to produce steroids, as described in patents above, is exposed to growth medium with increasing concentration of DHA and subjected to metabolic evolution. Strains that have gained ability to grow in the medium containing DHA as a major or sole source of carbon and still retain the ability to produce steroids are identified and subjected to whole genome sequencing to identify specific mutation associated with the ability to grow and produce steroids in a medium comprising DHA. Such a mutation will be introduced to the genetically modified host cells already known to produce steroids, as described in patents above, for the purpose of conferring the ability to use DHA as a source of organic carbon.

In another embodiment, the present invention provides methods for producing antibiotics such as penicillin, cephalosporin, griseofulvin, bacitracin, polymyxin, amphotericin, erythromycin, neomycin, streptomycin, tetracycline, venacomycin, gentamicin and rifamycin. In one aspect of the present invention, the microorganisms already used in the production of antibiotics are subjected to further genetic modifications to confer the ability to use DHA as a source of carbon. In another aspect of this invention, microorganisms that have ability to grow in a medium comprising DHA as a source of carbon is subjected to further genetic modifications in one or more enzymes functional in the penicillin biosynthetic pathway, cephalosporin biosynthetic pathway, griseofulvin biosynthetic pathway, bacitracin biosynthetic pathway, polymyxin biosynthetic pathway, amphotericin biosynthetic pathway, erythromycin biosynthetic pathway, neomycin biosynthetic pathway, streptomycin biosynthetic pathway, tetracycline biosynthetic pathway, venacomycin biosynthetic pathway, gentamicin biosynthetic pathway and rifamycin biosynthetic pathway. The list of the genetic manipulations that are suitable for enhancing the production of one or other antibiotics includes: enhancing the activities of one or more of the enzymes functional in the penicillin and cephalosporin biosynthetic pathway such as N-(5-amino-5-carboxypentanoyl)-L-cysteinyl-D-valine synthase, isopenicillin-N synthase, isopenicillin-N,N-acyltransferase, isopenicillin-N-epimerase and the proteins CefE, CefF, and CefG facilitating the conversion of penicillin N to cephalosporin C; enhancing activities of one or more proteins functional in the in the griseofulvin biosynthetic pathway such as GsfA, GsfB, GsfC, Gsfl, GsfF, GsfD and GsfE proteins facilitating the conversion of acetyl-Coa and malonyl-CoA to griseofulvin; enhancing activities of one or more of the proteins functional in the polymyxin biosynthetic pathway such as polymyxin synthetase coded by pmxB, pmxA and pmxE genes; enhancing activities of one or more of the enzymes functional in the erythromycin biosynthetic pathway such as the enzymes facilitating the conversion of propionyl-CoA and methylmalonyl-CoA to 6-deoxyerythronolide B coded by the genes eryA1, eryA2 and eryA3, the enzymes facilitating the conversion of 6-deoxyerythronolide B to 3-alpha-mycarosylerythronolide B coded by the genes eryF and eryBV, the enzymes facilitating the conversion of 3-alpha-mycarosylerythronolide B to erythromycin D coded by the genes eryC3 and eryC2, and the the enzyme facilitating the conversion of erythromycin D to erythromycin A coded by the genes eryK and eryG; enhancing activities of one or more of the enzymes in the neomycin biosynthetic pathway such as encoding enzymes facilitating the conversion of glucose-6-phosphate to paromamine encoded by genes neoC, neoB, neoA, neoD and neoL, enzymes facilitating the conversion of paromamine to ribostamycin coded by the genes neoG, neoN, neoM and neoL and the enzyme facilitating the conversion of ribostamycin to neomycin coded by the genes neoK, neoL, neoG and neoN; enhancing activities of one or more of the enzymes functional in the streptomycin biosynthetic pathway such as such as the enzymes facilitating the conversion of myo-inositol to 1,4-L-dihydrostreptosyl-streptidine-6-phosphate, N-methyl-L-glucosaminyltransferase, dihydrostreptomycin-6-phosphate oxidoreductase and streptomycin-6-phosphatase; enhancing activities of one or more of the enzymes functional in the tetracycline biosynthetic pathway such as amidotransferase, the enzymes facilitating the conversion of malonyl-CoA to pretetramide coded by the genes oxyA, oxyB, oxyC, oxyJ, oxyK and oxyN and the enzymes facilitating the conversion of pretetramide to tetracycline encoded by the genes OxyF, OxyE, OxyL, OxyQ, OxyT and OxyS genes; enhancing activities of one or more enzymes functional in the vancomycin biosynthetic pathway such as the enzymes facilitating the conversion of 4-hydroxyphenylpyruvate to 4-hydroxyphenylglycine encoded by the genes HmaS, Hmo and HpgT, the enzymes facilitating the conversion of malonyl-CoA to 3,5-dhydroxyphenylglycine encoded by the genes DpgA, DpgB, DpgD, DpgC and HpgT, the enzymes facilitating the conversion 4-hydroxyphenylglycine, 3,5-dihydroxyphenylglycine, leucine, aspartic acid and tyrosine to heptapeptide vancomycin aglycone encoded by the genes CepK, CepL, CepJ, CepC, CepH, CepB and CepA and the enzymes facilitating the conversion of dTDP-D-glucose to vancomycin encoded by the genes RfbB, EvaA, EvaB, EvaC, EvaD and VcaE; enhancing activities of one or more of the enzymes functional in the gentamicin biosynthetic pathway such as the enzymes facilitating the conversion of glucose-6-phosphate to paromamine encoded by the genes NeoC, NeoB, NeoA, NeoD and NeoL and the enzymes facilitating the conversion of paromamine to gentamicin encoded by the genes GntD, GntA, Gntl and GntK; enhancing activities of one or more of the enzymes functional in the rifamycin biosynthetic pathway facilitating the conversion of UDP-D-glucose to 3-amino-5-hydroxybenzoate encoded by the genes TktA, RifL, RifK, RifM, RifN, RifH, RifG, RilJ and RifK, and the enzymes facilitating the conversion of 3-amino-5-hydroxybenzoate to rifamycin encoded by the genes RifA, RifB, RifC, RifD, RifE, RifF, Rif-orf5, RifLorf20 and RifLorf14.

In some embodiments, microorganisms that have ability to grow in DHA in a medium comprising DHA as a source of carbon is subjected to further genetic modifications to include one or more nucleic acid comprising a nucleotide sequence encoding penicillin and cephalosporin biosynthetic pathway enzymes. Penicillin and cephalosporin is made from lysine degradation metabolite 2-amino adipate by a series of enzyme catalyzed biochemical reactions. Microorganisms that already has an ability to produce high amounts of lysine either natively or by one or more genetic modifications are a preferred host to produce penicillin and cephalosporin. On the other hand, penicillin and cephalosporin biosynthetic pathway can be directly engineered in the host cell that previously lack the ability to produce lysine but can be engineered to selectively produce desired penicillin or cephalosporin. Furthermore, in addition to the enzymes that catalyze the formation of lysine from DHA, one or more of the enzymes selected from a group of penicillin and cephalosporin biosynthetic pathway such as N-(5-amino-5-carboxypentanoyl)-L-cysteinyl-D-valine synthase, isopenicillin-N synthase, isopenicillin-N,N-acyltransferase, isopenicillin-N-epimerase and the genes CefE, CefF, CefG encoding enzymes facilitating the conversion of penicillin N to cephalosporin are overexpressed for the enhanced production of penicillin and cephalosporin from lysine. In addition, attenuation or deletion of one or more of enzymes methylglyoxal synthase (MgsA), acetate kinase (AckA), aldehyde dehydrogenase (AldA), phosphotransacylase (pta), pyruvate oxidase (PoxB), pyruvate-formate lyase (PflB), acetolactate synthase, acetyl-CoA carboxylase, alanine transaminase, lactate dehydrogenase (Ldh), hydroxymethylglutary-CoA synthase and β-ketothiolase (ThlAB) improves pyruvate utilization in penicillin and cephalosporin biosynthesis. Furthermore, blocking one or more of penicillin degradation pathways such as 6-aminopenicillanic acid synthesis catalyzed by penicillin amidase, penicilloic acid synthesis catalyzed by beta-lactamase improves penicillin accumulation. Blocking one or more of cephalosporin degradation pathways such as oxidation catalyzed by D-aminoacid oxidase or cephalosporin-N-transaminase and deacetylation of cephalosporin catalyzed by cephalosporin-C-deacylase improves cephalosporin accumulation. Furthermore, improving penicillin and cephalosporin transport, reducing penicillin and cephalosporin uptake from media and reducing feedback inhibition increases penicillin and cephalosporin productivity.

In some embodiments, microorganisms that have ability to grow in DHA in a medium comprising DHA as a source of carbon is subjected to further genetic modifications to include one or more nucleic acid comprising a nucleotide sequence encoding griseofulvin biosynthetic pathway enzymes. Griseofulvin is made from acetyl-CoA and malonyl-CoA by a series of enzyme catalyzed biochemical reactions. Microorganisms that already has an ability to produce high amounts of malonyl-CoA either natively or by one or more genetic modifications are a preferred host for the production of griseofulvin. On the other hand, griseofulvin biosynthetic pathway can be directly engineered in the host cell that previously lack the ability to produce malonyl-CoA but can be engineered to selectively produce griseofulvin. Furthermore, in addition to the enzymes that catalyze the formation of malonyl-CoA from DHA, one or more of the enzymes selected from a group of griseofulvin biosynthetic pathway such as genes GsfA, GsfB, GsfC, Gsfl, GsfF, GsfD and GsfE encoding enzymes facilitating the conversion of acetyl-CoA and malonyl-CoA to griseofulvin are overexpressed for the enhanced production of griseofulvin. In addition, attenuation or deletion of one or more of enzymes methylglyoxal synthase (MgsA), pyruvate carboxylase, pyruvateenolphosphate carboxylase, succinate reductase, citrate synthase, acetate kinase (AckA), aldehyde dehydrogenase (AldA), pyruvate oxidase (PoxB), pyruvate-formate lyase (PflB), acetolactate synthase, alanine transaminase, lactate dehydrogenase (Ldh), hydroxymethylglutary-CoA synthase and β-ketothiolase (ThlAB) improves pyruvate utilization in griseofulvin biosynthesis. Furthermore, blocking one or more of griseofulvin degradation pathways such as oxidation reaction and demethylation reactions improves griseofulvin accumulation. Furthermore, improving griseofulvin transport, reducing griseofulvin uptake from media and reducing feedback inhibition increases griseofulvin productivity.

In some embodiments, microorganisms that have ability to grow in DHA in a medium comprising DHA as a source of carbon is subjected to further genetic modifications to include one or more nucleic acid comprising a nucleotide sequence encoding polymyxin biosynthetic pathway enzymes. Polymyxin is made from 2,4-diaminobutanoate, L-threonine, L-leucine and 6-methyloctanoyl-CoA by a single step biochemical reaction catalyzed by polymyxin synthetase. Microorganisms such as Bacillus polymyxa that already has an ability to produce high amounts of 2,4-diaminobutanoate, L-threonine, L-leucine and 6-methylocatnoyl-CoA either natively or by one or more genetic modifications are a preferred host to produce polymyxin. On the other hand, polymyxin biosynthetic pathway can be directly engineered in to the host cell that previously lack the ability to produce 2,4-diaminobutanoate, L-threonine, L-leucine and 6-methylocatnoyl-CoA but can be engineered to selectively produce polymyxin. Furthermore, in addition to the enzymes catalyze the formation of 2,4-diaminobutanoate, L-threonine, L-leucine and 6-methylocatnoyl-CoA from DHA, one or more of the enzymes selected from a group of polymyxin biosynthetic pathway such as genes PmxB, PmxA and PmxE encoding enzyme polymyxin synthetase is overexpressed for the enhanced production of polymyxin. Furthermore, blocking one or more of polymyxin degradation pathways such as oxidation reaction and peptide hydrolysis reactions improves polymyxin accumulation. Furthermore, improving polymyxin transport, reducing polymyxin uptake from media and reducing feedback inhibition increases polymyxin productivity.

In some embodiments, microorganisms that have ability to grow in DHA in a medium comprising DHA as a source of carbon is subjected to further genetic modifications to include one or more nucleic acid comprising a nucleotide sequence encoding erythromycin biosynthetic pathway enzymes. Erythromycin is made from propionyl-CoA and (S)-methylmalonyl-CoA by a series of enzyme catalyzed biochemical reactions. Microorganisms that already has an ability to produce high amounts of (S)-methylmalonyl-CoA and propionyl-CoA either natively or by one or more genetic modifications are a preferred host to produce erythromycin. On the other hand, erythromycin biosynthetic pathway can be directly engineered in the host cell that previously lack the ability to produce methylmalonyl-CoA but can be engineered to selectively produce erythromycin. Furthermore, in addition to the enzymes that catalyze the formation of methylmalonyl-CoA and propionyl-CoA from DHA, one or more of the enzymes selected from a group of erythromycin biosynthetic pathway such as genes EryA1, EryA2 and EryA3 encoding enzymes facilitating the conversion of propionyl-CoA and methylmalonyl-CoA to 6-deoxyerythronolide B, genes EryF and EryBV encoding enzymes facilitating the conversion of 6-deoxyerythronolide B to 3-alpha-mycarosylerythronolide B, genes EryC3 and EryC2 encoding enzymes facilitating the conversion of 3-alpha-mycarosylerythronolide B to erythromycin D, genes EryK and EryG encoding enzymes facilitating the conversion of erythromycin D to erythromycin A are overexpressed for the enhanced production of erythromycin from propionyl-CoA and methylmalonyl-CoA. In addition, attenuation or deletion of one or more of enzymes methylglyoxal synthase (MgsA), acetate kinase (AckA), aldehyde dehydrogenase (AldA), pyruvate oxidase (PoxB), pyruvate-formate lyase (PflB), acetolactate synthase, alanine transaminase, lactate dehydrogenase (Ldh), acetyl-CoA carboxylase, hydroxymethylglutary-CoA synthase and β-ketothiolase (ThlAB) improves pyruvate utilization in erythromycin biosynthesis. Furthermore, blocking one or more of erythromycin degradation pathways such as oxidation reaction and hydrolysis reactions improves erythromycin accumulation. Furthermore, improving erythromycin transport, reducing erythromycin uptake from media and reducing feedback inhibition increases erythromycin productivity.

In some embodiments, microorganisms that have ability to grow in DHA in a medium comprising DHA as a source of carbon is subjected to further genetic modifications to include one or more nucleic acid comprising a nucleotide sequence encoding neomycin biosynthetic pathway enzymes. Neomycin is made from glucose-6-phosphate by a series of enzyme catalyzed biochemical reactions. Microorganisms are genetically engineered to produce glucose-6-phosphate from DHA by over expressing one or more of the enzymes such as dihydroxyacetone phosphate, triosephosphate isomerase, fructose bisphosphate aldolase and glucose phosphate isomerase. Furthermore, in addition to the enzymes catalyze the formation of glucose-6-phosphate from DHA, one or more of the enzymes selected from a group of neomycin biosynthetic pathway such as genes NeoC, NeoB, NeoA, NeoD and NeoL encoding enzymes facilitating the conversion of glucose-6-phosphate to paromamine, genes NeoG, NeoN, NeoM and NeoL encoding enzymes facilitating the conversion of paromamine to ribostamycin, genes NeoK, NeoL, NeoG and NeoN facilitating the conversion of ribostamycin to neomycin are overexpressed for the enhanced production of neomycin from glucose-6-phosphate. In addition, attenuation or deletion of one or more of enzymes methylglyoxal synthase (MgsA), glyceraldehyde-P-dehydrogenase, pyruvate carboxylase, pyruvate-enolphosphate carboxylase, succinate reductase, acetate kinase (AckA), aldehyde dehydrogenase (AldA), pyruvate oxidase (PoxB), pyruvate-formate lyase (PflB), acetolactate synthase, alanine transaminase, lactate dehydrogenase (Ldh), acetyl-CoA carboxylase, hydroxymethylglutary-CoA synthase and β-ketothiolase (ThlAB) improves DHA utilization in neomycin biosynthesis. Furthermore, blocking one or more of neomycin degradation pathways such as oxidation reaction and hydrolysis reactions improves neomycin accumulation. Furthermore, improving neomycin transport, reducing neomycin uptake from media and reducing feedback inhibition increases neomycin productivity.

In some embodiments, microorganisms that have ability to grow in DHA in a medium comprising DHA as a source of carbon is subjected to further genetic modifications to include one or more nucleic acid comprising a nucleotide sequence encoding streptomycin biosynthetic pathway enzymes. Streptomycin is made from myo-inositol by a series of enzyme catalyzed biochemical reactions. Microorganisms are genetically engineered to produce myo-inositol from DHA by overexpressing one or more of the enzymes such as dihydroxyacetone phosphate, triosephosphate isomerase, fructose bisphosphate aldolase, glucose phosphate isomerase, myo-inositol-1P-synthase and myo-inositol phosphatase. Furthermore, in addition to the enzymes catalyze the formation of myo-inositol from DHA, one or more of the enzymes selected from a group of streptomycin biosynthetic pathway such as EC 1.1.1.18, EC 2.6.1.50, EC 2.7.1.65, EC 2.1.4.2, EC 3.1.3.40, EC 2.6.1.56, gene StrB1, EC 2.4.2.27 encoding enzymes facilitating the conversion of myo-inositol to 1,4-L-dihydrostreptosyl-streptidine-6-phosphate, N-methyl-L-glucosaminyltransferase, dihydrostreptomycin-6-phosphate oxidoreductase and streptomycin-6-phosphatase are overexpressed for the enhanced production of streptomycin from myo-inositol. In addition, attenuation or deletion of one or more of enzymes methylglyoxal synthase (MgsA), glyceraldehyde-P-dehydrogenase, pyruvate carboxylase, pyruvate-enolphosphate carboxylase, succinate reductase, acetate kinase (AckA), aldehyde dehydrogenase (AldA), pyruvate oxidase (PoxB), pyruvate-formate lyase (PflB), acetolactate synthase, alanine transaminase, lactate dehydrogenase (Ldh), acetyl-CoA carboxylase, hydroxymethylglutary-CoA synthase and β-ketothiolase (ThlAB) improves DHA utilization in streptomycin biosynthesis. Furthermore, blocking one or more of streptomycin degradation pathways such as phosphorylation reaction catalyzed by streptomycin-6-kinase and hydrolysis reactions improves streptomycin accumulation. Furthermore, improving streptomycin transport, reducing streptomycin uptake from media and reducing feedback inhibition increases streptomycin productivity.

In some embodiments, microorganisms that have ability to grow in DHA in a medium comprising DHA as a source of carbon is subjected to further genetic modifications to include one or more nucleic acid comprising a nucleotide sequence encoding tetracycline biosynthetic pathway enzymes. Tetracycline is made from malonyl-CoA by a series of enzyme catalyzed biochemical reactions. Microorganisms that already has an ability to produce high amounts of malonyl-CoA either natively or by one or more genetic modifications are a preferred host for the production of tetracycline. On the other hand, tetracycline biosynthetic pathway can be directly engineered in to the host cell that previously lack the ability to produce malonyl-CoA but can be engineered to selectively produce tetracycline. Furthermore, in addition to the enzymes catalyze the formation of malonyl-CoA from DHA, one or more of the enzymes selected from a group of tetracycline biosynthetic pathway such as amidotransferase facilitating the conversion of malonyl-CoA to Malonamoyl-CoA; genes OxyA, OxyB, OxyC, OxyJ, OxyK and OxyN encoding enzymes facilitating the conversion of malonyl-CoA to pretetramide; genes OxyF, OxyE, OxyL, OxyQ, OxyT and OxyS encoding enzymes facilitating the conversion of pretetramide to tetracycline are overexpressed for the enhanced production of tetracycline from malonyl-CoA. In addition, attenuation or deletion of one or more of enzymes methylglyoxal synthase (MgsA), pyruvate carboxylase, pyruvate-enolphosphate carboxylase, succinate reductase, citrate synthase, acetate kinase (AckA), aldehyde dehydrogenase (AldA), pyruvate oxidase (PoxB), pyruvate-formate lyase (PflB), acetolactate synthase, alanine transaminase, lactate dehydrogenase (Ldh), hydroxymethylglutary-CoA synthase and β-ketothiolase (ThlAB) improves pyruvate utilization in tetracycline biosynthesis. Furthermore, blocking one or more of tetracycline degradation pathways such as oxidation reaction and hydrolysis reactions improves tetracycline accumulation. Furthermore, improving tetracycline transport, reducing tetracycline uptake from media and reducing feedback inhibition increases tetracycline productivity.

In some embodiments, microorganisms that have ability to grow in DHA in a medium comprising DHA as a source of carbon is subjected to further genetic modifications to include one or more nucleic acid comprising a nucleotide sequence encoding vancomycin biosynthetic pathway enzymes. Vancomycin is a heptapeptide antibiotic made from natural amino acids such as leucine, aspartic acid, tyrosine and unnatural aminoacid such as 4-hydroxyphenyl glycine and 3,5-dihydroxyphenyl glycine by a series of enzyme catalyzed biochemical reactions.

Microorganisms that already has an ability to produce necessary aminoacids required for vancomycin synthesis either natively or by one or more genetic modifications are a preferred host for the production of tetracycline. On the other hand, vancomycin biosynthetic pathway can be directly engineered in to the host cell that previously lack the ability to produce aminoacids required for vancomycin synthesis but can be engineered to selectively produce vancomycin. Furthermore, one or more of the enzymes required for the synthesis of 4-hydroxyphenyl glycine such as genes HmaS, Hmo and HpgT encoding enzymes facilitating the conversion of 4-hydroxyphenylpyruvate to 4-hydroxyphenylglycine; one or more of the enzymes required for the synthesis of 3,5-dihydroxyphenyl glycine such as genes DpgA, DpgB, DpgD, DpgC and HpgT encoding enzymes facilitating the conversion of malonyl-CoA to 3,5-dhydroxyphenylglycine; one or more of the enzymes required for the synthesis of heptapeptide vancomycin aglycone such as genes CepK, CepL, CepJ, CepC, CepH, CepB and CepA encoding enzymes facilitating the conversion 4-hydroxyphenylglycine, 3,5-dihydroxyphenylglycine, leucine, aspartic acid and tyrosine to heptapeptide vancomycin aglycone; one or more of the enzymes required for the synthesis of vancomycin such as genes RfbB, EvaA, EvaB, EvaC, EvaD and VcaE encoding enzymes facilitating the conversion of dTDP-D-glucose to vancomycin are overexpressed for the enhanced production of vancomycin. Furthermore, blocking one or more of vancomycin degradation pathways such as oxidation reaction and hydrolysis reactions improves vancomycin accumulation. Furthermore, improving vancomycin transport, reducing vancomycin uptake from media and reducing feedback inhibition increases vancomycin productivity.

In some embodiments, microorganisms that have ability to grow in DHA in a medium comprising DHA as a source of carbon is subjected to further genetic modifications to include one or more nucleic acid comprising a nucleotide sequence encoding gentamicin biosynthetic pathway enzymes. Gentamicin is made from glucose-6-phosphate by a series of enzyme catalyzed biochemical reactions. Microorganisms are genetically engineered to produce glucose-6-phosphate from DHA by over-expressing one or more of the enzymes such as dihydroxyacetone phosphate, triosephosphate isomerase, fructose bisphosphate aldolase and glucose phosphate isomerase. Furthermore, in addition to the enzymes catalyze the formation of glucose-6-phosphate from DHA, one or more of the enzymes selected from a group of gentamicin biosynthetic pathway such as genes NeoC, NeoB, NeoA, NeoD and NeoL encoding enzymes facilitating the conversion of glucose-6-phosphate to paromamine; genes GntD, GntA, Gntl and GntK encoding enzymes facilitating the conversion of paromamine to gentamicin are overexpressed for the enhanced production of gentamicin from DHA. In addition, attenuation or deletion of one or more of enzymes methylglyoxal synthase (MgsA), glyceraldehyde-P-dehydrogenase, pyruvate carboxylase, pyruvate-enolphosphate carboxylase, succinate reductase, acetate kinase (AckA), aldehyde dehydrogenase (AldA), pyruvate oxidase (PoxB), pyruvate-formate lyase (PflB), acetolactate synthase, alanine transaminase, lactate dehydrogenase (Ldh), acetyl-CoA carboxylase, hydroxymethylglutary-CoA synthase and β-ketothiolase (ThlAB) improves DHA utilization in gentamicin biosynthesis. Furthermore, blocking one or more of gentamicin degradation pathways such as oxidation reaction and hydrolysis reactions improves gentamicin accumulation. Furthermore, improving gentamicin transport, reducing gentamicin uptake from media and reducing feedback inhibition increases gentamicin productivity.

In some embodiments, microorganisms that have ability to grow in DHA in a medium comprising DHA as a source of carbon is subjected to further genetic modifications to include one or more nucleic acid comprising a nucleotide sequence encoding rifamycin biosynthetic pathway enzymes. Rifamycin is made from UDP-D-glucose by a series of enzyme catalyzed biochemical reactions. Microorganisms are genetically engineered to produce UDP-D-glucose from DHA by over-expressing one or more of the enzymes such as dihydroxyacetone phosphate, triosephosphate isomerase, fructose bisphosphate aldolase, glucose phosphate isomerase, phosphoglutomutase and UTP glucose-1P-uridylyltransferase. Furthermore, in addition to the enzymes catalyze the formation of UDP-D-glucose from DHA, one or more of the enzymes selected from a group of rifamycin biosynthetic pathway such as genes TktA, RifL, RifK, RifM, RifN, RifH, RifG, RifJ and RifK encoding enzymes facilitating the conversion of UDP-D-glucose to 3-amino-5-hydroxybenzoate; genes RifA, RifB, RifC, RifD, RifE, RifF, Rif-orf5, Rif-orf20 and Rif-orf14 encoding enzymes facilitating the conversion of 3-amino-5-hydroxybenzoate to rifamycin are overexpressed for the enhanced production of rifamycin from DHA. In addition, attenuation or deletion of one or more of enzymes methylglyoxal synthase (MgsA), glyceraldehyde-P-dehydrogenase, pyruvate carboxylase, pyruvate-enolphosphate carboxylase, succinate reductase, acetate kinase (AckA), aldehyde dehydrogenase (AldA), pyruvate oxidase (PoxB), pyruvate-formate lyase (PflB), acetolactate synthase, alanine transaminase, lactate dehydrogenase (Ldh), hydroxymethylglutary-CoA synthase and β-ketothiolase (ThlAB) improves DHA utilization in rifamycin biosynthesis. Furthermore, blocking one or more of rifamycin degradation pathways such as oxidation reaction and hydrolysis reactions improves rifamycin accumulation. Furthermore, improving rifamycin transport, reducing rifamycin uptake from media and reducing feedback inhibition increases rifamycin productivity.

Genetically modified microorganisms capable of producing “antibiotics” using conventional sugars such as glucose, sucrose or glycerol are already known in the art. U.S. Pat. No. 4,533,632 provides fermentative production of cephalosporin using Acremonium chrysogenum. U.S. Pat. Nos. 4,520,101, 3,139,388, 3,139,389, 3,816,257, 3,825,473, 2,831,797, 3,929,577, 3,082,155, 3,196,084, 2,883,328, 4,368,263 and 3,116,216 provide production of cephalosporin C under reduced phosphorous salts using Cephalosporium sp. U.S. Pat. No. 3,116,217 provides improving cephalosporin C production by the addition of norvaline and norleucine. International PCT Application Publication No. WO2007/096419 and U.S. Pat. Nos. 6,319,684, 6,995,003 and 7,553,635 provides improved cephalosporin production using Penicillium chrysogenum. U.S. Pat. No. 3,948,726 provides polyene antibiotics resistant Cephalosporium sp. used for the production of cephalosporin. U.S. Pat. Nos. 3,069,329, 3,152,150, 3,616,247, 3,038,839, 3,069,328, 3,095,360, 3,607,656, 2,843,527, and 2,986,496 provide fermentative production of griseofulvin by griseofulvin producing microorganism. U.S. Pat. No. 2,938,835 provides genetically modified Pencillum for the production of griseofulvin. U.S. Pat. No. 3,616,238 provides method for the production of 5-hydroxy griseofulvin using Streptomyces cinereocrocatus. U.S. Pat. Nos. 2,789,941, 2,498,165, 2,813,061, 2,567,698, 2,627,494 and 2,828,246 provides methods for the production of bacitracin using strains of Bacillus subtilis. International PCT Application Publication No. WO2007/136824 provides high-yield bacitracin producing microorganism using mutant Bacillus licheniformis. U.S. Pat. No. 5,589,381 provides Bacillus licheniformis to produce bacitracin. U.S. Pat. No. 2,571,104, U.S. Pat. No. 2,595,605, U.S. Pat. No. 8,119,371, U.S. Pat. No. 2,771,397, U.S. Pat. Nos. 2,602,041, 2,695,261, 2,565,057 and 2,599,950 provides polymyxin production using Bacillus polymyxa. International PCT Application Publication No. WO2010/058427 provides fermentation process to produce polymyxin using Bacillus polymyxa. U.S. Pat. No. 7,935,503 provides methods for producing polymyxin using Paenibacillus amylolyticus. U.S. Pat. No. 4,091,092 provides process for the production of polymyxin F using Bacillus circulans. U.S. Pat. No. 2,565,057 provides methods to produce polymyxin using Bacillus aerosporus and Bacillus polymyxa. U.S. Pat. No. 8,652,819 provides production of polymyxin using Paenibacillus spp. Chinese Patent Nos. CN 103540633 and CN 101235407 provide production of polymyxin using Bacillus polymyxa. U.S. Pat. Nos. 2,908,611 and 4,748,117 provide fermentative methods to produce amphotericin B using Streptomyces nodosus. U.S. Pat. No. 6,132,993 improves the production of amphotericin B by inhibiting the production of amphotericin A. U.S. Pat. Nos. 2,809,151, 2,806,024, 2,808,363, 2,653,899, 2,833,693 and 2,834,714 provides fermentative production of erythromycin using Streptomyces erythreus. U.S. Pat. Nos. 5,908,764, 5,976,836 and 2,834,714 provide methods for enhancing the erythromycin productivity using Saccaropolyspora erythraea. U.S. Patent Application Publication No. 2011/0262971 provides genetically modified Escherichia coli strains for the production of erythromycin. International PCT Application Publication No. WO2012/166408 provides genetically modified Escherichia coli for the production of erythromycin analogs. U.S. Pat. Nos. 5,824,513 and 6,004,787 provides recombinant DNA for producing erythromycin analogs. U.S. Pat. No. 3,551,294 provides production of erythromycin using Arthrobacter sp. U.S. Pat. Nos. 2,957,810, 2,799,620, 3,022,228, and 3,386,889, provides method for the fermentative production of neomycin using Streptomyces fradiae. U.S. Pat. Nos. 2,504,067, 2,538,942, 2,538,943, 2,571,693, 2,515,461, 2,875,136, 2,808,364, 2,576,513, and 3,130,131 provides fermentative methods for the production of streptomycin using Streptomyces griseus. U.S. Pat. Nos. 2,541,726, 2,516,682, and 2,449,866 provide fermentative production of streptomycin using Actinomyces griseus. U.S. Pat. No. 3,993,544 provides method for producing streptomycin derivatives using mutant Streptomyces griseus. U.S. Pat. No. 2,545,572 provides production of streptomycin using mutant Actinomyces griseus. U.S. Pat. No. 2,617,755 provides process for producing hydroxystreptomycin using Streptomyces griseo-carneus. U.S. Pat. Nos. 3,429,780, 3,429,781, 2,712,517, 3,434,930, 2,739,924, 3,037,916, 3,037,917, 3,190,810, 3,280,188, 3,432,394, 2,763,591, 2,866,738, 2,776,243, 2,940,907, 2,940,908, 2,940,909, 3,092,556, 3,019,173, 3,121,670, 2,911,339, 3,317,403, 3,516,909, 2,940,905, 2,940,906, 3,007,965, 3,398,057, and 3,425,911 provide fermentation process for the production of tetracycline using Streptomyces spp. U.S. Pat. No. 5,223,413 provides process for the fermentative production of vancomycin using Micropolyspora orientalis. U.S. Pat. No. 3,067,099 provides fermentative production of vancomycin using Streptomyces orientalis. U.S. Patent Application Publication No. 2008/0193986 provides mutant Amycolatopsis orientalis for the production of vancomycin. U.S. Pat. Nos. 3,091,572, 4,279,997 and 4,209,511 provides fermentative methods to produce gentamicin using Micromonospora spp. Chinese Patent Nos. CN 102363759 and CN 101979647 provide engineered Micromonospora purpurea to produce gentamicin. International PCT application. No. WO2009/008664 provides genetically engineered microorganism to produce gentamicin. U.S. Pat. Nos. 3,871,965, 4,267,274, 4,263,404, 3,150,046, 3,597,324, and 4,298,692 provide fermentative production of rifamycin using mutant strain of Streptomyces mediterranei. U.S. Pat. Nos. 3,884,763, and 3,901,764 provide production of rifamycin using Micromonospora spp. U.S. Pat. No. 4,042,683 provides fermentative production of rifamycins P, Q and U. U.S. Pat. Nos. 3,871,965 and 3,871,966 provide production of rifamycin using Streptomyces albovinaceus. U.S. Pat. No. 4,431,735 provides biological process for the preparation of rifamycin derivatives. International PCT Application Publication No. WO2012/166408 provides genetically modified Escherichia coli strains for producing erythromycin analogs. U.S. Pat. Nos. 2,448,790, 2,761,812, 2,442,141, 2,830,934, 2,641,567, 2,538,721, 2,424,832, 2,443,989, 2,445,748, 2,698,274 and 3,024,169 provide fermentative methods and processes for penicillin production using Penicillium chrysogenum. U.S. Pat. No. 2,685,554 provides penicillin production using potassium phthalate as a fermentation buffer. U.S. Pat. Nos. 2,448,791, 2,476,107, 2,423,873, 2,611,733, 2,493,625, 2,609,330, 2,437,918 and 2,527,304 provide process of producing penicillin using penicillin producing strains. International PCT Application Publication No. WO2014/003555 provides improved penicillin production using Penicillium chrysogenum. U.S. Patent Application Publication No. 2004/0043491 and U.S. Patent Application Publication No. 2002/0058302 provides penicillin production using genetically altered Penicillium chrysogenum. U.S. Pat. Nos. 2,768,117, 2,440,357 and 2,440,358 provides production of penicillin employing media containing cysteine and cytidine. U.S. Pat. Nos. 2,475,920 and 2,451,853 provides manufacture of penicillin. U.S. Patent Application Publication No. 2008/0131925 provide beta-lactam antibiotics producing genetically engineered microorganism. U.S. Pat. No. 2,871,164 provides production of penicillin in the presence of a polythionate.

In one method to enhance DHA uptake and utilization, the genetically modified host cells already known to produce antibiotics, as described in patents above, is subjected to further genetic engineering by means of expressing plasmids that code ATP and PEP dependent kinases that could phosphorylate DHA and facilitate its entry into glycolytic cycle. In another method to enhance DHA uptake and utilization, genetically modified host cells already known to produce antibiotics, as described in patents above, is subjected to chemical mutagenesis and the strains with the ability to grow and produce desired antibiotics with high enough titer and yield in a growth medium comprising DHA as a source of carbon will be identified and subjected to whole genome sequencing to identify specific mutation associated with the ability to grow and produce antibiotics in a medium comprising DHA. Such a mutation will be introduced to the genetically modified host cells already known to produce antibiotics for the purpose of conferring the ability to use DHA as a source of organic carbon. In another method to enhance DHA uptake and utilization, genetically modified host cells already known to produce antibiotics, as described in patents above, is exposed to growth medium with increasing concentration of DHA and subjected to metabolic evolution. Strains that have gained ability to grow in the medium containing DHA as a major or sole source of carbon and still retain the ability to produce antibiotics is identified and subjected to whole genome sequencing to identify specific mutation associated with the ability to grow and produce antibiotics in a medium comprising DHA. Such a mutation will be introduced to the genetically modified host cells already known to produce antibiotics, as described in patents above, for the purpose of conferring the ability to use DHA as a source of organic carbon.

In another embodiment, the present invention provides methods of producing olefins such as styrene, isoprene, squalene, 4-hydroxystyrene, 3,4-dihydroxystyrene, 3-methoxy-4-hydroxystyrene, 3-methoxy-4,5-dihyroxystyrene, 3,5-dimethoxy-4-hydroxystyrene, vinyl indole, vinyl imidazole, isobutene, 2-methyl-1-butene, ethylene, propylene, 1-butene, 1-pentene, 1-heptene, 1-nonene, 1-undecene, 1-tridecene, 1-pentadecene, 1-heptadecene, 1-nonadecene, 1-heneicosene, 1-tricosene, 1-pentacosene and butadiene. In one aspect of the present invention, genetically modified microorganisms producing olefins are subjected to further genetic modifications to confer the ability to use DHA as a source of carbon. In another aspect of the present invention, microorganisms that have ability to grow in a medium comprising DHA as a source of carbon is subjected to further genetic modifications to produce olefins. Such a genetic modification would involve overexpressing one or more enzymes functional in the of the aromatic olefins biosynthetic pathway, branched olefin biosynthetic pathway, acetyl-CoA to isoprene biosynthetic pathway, pyruvate to isoprene biosynthetic pathway, acetyl-CoA to squalene biosynthetic pathway, pyruvate to squalene biosynthetic pathway, acetyl-CoA to butadiene biosynthetic pathway, succinyl-CoA to butadiene biosynthetic pathway, malonyl-CoA to butadiene biosynthetic pathway, pyruvate to butadiene biosynthetic pathway and linear olefins biosynthetic pathway. The list of specific genetic manipulations that are useful in the construction of a microbial strain useful in the production of one or other olefins using DHA as a source of carbon includes: enhancing the activities of one or more of the enzymes functional in the acetyl-CoA to butadiene biosynthetic pathway such as acetyl-CoA:acetyl-CoA acyl transferase, acetoactyl-CoA reductase, 3-hydroxybutyryl-CoA-dehydratase, crotonyl-CoA reductase, crotonate reductase, glutaconyl-CoA-decarboxylase, glutaryl-CoA-dehydrogenase, 3-aminobutyryl-CoA-deaminase, 4-hydroxybutyryl-CoA-dehydratase, crotonaldehyde reductase, crotyl alcohol kinase, crotyl alcohol phosphokinase and butadiene synthase; enhancing activities of one or more of the enzymes functional in the succinyl-CoA to butadiene biosynthetic pathway such as succinyl-CoA:acetyl-CoA acyltransferase, 3-oxoadipyl-CoA transferase, 3-oxoadipate dehydrogenase, 2-fumarylacetate decarboxylase, 3-oxopent-4-enoate reductase, 3-hydroxy-4-pentenoate dehydratase, 2,4-pentadienoate decarboxylase, 3-oxoadipyl-CoA reductase, 3-hydroxyadipyl-CoA transferase, 3-hydroxyadipate dehydrogenase, 3-hydroxy hex-4-enoate decarboxylase and 3-hydroxypent-4-enedioate decarboxylase; enhancing the activities of one or more of the enzymes functional in the malonyl-CoA to butadiene biosynthetic pathway such as malonyl-CoA: acetyl-CoA acyltransferase, 3-oxoglutaryl-CoA reductase, 3-hydroxyglutaryl-CoA reductase, 3-hydroxy-5-oxopentanoate reductase, 3,5-dihydroxypentanoate dehydratase, 5-hydroxypent-2-enoate decarboxylase, 3-butene-1-ol dehydratase 3,5-dihydroxypentanoate decarboxylase. 5-hydroxypent-2-enoate dehydratase, 2,4-penadienoate decarboxylase, 3-oxoglutaryl-CoA reductase, 3,5-dioxopentanoate reductase, 5-hydroxy-3-oxo-pentanoate reductase, 3,5-dihydroxypentanoate kinase, 3-hydroxy-5-phosphate pentanoate kinase, 3-hydroxy-5-diphosphate pentanoate decarboxylase, butenyl-4-diphosphate isomerase and butadiene synthase; enhancing the activities of one or more of the enzymes functional in the pyruvate to butadiene biosynthetic pathway such as 4-hydroxy-2-oxovalerate aldolase, 4-hydroxy-2-oxovalerate dehydratase, 2-oxopentenoate decarboxylase, crotonaldehyde reductase, crotyl alcohol kinase, 2-butenyl-4-phosphate kinase and butadiene synthase; enhancing one or more of the enzymes functional in the branched olefins biosynthetic pathway and in the linear olefins biosynthetic pathway; enhancing one or more of the enzymes functional in the aromatic olefins biosynthetic pathway such as β-hydroxyacid kinase, β-hydroxyacid phosphate kinase and β-hydroxyacid diphosphate decarboxylase; enhancing one or more of the enzymes phenylalanine ammonia lyase, tyrosine ammonia lyase, cinnamic acid decarboxylase, coumaric acid decarboxylase, caffeic acid decarboxylase, ferulic acid decarboxylase, hydroxyl ferulic acid decarboxylase, sinapic acid decarboxylase, cinnamate monooxygenase, coumarate-3-hydroxylase, caffeic acid-3-O-methyl transferase, ferulate-5-hydroxylase and caffeic acid-3-O methyl transferase; enhancing one or more of the enzymes functional in the in the acetyl-CoA to squalene biosynthetic pathway such as acetyl-CoA acyl transferase, HMG-CoA synthase, HMG-CoA reductase, mevalonate kinase, phosphomevalonate kinase, mevalonate pyrophosphate decarboxylate, IPP isomerase, dimethylallyl transferase, geranyl transferase and farnesyl-P2-farnesyl transferase; enhancing the activities of one or more enzymes functional in the in the pyruvate to squalene biosynthetic pathway such as DXP synthase, DXP reductoisomerase, IPP isomerase, dimethylallyl transferase, geranyl transferase and farnesyl-P2-farnesyl transferase and the enzymes facilitating the conversion of 2-C-methyl-D-erythritol-4-P to dimethylallyl pyrophosphate encoded by the genes ispD, ispE, ispF and ispH; enhancing the activities of one or more enzymes functional in the acetyl-CoA to isoprene biosynthetic pathway such as acetyl-CoA acyl transferase, HMG-CoA synthase, HMG-CoA reductase, mevalonate kinase, phosphomevalonate kinase, mevalonate pyrophosphate decarboxylate, IPP isomerase and isoprene synthase; enhancing the activities of one or more enzymes functional in the pyruvate to isoprene biosynthetic pathway such as DXP synthase, DXP reductoisomerase, isoprene synthase and the enzymes facilitating the conversion of 2-C-methyl-D-erythritol-4-P to dimethylallyl pyrophosphate encoded by the genes ispD, ispE, ispF and ispH.

In some embodiments, microorganisms that have ability to grow in DHA in a medium comprising DHA as a source of carbon is subjected to further genetic modifications to include one or more nucleic acid comprising a nucleotide sequence encoding isoprene biosynthetic pathway enzymes. Isoprene is made from acetyl-CoA by a series of enzyme catalyzed biochemical reactions via mevalonate pathway. In another method, isoprene is also made from glyceraldehyde-3P and pyruvate by a series of enzyme catalyzed biochemical reactions via DXP pathway. Microorganisms that already has an ability to produce high amounts of acetyl-CoA or pyruvate either natively or by one or more genetic modifications are a preferred host to produceisoprene. On the other hand, isoprene biosynthetic pathway can be directly engineered in to the host cell that previously lack the ability to produce pyruvate or acetyl-CoA but can be engineered to selectively produce isoprene. Furthermore, in addition to the enzymes that catalyze the formation of acetyl-CoA from DHA, one or more of the enzymes selected from a group of acetyl-CoA to isoprene biosynthetic pathway such as acetyl-CoA acyl transferase, HMG-CoA synthase, HMG-CoA reductase, mevalonate kinase, phosphomevalonate kinase, mevalonate pyrophosphate decarboxylate, IPP isomerase and isoprene synthase are overexpressed for the enhanced production of isoprene from acetyl-CoA. In addition, attenuation or deletion of one or more of enzymes methylglyoxal synthase (MgsA), pyruvate carboxylase, pyruvate-enolphosphate carboxylase, succinate reductase, citrate synthase, acetate kinase (AckA), aldehyde dehydrogenase (AldA), pyruvate oxidase (PoxB), pyruvate-formate lyase (PflB), acetolactate synthase, alanine transaminase, lactate dehydrogenase (Ldh) and acetyl-CoA carboxylase improves pyruvate utilization in isoprene biosynthesis. In another method to produce isoprene, pyruvate is used for the production of isoprene via DXP pathway. In addition to the enzymes that catalyze production of pyruvate from DHA, one or more of the enzymes selected from a group of pyruvate to isoprene biosynthetic pathway such as DXP synthase, DXP reductoisomerase, isoprene synthase and genes IspD, IspE, IspF, IspH encoding enzymes facilitating the conversion of 2-C-methyl-D-erythritol-4-P to dimethylallyl pyrophosphate are overexpressed for the enhanced production of isoprene from pyruvate. In addition, attenuation or deletion of one or more of enzymes methylglyoxal synthase (MgsA), pyruvate carboxylase, pyruvate-enolphosphate carboxylase, succinate reductase, citrate synthase, acetate kinase (AckA), aldehyde dehydrogenase (AldA), pyruvate oxidase (PoxB), pyruvate-formate lyase (PflB), acetolactate synthase, alanine transaminase, lactate dehydrogenase (Ldh), β-ketothiolase, HMG-CoA synthase and acetyl-CoA carboxylase improves pyruvate utilization in isoprene biosynthesis. Furthermore, attenuation or deletion of one or more of dimethylallyl pyrophosphate utilization pathway such as synthesis of geranyl-PP catalyzed catalyzed by dimethylallyl transferase, synthesis of farnesyl-PP catalyzed by farnesyl diphosphate synthase and synthesis of 3-methyl-2-butene-1-ol catalyzed by phosphatase improves dimethylallyl pyrophosphate utilization in isoprene synthesis. Furthermore, blocking one or more of isoprene degradation pathways such as oxidation reaction and polymerization reactions improves isoprene accumulation. Furthermore, improving isoprene transport, reducing isoprene uptake from media and reducing feedback inhibition increases isoprene productivity.

In some embodiments, microorganisms that have ability to grow in DHA in a medium comprising DHA as a source of carbon is subjected to further genetic modifications to include one or more nucleic acid comprising a nucleotide sequence encoding squalene biosynthetic pathway enzymes. Squalene is made from acetyl-CoA by a series of enzyme catalyzed biochemical reactions via mevalonate pathway. In another method, squalene is also made from glyceraldehyde-3P and pyruvate by a series of enzyme catalyzed biochemical reactions via DXP pathway. Microorganisms that already has an ability to produce high amounts of acetyl-CoA or pyruvate either natively or by one or more genetic modifications are a preferred host to produce squalene. On the other hand, squalene biosynthetic pathway can be directly engineered in to the host cell that previously lack the ability to produce pyruvate or acetyl-CoA but can be engineered to selectively produce squalene. Furthermore, in addition to the enzymes that catalyze the formation of acetyl-CoA from DHA, one or more of the enzymes selected from a group of acetyl-CoA to squalene biosynthetic pathway such as acetyl-CoA acyl transferase, HMG-CoA synthase, HMG-CoA reductase, mevalonate kinase, phosphomevalonate kinase, mevalonate pyrophosphate decarboxylate, IPP isomerase, dimethylallyl transferase, geranyl transferase and farnesyl-P2-farnesyl transferase are overexpressed for the enhanced production of squalene from acetyl-CoA. In addition, attenuation or deletion of one or more of enzymes methylglyoxal synthase (MgsA), pyruvate carboxylase, pyruvate-enolphosphate carboxylase, succinate reductase, citrate synthase, acetate kinase (AckA), aldehyde dehydrogenase (AldA), pyruvate oxidase (PoxB), pyruvate-formate lyase (PflB), acetolactate synthase, alanine transaminase, lactate dehydrogenase (Ldh) and acetyl-CoA carboxylase improves pyruvate utilization in squalene biosynthesis. In another method to produce squalene, pyruvate is used to produce squalene via DXP pathway. In addition to the enzymes that catalyze production of pyruvate from DHA, one or more of the enzymes selected from a group of pyruvate to squalene biosynthetic pathway such as DXP synthase, DXP reductoisomerase, IPP isomerase, dimethylallyl transferase, geranyl transferase and farnesyl-P2-farnesyl transferase and genes IspD, IspE, IspF, IspH encoding enzymes facilitating the conversion of 2-C-methyl-D-erythritol-4-P to dimethylallyl pyrophosphate are overexpressed for the enhanced production of squalene from pyruvate. In addition, attenuation or deletion of one or more of enzymes methylglyoxal synthase (MgsA), pyruvate carboxylase, pyruvate-enolphosphate carboxylase, succinate reductase, citrate synthase, acetate kinase (AckA), aldehyde dehydrogenase (AldA), pyruvate oxidase (PoxB), pyruvate-formate lyase (PflB), acetolactate synthase, alanine transaminase, lactate dehydrogenase (Ldh), β-ketothiolase, HMG-CoA synthase and acetyl-CoA carboxylase improves pyruvate utilization in squalene biosynthesis. Furthermore, attenuation or deletion of one or more of farnesyl pyrophosphate utilization pathway such as synthesis of geranylgeranyl-PP catalyzed catalyzed by farnesyl-trans-transferase, synthesis of sesquiterpenes and synthesis of dehydrodolichol-PP catalyzed by polyprenyl-cis-transferase improves farnesyl pyrophosphate utilization in squalene synthesis. Furthermore, blocking one or more of squalene degradation pathways such as oxidation reaction catalyzed by squalene monooxygenase and polymerization reactions improves squalene accumulation. Furthermore, improving squalene transport, reducing squalene uptake from media and reducing feedback inhibition increases squalene productivity.

In some embodiments, microorganisms that have ability to grow in DHA in a medium comprising DHA as a source of carbon is subjected to further genetic modifications to include one or more nucleic acid comprising a nucleotide sequence encoding aromatic olefins biosynthetic pathway enzymes. Aromatic olefins are made from aromatic amino acids such as phenylalanine and tyrosine by a series of enzyme catalyzed biochemical reactions. Microorganisms that already has an ability to produce high amounts of aromatic amino acids either natively or by one or more genetic modifications are the preferred host to produce aromatic olefins. On the other hand, aromatic olefins biosynthetic pathway can be directly engineered in to the host cell that previously lack the ability to produce aromatic amino acids but can be engineered to selectively produce aromatic olefins. Furthermore, in addition to the enzymes that catalyze the formation of phenylalanine and tyrosine from DHA, one or more of the enzymes selected from a group of aromatic olefins biosynthetic pathway such as phenylalanine ammonia lyase, tyrosine ammonia lyase, cinnamic acid decarboxylase, coumaric acid decarboxylase, caffeic acid decarboxylase, ferulic acid decarboxylase, hydroxyl ferulic acid decarboxylase, sinapic acid decarboxylase, cinnamate monooxygenase, coumarate-3-hydroxylase, caffeic acid-3-O-methyl transferase, ferulate-5-hydroxylase and caffeic acid-3-O methyl transferase are overexpressed for the enhanced production of aromatic olefins from phenylalanine and tyrosine. In addition, attenuation or deletion of one or more of enzymes methylglyoxal synthase (MgsA), pyruvate carboxylase, phopsphoenolpyruate carboxylase, succinate reductase, citrate synthase, acetate kinase (AckA), aldehyde dehydrogenase (AldA), pyruvate oxidase (PoxB), pyruvate-formate lyase (PflB), acetolactate synthase, alanine transaminase, lactate dehydrogenase (Ldh), β-ketothiolase, HMG-CoA synthase and acetyl-CoA carboxylase improves pyruvate utilization in aromatic aminoacid biosynthesis. Furthermore, attenuation or deletion of one or more of aromatic aminoacids degradation pathway such as synthesis of pyruvates catalyzed by aspartate amino transferase, synthesis of amines catalyzed by aromatic amino acid decarboxylase and synthesis of β-aminoacids catalyzed by amino acid 2,3 amino mutase improves aromatic amino acid utilization in aromatic olefins synthesis. Furthermore, blocking one or more of aromatic olefins degradation pathways such as oxidation reaction and polymerization reactions improves aromatic olefins accumulation. Furthermore, improving aromatic olefins transport, reducing aromatic olefins uptake from media and reducing feedback inhibition increases aromatic olefins productivity.

In some embodiments, microorganisms that have ability to grow in DHA in a medium comprising DHA as a source of carbon is subjected to further genetic modifications to include one or more nucleic acid comprising a nucleotide sequence encoding branched olefins biosynthetic pathway enzymes. Branched olefins are made from β-hydroxy carboxylicacids by a deoxydecarboxylation reaction catalyzed by β-hydroxyacids diphosphate decarboxylase. Microorganisms that already has an ability to produce high amounts of β-hydroxy acids either natively or by one or more genetic modifications are a preferred host to produce branched olefins. On the other hand, branched olefins biosynthetic pathway can be directly engineered in to the host cell that previously lack the ability to produce β-hydroxy acids but can be engineered to selectively produce branched olefins. Furthermore, in addition to the enzymes that catalyze the formation of β-hydroxy carboxylicacids from DHA, one or more of the enzymes selected from a group of branched olefins biosynthetic pathway such as β-hydroxyacid kinase, β-hydroxyacid phosphate kinase and β-hydroxyacid diphosphate decarboxylase are overexpressed for the enhanced production of branched olefins. In another method, 3-hydroxypropionic acid is converted to ethylene by means of expressing one or more enzymes selected from branched chain biosynthetic pathway such as 3HP-kinase, 3HP phosphate kinase and β-hydroxyacid diphosphate decarboxylase. In another method, 3-hydroxybutyric acid is converted to propylene by means of expressing one or more enzymes selected from branched chain biosynthetic pathway such as 3HB-kinase, 3HB phosphate kinase and β-hydroxyacid diphosphate decarboxylase. In another method, β-hydroxyacid derived from fatty acid synthesis cycle is used to express one or more or enzymes selected from branched olefins biosynthetic pathway such as β-hydroxyacid kinase, β-hydroxyacid phosphate kinase and β-hydroxyacid diphosphate decarboxylase for the enhanced production of linear olefins from β-hydroxyfattyacid. In another method, 3-methyl-3-hydroxybutanoate derived from 2-ketoisovalerate using enzymes such as 2-hydroxyacid dehydrogenase, butyrate-CoA ligase, 2-hydroxy isovaleryl-CoA dehydratase, enoyl-CoA hydratase and thioesterase is used to express one or more or enzymes selected from branched olefins biosynthetic pathway such as β-hydroxyacid kinase, β-hydroxyacid phosphate kinase and β-hydroxyacid diphosphate decarboxylase for the enhanced production of isobutene from 3-methyl-3-hydroxybutanoate. In another method, 3-methyl-3-hydroxypentanoate derived from 2-keto-3-methylvalerate using enzymes such as 2-hydroxyacid dehydrogenase, butyrate-CoA ligase, 2-hydroxy isovaleryl-CoA dehydratase, enoyl-CoA hydratase and thioesterase is used to express one or more or enzymes selected from branched olefins biosynthetic pathway such as β-hydroxyacid kinase, β-hydroxyacid phosphate kinase and β-hydroxyacid diphosphate decarboxylase for the enhanced production of 2-methyl-1-butene from 3-methyl-3-hydroxypentanoate. In addition, attenuation or deletion of one or more of enzymes methylglyoxal synthase (MgsA), succinate reductase, citrate synthase, acetate kinase (AckA), aldehyde dehydrogenase (AldA), pyruvate oxidase (PoxB), pyruvate-formate lyase (PflB), alanine transaminase, lactate dehydrogenase (Ldh), β-ketothiolase, HMG-CoA synthase and acetyl-CoA carboxylase improves pyruvate utilization in 3-methyl-3-hydroxy butanoate and 3-methyl-3-hydroxy pentanoate biosynthesis. Furthermore, attenuation or deletion of one or more of 2-ketoacids degradation pathway such as synthesis of valine and isoleucine catalyzed by branched chain amino acid transaminase, synthesis of acyl-CoA catalyzed by 2-ketoacid dehydrogenase and synthesis of pantothenate catalyzed by 2-oxoisovalerate hydroxylmethyl transferase improves 2-ketoacid utilization in branched olefins synthesis. Furthermore, blocking one or more of branched olefins degradation pathways such as oxidation reaction and polymerization reactions improves branched olefins accumulation. Furthermore, improving branched olefins transport, reducing branched olefins uptake from media and reducing feedback inhibition increases branched olefins productivity.

In some embodiments, microorganisms that have ability to grow in DHA in a medium comprising DHA as a source of carbon is subjected to further genetic modifications to include one or more nucleic acid comprising a nucleotide sequence encoding linear olefins biosynthetic pathway enzymes. Linear olefins are made from α,β-unsaturated acids such as but not limited to acrylate, crotonoate, 2-hexenoate, 2-octanoate by a decarboxylation reaction catalyzed by aromatic α,β-unsaturated acids decarboxylase. Microorganisms that already has an ability to produce high amounts of α,β-unsaturated acids either natively or by one or more genetic modifications are a preferred host to produce linear olefins. On the other hand, linear olefins biosynthetic pathway can be directly engineered in to the host cell that previously lack the ability to produce α,β-unsaturated acids but can be engineered to selectively produce linear olefins. One or more of the enzymes selected from a group of linear olefins biosynthetic pathway such as cinnamic acid decarboxylase, ferulic acid decarboxylate, coumaric acid decarboxylate and caffeic acid decarboxylase are generally classified as α,β-unsaturated acid decarboxylase. In addition to the enzymes catalyze the formation of α,β-unsaturated acids from DHA, α,β-unsaturated acid decarboxylases are overexpressed for the enhanced production of linear olefins from α,β-unsaturated acids. In one method, α,β-unsaturated fatty acids derived from fatty acid synthesis cycle is used to express one or more or enzymes selected from linear olefins biosynthetic pathway such as α,β-unsaturated acid decarboxylase for the enhanced production of linear olefins from α,β-unsaturated fatty acids. In another method, 3-methyl crotonate derived from HMG-CoA using enzymes such as 3-methyl glutaconyl-CoA hydratase, 3-methyl crotonyl-CoA carboxylase and CoA hydroxylase is used to express one or more or enzymes selected from linear olefins biosynthetic pathway such as α,β-unsaturated acid decarboxylase for the enhanced production of isobutene from 3-methyl crotonate. In another method, 2-pentenoate derived from propionyl-CoA using enzymes such as acetyl-CoA-C-acyl transferase, 3-ketovaleryl-CoA reductase, 3-hydroxyvaleryl-CoA dehydrogenase and CoA hydrolase is used to express one or more or enzymes selected from linear olefins biosynthetic pathway such as α,β-unsaturated acid decarboxylase for the enhanced production of 1-butene from 3-methyl crotonate. In addition, attenuation or deletion of one or more of enzymes methylglyoxal synthase (MgsA), pyruvate carboxylase, phosphoenolpyruvate carboxylase, succinate reductase, citrate synthase, acetate kinase (AckA), aldehyde dehydrogenase (AldA), pyruvate oxidase (PoxB), pyruvate-formate lyase (PflB), alanine transaminase, lactate dehydrogenase (Ldh), β-ketothiolase and HMG-CoA synthase improves pyruvate utilization in α,β-unsaturated acids biosynthesis. Furthermore, blocking one or more of linear olefin degradation pathways such as oxidation reaction and polymerization reactions improves linear olefin accumulation. Furthermore, improving linear olefin transport, reducing linear olefin uptake from media and reducing feedback inhibition increases linear olefin productivity.

In some embodiments, microorganisms that have ability to grow in DHA in a medium comprising DHA as a source of carbon is subjected to further genetic modifications to include one or more nucleic acid comprising a nucleotide sequence encoding butadiene pathway enzymes. Butadiene is made from acetyl-CoA by a series of enzyme catalyzed biochemical reactions. Butadiene is also made from succinyl-CoA by a series of enzyme catalyzed biochemical reactions. In another method to produce butadiene, malonyl-CoA is converted to butadiene by a series of enzyme catalyzed biochemical reactions. In yet another method to produce butadiene, metabolite pyruvate is converted to butadiene by a series of enzyme catalyzed biochemical reactions. Microorganisms that already has an ability to produce high amounts of acetyl-CoA, succinyl-CoA, malonyl-CoA and pyruvate either natively or by one or more genetic modifications are a preferred host to produce butadiene. On the other hand, butadiene biosynthetic pathway can be directly engineered in to the host cell that previously lack the ability to produce acetyl-CoA, succinyl-CoA, malonyl-CoA and pyruvate but can be engineered to selectively produce butadiene. Furthermore, in addition to the enzymes required for the production of acetyl-CoA from DHA, one or more of the enzymes selected from a group of acetyl-CoA to butadiene biosynthetic pathway such as acetyl-CoA:acetyl-CoA acyl transferase, acetoactyl-CoA reductase, 3-hydroxybutyryl-CoA-dehydratase, crotonyl-CoA reductase, crotonate reductase, glutaconyl-CoA-decarboxylase, glutaryl-CoA-dehydrogenase, 3-aminobutyryl-CoA-deaminase, 4-hydroxybutyryl-CoA-dehydratase. crotonaldehyde reductase, crotyl alcohol kinase, crotyl alcohol phosphokinase and butadiene synthase are overexpressed for the enhanced production of butadiene from acetyl-CoA. In addition, attenuation or deletion of one or more of methylglyoxal synthase (MgsA), phosphoenolpyruvate carboxylate, pyruvate carboxylase, acetate kinase (AckA), aldehyde dehydrogenase (AldA), pyruvate oxidase (PoxB), succinate dehydrogenase (FrdBC), acetyl-CoA carboxylase, hydroxymethyl-CoA glutaryl synthase, citrate synthase, alanine transaminase and lactate dehydrogenase improves pyruvate utilization in butadiene synthesis. In another method to produce butadiene, metabolite succinyl-CoA is used to produce butadiene. In addition to the enzymes catalyze the production of succinyl-CoA from DHA, one or more of the enzymes selected from a group of succinyl-CoA to butadiene biosynthetic pathway such as succinyl-CoA: acetyl-CoA acyltransferase, 3-oxoadipyl-CoA transferase, 3-oxoadipate dehydrogenase, 2-fumarylacetate decarboxylase, 3-oxopent-4-enoate reductase, 3-hydroxy-4-pentenoate dehydratase, 2,4-pentadienoate decarboxylase, 3-oxoadipyl-CoA reductase, 3-hydroxyadipyl-CoA transferase, 3-hydroxyadipate dehydrogenase, 3-hydroxy hex-4-enoate decarboxylase and 3-hydroxypent-4-enedioate decarboxylase are overexpressed for the enhanced production of butadiene from succinyl-CoA Moreover, improvement of succinate production by overexpressing one or more enzymes in succinate biosynthetic pathway enhances the productivity of butadiene from succinyl-CoA. In addition, attenuation or deletion of one or more of methylglyoxal synthase (MgsA), pyruvate dehydrogenase, acetate kinase (AckA), aldehyde dehydrogenase (AldA), pyruvate oxidase (PoxB), acetyl-CoA carboxylase, beta-ketothiolase, hydroxymethyl-CoA glutaryl synthase, citrate synthase, alanine transaminase, acetolactate synthase and lactate dehydrogenase improves pyruvate utilization in butadiene synthesis. In another method to produce butadiene, metabolite malonyl-CoA is used for the production of butadiene. In addition to the enzymes catalyze production of malonyl-CoA from DHA, one or more of the enzymes selected from a group of malonyl-CoA to butadiene biosynthetic pathway such as malonyl-CoA:acetyl-CoA acyltransferase, 3-oxoglutaryl-CoA reductase, 3-hydroxyglutaryl-CoA reductase, 3-hydroxy-5-oxopentanoate reductase, 3,5-dihydroxypentanoate dehydratase, 5-hydroxypent-2-enoate decarboxylase, 3-butene-1-ol dehydratase 3,5-dihydroxypentanoate decarboxylase. 5-hydroxypent-2-enoate dehydratase, 2,4-penadienoate decarboxylase, 3-oxoglutaryl-CoA reductase, 3,5-dioxopentanoate reductase, 5-hydroxy-3-oxo-pentanoate reductase, 3,5-dihydroxypentanoate kinase, 3-hydroxy-5-phosphate pentanoate kinase, 3-hydroxy-5-diphosphate pentanoate decarboxylase, butenyl-4-diphosphate isomerase and butadiene synthase are overexpressed for the enhanced production of butadiene from malonyl-CoA. In addition, attenuation or deletion of one or more of methylglyoxal synthase (MgsA), pyruvate carboxylase, phosphoenolpyruvate carboxylase, succinate reductase, acetate kinase (AckA), aldehyde dehydrogenase (AldA), pyruvate oxidase (PoxB), citrate synthase, alanine transaminase and lactate dehydrogenase improves pyruvate utilization in butadiene synthesis. In another method to produce butadiene, pyruvate is used to produce butadiene. In addition to the enzymes catalyze production of pyruvate from DHA, one or more of the enzymes selected from a group of pyruvate to butadiene biosynthetic pathway such as 4-hydroxy-2-oxovalerate aldolase, 4-hydroxy-2-oxovalerate dehydratase, 2-oxopentenoate decarboxylase, crotonaldehyde reductase, crotyl alcohol kinase, 2-butenyl-4-phosphate kinase and butadiene synthase are overexpressed for the enhanced production of butadiene from pyruvate. In addition, attenuation or deletion of one or more of methylglyoxal synthase (MgsA), pyruvate carboxylase, phosphoenolpyruvate carboxylase, succinate reductase, acetate kinase (AckA), aldehyde dehydrogenase (AldA), pyruvate oxidase (PoxB), citrate synthase, alanine transaminase, acetyl-CoA carboxylase, β-ketothiolase, HMG-CoA synthase, acetolactate synthase and lactate dehydrogenase improves pyruvate utilization in butadiene synthesis. Furthermore, blocking one or more of butadiene degradation pathways such as oxidation reaction and polymerization reactions improves butadiene accumulation. Furthermore, improving butadiene transport, reducing butadiene uptake from media and reducing feedback inhibition increases butadiene productivity.

Genetically modified microorganisms capable of producing “olefins” using conventional sugars such as glucose, sucrose or glycerol are already known in the art. U.S. Patent Application Publication No. 2011/0243969 provides production of squalene from Saccharomyces spp. U.S. Patent application publication No. 2009/0298150 provides production of squalene from a genetically modified yeast. U.S. Pat. No. 9,249,429 provides strain belonging to Aurantiochytrium for the squalene production process. Chinese Patent No. CN 103266137 provides exogenous squalene synthetase expression in Escherichia coli for the production of squalene. U.S. Patent Application Publication No. 2011/0243969 provides production of squalene from hyper-producing yeasts. U.S. Pat. No. 8,470,568 provides Yarrowia lipolytica for the production of squalene. U.S. Pat. No. 8,679,804 provides modified yeast strain for the production of squalene. U.S. Patent Application Publication No. 2013/0288327 provides novel microorganism belonging to the genus Aurantiochytrium for the high squalene producing ability. U.S. Patent Application Publication No. 2014/0113015 provides novel strain of Schizochytrium sp. to produce squalene. U.S. Patent Application Publication No. 2010/0184178 provides increased isoprene production using genetically engineered mevalonate kinase and isoprene synthase comprising microorganism. U.S. Patent application publication No. 2014/0234926 provides acetogenic bacteria to produce isoprene from syngas. U.S. Patent Application Publication No. 2016/0017374 provides methanotrophic bacterium to produce isoprene from methane. U.S. Pat. No. 5,849,970 provides Bacillus to produce isoprene. U.S. Pat. No. 8,975,051 provides increased isoprene production by decreasing IspA activity. U.S. Pat. Nos. 8,173,410, 9,163,263 and 8,735,134 provide variants of isoprene synthase for the improved isoprene production. U.S. Pat. Nos. 8,815,548 and 8,361,762 provide Archaeal mevalonate kinase polypeptide that are feedback resistant to produce isoprene. U.S. Patent Application Publication No. 2014/0335576 provides increased isoprene production using glucose and acetate co-metabolism. U.S. Patent Application Publication No. 2011/0045563 provides isoprene production using Ascomycota microorganism. U.S. Pat. No. 8,507,235 provides improved isoprene production using both DXP and MVA pathway. U.S. Patent Application Publication No. 2014/0377845 provides microorganism comprising isoprene synthase and isopentenyl-PP isomerase for improved isoprene production. U.S. Patent Application Publication No. 2014/0234937 provides production of isoprene from lignocellulosic plant biomass. U.S. Patent Application Publication No. 2015/0284742 provides recombinant cell to produce isoprene. International PCT application. No. WO2013/119340 provides microorganisms that has been modified to overexpress genes PntA and PntB to produce isoprene. U.S. Pat. No. 8,741,612 and U.S. Pat. No. 8,741,613 provides microorganisms comprising isoprene biosynthetic pathway to produce isoprene. U.S. Pat. Nos. 8,569,026 and 9,121,039 provide production of isoprene using cultured cells. U.S. Pat. No. 8,470,581 provides reduction of carbondioxide during isoprene production by fermentation. U.S. Patent Application Publication No. 2011/0039323 provides transgenic isoprene producing host microorganism. U.S. Patent Application Publication No. 2014/0273145 provides production of isoprene under neutral pH conditions. U.S. Pat. No. 8,685,702 provides improved isoprene production using two types of IspG enzymes. U.S. Pat. Nos. 8,709,785, 9,260,727 and 8,288,148 provides recombinant bacterial cells capable of producing enhanced isoprene. U.S. Pat. No. 8,993,305 provides utilization of phosphoketolase to produce isoprene. U.S. Pat. No. 8,962,296 provides method of producing isoprene monomer using gene encoding isoprene synthase. U.S. Pat. No. 8,916,370 provides isoprene synthase variants for the improved isoprene production. International PCT Application Publication No. WO2014/202838 provides methods of producing styrene using genetically modified microorganisms. U.S. Pat. No. 9,157,099 provides methods of preparing hydrocarbons via decarboxylation of alpha-beta-unsaturated carboxylic acids. U.S. Pat. No. 9,150,884 provides microbial conversion of glucose to styrene and its derivatives using recombinant Escherichia coli. U.S. Patent Application Publication No. 2015/0337336 provides enzymes and methods to produce styrene. U.S. Pat. No. 7,229,806 provides methods to produce 4-hydroxystyrene from glucose using genetically engineered microorganisms. U.S. Pat. No. 8,580,543 provides methods and microorganisms to produce butadiene. U.S. Patent Application Publication No. 2015/0017698 provides recombinant host cells to produce 1,3-butadiene. U.S. Patent Application Publication No. 2016/0032325 provides modified method and microorganisms to produce butadiene. U.S. Pat. Nos. 9,169,486, 8,580,543, and 8,715,957 and U.S. Patent Application Publication Nos. 2015/0064760, 2015/0064750 and 2013/0109064 provide various methods and microorganisms to produce 1,3-butadiene. U.S. Patent Application Publication Nos. 2014/0141482 and 2013/0189753 provide novel biochemical pathways for producing butadiene. U.S. Patent Application Publication No. 2012/0225466 provides non-naturally occurring microorganisms having butadiene pathway. International PCT Application Publication No. WO2013/082542 provides methods involving mevalonate diphosphate decarboxylate pathway to produce butadiene. International PCT Application. Publication Nos. WO2015/041776 and WO2014/063156 provides enzymes and microorganisms to produce butadiene. International PCT Application Publication No. WO2016/034691 provides recombinant microorganism to produce alkenes from acetyl-CoA. International PCT Application Publication No. WO2013/188546 provides methods for biosynthesizing butadiene. U.S. Patent Application Publication No. 2014/0186913 provides methods for the biosynthesis of isobutene. International PCT Application Publication No. WO2013/071074 provides methods for producing butadiene. U.S. Patent Application Publication No. 2014/0134687 provides modified microorganisms and methods of using same for producing butadiene and succinate. U.S. Patent Application Publication No. 2011/0165644 and U.S. Pat. No. 9,193,978 provide methods for production of alkenes by enzymatic decarboxylation of 3-hydroxy alkanoic acid. International PCT Application. Publication No. WO2016/042011 provides methods for producing isobutene from 3-methylcrtonyl-CoA. U.S. Pat. No. 9,023,636 provides microorganisms and methods for the biosynthesis of propylene. U.S. Pat. No. 8,617,862 provides methods and microorganism for producing propylene.

In one method to enhance DHA uptake and utilization, the genetically modified host cells already known to produce olefins, as described in patents above, is subjected to further genetic engineering by means of expressing plasmids that code ATP and PEP dependent kinases that could phosphorylate DHA and facilitate its entry into glycolytic cycle. In another method to enhance DHA uptake and utilization, genetically modified host cells already known to produce olefins, as described in patents above, is subjected to chemical mutagenesis and the strains with the ability to grow and produce desired antibiotics with high enough titer and yield in a growth medium comprising DHA as a source of carbon will be identified and subjected to whole genome sequencing to identify specific mutation associated with the ability to grow and produce olefins in a medium comprising DHA. Such a mutation will be introduced to the genetically modified host cells already known to produce olefins to confer the ability to use DHA as a source of organic carbon. In another method to enhance DHA uptake and utilization, genetically modified host cells already known to produce olefins, as described in patents above, is exposed to growth medium with increasing concentration of DHA and subjected to metabolic evolution. Strains that have gained ability to grow in the medium containing DHA as a major or sole source of carbon and still retain the ability to produce olefins is identified and subjected to whole genome sequencing to identify specific mutation associated with the ability to grow and produce olefins in a medium comprising DHA. Such a mutation will be introduced to the genetically modified host cells already known to produce olefins, as described in patents above, to confer the ability to use DHA as a source of organic carbon.

In another embodiment, the present invention provides methods for producing flavonoids using DHA as a source of organic carbon in a biological fermentation. In one aspect, genetically modified microorganisms producing flavonoids are subjected to further genetic modifications to confer the ability to use DHA as a source of carbon. In another aspect of this invention, microorganisms that have ability to grow in a medium comprising DHA as a source of carbon is subjected to further genetic modifications to produce flavonoids. The genetic modification required to construct a microbial organism to produce one or other type of flavonoids would involve overexpressing one or more components of the phenylpropanoid biosynthetic pathway, vanillin and capsaicin biosynthetic pathway, flavanone and flavonol biosynthetic pathway, isoflavonoid biosynthetic pathway, flavonoid biosynthetic pathway, stilbenoid, gingerol and diarylheptanoid biosynthetic pathway. The list of genetic modifications required to construct a microbial strain to produce one or other flavonoids includes: enhancing activities of one or more of the enzymes functional in the vanillin and capsaicin biosynthetic pathway such as feruloyl-CoA hydratase, vanillin synthase, vanillin aminotransferase and capsaicin synthase; enhancing activities of one or more of the enzymes functional in the in the stilbenoid, diarylheptanoid and gingerolbiosynthetic pathway such as resveratrol di-O-methyltransferase, stilbene synthase, pinosylvin synthase, phenylpropanoylacetyl-CoA synthase and gingerol synthase; enhancing activities of one or more enzymes in the flavanone and flavonol biosynthetic pathway such as flavonoid 3′, 5′-hydroxylase, flavone-3-O-methyltransferase, flavonol-3-O-methyltransferase, flavone-7-O-methyltransferase, flavonol-4′-O-methyltransferase and flavonoid-O-methyltransferase; enhancing activities of one or more enzymes functional in the isoflavonoid biosynthetic pathway such as 2-hydroxyisoflavanone synthase, 2-hydroxyisoflavanone dehydratase, isoflavone-2′-hydroxylase, isoflavone-7-O-methyltransferase, isoflavone-3′-hydroxylase and 2′-hydroxy isoflavone reductase; enhancing activities of one or more enzymes functional in the flavonoid biosynthetic pathway such as chalcone synthase, chalcone isomerase, naringenin 3-dioxygenase, dihydroflavinol-4-reductase, leucoanthocyanin dioxygenase, anthocyanidin reductase, leucoanthocyanidin reductase, flavonol synthase and flavonone synthase; enhancing the activities of one or more enzymes functional in the phenylpropanoid biosynthetic pathway such as phenylalanine ammonia lyase, tyrosine ammonia lyase, cinnamate monooxygenase, coumarate-3-hydroxylase, caffeic acid-3-O methyl transferase, ferulate-5-hydroxylase α,β-unsaturated acid CoA ligase, α,β-unsaturated acid CoA reductase, α,β-unsaturated alcohol dehydrogenase, coniferyl alcohol acyl transferase, eugenol synthase, eugenol-O-methyl transferase and isoeugenol synthase

In one aspect of the present invention, microorganisms that have ability to grow in a medium comprising DHA as a source of carbon is subjected to further genetic modifications to include one or more nucleotide sequences encoding flavonoid biosynthetic pathway enzymes. Flavonoids are made from α,β-unsaturated aromatic acyl-CoA such as cinnamyl-CoA, p-coumaroyl-CoA, caffeoyl-CoA and feruloyl-CoA by a series of enzyme catalyzed biochemical reactions. Microorganisms that already has an ability to produce high amounts of α,β-unsaturated aromatic acyl-CoA either natively or by one or more genetic modifications are a preferred host to produce flavonoids. On the other hand, flavonoid biosynthetic pathway can be directly engineered in the host cell that previously lack the ability to produce α,β-unsaturated aromatic acyl-CoA but can be engineered to selectively produce flavonoids. Furthermore, in addition to the enzymes that catalyze the formation of α,β-unsaturated aromatic acyl-CoA from DHA, one or more of the enzymes selected from a group of flavonoid biosynthetic pathway such as chalcone synthase, chalcone isomerase, naringenin 3-dioxygenase, dihydroflavonol 4-reductase, leucoanthocyanidin dioxygenase and anthocyanidin reductase are overexpressed for the enhanced production of flavonoid from α,β-unsaturated aromatic acyl-CoA. In one method, cinnamoyl-CoA is converted to pinocembrin and pinobanksin by one or more of the enzymes from flavonoids biosynthetic pathway such as chalcone synthase, chalcone isomerase and naringenin 3-dioxygenase. These enzymes functional in the flavonoid pathway are overexpressed for the enhanced production of pinocembrin and pinobanksin from cinnamoyl-CoA. In another method, butin, liquiritigenin, garbanzol, dihydrofisetin, 5-deoxyleucopelargonidin and 5-dexoyleucocyanidin are produced from p-coumaroyl-CoA by one or more of the enzymes from flavonoids biosynthetic pathway such as chalcone synthase, chalcone isomerase, naringenin 3-dioxygenase and dihydroflavinol which are overexpressed for the enhanced production of flavonoids from p-coumaroyl-CoA. In another method, naringenin, dihydrokaempferol, apiforol, apigenin, kaempferol, leucopelargonidin and epiafzelechin are produced from p-coumaroyl-CoA by one or more of the enzymes from flavonoids biosynthetic pathway such as chalcone synthase, chalcone isomerase, naringenin 3-dioxygenase, dihydroflavinol-4-reductase, leucoanthocyanin dioxygenase, anthocyanidin reductase and flavonol synthase which are overexpressed for the enhanced production of flavonoids from p-coumaroyl-CoA. In another method, eriodictyolluteolin, luteoforol, dihydroquercetin, quercetin, leucocyanidin, cyaniding, catechin and epicatechin are produced from caffeoyl-CoA by one or more of the enzymes from flavonoids biosynthetic pathway such as chalcone synthase, chalcone isomerase, naringenin 3-dioxygenase, dihydroflavinol-4-reductase, leucoanthocyanin dioxygenase, anthocyanidin reductase, leucoanthocyanidin reductase and flavonone synthase which are overexpressed for the enhanced production of flavonoids from caffeoyl-CoA. In another method, homoeriodictyol, dihydrotricetin, tricetin, dihydromyricetin, leucodelphinidin, delphinidin, gallocatechin, and epigallocatechin are produced from feruloyl-CoA by one or more of the enzymes from flavonoids biosynthetic pathway such as chalcone synthase, chalcone isomerase, naringenin 3-dioxygenase, dihydroflavinol-4-reductase, leucoanthocyanin dioxygenase, anthocyanidin reductase, leucoanthocyanidin reductase, flavonol synthase and flavonone synthase which are overexpressed for the enhanced production of flavonoids from feruloyl-CoA. In addition, attenuation or deletion of one or more of enzymes such as methylglyoxal synthase, pyruvate carboxylase, phosphoenolpyruvate carboxylase, succinate reductase, citrate synthase, acetate kinase, aldehyde dehydrogenase, pyruvate oxidase, pyruvate-formate lyase, alanine transaminase, lactate dehydrogenase, β-ketothiolase, acetyl-CoA carboxylase and HMG-CoA synthase improves pyruvate utilization in flavonoid biosynthesis. Furthermore, enhancing one or more of the enzymes from aromatic amino acid biosynthetic pathways and reducing aromatic amino acids degradation pathways improves aromatic amino acid availability for the flavonoid synthesis. Furthermore, blocking one or more of flavonoid degradation pathways such as oxidation reaction, glucoside formation and lignin synthesis improves flavonoid accumulation. Furthermore, improving flavonoid transport, reducing flavonoid uptake from media and reducing feedback inhibition increases flavonoid productivity.

In some embodiments, microorganisms that have ability to grow in DHA in a medium comprising DHA as a source of carbon is subjected to further genetic modifications to include one or more nucleotide sequences encoding isoflavonoid biosynthetic pathway enzymes. Isoflavonoids are made from liquiritigenin and naringenine by a series of enzyme catalyzed biochemical reactions. Microorganisms that already has an ability to produce high amounts of liquiritigenin and naringenine either natively or by one or more genetic modifications are a preferred host for the production of isoflavonoids. On the other hand, isoflavonoid biosynthetic pathway can be directly engineered in the host cell that previously lack the ability to produce liquiritigenin and naringenine but can be engineered to selectively produce isoflavonoids. Furthermore, in addition to the enzymes that catalyze the formation of liquiritigenin and naringenine from DHA, one or more of the enzymes selected from a group of isoflavonoid biosynthetic pathway such as 2-hydroxyisoflavanone synthase, 2-hydroxyisoflavanone dehydratase, isoflavone-2′-hydroxylase, isoflavone-7-O-methyltransferase, isoflavone-3′-hydroxylase and 2′-hydroxy isoflavone reductase are overexpressed for the enhanced production of isoflavonoid from liquiritigenin and naringenine. In one method, naringenine is converted to apigenin, genistein, 2′-hydroxy genistein, biochain A, pretensein, prunetin and 2,3-dihydrobiochain A by one or more of the enzymes from isoflavonoids biosynthetic pathway such as flavone synthase, 2-hydroxyisoflavanone synthase, 2-hydroxyisoflavanone dehydratase, isoflavone-2′-hydroxylase, isoflavone-7-O-methyltransferase and isoflavone-4′-O-methyltransferase which are overexpressed for the enhanced production of isoflavonoids from naringenine. In another method, liquiritigenin is converted to daidzein, (−) vestitone, (−) sophorol, (+) sophorol, (−) maackiain, (+) maackiain, (+) pisatin, (−) medicarpin, (−) vestitol, glyceocarpin, 4-dimethylallylglycinol by one or more of the enzymes from isoflavonoids biosynthetic pathway such as flavone synthase, 2-hydroxyisoflavanone synthase, 2-hydroxyisoflavanone dehydratase, isoflavone-2′-hydroxylase, isoflavone-7-O-methyltransferase, isoflavone-4′-O-methyltransferase, isoflavone-3′-hydroxylase and 2′-hydroxy isoflavone reductase which are overexpressed for the enhanced production of isoflavonoids from liquiritigenin. In addition, attenuation or deletion of one or more of enzymes methylglyoxal synthase, pyruvate carboxylase, phosphoenolpyruvate carboxylase, succinate reductase, citrate synthase, acetate kinase, aldehyde dehydrogenase, pyruvate oxidase, pyruvate-formate lyase, alanine transaminase, lactate dehydrogenase, β-ketothiolase, acetyl-CoA carboxylase and HMG-CoA synthase improves pyruvate utilization in isoflavonoid biosynthesis. Furthermore, enhancing one or more of the enzymes from aromatic amino acid biosynthetic pathways and reducing aromatic amino acids degradation pathways improves aromatic amino acid availability for the isoflavonoid synthesis. Furthermore, blocking one or more of isoflavonoid degradation pathways, improving isoflavonoid transport, reducing isoflavonoid uptake from media and reducing feedback inhibition increases isoflavonoid productivity.

In some embodiments, microorganisms that have ability to grow in DHA in a medium comprising DHA as a source of carbon is subjected to further genetic modifications to include one or more nucleotide sequences encoding flavone and flavonol biosynthetic pathway enzymes. Flavone and flavonol are made from kaemferol and apigenin by a series of enzyme catalyzed biochemical reactions. Microorganisms that already has an ability to produce high amounts of kaemferol and apigenin either natively or by one or more genetic modifications are a preferred host for the production of flavone and flavonol. On the other hand, flavone and flavonol biosynthetic pathway can be directly engineered in the host cell that previously lack the ability to produce kaemferol and apigenin but can be engineered to selectively produce flavone and flavonol. Furthermore, in addition to the enzymes that catalyze the formation of kaemferol and apigenin from DHA, one or more of the enzymes selected from a group of flavone and flavonol biosynthetic pathway such as flavonoid 3′, 5′-hydroxylase, flavone-3-O-methyltransferase, flavonol-3-O-methyltransferase, flavone-7-O-methyltransferase, flavonol-4′-O-methyltransferase and flavonoid-O-methyltransferase are overexpressed for the enhanced production of flavone and flavonol from kaemferol and apigenin. In one method, apigenin is converted to luteolin, 3-O-methylluteolin by one or more of the enzymes from flavone and flavonol biosynthetic pathway such as flavonoid 3′, 5′-hydroxylase and flavone-3-O-methyltransferase are overexpressed for the enhanced production of flavone and flavonol from apigenin. In one method, kaempferol is converted to quercetin, myricetin, laricitrin, syringetin and ayarin by one or more of the enzymes from flavone and flavonol biosynthetic pathway such as flavonoid 3′, 5′-hydroxylase and flavonol-3-O-methyltransferase, flavonoid-O-methyltransferase, flavone-7-O-methyltransferase and flavonol-4′-O-methyltransferase are overexpressed for the enhanced production of flavone and flavonol from kaempferol. In addition, attenuation or deletion of one or more of enzymes methylglyoxal synthase, pyruvate carboxylase, phosphoenolpyruvate carboxylase, succinate reductase, citrate synthase, acetate kinase, aldehyde dehydrogenase, pyruvate oxidase (PoxB), pyruvate-formate lyase, alanine transaminase, lactate dehydrogenase, β-ketothiolase, acetyl-CoA carboxylase and HMG-CoA synthase improves pyruvate utilization in flavone and flavonol biosynthesis. Furthermore, enhancing one or more of the enzymes from aromatic amino acid biosynthetic pathways and reducing aromatic amino acids degradation pathways improves aromatic amino acid availability for the flavone and flavonol synthesis. Furthermore, blocking one or more of flavone and flavonol degradation pathways, improving flavone and flavonol transport, reducing flavone and flavonol uptake from media and reducing feedback inhibition increases flavone and flavonol productivity.

In some embodiments, microorganisms that have ability to grow in DHA in a medium comprising DHA as a source of carbon is subjected to further genetic modifications to include one or more nucleotide sequences encoding stilbenoid, diarylheptanoid and gingerol biosynthetic pathway enzymes. Stilbenoid, diarylheptanoid and gingerol are made from α,β-unsaturated aromatic acyl-CoA such as cinnamyl-CoA, p-coumaroyl-CoA, caffeoyl-CoA and feruloyl-CoA by a series of enzyme catalyzed biochemical reactions. Microorganisms that already has an ability to produce high amounts of α,β-unsaturated aromatic acyl-CoA either natively or by one or more genetic modifications are a preferred host for the production of stilbenoid, diarylheptanoid and gingerol. On the other hand, stilbenoid, diarylheptanoid and gingerol biosynthetic pathway can be directly engineered in the host cell that previously lack the ability to produce α,β-unsaturated aromatic acyl-CoA but can be engineered to selectively produce stilbenoid, diarylheptanoid and gingerol. Furthermore, in addition to the enzymes that catalyze the formation of α,β-unsaturated aromatic acyl-CoA from DHA, one or more of the enzymes selected from a group of stilbenoid, diarylheptanoid and gingerol biosynthetic pathway such as resveratrol di-O-methyltransferase, stilbene synthase, pinosylvin synthase, phenylpropanoylacetyl-CoA synthase and gingerol synthase are overexpressed for the enhanced production of stilbenoid, diarylheptanoid and gingerol from α,β-unsaturated aromatic acyl-CoA. In one method, cinnamyl-CoA is converted to pinosylvin by pinosylvin synthase. In another method, p-coumaroyl-CoA is converted to resveratrol and pterostilbene by stilbene synthase, resveratrol di-O-methyltransferase. In another method, feruloyl-CoA is converted to curcumin by phenylpropanoylacetyl-CoA and curcumin synthase. In another method, feruloyl-CoA is converted to 6-gingerol by unclassified enzymes via 1-dehydro-6-gingerdione. In addition, attenuation or deletion of one or more of enzymes methylglyoxal synthase, pyruvate carboxylase, phosphoenolpyruvate carboxylase, succinate reductase, citrate synthase, acetate kinase, aldehyde dehydrogenase, pyruvate oxidase, pyruvate-formate lyase, alanine transaminase, lactate dehydrogenase, β-ketothiolase, acetyl-CoA carboxylase and HMG-CoA synthase improves pyruvate utilization in stilbenoid, diarylheptanoid and gingerol biosynthesis. Furthermore, enhancing one or more of the enzymes from aromatic amino acid biosynthetic pathways and reducing aromatic amino acids degradation pathways improves aromatic amino acid availability for the stilbenoid, diarylheptanoid and gingerol synthesis. Furthermore, blocking one or more of stilbenoid, diarylheptanoid and gingerol degradation pathways, improving transport, reducing uptake from media and reducing feedback inhibition increases stilbenoid, diarylheptanoid and gingerol productivity.

In some embodiments, microorganisms that have ability to grow in DHA in a medium comprising DHA as a source of carbon is subjected to further genetic modifications to include one or more nucleotide sequences encoding vanillin and capsaicin biosynthetic pathway enzymes. Vanillin and capsaicin are made from feruloyl-CoA by a series of enzyme catalyzed biochemical reactions. Microorganisms that already has an ability to produce high amounts of feruloyl-CoA either natively or by one or more genetic modifications are a preferred host for the production of vanillin and capsaicin. On the other hand, vanillin and capsaicin biosynthetic pathway can be directly engineered in to the host cell that previously lack the ability to produce feruloyl-CoA but can be engineered to selectively produce vanillin and capsaicin. Furthermore, in addition to the enzymes catalyze the formation of feruloyl-CoA from DHA, one or more of the enzymes selected from a group of vanillin and capsaicin biosynthetic pathway such as feruloyl-CoA hydratase, vanillin synthase, vanillin aminotransferase and capsaicin synthase are overexpressed for the enhanced production of capsaicin from feruloyl-CoA. In addition, attenuation or deletion of one or more of enzymes methylglyoxal synthase, pyruvate carboxylase, phosphoenolpyruvate carboxylase, succinate reductase, citrate synthase, acetate kinase, aldehyde dehydrogenase, pyruvate oxidase, pyruvate-formate lyase, alanine transaminase, lactate dehydrogenase, β-ketothiolase, acetyl-CoA carboxylase and HMG-CoA synthase improves pyruvate utilization in vanillin and capsaicin biosynthesis. Furthermore, enhancing one or more of the enzymes from aromatic amino acid biosynthetic pathways and reducing aromatic amino acids degradation pathways improves aromatic amino acid availability for the vanillin and capsaicin synthesis. Furthermore, blocking one or more of vanillin and capsaicin degradation pathways, improving transport, reducing uptake from media and reducing feedback inhibition increases vanillin and capsaicin productivity.

Genetically modified microorganisms capable of producing “flavonoids” using conventional sugars such as glucose, sucrose or glycerol are already known in the art. U.S. Pat. Nos. 7,338,791, 7,034,203 and 7,807,422 provide methods and composition for the production of flavonoids in microbial hosts. U.S. Pat. No. 9,181,539 provides production of flavonoids and precursors through recombinant expression of tyrosine ammonia lyase. U.S. Pat. No. 7,750,211 provides methods and compositions for the production of flavonoid based nutraceuticals. U.S. Pat. No. 7,582,675 and U.S. Pat. No. 7,138,429 provides microbial fermentation for the production of flavonoid using Aspergillus saitoi. U.S. Pat. No. 7,604,968 provides microorganism for the recombinant production of flavonoids. U.S. Pat. Nos. 8,895,287 and 9,040,269 provide the process to produce resveratrol by microbial cells. U.S. Pat. No. 7,772,444 provides the production of resveratrol in a recombinant oleaginous microorganism. U.S. Pat. No. 8,569,024 provides production of stilbenoids using genetically modified yeast. U.S. Pat. No. 8,343,739 provides metabolically engineered cells to produce pinosylvin. U.S. Patent Application Publication No. 2007/0031951 provides method to produce resveratrol in a recombinant bacterial cell. International PCT Application Publication No. WO2015/028324 provides methods for producing modified resveratrol. U.S. Patent Application Publication No. 2014/0245496 provides compositions and methods for the biosynthesis of vanillin. U.S. Pat. No. 6,372,461 provides synthesis of vanillin from sugar based carbon source using Escherichia coli. International PCT Application Publication No. WO2014/128252 provides biosynthesis of O-methylated phenolic compounds. International PCT Application Publication No. WO2015/009558 provides methods for the biosynthesis of vanillin. International PCT Application Publication No. WO2015/121379 provides process to produce vanillin. International PCT Application Publication No. WO2015/193371 provides improved selectivity to produce vanilloids. International PCT Application Publication No. WO2015/193348 provides improved production of vanilloids. U.S. Patent Application Publication No. 2006/0172402 provides production of vanillin in microbial cells. U.S. Pat. No. 6,133,003 provides process for the preparation of vanillin by microbial fermentation. U.S. Pat. No. 6,235,507 provides process for the vanillin production by microbial fermentation. U.S. Pat. No. 9,115,377 provides Amycolatopsis sp. to produce vanillin. U.S. Pat. No. 7,462,470 provides method to produce vanillic acid and vanillin. U.S. Pat. Nos. 8,986,950 and 7,846,697 provide method for higher production of vanillin. International PCT Application Publication No. WO2014/122227 provides improved production of rebaudiosides. U.S. Patent Application Publication Nos. 2013/0171328 and 2008/00640063 and U.S. Pat. No. 9,284,570 provide production of steviol glycosides in microorganism. U.S. Pat. Nos. 4,981,795 and 4,874,701 provides microorganism to produce coniferylalcohol. U.S. Pat. No. 5,344,994 provides process for the preparation of coniferyl aldehyde. International PCT Application Publication No. WO2015/132411 and U.S. Patent Application Publication No. 2014/0248668 provides production of saffron compounds using recombinant microorganism. International PCT Application Publication No. WO2015/109168 provides methods of using capsaicin synthase for the microbial production of capsaicinoids. International PCT Application. Publication No. WO2010/106189 provides methods of making vanillin via microbial fermentation. U.S. Pat. No. 7,309,591 provides production of resveratrol in cultured cell. U.S. Patent Application Publication No. 2010/0068775 and U.S. Patent Application Publication No. 2010/0143990 provide fermentative production of hydroxytyrosol using engineered microorganism. U.S. Patent Application Publication No. 2006/0141587 provides process for the preparation of 3,4-dihydroxyphenylalanine. International PCT Application. Publication No. WO2015/066609 provides methods of using O-methyltransferase for the biosynthetic production of pterostilbene. U.S. Pat. No. 8,344,119 provides system to produce aromatic molecules in Streptomyces. Chinese Patent No. CN102936577 provides engineered bacterium to produce pinocembrin. Chinese Patent No. CN103865864 provides engineered bacterium to produce eriodictyol. Chinese Patent No. CN103484420 provides engineered bacterium to produce naringenin.

In one method to enhance DHA uptake and utilization, the genetically modified host cells already known to produce flavonoids, as described in patents above, is subjected to further genetic engineering by means of expressing plasmids that code ATP and PEP dependent kinases that could phosphorylate DHA and facilitate its entry into glycolytic cycle. In another method to enhance DHA uptake and utilization, genetically modified host cells already known to produce flavonoids, as described in patents above, is subjected to chemical mutagenesis and the strains with the ability to grow and produce desired flavonoids with high enough titer and yield in a growth medium comprising DHA as a source of carbon will be identified and subjected to whole genome sequencing to identify specific mutation associated with the ability to grow and produce flavonoids in a medium comprising DHA. Such a mutation will be introduced to the genetically modified host cells already known to produce flavonoids to confer the ability to use DHA as a source of organic carbon. In another method to enhance DHA uptake and utilization, genetically modified host cells already known to produce flavonoids, as described in patents above, is exposed to growth medium with increasing concentration of DHA and subjected to metabolic evolution. Strains that have gained ability to grow in the medium containing DHA as a major or sole source of carbon and still retain the ability to produce flavonoids is identified and subjected to whole genome sequencing to identify specific mutations. Such specific mutation associated with the ability to grow and produce flavonoids in a medium comprising DHA will be introduced to the genetically modified host cells already known to produce flavonoids, as described in patents above, for the purpose of conferring the ability to use DHA as a source of organic carbon.

### Analytical Method

Wild-type Clostridium acetobutylicum ATCC 824 is maintained as spores and suspended in Clostridial growth medium (CGM) containing (per liter) 0.75 g KH₂PO₄, 0.75 g K₂HPO₄, 1.0 g NaCl, 0.017 g MnSO₄, 0.7 g MgSO₄, 0.01 g FeSO₄, 2.0 g L-asparagine, 5.0 g yeast extract, 2.0 g (NH₄)₂SO₄ and 80 g sugar substrate supplemented with 15% glycerol. Spores are generated by heating them at 70 to 80° C. for 10 min after inoculation. For growth on solid medium, C. acetobutylicum is grown anaerobically at 37° C. on 2×YTG (pH 5.8, 16 g bacto tryptone, 10 g yeast extract, 4 g NaCl, 5 g carbon source per liter) agar plates. For recombinant strains, erythromycin is added to liquid and solid media at concentration of 40 μg/ml and chloramphenicol is added at the final concentration of 5 μg/ml. Electro transformation of C. acetobutylicum is performed by following known procedures. Frozen stock of recombinant strains is prepared by mixing 1 ml of CGM culture with 0.5 ml of 50% glycerol and stored at −80° C. Single colony is inoculated into a test tube containing 10 ml of CGM and anaerobically cultured at 37° C. until the OD₆₀₀ reached 2.0. Capped flask containing 150 ml CGM supplemented with 60 g/L sugar substrate are inoculated with 5 ml of this culture and grown at 37° C. After 48 h, the samples are taken from the flask and used in analysis. DHA and other chemicals are obtained from Fisher Scientific and Sigma-Aldrich Co.

Batch fermentation is carried out in a Liflus GX bioreactor containing 1.8 liters of CGM supplemented with 80 g/L sugar substrate. The spore suspension will be inoculated into a capped tube containing 10 ml CGM, heat shocked and grown vigorously. This pre-culture is inoculated into a 500-ml flask containing 200 ml of CGM. When the cell density in the flask reaches an OD₆₀₀ of 1-2, the bioreactor is inoculated with flask culture. The pH is maintained above 5.0 by using ammonia solution.

Escherichia coli bacterial strains used in the present study are grown in minimal medium. The minimal medium designed for this study is supplemented with 2 g/L tryptone, 5 μM sodium selenide, 1.32 mM Na2HPO4. Morpholino-propanesulfonic (MOPS) acid is used for the inoculum preparation phase when conducted in tubes with no external control of pH. Varying concentration of DHA is used as per the experimental requirements. The medium is also supplemented with specified concentrations of monobasic and dibasic sodium, potassium and ammonium phosphate, sodium and potassium chloride, sodium and potassium sulfate.

Fermentations are conducted in Infors AG Sixfors six channel multi-fermenter system with six 500 ml working volume vessels and independent control of temperature (37° C.), pH (externally controlled with NaOH and H₂SO₄) and stir speed (200 rpm). The system is also equipped with computer controlled gas analysis and each vessel is fitted with a condenser operated with a 2° C. cooling system to minimize product evaporation. Anaerobic conditions are maintained by initially sparging the medium with high purity argon and thereafter flushing the head space with the same gas. Experiments in tubes are conducted using 20-ml corning tubes, which are modified by piercing the septa with two needles, one for oxygen free argon sparging and another for gas efflux.

Prior to use, the cultures stored at −80° C. are streaked onto LB plates and incubated overnight at 37° C. A single colony is used to inoculate 20-ml corning tubes filled with minimal medium (supplemented with 10 g/L tryptone, 5 g/l yeast extract and 10 g/L DHA). The tubes are incubated at 37° C. until an OD₅₅₀ of −0.4 is reached. A portion of this actively growing pre-culture is centrifuged, and the pellet is washed and used to inoculate 350 ml of medium in each fermenter, with a target initial optical density of 0.05 at 550 nm.

Optical density of the microbial cultures are measured at 550 nm and used to determine the cell mass (1 OD=0.34 g dry weight/L). The microbial cultures are centrifuged to remove the microbial cells and the supernatant is stored at −20° C. for HPLC analysis. DHA, butyric acid, acetic acid, isopropanol, ethanol, acetone and butanol are quantified with ion-exclusion HPLC using a Shimadzu Prominence SIL 20 system equipped with an HPX-87H organic acid column operating under conditions optimized for peak separation (30 mM H₂SO₄ in mobile phase at 0.3 ml/min, column temperature 42° C.). Off gas concentrations of N₂, O₂, H₂ and CO₂ are determined by a benchtop gas analysis system. The identity of the fermentation products is determined by the NMR experiments. Enzymes are assayed by known procedures.

Data from cell growth, DHA consumption and product synthesis is used to calculate productivity (mmol/L/h or g/L/h), titer (g/L) and yield (g product produced/g DHA consumed) for each 12-h interval. Specific rates (mmol/gcell/h) are calculated by dividing the volumetric rates by the time average concentration of the cells over the same period of time.

The following Examples are included to illustrate certain aspects of the present invention, and should not be viewed as exclusive embodiments. The subject matter disclosed is capable of considerable modifications, alterations, combinations, and equivalents in form and function, as will occur to those skilled in the art and having the benefit of this disclosure.

### Example 1

### Production of Methanol from Methane

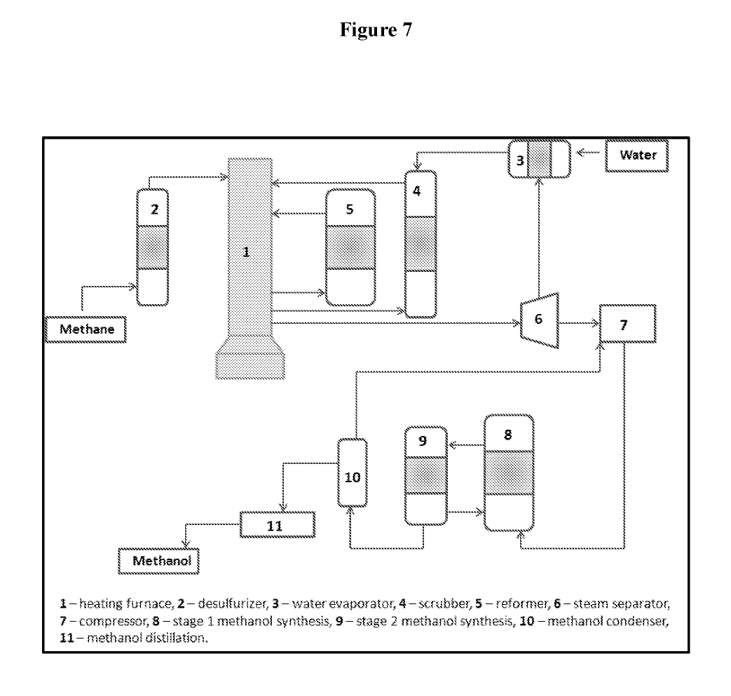

A diagram of the complete methanol plant, based on methane, natural gas or biogas is shown in FIG. 7. The methane or biogas is passed through desulfurization reactor (2) packed with zinc oxide beads. The gas is then heated in the central furnace (1) to approx. 420° C. From the furnace, the gas flows into the scrubber (4) to be saturated with the steam generated by water evaporator (3). Methane is heated in the furnace to 800° C. after being saturated with more steam to form desired mixture, and then directed into the reforming unit (5) for steam reformation reaction. Syngas at nearly 900° C. is then separated from excess steam by steam separator (6) and compressed to 60 atm by compressor (7) and is then combined with the unreacted reactants being recirculated from the methanol condenser and sent to two serially connected methanol synthesis reactors (8, 9). After the second synthesis reactor (9) the post reaction mixture is passed through water-cooled methanol condenser (10). From the bottom part of the methanol separator, a crude methanol flows into serially connected methanol distillation columns. After the last distillation column, the purity of the methanol product is a minimum of 99.99%. A plant production of methanol from methane is operating at 60 atm pressure and at a production rate of 1000 metric tons per day uses about 910 metric tons per day of steam, which is generated by boiling of water and compressed by turbocompressor driven by the electric motors. Electricity required for the process is generated by hydrogen fuel cell. Hydrogen required for the fuel cell is obtained from methane steam reforming process. The total yield of methanol from this process is over 94% of the total and fuel methane, including the provision of all required fuel to the furnace. This yield corresponds to less than about 23.2 million BTU of lower heating value of feed and fuel per short ton of product methanol.

### Example 2

### Production of Formaldehyde from Methanol

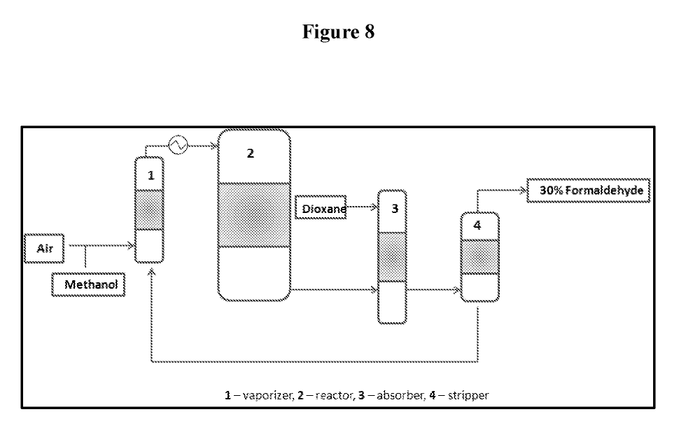

A diagram of the complete formaldehyde production plant, based on methanol is shown in FIG. 8. Air is compressed to pressure by an air compressor and feed to the bottom of the methanol vaporizer (1). The ratio of methanol and air is maintained about 35-45%. This mixture is heated to the reaction temperature 550-600° C. by series of preheater before entering the reactor (2). The reactor is a fixed bed type filled with silver catalyst used for converting methanol to formaldehyde. The product stream from the reactor is sent to the absorber (3) where the gaseous formaldehyde is absorbed into dioxane to form 30% formaldehyde solution. The product stream is sent to purification and recovery section (4). Unreacted methanol is fed back to the process at methanol vaporizer. Formaldehyde is obtained as heavy end of the alcohol stripper column. The final formaldehyde preparation is in the form of 30% solution in dioxane. This process gives an overall process yield of 95% on weight basis.

### Example 3

### Production of DHA from Formaldehyde

3-hexylbenzothiazolium bromide (5.0 g, 16.7 mmol), triethylamine (2.3 ml, 16.7 mmol) and dioxane (50 ml) are heated at 80° C. under nitrogen and stirred for 12 h. After cooling, the precipitated triethylammonium bromide is filtered. The filtrate is used as a catalyst solution. 30% formaldehyde solution in dioxane (450 ml) is heated to 100° C. under nitrogen and then catalyst solution (50 ml) is added. The mixture is stirred at 100° C. for 1 h. After 1 h, dioxane was removed and the reaction mixture was analyzed by HPLC. Analysis of the reaction mixture showed that the yield of DHA is 85% and the conversion of formaldehyde is 99%. The reaction mixture is evaporated to remove the solvent. The residue is poured into water (500 ml) and extracted with dichloromethane (100 ml) three times to recycle the catalyst. The aqueous solution is used directly in the following step.

### Example 4

### Development of Escherichia coli Strains for Producing Ethanol Using DHA as a Source of Carbon

Four different E. coli strains are used to demonstrate the possibility of using DHA as a sole or major source of carbon and energy in the fermentative production of ethanol. Within the wild type E. coli bacterial cells there are several competing metabolic pathways for the production of lactic acid, succinic acid, acetic acid, hydrogen, pyruvic acid and formic acid. During the last two decades several groups have genetically engineered wild type E. coli cells to produce commercially acceptable levels of ethanol using either glucose or glycerol as the sole source of carbon and energy. The following bacterial strains will be used to test the possibility of using DHA as a sole or major source of carbon and energy in the fermentative production of ethanol. (1) E. coli strain K12 MG1655 (ΔfrdA, Δpta) genetically engineered to produce ethanol from glycerol. (2) E. coli strain ATCC 11303 (pLO11297) genetically engineered to produce ethanol from glucose. (3) Klebsiella Oxytoca M5A1 (pLOI555) strain genetically engineered to produce ethanol from glucose. (4) E. coli ATCC 8739 (ΔfrdABCD, ΔackA, Δpta, ΔldhA, ΔpoxB) strain genetically engineered to produce ethanol from glycerol. Following two different approaches, these four bacterial strains are tested for their ability to utilize DHA as a sole or major source of carbon and energy. Under the first of the two approaches, these four bacterial strains are subjected to metabolic evolution in a growth medium comprising increased levels of DHA. Those isolates with the ability to grow in the presence of high concentration of DHA are tested for the capacity to produce ethanol. Those isolates which have acquired the ability to tolerate DHA and still retains the ability to produce ethanol at high enough titer and yield as the parent strain are subjected to whole genome sequencing to identify the genetic modifications that have conferred the ability to use DHA as a sole or major source of energy and carbon in ethanol production.

In the second approach, the four bacterial strains genetically engineered to produce ethanol using glucose or glycerol are transformed with the plasmid containing genes encoding for exogenous DHA kinase and/or triose isomerase. Enhancing the DHA kinase enzyme activity and/or triose isomerase would increase the detoxification of DHA within the cell leading to the ability to use DHA as a sole or major source of carbon and energy in the production of ethanol. The following three plasmid constructs are tested: (1). Plasmid expressing Saccharomyces cervisiae DHA kinase gene dak1 and dak2 under a constitutive promoter. (2). Plasmid expressing E. coli dhaKLM operon under a constitutive promoter (3). Plasmid expressing ATP dependent DHA kinase from Citrobacter freundii under a constitutive promotor (4). Plasmid expressing Lactococcus lactis triosephosphate isomerase gene tpi under a constitutive promoter. The three plasmid constructs listed above containing one or other DHA kinase gene are prepared and tested for their ability to confer DHA tolerance to the bacterial strains genetically engineered to produce ethanol. A fourth plasmid construct containing triosephosphate isomerase gene (tpiA) is prepared and tested for their ability to isomerize DHA phosphate to glycolytic intermediate glyceraldehyde phosphate.

### Example 5

### Development of E. coli Strains for Producing Lactic Acid Using DHA as a Source of Carbon

The following four bacterial strains are used: (1) E. coli strain CICIM B0013 (ΔackA Δpta Δpps ΔpflB Δdld ΔpoxB ΔadhE ΔfrdA) genetically engineered to produce lactic acid from glycerol. (2). E. coli strain YYC202 (ΔaceEF Δpfl Δpps ΔpoxB) genetically engineered to produce lactic acid with a yield of 0.99 g/g from glucose. (3). E. coli strainW3110 (ΔfocA ΔfrdBC ΔadhE ΔackA ΔmgsA) genetically engineered to produce lactic acid from glucose. (4) E. coli MG1655 strain (Δpta Δdld ΔadhE ΔfrdA) strain genetically engineered to produce lactic acid from glycerol. These four E. coli strains are tested using two different approaches for their ability to utilize DHA as a sole or major source of carbon and energy.

Under the first of the two approaches, these four E. coli strains are subjected to metabolic evolution in a growth medium comprising increasing levels of DHA. Those isolates with the ability to grow in the presence of high concentration of DHA are tested for the capacity to produce lactic acid. Those isolates which have acquired the ability to tolerate DHA and still retains the ability to produce lactic acid at high enough titer and yield as the parent strain are subjected to whole genome sequencing to identify the genetic modifications that have conferred the ability to use DHA as a sole or major source of energy and carbon in lactic acid production.

In the second approach, the four E. coli strains genetically engineered to produce lactic acid using glucose or glycerol are transformed with the plasmid containing genes encoding for exogenous DHA kinase and/or triose isomerase. The following four plasmids are tested: (1). Plasmid expressing Saccharomyces cervisiae DHA kinase gene dak1 and dak2 under a constitutive promoter (2). Plasmid expressing E. coli dhaKLM operon under a constitutive promoter (3). Plasmid expressing ATP dependent DHA kinase from Citrobacter freundii under a constitutive promotor (4). Plasmid expressing Lactococcus lactis triosephosphate isomerase gene tpi under a constitutive promoter. These set of experiments are carried out to test the hypothesis that enhancing the DHA kinase enzyme activity and/or triose isomerase would increase the detoxification of DHA within the cell leading to the ability to use DHA as a sole or major source of carbon and energy in the production of lactic acid. The fourth plasmid construct containing triosephosphate isomerase gene (tpiA) is used to test its ability to isomerize DHA phosphate to glycolytic intermediate glyceroldehyde 3-phosphate.

### Example 6

### Development of Bacterial Strains for Producing Butanol Using DHA as a Source of Carbon

The following four Clostridium strains are used to demonstrate the possibility of using DHA as a sole or major source of carbon and energy in the fermentative production of butanol. (1) Wild-type C. acetobutylicum (ATCC 824) producing acetone, butanol and ethanol (ABE); this organism uses a broad spectrum of sugars (C5 and C6) as carbon and energy source, and also co-utilize glycerol. This strain will be tasted for utilization and growth on DHA. (2) C. acetobutylicum PJC4BK genetically engineered to produce higher levels of butanol from glucose and the broad spectrum of other substrates. (3) Wild-type C. pasteurianum (ATCC 6013) producing butanol from glycerol as well as glucose and many other sugars. (4) C. pasteurianum ATCC 6013ΔSpo0A strain genetically engineered to produce higher levels butanol from glycerol; this bacterial strain has developed tolerance to high concentrations of crude glycerol.

These four bacterial strains are tested for their ability to grow on DHA as the sole carbon and energy source or in the presence of small amounts of glucose (for C. acetobutylicum) or glycerol (for C. pasteurianum). Chemical mutagenesis is followed by selection and strain evolution to enhance their ability to utilize DHA as a major source of carbon and energy, and also to tolerate higher levels of DHA.

### REFERENCES

All patent and scientific references listed under this section and in the rest of the specification of this patent application are provided for the convenience of the reader. Each patent reference provided in this patent application is incorporated by reference in its entity.
U.S. Pat. No. 4,539,293
U.S. Pat. No. 4,757,010
U.S. Pat. No. 4,777,135
U.S. Pat. No. 5,000,000
U.S. Pat. No. 5,028,539
U.S. Pat. No. 5,162,516
U.S. Pat. No. 5,192,673
U.S. Pat. No. 5,210,032
U.S. Pat. No. 5,424,202
U.S. Pat. No. 5,821,093
U.S. Pat. No. 6,358,717 B1
U.S. Pat. No. 6,949,491B2
U.S. Pat. No. 7,326,551 B2
U.S. Pat. No. 7,381,851 B2
U.S. Pat. No. 7,629,162 B2
U.S. Pat. No. 7,993,889 B2
U.S. Pat. No. 8,129,157 B2
U.S. Pat. No. 8,129,157 B2
U.S. Pat. No. 8,178,328 B2
U.S. Pat. No. 8,426,191 B2
U.S. Pat. No. 8,465,953 B2
U.S. Pat. No. 8,652,817 B2
U.S. Pat. No. 8,691,539 B2
U.S. Pat. No. 8,691,552 B2
U.S. Pat. No. 8,716,002 B2
U.S. Pat. No. 8,735,114 B2
U.S. Pat. No. 8,759,044 B2
U.S. Pat. No. 8,765,425 B2
U.S. Pat. No. 9,040,263 B2
U.S. Pat. No. 9,040,267 B2
U.S. Pat. No. 9,062,340 B2
U.S. Pat. No. 9,175,315 B2
U.S. Patent Application Publication No. 2002/0137154
U.S. Patent Application Publication No. 2009/0155869
U.S. Patent Application Publication No. 20090186392
U.S. Patent Application Publication No. 20140273128
EP 0474387
EP 0480646
International Patent Application Publication No. WO 2012082720
International Patent Application Publication No. WO 2013015770
International Patent Application Publication No. WO 2014165763
International Patent Application Publication No. WO2015013334
Adekunle, K. F., Okolie, J. A., A review of biochemical process of anaerobic digestion.
Advances in Bioscience and Biotechnology. 2015, 6, 205-212.
Alterthum F., Ingram L. O. Efficient ethanol production from glucose, lactose and xylose by recombinant Escherichia coli. Appl. Environ. Microbiol. 1989, 55, 1943-1948.
Bachler C., Schneider P., Bahler P., Lustig A., Erni B. Escherichia coli DHA kinase controls gene expression by binding to transcription factor DhaR. EMBO journal, 2005, 24, 283-293.
Bachler, C., Flukiger-Bruhwiler, K., Schneider, P., Bahler, P., Erni, B. From ATP as sunstrate to ADP as coenzyme. J. Biol. Chem. 2005, 280(18), 18321-18325.
Bansode A., Urakawa A. Towards full one-pass conversion of CO₂ to methanol and methanol derivatives. Journal of Catalysis, 2014, 309, 66-70.
Bari D., Glatzer E. Studies to evaluate value-added market opportunities for North Dakota. Technology Analytics Group, North Dakota. Jun. 5, 2014
Beuhler R. J., Rao R. M., Hrbek J., White M. G. Study of partial oxidation of methanol to formaldehyde on a polycrystalline Ag foil. J. Phy. Chem. B. 2001, 105(25), 5950-5956.
Biernat, K., Samson-Brek, I. Review of technology for cleaning biogas to natural gas quality. Chemik, 2011, 65(5), 435-444.
Bio-renewable Insights n-Butanol. Nexant, May 2015.
Bracmort, K. The renewable fuel standard: cellulosic biofuels. Congressional Research Service. Jan. 14, 2015.
Breslow, R., Zhan-Ling, C. L-amino acids catalyze the formation of an excess of D-glyceraldehyde, and thus of other D sugars, under credible prebiotic conditions. Proc. of the Nat. Aca. Sci. USA 2010, 107(13), 5723-5725.
Buisson, R. S. K., Kirk, P. W. W., Lester, J. N. Fate of selected chlorinated organic compounds during semi-continuous anaerobic sludge digestion. Archives of Environmental Contamination and Toxicology. 1990, 19(3), 428-432.
Bullin K., Krouskop P. Composition variety complicates processing plants for US shale gas. Oil&Gas Journal, 2009, 107(10), 18-20.
Cancilla, M. R., Davidson, B. E., Hillier, A. J., Nguyen, N. Y., Thompson, J. The lactococcus lactis triosephospahete isomerase gene, tpi, is monocistronic Microbiology. 1995, 141, 229-238.
Cao W., Chen G., Li S., Yuan Q. Methanol-steam reforming over a ZnO—Cr₂O₃/CeO₂—ZrO₂/Al₂O₃ catalyst. Chem. Eng. J. 2006, 119(2-3), 93-98.
Chawl, S. K., George, M., Patel, F., Patel, S. Production of synthesis gas by carbon dioxide reforming of methane over nickel based and perovskite catalyst. Procedia Engineering. 2013, 51, 461-466.
Chen L., Ren S., Ye X. P. Lactic acid production from glycerol using CaO as solid base catalyst. Fuel Processing Technology, 2014, 120, 40-47.
Choudhary V. R., Rajput A. M., Prabhakar B. Low temperature oxidative conversion of methane to syngas over NiO—CaO catalyst. Catalysts Letters, 1992, 15(4), 363-370.
Clomburg, J. M., Gonzalez, R. Metabolic engineering of Escherichia coli for the production of 1,2-propanediol from glycerol. Biotechnol Bioeng. 2011, 108(4), 867-879.
Coleman W. J., Vidanes G. M., Cottarel G., Muley S., Kamimura R., Javan A. F., Sun J., Groban E. S. Biological conversion of multi-carbon components from methane. U. S Patent, US 20140273128 A1. 2014.
Dahl, P. J., Christensen T. S., Winter-Madsen, S., King, S. M. Proven autothermal reforming technology for modern large-scale methano plants. Nitogen+Syngas Internation Conference and Exibition 2014, Haldor-Topsoe, 1-12.
Deng J., Pan T., Xu Q., Chen M., Zhang Y., Guo Q., Fu Y. Linked strategy for the production of fuels via formose reaction. Nature, 2013, 3, 1244.
Deutscher J., Sauerwald H., Stimulation of DHA and glycerol kinase activity in streptococcus faecalis by phosphoenolpyruvate-dependent phosphorylation catalyzed by enzyme I and HPr of the phosphotransferase system. J. Bacteriol, 1986, 166, 829-836
Deutscher, J., Francke, C. and Postma, P. W. (2006) How phosphotransferase system-related protein phosphorylation regulates carbohydrate metabolism in bacteria. Microbiol. Mol. Bio. Rev. 70: 939-1031.
Drago R., Jurczyk K., Kob N., Bhattacharyya A., Masin J. Partial oxidation of methane to syngas using NiO-supported catalyst. Catalysts Letters, 1998, 51(3), 177-181.
Durnin, G., Clomburg, J., Yeates, Z., Alvarez, P. J. J., Zygourakis, K., Campbell, P., Gonzalez, R. Understanding and harnessing the microaerobic metabolism of glycerol in Escherichia coli. Biotechnol. Bioengineer. 2008, 103, 148-161.
Eiteman M. A., Ramalingam S. Microbial production of lactic acid. Biotechnol Lett, 2015, 37, 955-972.
Erni, B., Siebold, C., Christen, S., Srinivas, A., Oberholzer, A., Baumann, U. small substrate, big surprise: fold, function and phylogeny of DHA kinases. Cell Mol. Life Sci. 2006, 63, 890-900.
Ezeji, T. C., Qureshi, N., Blaschek, H. P. Butanol fermentation research: upstream and downstream manipulations. The Chemical Record, 2004, 4, 305-314
Fei Q., Guarnieri M. T., Tao L., Laurens M. L., Dowe N., Pienkos P. T. Bioconversion of natural gas to liquid fuel: opportunities and challenges. Biotechnology Advances, 2014, 32, 596-614.
Fermentation Chemicals Market Forecasts to 2020 http://www.researchandmarkets.com/publication/mris6b2/fermentation_chemicals_market.
Folger, P. Carbon capture and sequestration: A primer. Congressional Research Service. Jul. 16, 2013.
Folger, P. Carbon capture: A technology assessment. Congressional Research Service. Nov. 5, 2013.
Folger, P. Carbon capture and sequestration: research, development and demonstration at the U.S. department of energy. Congressional Research Service. Feb. 10, 2014
Garrabou, X., Castillo J. A., Guerard-Helaine, C., Parella, T., Joglar, J., Lemaire, M., Clapes, P. Asymmetric Self- and Cross-Aldol Reactions of Glycolaldehyde Catalyzed by D-Fructose-6-phosphate Aldolase, Angew. Chem. Int. Ed., 2009, 48, 5521-5525
Ge X., Yang L., Sheets J. P., Yu Z., Li Y. Biological conversion of methane to liquid fuels: status and opportunities. Biotechnology Advances, 2014, 32, 1460-1475.
Graber T. B., Zhou S., Shanmugam K. T., Yomano L. P., Ingram L. O. Methylglyoxal bypass identified as source of chiral contamination in L(+) and D(−)-lactate fermentations by recombinant E. coli. Biotechnol Lett, 2006, 28, 1527-1535.
Girbal L., Soucaille P. Regulation of Clostridium acetobutylicum metabolism as revealed by mixed-substrate steady-state continuous cultures. Role of NADH/NAD ratio and ATP pool. J. Bacteriol, 1994, 176, 6433-6438.
Goellner J. F., Shah V., Turner M. J., Kuehn N. J., Littlefield J., Cooney G., Marriott J. Analysis of natural gas-to-liquid transportation fuels via Fischer-Tropsch. National Energy Technology Laboratory, September 2013.
Gonzalez, R., Murarka, A., Dharmadi, Y., Yazdani, S. S. A new model frot he anaerobic fermentation of glycerol in enteric bacteria: Trunk and auxiliary pathways in Escherichia coli. Metabolic Engineering, 2008 10, 234-245.
Green, E. M., Boynton, Z. L., Harris, L. M., Rudolph, F. B., Papoutsakis, E. T., Bennett, G. N. Genetic manipulation of acid formation pathways by gene inactivation in clostridium acetobutylicum ATCC 824. Microbiology, 1996, 142, 2079-2086.
Gutknecht R., Beutler R., Garcia-alles L. F., Baumann U., Erni B. The diydroxyacetone kinase of Escherichia coli utilizes a phosphoprotein instead of ATP as phosphoryl donor. EMBO journal, 2001, 20(10), 2480-2486.
Harris, L. M., Desai, R. P., Welker, N. E., Papoutsakis, E. T. Characterization of recombinant strins of the Clostridium acetobutylicum butyrate kinase inactivation mutant: Need for phenomenological models for solventogenesis and butanol inhibition. Biotechnology and Bioengineering 2000, 67(1), 1-11.
Hasona A., Kim Y., Healy F. G., Ingram L. O., Shanmugam K. T. Pyruvate formate lyase and acetate kinase are essential for anaerobic growth of E. coli on xylose. J. Bacteriol, 2004, 186, 7593-7600.
Heller K. B., Lin E. C. C., Wilson T. H. Substrate specificity and transport properties of the glycerol facilitator of Escherichia coli. J Bacteriol., 1980, 144, 274-278.
Herout, M., Malatak, J Kucera, L., Dlabaja, T. Biogas composition depending on the type of plant biomass used. Res. Agr. Eng. 2011, 57(4), 137-143.
Humphries M. U.S crude oil and natural gas production in federal and non-federal areas. Congressional Research Service. Apr. 3, 2015.
Industrialization of Biology. A Roadmap to accelerate the advanced manufacturing of chemicals. National Research Council of the National Academies. The National Academies Press, Washington, D.C.
Jensen, T. O., Kvist, T., Mikkelsen, M. J. Westermann, P. Production of 1,3-PDO and butanol by a mutant strain of Clostridium pasteurianum with increase tolerance towards crude glycerol. AMP Express, 2012 2:44
Jin R. Z., Forage R. G., Lin E. C. C. Glycerol kinase as a substitute for DHA kinase in a mutant of Klebsiella pneumoniae. J. Bacteriol, 1982, 152, 1303-1307.
Jin, R. Z. and Lin, E. C. C. (1984) An inducible phosphoenolpyruvate: DHA phosphotransferase system in Escherichia coli. J. Gen. Microbiol. 130: 83-88.
Johnson, E. A., Burke, S. K., forage, R. G., Lin, E. C. C. Purification and properties of DHA kinase from Klebsiella pneumoniae. J Bacteriol. 1984, 160(1), 55-60.
Kangle, K. M., Kore, S. V., Kore, V. S., Kulkarni, G. S. Recent trends in anaerobic codigestion: A review. Universal Journal of Environmental Research and Technology. 2012, 2(4), 210-219.
Kolek J., Sedlar K., Provaznik I., Patakova P. Dam and Dcm methylatations prevent gene transfer into Clostridium pasteurianum NRRL B-598: development of methods for electrotransformation, conjugation, and sonoporation. Biotechnol. Biofuels, 2016, 9, 14-28.
Lee, S. Y., Park, J. H., Jang, S. H., Nielsen, L. K., Kim, J., Jung, K. S. Fermentative butanol production by clostridia. Biotechnology and Bioengineering 2008, 101(2), 209-228.
Lee, J., Jang, Y., Choi, S. J., Im, J. A., Song, H., Cho, J. H., Seung, D. Y., Papoutsakis, E. T., Bennett G. N., Lee S. Y. Metabolic engineering of clostridium acetobutylicum ATCC 824 for isopropanol-butanol-ethanol fermentation Appl. Environ. Microbiol. 2012, 78(5), 1416-1423.
Lin E. C. C., Jin R. Z. An inducible phosphoenolpyruvate: DHA phosphotrasferase system in Escherichia Coli. Journal of general Microbiology, 1984, 130, 83-88.
Luo. Y., Zhang, T. and Wu, H. (2014) The transport and mediation mechanisms of the common sugars in Escherichia coli. Biotech. Adv. 32: 905-919.
Lutke-Eversloh, T. Application of new metabolic engineering tools for Clostridium acetobutylicum. Appl. Microbiol Biotechnol 2014, 98(13), 5823-5837.
Lynch M. Microorganisms for the conversion of methane and methanol to higher value chemicals and fuels. WO 2014165763 A1, 2014.
Lyubovsky M., Roychoudhury S., Lapierre R. Catalytic partial oxidation of methane to syngas at elevated pressures. Catalysis Letters, 2005, 99(3-4), 113-118.
Martin, M. R., Fornero, J. J., Stark, R., Mets, L., Angenebt, L. T. A single culture bioprocess of Methanothermobacter thermautotrophicus to upgrade digester biogas by CO₂ to CH₄ conversion with H₂. Archaea. 2013, 1-11.
Matsumoto T., Yamamoto H., Inoue S. Selective formation of triose from formaldehyde catalyzed by thiazolium salt. J. Am. Chem. Soc. 1984, 106, 4829-4832.
Mazumdar S., Clomburg J. M., Gonzalez R. Escherichia coli strains engineered for homofermentative production of D-lactic acid from glycerol. Appl. Environ. Microbiol. 2010, 76(13), 4327-4336.
Mazumdar, S., Blankschien, M. D., Clomburg, J. M., Gonzalez, R. Efficient synthesis of L-lactic acid from glycerol by metabolically engineered Escherichia coli. Microbial Cell Factories, 2013, 12, 7
Mermelstein, L. D., Welker, N. E., Bennet, G. N., Papoutsakia, E. T. Expression of cloned homologous fermentative genes in clostridium acetobutylicum ATCC 824 Biotechnology 1992, 10, 190-195.
McPhail, L., Westcott, P., Lutman, H. The renewable identification number system and U.S. Biofuel mandate. Economic Research Service/USDA, November 2011, 1-24
Molin M., Norbeck J., Blomberg A. DHA kinases in saccharomyces cerevisiae are involved in detoxification of dihydroxyactone. Journal of Biological Chemistry, 2003, 278(3), 1415-1423.
Molin, M., Blomberg, A. DHA detoxification in Saccharomyces cerevisiae involves formaldehyde dissimilation. Mol. Microbiol. 2006, 60(4), 925-938.
Murarka, A., Dharmadi, Y., Yazdani, S. S. Gonzalez, R. Fermentative utilization of glycerol by Escherichia coli and its implications for the production of fuels and chemicals. App. Environ. Micorbiol. 2008, 74(4), 1124-1135.
Natural gas Spot and future price from U.S. Energy Information Administration, January 2016.
Ohta K., Beall D. S., Mejia J. P., Shanmugam K. T., Ingram L. O. Metabolic engineering of Klebsiella Oxytoca M5A1 for ethanol production from xylose and glucose Appl. Environ. Microbiol. 1991, 57(10), 2810-2815.
Ohta K., Beall D. S., Mejia J. P., Shanmugam K. T., Ingram L. O. Genetic improvement of Escherichia coli for ethanol production: Chromosomal integration of Zymomonous mobilis genes encoding pyruvate decarboxylase and alcohol dehydrogenase Appl. Environ. Microbiol. 1991, 57(4), 893-900.
Ouellette M., Makkay A. M., Papake R. T. DHA metabolism in Haloferax volcanii. Frontiers in Microbiology, 2013, 4, 1-11.
Pajuelo M. G., Salles I. M., Mendes F., Soucaille P., Vasconcelos I. Microbial conversion of glycerol to 1,3-propanediol. Physiological comparison of a natural producer, Clostridium butyricum VPI 3266, and an engineered strain, Clostridium acetobutylicum DG1 (pSPD5). Appl. Environ. Microbiol. 2006, 72(1), 96-101.
Papoutsakis, E. T. Engineering solventogenic clostridia. Curr. Opin. Biotechnol 2008, 19(5), 420-429.
Paulson I. I., Reizer J., Jin R. Z., Lin E. C. C., Saier M. H. (2000) Functional genomic studies of DHA utilization in Escherichia coli. Microbiology, 146, 2343-2344.
Pfromm, P. H., amanor-Boadu, V., Nelson, R., Vadlani, P., Madl, R. Bio butanol bio ethanol: a technical and economic assessment for corn and switchgrass fermented by yeast or clostridium acetobutylicum. Biomass Bioenergy 2010, 34, 515-524.
Poschl, M., Ward, S., Owende, P. Evaluation of energy efficiency of various biogas production and utilization pathways. Applied Energy. 2010, 87(11), 3305-3321.
Postma, P. W. and Lengeler, J. W. (1985) Phosphoenolpyruvate: Carbohydrate phosphotransferase system in bacteria. Microbiol. Rev. 49: 232-269.
Postma, P. W., Lengeler, J. W. and Jacobson, G. R. (1993) Phosphoenolpyruvate: Carbohydrate phosphotransferase systems of bacteria. Microbiol. Rev. 57: 543-594.
Ramseur, J. L. U.S greenhouse gas emissions: recent trends and factors. Congressional Research Service. Nov. 24, 2014.
Ratner M., Tiemann M. An overview of unconventional oil and natural gas: Resources and federal actions. Congressional Research Service. Apr. 22, 2015.
Reed G., Rehm H. J. Biotechnology, Biotransformations I Volume 8a, Wiley-VCH, 2008, ISBN: 9783527283187.
Roth, J. A., Orchin, M. Hydroformylation of formaldehyde. J. Organometallic Chemistry, 1979, 17(2), C27-C28.
Sandoval, N. R., Venkataramanan, K. P., Groth, T. S., Papoutsakis, E. T. Whole-genome sequence of an evolved clostridium pasteurianum strain reveals Spo0A deficiency responsible for increased butanol production and superior growth Biotechnol. Biofuels, 2015, 8, 227-245.
Saier, Jr. M. H., Hvorup, R. N. and Barabote, R. D. (2005) Evolution of the bacterial phosphotransferase system: from carriers and enzymes to group translocators. Biochem. Soc. Trans. 23: 220-224.
Saier Jr. M. H. (2015) The bacterial phosphotransferase system: new frontiers 50 years after its discovery. J. Mol. Microbiol. Biotechnol. 25: 73-78.
Shams, S. Y., Gonzalez, R. Anaerobic fermentation of glycerol: a path to economic viability fort the biofuels industry. Curr. Opin. Biotechnol. 2007, 18, 213-219.
Sharninghausen L. S., Campos J., Manas M. G., Crabtree R. H. Efficient selective and atom economic catalytic conversion of glycerol to lactic acid. Nature Communications, 2014, 5:5084.
Shen, C. R., Dekishima, Y., Lan, E. I., Baez, A., Cho, K. M., Liao, J. C. Driving forces enable high-titer anaerobic 1-butanol synthesis in E. Coli. Appl. Environ. Microbiol. 2011, 77(9), 2905-2915.
Shi R., Mcdonald L., Cui Q., Matte A., Cygler M., Ekiel I. Structural and mechanistic insight into covalent substrate binding by Escherichia coli DHA kinase. Proc. Natl. Acad. Sci. U.S.A 2011, 108(4), 1302-1307.
Siebold, C., Flukiger, K., Beutler, R. and Erni, B. (2001) Carbohydrate transporters of the bacterial phosphoenolpyruvate: sugar phosphotransferase system (PTS). FEBS Lett. 504:104-111.
Siebold C., Arnold I., Garcia-alles L. F., Baumann U., Erni B. Crystal structure of the citrobacter freundii DHA kinase reveals an eight standard alpha-helical barrel ATP binding domain. Journal of Biological Chemistry, 2003, 278(48), 48236-48244.
Siebold C., Garcia-alles L. F., Erni B., Baumann U. A mechanism of covalent substrate binding in the x-ray structure of subunit K of the Escherichia coli DHA kinase Proc. Natl. Acad. Sci. U.S.A 2003, 100(14), 8188-8192.
Sillers, R., Al-hinai, M. A., Papoutsakis, E. T. Aldehdye-alcohol dehydrogenase and/or thiolase overexpression coupled with CoA trasfease downregulation lead to higher alcohol titers and selectivity in clostridium acetobutylicum fermentations. Biotechnol. Bioeng. 2009 102, 38-49.
Subedi K. P., Kim I., Kim J., Min B., Park C. Role of GldA in DHA and methylglyoxal metabolism of Escherichia coli K12. FEMS Microbiol. Lett. 2008, 279, 180-187.
Tchieu, J. H., Norris, V., Edwards, J. S. and Saier Jr., M. H. (2001) The complete phosphotransferase system in Escherichia coli. J. Mol. Microbiol. Biotechnol. 3: 329-346.
Tian K., Chen X., Shen W., Prior B. A., Shi G., Singh S., Wang Z. High-efficiency conversion of glycerol to D-lactic acid with metabolically engineered E. Coli. Afr. J. Biotechnol, 2012, 11(21), 4860-4867.
Trinh, C., Srienc, F., Metabolic engineering of Escherichia coli for efficient conversion of glycerol to ethanol. Appl. Environ. Microbiol. 2009, 75(21), 6696-6705.
Vasconcelos I., Girbal L., Soucaille P. Regulation of carbon and electron flow in Clostridium acetobutylicum grown in chemostat culture at neutral pH on mixtures of glucose and glycerol. J. Bacteriol, 1994, 176, 1443-1450.
Wang, Q., Ingram, L. O. Shanmugham, K. T. Evolution of D-lactate dehydrogenase activity from glycerol dehydrogenase and its utility for D-lactate production from lignocellulose. Proc. Natl. Acad. Sci. USA 2011, 108(47), 18920-18925.
Wong, M., Li, M., Black, R. W., Le, T. Q., Puthli, S., Campbell, P., Monticello, D. J. Microaerobic conversion of glycerol to ethanol in Escherichia coli. Appl. Environ. Microbiol. 2014, 80(10), 3276-3282.
Yazdani, S. S., Gonzalez, R. Engineering Escherichia coli for the efficient conversion of glycerol to ethanol and co-products. Metab. Eng. 2008, 10, 340-351.
Zhou S., Causey T. B., Hasona A., Shanmugam K. T., Ingram L. O. Production of optically pure D-lactic acid in mineral salts medium by metabolically engineered E. Coli W3110. Appl. Environ. Microbiol. 2003, 69(1), 399-407.
Zhou, Y. J., Yang, W., Wang, L., Zhang, S., Zhao, Z. K. (2013) Engineering NDA+availability for Eschericha coli whole-cell biocatlysis: a case study for DHA production. Microbial Cell Factories 2013, 12, 103-
Zhou, P., Zhang, Y., Wang, P., Xie, J., Ye, Q. Butanol production from glycerol by recombinant E. Coli. Annals of Microbiology 2014, 64(1), 219-227.
Zhu Y., Eiteman M. A., Dewitt K., Altman E. Homolactate fermentation by metabolically engineered E. Coli Strains. Appl. Environ. Microbiol. 2007, 73(2), 456-464.
Zurbriggen A., Jeckelmann J. M., Christen S., Bieniossek C., Baumann U., Erni B. X-ray structures of the three lactococcus lactis DHA kinase subunit and of a transient intersubunit complex. Journal of Biological Chemistry, 2008, 283(51), 35789-35796.

What is claimed:

1. A microbial biocatalyst useful in producing a biochemical in commercial quantity in a fermentation medium comprising dihydroxyacetone as a major source of carbon and energy.

2. The microbial biocatalyst as in claim 1, wherein said biocatalyst is selected from a group consisting of gram negative bacteria, gram positive bacteria, algae, archaea, cyanobacteria, yeast and filamentous fungi.

3. The microbial biocatalyst as in claim 1, wherein said biochemical is selected from a group comprising, organic acids, C2-C3 alcohols, C4-C10 alcohols, diols, isoprenoids, terpenoids, fatty acids and its derivatives, amino acids and its derivatives, vitamins, sterols, antibiotics, olefins and flavonoids.

4. The microbial biocatalyst as in claim 1, wherein said fermentation medium is kept in aerobic or microaerobic or anaerobic condition.

5. The microbial biocatalyst as in claim 1, wherein said biocatalyst is genetically modified and said genetic modification causes an increase in the activity of an enzyme responsible for the phosphorylation of dihydroxyacetone.

6. The microbial biocatalyst as in claim 5, wherein said enzyme responsible for the phosphorylation of dihydroxyacetone is DHA kinase.

7. The microbial biocatalyst as in claim 6, wherein said DHA kinase with an increase phosphorylation activity is coded by an endogenous gene.

8. The microbial biocatalyst as in claim 6, wherein said DHA kinase with an increase phosphorylation activity is coded by an exogenous gene.

9. The microbial biocatalyst as in claim 5, wherein said DHA kinase uses phosphoenolpyruvate as a source of phosphate in the phosphorylation reaction.

10. The microbial biocatalyst as in claim 5, wherein said DHA kinase uses adenosine triphosphate as a source of phosphate in the phosphorylation reaction.

11. The microbial biocatalyst as in claim 5, wherein said enzyme responsible for the phosphorylation of dihydroxyacetone is glycerol kinase.

12. The microbial biocatalyst as in claim 1, wherein said medium comprising DHA as a source of carbon further comprises at least one additional source of carbon.

13. The microbial biocatalyst as in claim 12, wherein said additional source of carbon is selected from a group comprising hexose, pentose, tetrose, triose, glycerol, hydrocarbons, carboxylic acid and cellulosic hydrolysate.

14. The microbial biocatalyst as in claim 1, wherein said dihydroxyacetone is derived from a group consisting of methane, natural gas, biogas, biomass, syngas and carbon dioxide.

15. (canceled)

16. (canceled)

17. (canceled)

18. (canceled)

19. (canceled)

20. The microbial biocatalyst as in claim 1, wherein said biocatalyst is genetically modified and said genetic modification causes an increase in the activity of an enzyme responsible for the isomerization of DHA to glyceraldehyde.

21. The microbial biocatalyst as in claim 20, wherein said enzyme responsible for the isomerization of DHA to glyceraldehyde is triose isomerase.

22. The microbial biocatalyst as in claim 21, wherein said triose isomerase enzyme responsible for the isomerization of DHA to glyceraldehyde is coded by an endogenous gene.

23. The microbial biocatalyst as in claim 21, wherein said triose isomerase enzyme responsible for the isomerization of DHA to glyceraldehyde is coded by an exogenous gene.

24. A fermentation process to produce a value added biochemical selected from a group comprising, organic acids, C2-C3 alcohols, C4-C10 alcohols, diols, isoprenoids, terpenoids, fatty acids and its derivatives, amino acids and its derivatives, vitamins, sterols, antibiotics, olefins and flavonoids, wherein the process comprising step of:
a. selecting a microbial biocatalyst suitable for the production of said value added biochemical;
b. growing said microbial biocatalyst in a medium comprising DHA as a source of organic carbon; and
c. harvesting said value added biochemical at the end of said fermentation process.

25. (canceled)

26. A fermentation process as in claim 24, wherein said microbial biocatalyst is selected from a group consisting of bacterium, archaea, algae, cyanobacterium, fungi and yeast.

27. (canceled)

28. (canceled)

29. (canceled)
